(19) United States
(12) Patent Application Publication
(10) Pub. No.: US 2026/0347356 A1
(43) Pub. Date: Nov. 5, 2026

(54) PROCESSES FOR PREPARING AN ALPHA4BETA7 INHIBITOR

(71) Applicant: Gilead Sciences, Inc., Foster City, CA (US)

(72) Inventors: Peter A. Blomgren, Issaquah, WA (US); Andre Costisella, Edmonton (CA); Jason A. Davy, Edmonton (CA); Darryl D. Dixon, San Mateo, CA (US); Ian J. Doxsee, San Mateo, CA (US); Joshua R. Dunetz, Burlingame, CA (US); Justin E. Jurczyk, San Francisco, CA (US); Mark G. Martell, Edmonton (CA); Jeffrey D. Ng, South San Francisco, CA (US); Astrid Parsons, San Carlos, CA (US); Adam B. Weinstein, Belmont, CA (US); Erik W. Werner, Castro Valley, CA (US); Tyler W. Wilson, San Ramon, CA (US); Li Zhang, San Mateo, CA (US); Zhongdong Zhao, Bellevue, WA (US)

(21) Appl. No.: 19/550,581

(22) Filed: Feb. 26, 2026

Related U.S. Application Data
(60) Provisional application No. 63/764,865, filed on Feb. 28, 2025.

(51) Int. Cl. C07D 471/04; C07C 55/08; C07D 215/40; C07D 265/30; C12N 9/88; C12P 17/12

(52) U.S. Cl. CPC C07D 471/04; C07C 55/08; C07D 215/40; C07D 265/30; C12N 9/88; C12P 17/12; C07B 2200/13; C12Y 401/99002

(57) ABSTRACT

The present disclosure relates generally to processes for preparing a compound that is an inhibitor for α4β7 integrin and the synthetic intermediates prepared thereby.

### CROSS REFERENCE TO RELATED APPLICATIONS

This application claims the benefit of U.S. Provisional Application No. 63/764,865, filed on Feb. 28, 2025, the content of which is hereby incorporated herein in its entirety.

### FIELD

The present disclosure relates generally to processes for preparing a compound that is an inhibitor for α4β7 integrin and the synthetic intermediates prepared thereby.

### BACKGROUND

Ethyl (2S)-2-{2-fluoro-6-methyl-4-[(3R)-3-(trifluoromethyl)morpholin-4-yl]benzamido}-3-[(8M)-8-(1-methyl-2,4-dioxo-1,4-dihydropyrido[3,4-d]pyrimidin-3(2H)-yl)quinolin-5-yl]propanoate (Compound I):

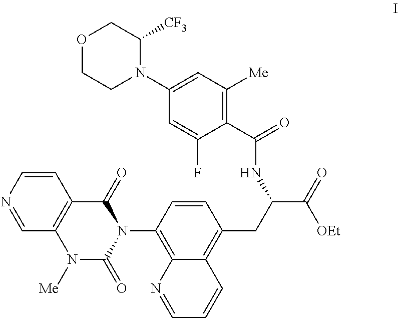

is an inhibitor for α4β7 integrin.

Integrins are heterodimeric cell surface proteins involved in numerous cellular processes including cell-cell and cell-extracellular matrix interactions. Upon binding of an extracellular ligand, integrins mediate signal transduction to the cell interior resulting in lymphocyte cell capture, adhesion, and infiltration into the tissue. Integrins are heterodimeric proteins consisting of an alpha and a beta subunit. There are 18 known alpha subunits and 8 known beta subunits. The α4β7 integrin is expressed on the surface of lymphocytes and recognizes the extracellular ligand mucosal addressing cell adhesion molecule-1 (MAdCAM-1). α4β7 integrin governs lymphocyte trafficking to and retention in gut tissues through its interaction with MAdCAM-1, which is expressed on venules in the intestinal mucosa and high endothelial venules (HEY) in the gut associated lymphoid tissues (GALT). Inhibiting the interactions of integrins with their respective ligands has been proposed as an effective method of treating a variety of autoimmune and inflammatory diseases, and blocking the α4β7-MAdCAM-1 interaction has shown therapeutic benefit in inflammatory bowel disease (Crohn's disease and ulcerative colitis).

Accordingly, there is a continued need to improve synthetic processes for preparing α4β7 inhibitors such as ethyl (2S)-2-{2-fluoro-6-methyl-4-[(3R)-3-(trifluoromethyl)morpholin-4-yl]benzamido}-3-[(8M)-8-(1-methyl-2,4-dioxo-1,4-dihydropyrido[3,4-d]pyrimidin-3(2H)-yl)quinolin-5-yl]propanoate (Compound I) and structurally related compounds, for example, to improve yields, purity, and scaling.

### SUMMARY

Provided are processes for preparing Compound I and the synthetic intermediates prepared thereby.

In some embodiments, the present disclosure provides a process for preparing Compound I

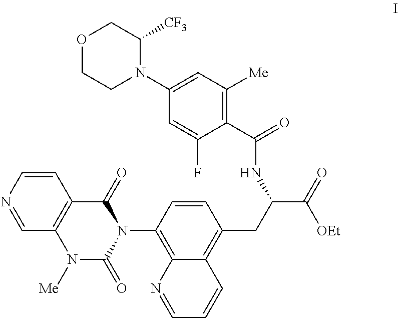

or a salt thereof, comprising:
coupling Compound II

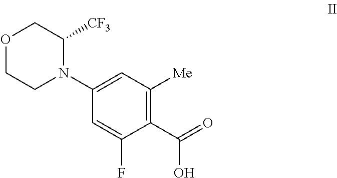 [SMILES: Cc1cc(N2CCOC[C@@H]2C(F)(F)F)cc(F)c1C(=O)O]

or a salt thereof with Compound III

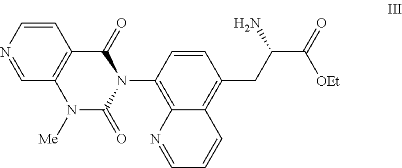

or a salt thereof in the presence of a coupling agent to form Compound I or a salt thereof.

In some embodiments, the present disclosure provides a process for preparing Compound III

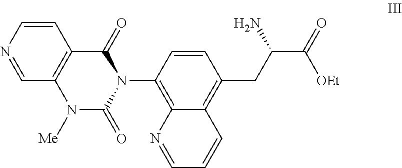

or a salt thereof, comprising:
(a) contacting Compound III-1a

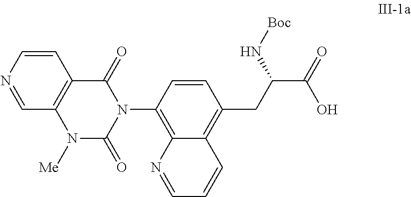 [SMILES: Cn1c(=O)n(-c2ccc(C[C@H](NC(=O)OC(C)(C)C)C(=O)O)c3cccnc23)c(=O)c2ccncc21]

or a salt thereof with ethanol in the presence of an acid to form Compound Ilia

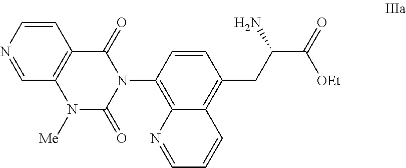 [SMILES: CCOC(=O)[C@@H](N)Cc1ccc(-n2c(=O)c3ccncc3n(C)c2=O)c2ncccc12]

or a salt thereof; and
(b) crystallizing Compound Ilia from a solvent to provide Compound III as a single atropisomer.

In some embodiments, the present disclosure provides a process for preparing Compound III-1a

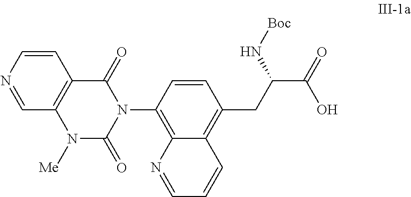 [SMILES: Cn1c(=O)n(-c2ccc(C[C@H](NC(=O)OC(C)(C)C)C(=O)O)c3cccnc23)c(=O)c2ccncc21]

or a salt thereof, comprising:
(a) contacting a compound of Formula IV-1

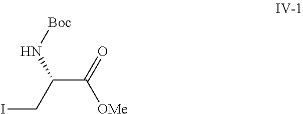 [SMILES: COC(=O)[C@H](CI)NC(=O)OC(C)(C)C]

or a salt thereof with zinc; and
(b) contacting a product from step (a) with a compound of Formula Va

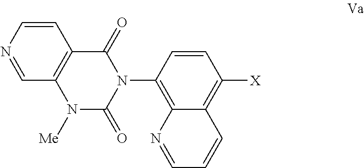

wherein X is Br or I,
or a salt thereof in the presence of a catalytic system to form Compound III-2a

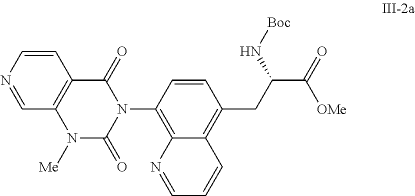 [SMILES: COC(=O)[C@H](Cc1ccc(-n2c(=O)c3ccncc3n(C)c2=O)c2ncccc12)NC(=O)OC(C)(C)C]

or a salt thereof.

In some embodiments, the present disclosure provides a process for preparing a compound of Formula Va

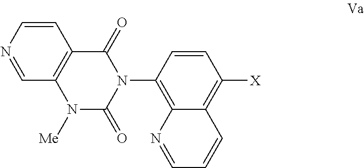

wherein X is Br or I,
or a salt thereof, comprising:
(a) contacting Compound VI

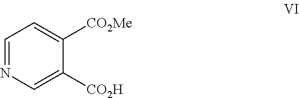 [SMILES: COC(=O)c1ccncc1C(=O)O]

or a salt thereof with a compound of Formula VII

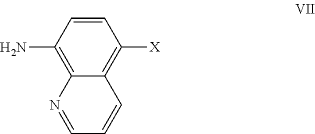

wherein X is Br or I,
or a salt thereof in the presence of an azide source or a deoxo-fluor to form a compound of Formula V-3

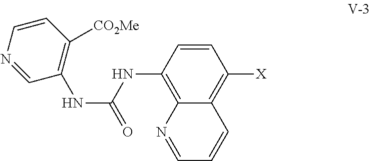

wherein X is Br or I,
or a salt thereof;
(b) subjecting the compound of Formula V-3 or a salt thereof into a cyclization condition to provide a compound of Formula V-1

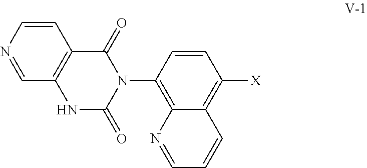

wherein X is Br or I,
or a salt thereof; and
(c) contacting the compound of Formula V-1 or a salt thereof with a methylation agent to form Compound Va or a salt thereof.

Also provided is a process for preparing Compound III

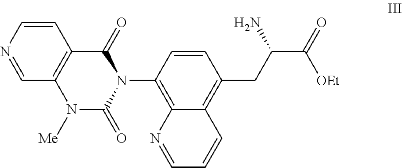

or a salt thereof, comprising:
(a) contacting Compound IV-3

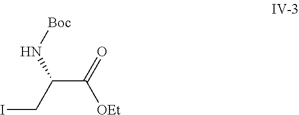 [SMILES: CCOC(=O)[C@H](CI)NC(=O)OC(C)(C)C]

or a salt thereof with zinc;
(b) contacting a product from step (a) with Compound V-I

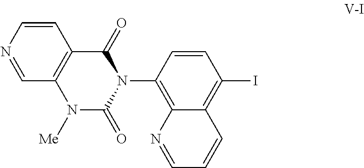

or a salt thereof in the presence of a catalytic system to form Compound III-3

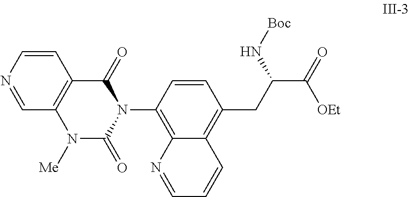

or a salt thereof; and
(c) subjecting Compound III-3 or a salt thereof to a deprotection condition to form Compound III or a salt thereof.

Also provided is a process for preparing Compound III-3

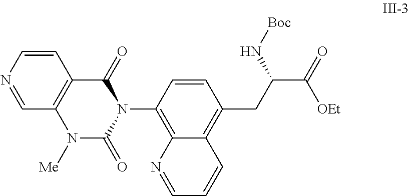

or a salt thereof, comprising:
(a) contacting Compound IV-5

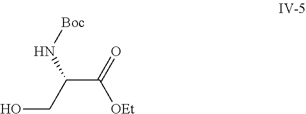 [SMILES: CCOC(=O)[C@H](CO)NC(=O)OC(C)(C)C]

or a salt thereof with a benzoxazolium salt activator;
(b1) contacting a product from step (a) with Compound V-1-Br

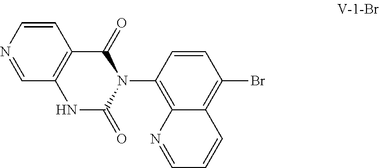

or a salt thereof in the presence of a photosensitizer to provide Compound III-4

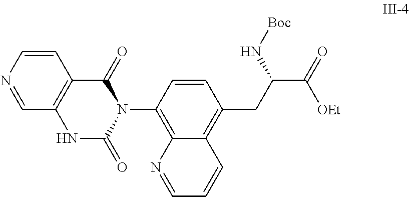

or a salt thereof; or
(b2) contacting the product from step (a) with Compound V-Br

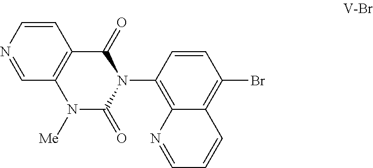

or a salt thereof in the presence of a photosensitizer to provide Compound III-3

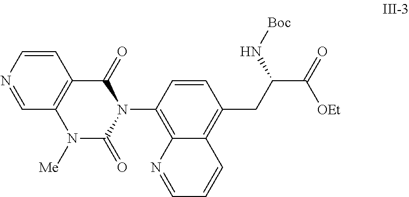

or a salt thereof; and
(c) when step (b1) occurs, contacting Compound III-4 or a salt thereof with a methylation agent to form
Compound III-3 or a salt thereof.

Also provided is a process for preparing a compound of Formula V-1a

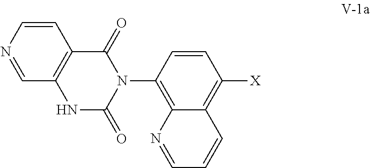

wherein X is Br or I,
or a salt thereof, comprising:
subjecting a compound of Formula V-2

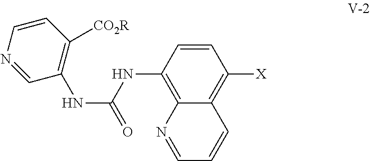

wherein R is a C₁₋₃ alkyl and X is Br or I,
or a salt thereof into a cyclization condition to form a compound of Formula V-1a or a salt thereof.

Also provided is a process for preparing Compound III-4

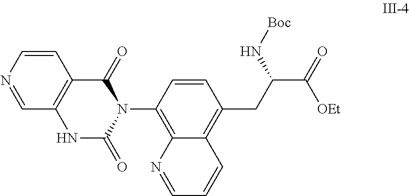

or a salt thereof, comprising:
(a) subjecting Compound III-5

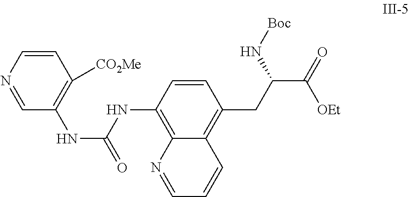 [SMILES: CCOC(=O)[C@H](Cc1ccc(NC(=O)Nc2cnccc2C(=O)OC)c2ncccc12)NC(=O)OC(C)(C)C]

or a salt thereof into a cyclization condition to form Compound III-4 or a salt thereof.

Also provided is a process for preparing Compound II

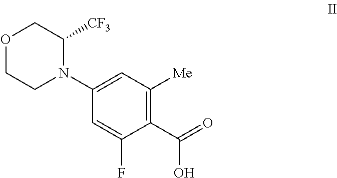 [SMILES: Cc1cc(N2CCOC[C@@H]2C(F)(F)F)cc(F)c1C(=O)O]

or a salt thereof, comprising:
(a) contacting Compound VIII

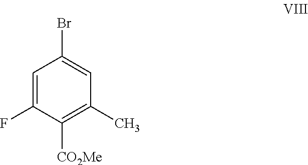

or a salt thereof with Compound IX

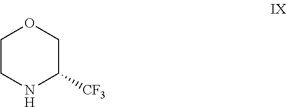

or a salt thereof in the presence of a catalytic system to provide Compound II-2

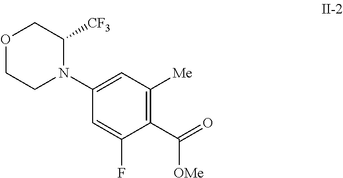 [SMILES: COC(=O)c1c(C)cc(N2CCOC[C@@H]2C(F)(F)F)cc1F]

or a salt thereof; and
(b) subjecting Compound II-2 or a salt thereof into a hydrolysis condition to form Compound II or a salt thereof.

Also provided is a process for preparing Compound IX

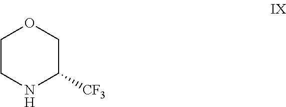

or a salt thereof, comprising:
(a) contacting a compound of Formula IX-1

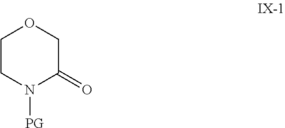

wherein PG is benzyl or

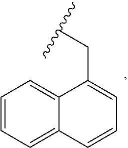 [SMILES: *Cc1cccc2ccccc12]

or a salt thereof with a trifluoromethylation agent to form a compound of Formula IX-2

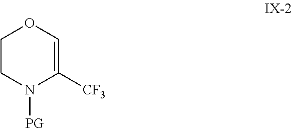

or a salt thereof;
(b) subjecting the compound of Formula IX-2 or a salt thereof to an asymmetric hydrogenation condition to form a compound of Formula IX-3

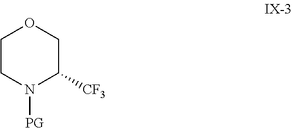

or a salt thereof; and
(c) subjecting the compound of formula IX-3 or a salt thereof to a deprotection condition to form Compound IX or a salt thereof, wherein the deprotection condition in step (c) comprises a chiral acid.

Also provided is a process for preparing Compound II,

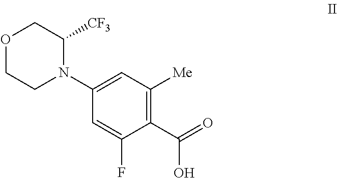 [SMILES: Cc1cc(N2CCOC[C@@H]2C(F)(F)F)cc(F)c1C(=O)O]

or a salt thereof, comprising:
(a) contacting Compound VIII

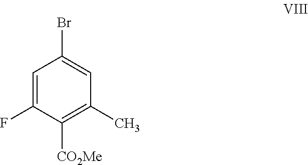

or a salt thereof with morpholin-3-one in the presence of a catalytic system to form Compound II-3

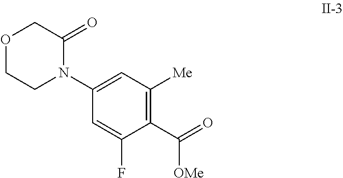 [SMILES: COC(=O)c1c(C)cc(N2CCOCC2=O)cc1F]

or a salt thereof;
(b) contacting Compound II-3 with a trifluoromethylation agent;
(c) subjecting a product from step (b) into an elimination condition to form Compound II-4

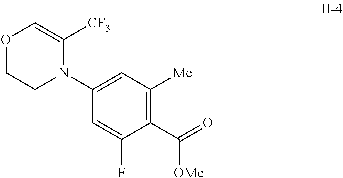 [SMILES: COC(=O)c1c(C)cc(N2CCOC=C2C(F)(F)F)cc1F]

or a salt thereof;
(d) subjecting Compound II-4 into an asymmetric hydrogenation condition to form Compound II-2

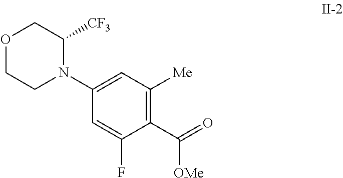 [SMILES: COC(=O)c1c(C)cc(N2CCOC[C@@H]2C(F)(F)F)cc1F]

or a salt thereof.

Also provided is a process for preparing Compound X

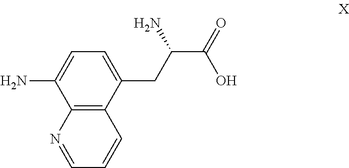 [SMILES: Nc1ccc(C[C@H](N)C(=O)O)c2cccnc12]

or a salt thereof, comprising:
contacting quinolin-8-amine with an ammonia source and a pyruvate source to form Compound X or a salt thereof.

Also provided is a process for preparing Compound X-3

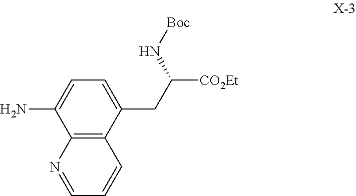 [SMILES: CCOC(=O)[C@H](Cc1ccc(N)c2ncccc12)NC(=O)OC(C)(C)C]

or a salt thereof, comprising:
(a) contacting Compound IV-4

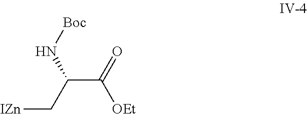

or a salt thereof with Compound VII-2

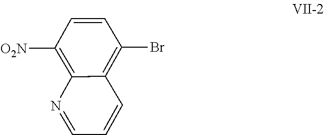 [SMILES: O=[N+]([O-])c1ccc(Br)c2cccnc12]

or a salt thereof in the presence of a catalytic system to provide Compound X-4

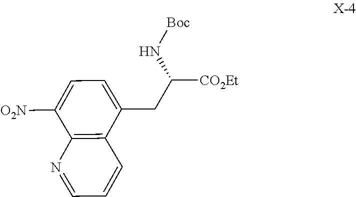 [SMILES: CCOC(=O)[C@H](Cc1ccc([N+](=O)[O-])c2ncccc12)NC(=O)OC(C)(C)C]

or a salt thereof; and
(b) subjecting Compound X-4 or a salt thereof to a reductive condition to provide Compound X-3 or a salt thereof.

### DESCRIPTION

### Definitions

As used in the present specification, the following words and phrases are generally intended to have the meanings as set forth below, except to the extent that the context in which they are used indicates otherwise.

The term “comprise” and variations thereof, such as, “comprises” and “comprising” are to be construed in an open, inclusive sense, that is, as “including, but not limited to.” Further, the singular forms “a,” “an,” and “the” include plural references unless the context clearly dictates otherwise. Thus, reference to “the compound” includes a plurality of such compounds.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±2.5%. In certain other embodiments, the term “about” includes the indicated amount ±1%. Also, to the term “about X” includes description of “X”.

Recitation of numeric ranges of values throughout the disclosure is intended to serve as a shorthand notation of referring individually to each separate value falling within the range inclusive of the values defining the range, and each separate value is incorporated in the specification as it were individually recited herein.

As used herein, the term “contacting” refers to the process of bringing into contact at least two distinct species such that they can react. It should be appreciated, however, that the resulting reaction product can be produced directly from a reaction between the added reagents or from an intermediate from one or more of the added reagents which can be produced in the reaction mixture.

The term “reaction conditions” is intended to refer to the physical and/or environmental conditions under which a chemical reaction proceeds. Examples of reaction conditions include, but are not limited to, one or more of following: reaction temperature, solvent, pH, pressure, reaction time, mole ratio of reactants, the presence of a base or acid or catalyst, radiation, etc. Reaction conditions may be named after the particular chemical reaction in which the conditions are employed, such as, coupling conditions, hydrogenation conditions, acylation conditions, reduction conditions, etc. Reaction conditions for most reactions are generally known to those skilled in the art or can be readily obtained from the literature. Exemplary reaction conditions sufficient for performing the chemical transformations provided herein can be found throughout, and in particular, the examples below. It is also contemplated that the reaction conditions can include reagents in addition to those listed in the specific reaction.

“Catalyst” refers to a chemical reactant that increases the rate of a reaction without itself being consumed. Similarly, “cocatalyst” or “co-catalyst” refers to a substance that works with another as a catalyst.

A “ligand” as used herein refers to an ion or molecule that binds to a metal atom to form a coordination complex. Denticity of a ligand refers to the number of times a ligand bonds to a metal through noncontiguous donor sites. Ligands may be monodentate (i.e., they possess one binding site) or bidentate (i.e., they possess two binding sites).

“Hydrolysis” as used herein refers to the cleavage by water of a carboxylic ester into a carboxylic acid and an alcohol.

As used herein, the term “salt” refers to a compound formed by the reaction of an acid and a base, resulting in the formation of a positively charged cation and a negatively charged anion. In general, a salt is defined as a compound that is formed by the combination of positively and negatively charged ions, where the charges of the ions result in a neutral compound. Salts can be either inorganic or organic. As used herein, the term “salt” includes partially or fully ionized salt forms. In some embodiments, the salt is fully ionized.

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987; the entire contents of each of which are incorporated herein by reference.

The term “alkyl” as used herein, means a straight or branched, saturated hydrocarbon chain containing from 1 to 30 carbon atoms. The term “C₁-C₃-alkyl” means a straight or branched chain hydrocarbon containing from 1 to 6 carbon atoms. The term “C₁-C₃-alkyl” means a straight or branched chain hydrocarbon containing from 1 to 3 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “aromatic” refers to the ring moieties which satisfy the Hückel 4n+2 rule for aromaticity, and includes both aryl (i.e., carbocyclic) and heteroaryl (also called heteroaromatic) structures.

The term “aryl” as used herein, refers to a phenyl group, or bicyclic aryl or tricyclic aryl fused ring systems. Bicyclic fused ring systems are exemplified by a phenyl group appended to the parent molecular moiety and fused to a phenyl group. Tricyclic fused ring systems are exemplified by a phenyl group appended to the parent molecular moiety and fused to two other phenyl groups. Representative examples of bicyclic aryls include, but are not limited to, naphthyl. Representative examples of tricyclic aryls include, but are not limited to, anthracenyl. The monocyclic, bicyclic, and tricyclic aryls are connected to the parent molecular moiety through any carbon atom contained within the rings, and can be unsubstituted or substituted.

The term “halogen” or “halo” as used herein, means Cl, Br, I, or F.

The term “amine” or “amine base” as used herein generally refers to a primary, secondary, or tertiary amine, such as a alkyl amine, dialkyl amine, trialkyl amine, or nitrogen-containing heterocycle, wherein each of which is optionally substituted, e.g., by alkyl.

The term “nitrogen-containing heterocyclic” base as used herein, refers to a saturated or partially unsaturated cyclic alkyl group, with at least one ring nitrogen atom, and optionally one or more additional ring heteroatoms independently selected from nitrogen, oxygen, and sulfur. The term “heterocyclic” includes heterocycloalkenyl rings (i.e., a heterocyclic ring having at least one double bond), bridged-heterocyclic rings, fused-heterocyclic rings, and spiro-heterocyclic rings. A heterocycle may be a single ring or multiple rings wherein the multiple rings may be fused, bridged or spiro, and may comprise one or more (e.g., one to three or one or two) oxo (═O) or N-oxide (—O⁻) moieties. Any non-aromatic ring containing at least one nitrogen atom capable of accepting a proton is considered a nitrogen-containing heterocyclic base. Further, the term heterocyclic is intended to encompass any non-aromatic ring containing at least one heteroatom, which ring may be fused to a cycloalkyl, aryl or heteroaryl ring, regardless of the position of the heteroatom. As used herein, a nitrogen-containing heterocyclic has 2 to 20 ring carbon atoms (i.e., C₂₋₂₀ heterocycle), 2 to 12 ring carbon atoms (i.e., C₂₋₁₂ heterocycle), 2 to 10 ring carbon atoms (i.e., C₂₋₁₀ heterocycle), 2 to 8 ring carbon atoms (i.e., C₂₋₈ heterocycle), 3 to 12 ring carbon atoms (i.e., C₃₋₁₂ heterocycle), 3 to 8 ring carbon atoms (i.e., C₃₋₈ heterocycle), or 3 to 6 ring carbon atoms (i.e., C₃₋₆ heterocycle); having at least 1 nitrogen atom capable of accepting a proton, and optionally an additional 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen.

The term “nitrogen-containing heteroaryl” base refers to an aromatic group having a single ring, multiple rings or multiple fused rings, with at least one nitrogen atom capable of accepting a proton, and optionally one or more additional ring heteroatoms independently selected from nitrogen, oxygen and sulfur. As used herein, nitrogen-containing heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl); and having at least 1 nitrogen atom, and optionally an additional 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen and sulfur. In certain instances, heteroaryl includes 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen and sulfur.

The term “basic aromatic compound” refers to an aromatic group having a single ring, multiple rings or multiple fused rings, with at least one atom capable of accepting a proton. In some embodiments, the atom capable of accepting a proton is a nitrogen atom. In some embodiments, the basic aromatic compound is a nitrogen-containing heteroaryl base.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Any formula given herein is intended to represent compounds having structures depicted by the structural formula as well as certain variations or forms unless specified otherwise. In particular, compounds of any formula given herein may have asymmetric centers or axial chirality and therefore exist in different stereoisomers. All optical isomers and stereoisomers of the compounds of a formula, and mixtures thereof, are considered within the scope of the present disclosure. Furthermore, certain structures may exist as geometric isomers (i.e., cis and trans isomers) or as tautomers. All geometric isomers and tautomers of the compounds of a formula, and mixtures thereof, are considered within the scope of the present disclosure.

The compound disclosed herein may be depicted by a structure comprising a wedged bold and/or a dashed bond. It can be appreciated that the wedged bold or dashed bond is used to indicate absolute stereochemistry. Where the composition is identified as stereochemically enriched, it is intended that the composition comprises more than 50% of a single stereoisomer, or at least about 55%, or at least about 60%, or at least about 65%, or at least about 70%, or at least about 75%, or at least about 80%, or at least about 85%, or at least about 90%, or at least about 95%, or at least about 97%, or about 99% of a single stereoisomer.

For example, a compound according to the following structure:

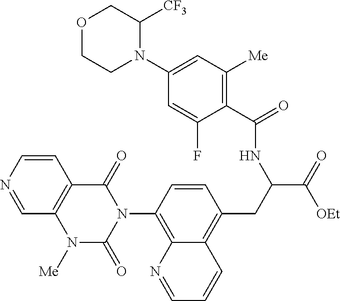 [SMILES: CCOC(=O)C(Cc1ccc(-n2c(=O)c3ccncc3n(C)c2=O)c2ncccc12)NC(=O)c1c(C)cc(N2CCOCC2C(F)(F)F)cc1F]

have asymmetric centers and therefore may exist in different stereoisomers, such as

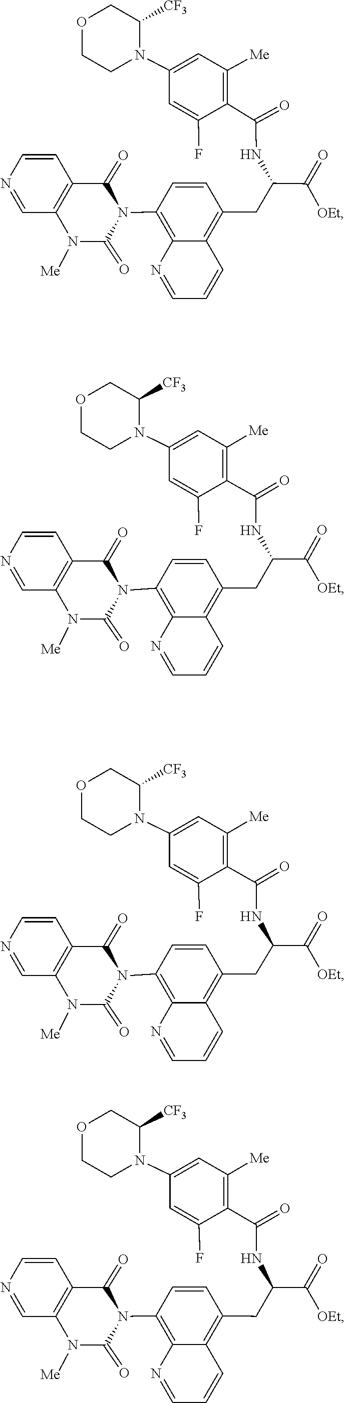

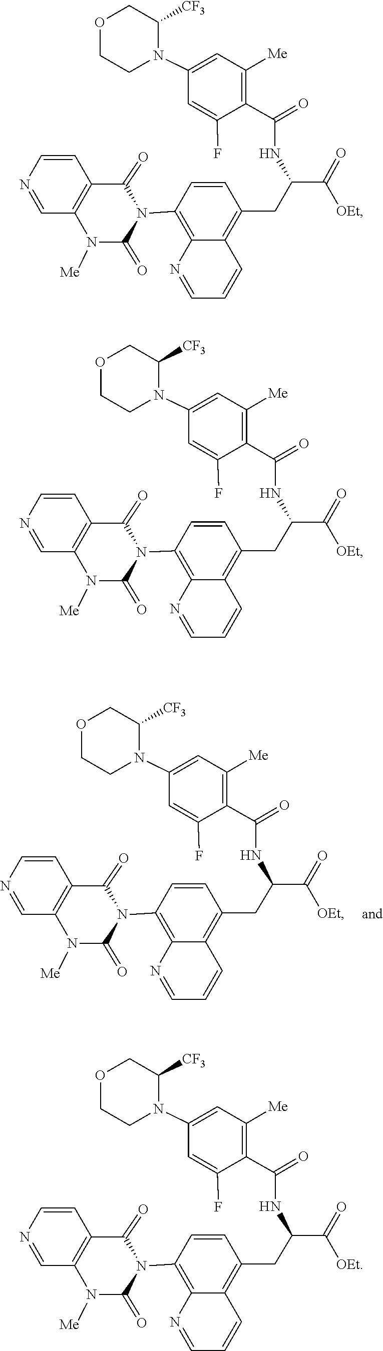

As used herein, “Compound I” is intended to represent a stereochemically enriched compound as shown in the following structure:

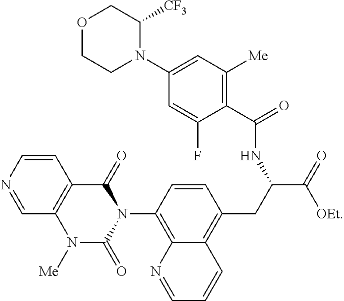

It is to be understood that all optical isomers and stereoisomers of Compound I (e.g., the stereoisomers shown above), and mixtures thereof, are considered within the scope of the present disclosure.

Additionally or alternatively, a compound according to the following structure:

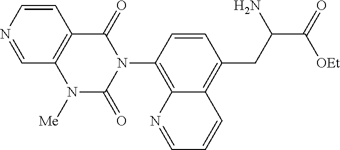 [SMILES: CCOC(=O)C(N)Cc1ccc(-n2c(=O)c3ccncc3n(C)c2=O)c2ncccc12]

have asymmetric centers and therefore may exist in different stereoisomers, such as

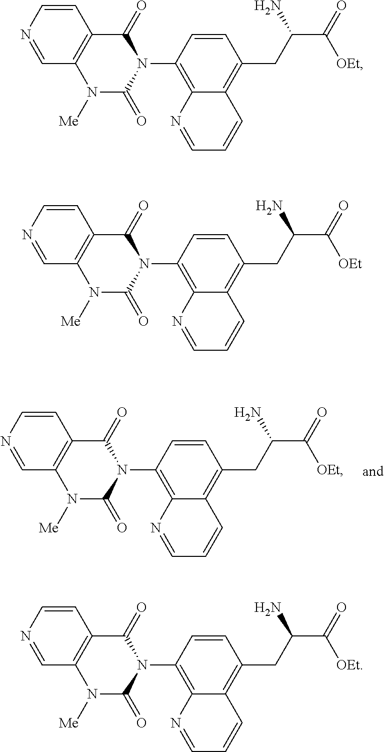

As used herein, “Compound III” is intended to represent a stereochemically enriched compound as shown in the following structure:

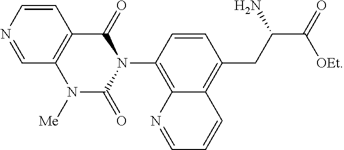

It is to be understood that all optical isomers and stereoisomers of Compound III (e.g., the stereoisomers shown above), and mixtures (including racemic mixtures) thereof, are considered within the scope of the present disclosure.

Further, it is to be understood that processes used for the preparation of a stereochemically enriched compound as described herein may be used for preparing a stereoisomer thereof or a mixture of stereoisomers (including racemic mixtures). For example, processes used for the preparation of Compound I (which is stereochemically enriched) may be used for preparing a stereoisomer thereof or a mixture of stereoisomers. Likewise, processes used for the preparation of Compound III (which is stereochemically enriched) may be used for preparing a stereoisomer thereof or a mixture of stereoisomers. For instance, processes used for the preparation of Compound III (which is stereochemically enriched) may be used for preparing Compound IIIa:

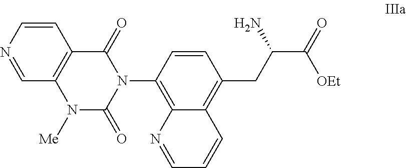 [SMILES: CCOC(=O)[C@@H](N)Cc1ccc(-n2c(=O)c3ccncc3n(C)c2=O)c2ncccc12]

Additionally or alternatively, processes used for the preparation of a mixture of stereoisomers (including a racemic mixture) as described herein may be used for preparing a stereochemically enriched compound. For example, processes used for the preparation of Compound IIIa may be used for preparing Compound III.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are embraced by the present disclosure and are disclosed herein just as if each and every combination was individually and explicitly disclosed, to the extent that such combinations embrace subject matter that are, for example, compounds that are stable compounds (i.e., compounds that can be made, isolated, characterized, and tested for biological activity). In addition, all sub-combinations of the various embodiments and elements thereof (e.g., elements of the chemical groups listed in the embodiments describing such variables) are also embraced by the present disclosure and are disclosed herein just as if each and every such sub-combination was individually and explicitly disclosed herein.

### Processes

The processes described herein provide Compound I or a salt thereof, as well as the corresponding synthetic intermediates or a salt thereof.

In certain embodiments, the processes disclosed herein can take place concurrently, in a sequential order as described herein, or in any possible order thereof.

It can be appreciated that the wedged bolded or dashed bond is used to indicate absolute stereochemistry. Where the composition is identified as stereochemically enriched, it is intended that the composition comprises more than 50% of a single stereoisomer, or at least about 55%, or at least about 60%, or at least about 65%, or at least about 70%, or at least about 75%, or at least about 80%, or at least about 85%, or at least about 90%, or at least about 95%, or at least about 97%, or about 99% of a single stereoisomer.

As noted above, it is to be understood that processes used for the preparation of a stereochemically enriched compound as described herein may be used for preparing a mixture of stereoisomers (including a racemic mixture). Additionally or alternatively, processes used for the preparation of a mixture of stereoisomers (including a racemic mixture) as described herein may be used for preparing a stereochemically enriched compound.

Ethyl (2S)-2-{2-fluoro-6-methyl-4-[(3R)-3-(trifluoromethyl)morpholin-4-yl]benzamido}-3-[(8M)-8-(1-methyl-2,4-dioxo-1,4-dihydropyrido[3,4-d]pyrimidin-3(2H)-yl)quinolin-5-yl]propanoate (Compound I) is an inhibitor for α4β7 integrin and has the following structure:

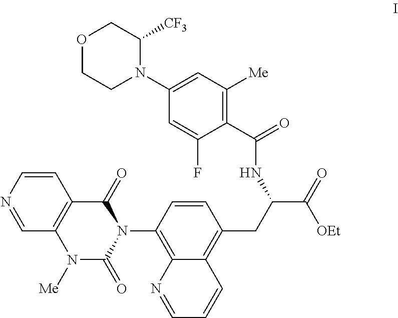

Compound I is disclosed in International Publication No. WO 2020092375, which is incorporated herein by reference in its entirety.
Synthesis of Compound I from Compound II and Compound III

Provided herein are processes for preparing Compound I or a salt thereof.

In some embodiments, provided is a process for preparing Compound I

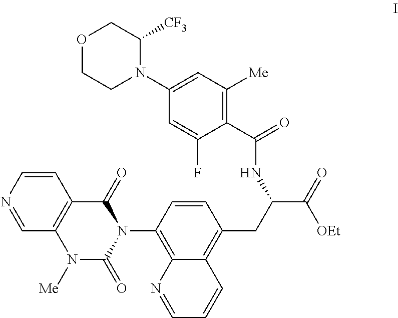

or a salt thereof, comprising:
coupling Compound II

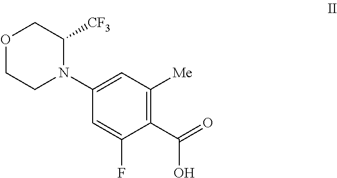 [SMILES: Cc1cc(N2CCOC[C@@H]2C(F)(F)F)cc(F)c1C(=O)O]

or a salt thereof with Compound III

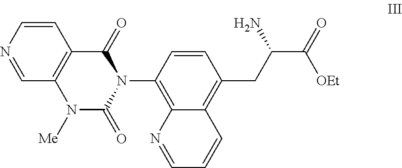

or a salt thereof in the presence of a coupling agent to form Compound I.

Suitable coupling agents (e.g., coupling agents used to provide Compound I from Compound II and Compound III) include, but are not limited to, acid halide forming agents (e.g., thionyl chloride, oxalyl chloride, phosgene, triphosgene, etc.), 1,1′-carbonyldiimidazole (CDI), alkyl chloroformates (e.g., ethyl chloroformate, isobutyl chloroformate, etc.), carbodiimides (e.g., N,N′-dicyclohexylcarbodiimide (DCC), N,N′-diisopropylcarbodiimide (DIC), 1-ethyl-3-(3-dimethylaminopropyl)carbodiimide (EDC), etc.), propanephosphonic acid anhydride (T3P), and peptide coupling reagents (e.g., hexafluorophosphate azabenzotriazole tetramethyl uronium (HATU), hexafluorophosphate benzotriazole tetramethyl uronium (HBTU), 2-(3h-[1,2,3]triazolo[4,5-b]pyridin-3-yl)-1,1,3,3-tetramethyluronium tetrafluoroborate (TATU), 2-(1H-benzotriazole-1-yl)-1,1,3,3-tetramethyluronium tetrafluoroborate (TBTU), 0-(6-chlorobenzotriazol-1-yl)-N,N,N′,N′-tetramethyluronium hexafluorophosphate (HCTU), benzotriazol-1-yloxytris(dimethylamino)phosphonium hexafluorophosphate (BOP), benzotriazol-1-yloxytripyrrolidinophosphonium hexafluorophosphate (PyBOP), [[(Z)-(1-cyano-2-ethoxy-2-oxoethylidene)amino]oxy-morpholin-4-ylmethylidene]-dimethylazanium hexafluorophosphate (COMU), etc.).

In some embodiments, the coupling agent is an acid halide forming agent. In some embodiments, the coupling agent is thionyl chloride.

In some embodiments, the coupling agent is an acid halide forming agent (e.g., thionyl chloride), and Compound II is converted to Compound II-1

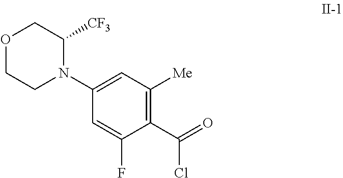 [SMILES: Cc1cc(N2CCOC[C@@H]2C(F)(F)F)cc(F)c1C(=O)Cl]

by the coupling agent prior to reacting with Compound III.

In some embodiments, the coupling of Compound II or a salt thereof and Compound III or a salt thereof occurs in the presence of a base. Suitable bases (e.g., bases used to provide Compound I from Compound II and Compound III) include, but are not limited to, amines (e.g., N,N-diisopropylethylamine, triethylamine, tripropylamine, tributylamine, 4-methylmorpholine, etc.), basic aromatic compounds (e.g., pyridine, 2,6-lutidine, imidazole, etc.), carbonates (e.g., lithium carbonate, sodium carbonate, potassium carbonate, cesium carbonate, etc.), bicarbonates (e.g., lithium bicarbonate, sodium bicarbonate, potassium bicarbonate, etc.), and phosphates (e.g., sodium phosphate, potassium phosphate, etc.).

In some embodiments, the base is an amine base. In some embodiments, the base is triethylamine.

In some embodiments, the coupling of Compound II or a salt thereof and Compound III or a salt thereof occurs in the presence of a solvent. Suitable solvents include, but are not limited to, esters (e.g., ethyl acetate, isopropyl acetate, etc.), ethers (e.g., tetrahydrofuran, 2-methyltetrahydrofuran, tert-butyl methyl ether, etc.), halogenated solvents (e.g., dichloromethane, 1,2-dichloroethane, chlorobenzene, etc.), hydrocarbons (e.g., benzene, toluene, n-heptane, etc.), nitriles (e.g., acetonitrile, benzonitrile, etc.), and polar aprotic solvents (e.g., N,N-dimethylformamide, N,N-dimethylacetamide, N-methyl-2-pyrrolidone, etc.).

In some embodiments, the solvent is a nitrile solvent. In some embodiments, the solvent is acetonitrile.

In some embodiments, the coupling of Compound II or a salt thereof and Compound III or a salt thereof occurs in the presence of a catalyst. Suitable catalysts include, but are not limited to, amide catalysts (e.g., dimethylacetamide, N-methyl-2-pyrrolidone, N,N-dimethylformamide, etc.), 4-(dimethylamino)pyridine, 1-hydroxybenzotriazole (HOBt), and 1-hydroxy-7-azabenzotriazole (HOAt).

In some embodiments, the coupling of Compound II or a salt thereof and Compound III or a salt thereof occurs in the absence of a catalyst.

In some embodiments, the coupling of Compound II or a salt thereof and Compound III or a salt thereof occurs at a temperature of about −15° C. to about 80° C. In some embodiments, the coupling occurs at a temperature of about 5° C. to about 25° C. In some embodiments, the coupling occurs at a temperature of about 15° C.

In some embodiments, the coupling of Compound II or a salt thereof and Compound III or a salt thereof occurs in the presence of a coupling agent such as an acid halide forming agent (e.g., thionyl chloride), a base such as an amine base (e.g., triethylamine), a solvent such as a nitrile solvent (e.g., acetonitrile), and at a suitable temperature such as a temperature of about 5° C. to about 25° C. (e.g., about 15° C.).

The Compound I may form a complex (e.g., a salt of Compound I and/or a cocrystal of Compound I) with an acid. Alternatively or additionally, the Compound I may exist in crystalline form (e.g., Compound I Form I) or amorphous form (e.g., Compound I Amorphous). Accordingly, provided are processes for the corresponding transformations.

For example, in some embodiments, the process may comprise crystallizing Compound I from a solvent in the presence of an acid.

In some embodiments, the acid is malonic acid. In some embodiments, the process comprises crystallizing Compound I from a solvent in the presence of malonic acid to provide Compound I Mono-Malonate:

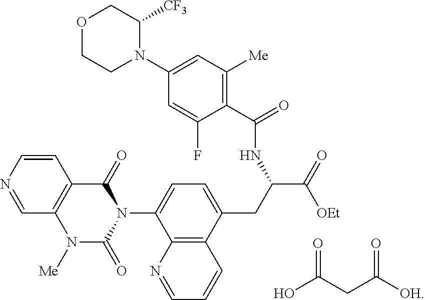

Suitable solvents (e.g., solvents used to provide Compound I Mono-Malonate from Compound I) include, but are not limited to, alcohols (e.g., methanol, ethanol, 2-propanol, etc.), ethers (e.g., tert-butyl methyl ether, tetrahydrofuran, 2-methyltetrahydrofuran, cyclopentyl methyl ether, diisopropyl ether, di-n-butyl ether, 1,4-dioxane, etc.), ketones (e.g., methyl ethyl ketone, methyl isobutyl ketone, acetone, etc.), esters (e.g., ethyl acetate, isopropyl acetate, methyl acetate, etc.), hydrocarbons (e.g., toluene, n-heptane, etc.), nitriles (e.g., acetonitrile, propionitrile, butyronitrile, benzonitrile, etc.), water, and any combination thereof.

In some embodiments, the solvent is a mixed solvent. In some embodiments, the solvent is a mixture of acetonitrile, tert-butyl methyl ether, and water.

In some embodiments, the crystallization (e.g., the crystallization of Compound I) occurs at a temperature of about 0° C. to about 70° C. In some embodiments, the temperature is about 40° C. to about 20° C. In some embodiments, the crystallization comprises cooling a solution comprising Compound I and malonic acid from about 30-50° C. (e.g., about 40° C.) to about 10-30° C. (e.g., about 20° C.).

In some embodiments, the process comprises crystallizing Compound I from a solvent such as a mixed solvent (e.g., a mixture of acetonitrile, tert-butyl methyl ether, and water), by cooling a solution comprising Compound I and malonic acid from about 30-50° C. (e.g., about 40° C.) to about 10-30° C. (e.g., about 20° C.), to provide Compound I Mono-Malonate.

In some embodiments, the acid is gentisic acid. In some embodiments, the process comprises crystallizing Compound I from a solvent in the presence of gentisic acid to provide Compound I Gentisate:

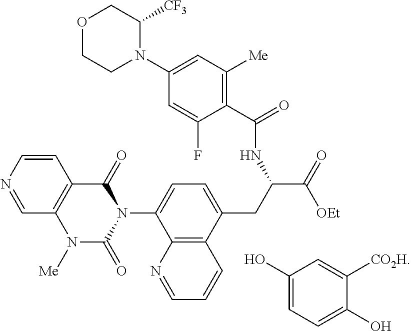

Suitable solvents (e.g., solvents used to provide Compound I Gentisate from Compound I) include, but are not limited to, alcohols (e.g., methanol, ethanol, 2-propanol, etc.), ethers (e.g., tetrahydrofuran, 2-methyltetrahydrofuran, cyclopentyl methyl ether, diisopropyl ether, di-n-butyl ether, 1,4-dioxane, etc.), ketones (e.g., methyl ethyl ketone, methyl isobutyl ketone, acetone, etc.), esters (e.g., ethyl acetate, isopropyl acetate, methyl acetate, etc.), hydrocarbons (e.g., toluene, n-heptane, etc.), nitriles (e.g., acetonitrile, propionitrile, butyronitrile, benzonitrile, etc.), water, and any combination thereof.

In some embodiments, the solvent is a nitrile solvent. In some embodiments, the solvent is acetonitrile.

In some embodiments, the crystallization (e.g., the crystallization of Compound I) occurs at a temperature of about 0° C. to about 70° C. In some embodiments, the temperature is about 40° C. to about 20° C. In some embodiments, the crystallization comprises cooling a solution comprising Compound I and malonic acid from about 30-50° C. (e.g., about 40° C.) to about 10-30° C. (e.g., about 20° C.).

In some embodiments, the process comprises crystallizing Compound I from a solvent such as a nitrile solvent (e.g., acetonitrile), by cooling a solution comprising Compound I and gentisic acid from about 30-50° C. (e.g., about 40° C.) to about 10-30° C. (e.g., about 20° C.), to provide Compound I Gentisate.

Also provided are processes for providing Compound I Form I. For example, the process may comprise converting Compound I or a salt thereof to Compound I Form I.

In some embodiments, the process comprises crystallizing Compound I from a solvent to provide Compound I Form I. In some embodiments, the Compound I is amorphous (e.g., Compound I Amorphous), and the process comprises crystallizing Compound I Amorphous from a solvent to provide Compound I Form I. In some embodiments, the Compound I is obtained from a process described herein and is subject to the reaction without isolation and/or purification.

Suitable solvents (e.g., solvents used to provide Compound I Form I from Compound I) include, but are not limited to, alcohols (e.g., methanol, ethanol, 2-propanol, etc.), ethers (e.g., tetrahydrofuran, 2-methyltetrahydrofuran, cyclopentyl methyl ether, diisopropyl ether, di-n-butyl ether, 1,4-dioxane, etc.), ketones (e.g., methyl ethyl ketone, methyl isobutyl ketone, acetone, etc.), esters (e.g., ethyl acetate, isopropyl acetate, methyl acetate, etc.), hydrocarbons (e.g., toluene, n-heptane, etc.), nitriles (e.g., propionitrile, butyronitrile, benzonitrile, etc.), water, and any combination thereof.

In some embodiments, the solvent is an alcohol solvent. In some embodiments, the solvent is ethanol.

In some embodiments, the crystallization (e.g., the crystallization of Compound I) occurs at a temperature of about −10° C. to about 70° C. In some embodiments, the temperature is about 50° C. to about 20° C. In some embodiments, the crystallization comprises cooling a solution comprising Compound I from about 40-60° C. (e.g., about 50° C.) to about 10-30° C. (e.g., about 20° C.).

In some embodiments, the process comprises crystallizing Compound I from a solvent such as an alcohol solvent (e.g., ethanol), by cooling a solution (e.g., an ethanol solution) of Compound I from about 40-60° C. (e.g., about 50° C.) to about 10-30° C. (e.g., about 20° C.), to provide Compound I Form I.

Alternatively or additionally, a salt or a cocrystal of Compound I may be converted to Compound I Form I.

In some embodiments, the process comprises subjecting Compound I Mono-Malonate to a suitable condition to provide Compound I Form I.

In some embodiments, the condition (e.g., the condition for converting Compound I Mono-Malonate to Compound I Form I) comprises a solvent. Suitable solvents include, but are not limited to, alcohols (e.g., methanol, ethanol, 2-propanol, etc.), ethers (e.g., tetrahydrofuran, 2-methyltetrahydrofuran, cyclopentyl methyl ether, diisopropyl ether, di-n-butyl ether, 1,4-dioxane, etc.), ketones (e.g., methyl ethyl ketone, methyl isobutyl ketone, acetone, etc.), esters (e.g., ethyl acetate, isopropyl acetate, methyl acetate, etc.), hydrocarbons (e.g., toluene, n-heptane, etc.), nitriles (e.g., propionitrile, butyronitrile, benzonitrile, etc.), water, and any combination thereof.

In some embodiments, the solvent is an alcohol solvent. In some embodiments, the solvent is ethanol.

In some embodiments, the condition (e.g., the condition for converting Compound I Mono-Malonate to Compound I Form I) comprises a temperature of about −10° C. to about 70° C. In some embodiments, the temperature is about 50° C. to about 20° C. In some embodiments, the temperature is about 20° C.

In some embodiments, the process comprises subjecting Compound I Mono-Malonate to a solvent such as an alcohol solvent (e.g., ethanol), and at a suitable temperature such as a temperature of about 20° C. to about 50° C. (e.g., about 20° C.) to provide Compound I Form I.

Also provided are processes for providing Compound I Amorphous.

A salt or cocrystal of Compound I may be converted to Compound I Amorphous under suitable conditions.

In some embodiments, the process comprises subjecting Compound I Mono-Malonate to a suitable condition to provide Compound I Amorphous.

In some embodiments, the condition (e.g., the condition for converting Compound I Mono-Malonate to Compound I Amorphous) comprises a solvent. Suitable solvents include, but are not limited to, ethers (e.g., tetrahydrofuran, 2-methyltetrahydrofuran, tert-butyl methyl ether, etc.), ketones (e.g., acetone, 2-butanone, 4-methyl-2-pentanone, etc.), esters (e.g., ethyl acetate, isopropyl acetate, methyl acetate, etc.), hydrocarbons (e.g., n-heptane, etc.), nitriles (e.g., acetonitrile, etc.), water, and any combination thereof.

In some embodiments, the solvent is a mixed solvent. In some embodiments, the solvent is a mixture of ethyl acetate, n-heptane, and water.

In some embodiments, the condition (e.g., the condition for converting Compound I Mono-Malonate to Compound I Amorphous) comprises a base. Suitable bases include, but are not limited to, amines (e.g., triethylamine, N,N-diisopropylethylamine, tripropylamine, tributylamine, 1,4-diazabicylo[2.2.2]-octane, 1,8-diazabicyclo[5.4.0]undec-7-ene, etc.), carbonates (e.g., lithium carbonate, sodium carbonate, potassium carbonate, cesium carbonate, etc.), bicarbonates (e.g., lithium bicarbonate, sodium bicarbonate, potassium bicarbonate, cesium bicarbonate, etc.), phosphates (e.g., sodium phosphate, disodium phosphate, potassium phosphate, dipotassium hydrogen phosphate, cesium phosphate, etc.), basic aromatic compounds (e.g., pyridine, 2,6-lutidine, imidazole, etc.), and hydroxides (e.g., sodium hydroxide, potassium hydroxide, etc.).

In some embodiments, the base is a phosphate base. In some embodiments, the base is dipotassium hydrogen phosphate.

In some embodiments, the condition (e.g., the condition for converting Compound I Mono-Malonate to Compound I Amorphous) comprises a temperature of about 0° C. to about 70° C. In some embodiments, the temperature is about 10° C. to about 30° C. In some embodiments, the temperature is about 20° C.

In some embodiments, the process comprises subjecting Compound I Mono-Malonate to a solvent such as mixture of ethyl acetate, n-heptane, and water in the presence of a base such as a phosphate base (e.g., dipotassium hydrogen phosphate), and at a suitable temperature such as a temperature of about 10° C. to about 30° C. (e.g., about 20° C.) to provide Compound I Amorphous.

The Compound I Amorphous may be further converted to Compound I Form I according to a process described anywhere herein.

### Synthesis of Compound III and Synthetic Intermediates

Also provided are processes for preparing Compound III or a salt thereof (or a precursor for preparing Compound III or a salt thereof). In some embodiments, the Compound III or a salt thereof (or a precursor for preparing Compound III or a salt thereof) prepared according to a process described herein may be used for preparing Compound I or a salt thereof.

### Compound III-1a to Compound III

In some embodiments, provided herein is a process for preparing Compound III

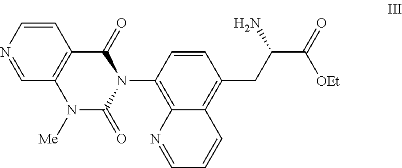

or a salt thereof, comprising:
(a) contacting Compound III-1a

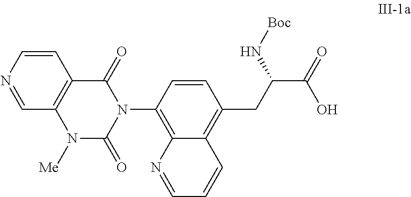 [SMILES: Cn1c(=O)n(-c2ccc(C[C@H](NC(=O)OC(C)(C)C)C(=O)O)c3cccnc23)c(=O)c2ccncc21]

or a salt thereof with ethanol in the presence of an acid to form Compound IIIa

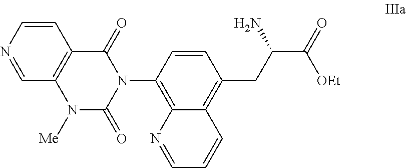 [SMILES: CCOC(=O)[C@@H](N)Cc1ccc(-n2c(=O)c3ccncc3n(C)c2=O)c2ncccc12]

or a salt thereof; and
(b) crystallizing Compound IIIa from a solvent to provide Compound III as a single atropisomer.

In some embodiments, the process comprises contacting Compound III-1a or a salt thereof with ethanol in the presence of an acid. Suitable acids include, but are not limited to, sulfonic acids (e.g., methanesulfonic acid, p-toluenesulfonic acid, etc.), mineral acids (e.g., hydrochloric acid, hydrobromic acid, sulfuric acid, phosphoric acid, etc.), and carboxylic acids (e.g., acetic acid, pivalic acid, trifluoroacetic acid, etc.).

In some embodiments, the acid is a sulfonic acid. In some embodiments, the acid is methanesulfonic acid.

In some embodiments, the reaction between Compound III-1a or a salt thereof and ethanol (step (a)) occurs in the presence of a solvent. In some embodiments, the solvent is selected from ethers (e.g., tetrahydrofuran, 2-methyltetrahydrofuran, 1,4-dioxane, di-n-butyl ether, etc.), halogenated solvents (e.g., dichloromethane, 1,2-dichloroethane, etc.), nitriles (e.g., acetonitrile), and a combination of any of the foregoing with ethanol. In some embodiments, the solvent is ethanol.

In some embodiments, the reaction between Compound III-1a or a salt thereof and ethanol occurs in the presence of an activating agent. The activating agent may be an acid halide forming agent such as oxalyl chloride.

In some embodiments, the reaction between Compound III-1a or a salt thereof and ethanol occurs in the absence of an activating agent.

In some embodiments, the reaction between Compound III-1a or a salt thereof and ethanol occurs at a temperature of about 20° C. to about 120° C. In some embodiments, the temperature is about 65° C. to about 85° C. In some embodiments, the temperature is about 75° C.

In some embodiments, the reaction between Compound III-1a or a salt thereof and ethanol occurs in the presence of an acid such as a sulfonic acid (e.g., methanesulfonic acid), a solvent (e.g., ethanol), and a suitable temperature such as a temperature of about 65° C. to about 85° C. (e.g., about 75° C.).

In some embodiments, Compound III-6a

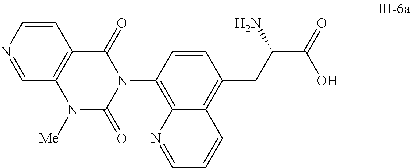 [SMILES: Cn1c(=O)n(-c2ccc(C[C@H](N)C(=O)O)c3cccnc23)c(=O)c2ccncc21]

or a salt thereof, is formed in step (a), which is subsequently converted to Compound IIIa or a salt thereof.

In some embodiments, Compound III-3a

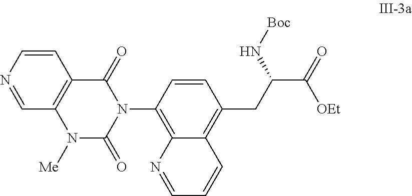 [SMILES: CCOC(=O)[C@H](Cc1ccc(-n2c(=O)c3ccncc3n(C)c2=O)c2ncccc12)NC(=O)OC(C)(C)C]

or a salt thereof, is formed in step (a), which is subsequently converted to Compound IIIa or a salt thereof.

In some embodiments, the process comprises crystallizing Compound IIIa or a salt thereof from a solvent to provide Compound III or a salt thereof as a single atropisomer. Suitable solvents include, but are not limited to, alcohols (e.g., methanol, ethanol, 2-propanol, etc.), ethers (e.g., tetrahydrofuran, 2-methyltetrahydrofuran, tert-butyl methyl ether, etc.), ketones (e.g., acetone, 2-butanone, 4-methyl-2-pentanone, etc.), esters (e.g., ethyl acetate, isopropyl acetate, etc.), hydrocarbons (e.g., benzene, toluene, etc.), halogenated solvents (e.g., dichloromethane, 1,2-dichloroethane, chlorobenzene, etc.), nitriles (e.g., acetonitrile), and any combination thereof.

In some embodiments, the solvent is a nitrile solvent. In some embodiments, the solvent is acetonitrile.

In some embodiments, the crystallization (e.g., crystallization of Compound IIIa) occurs at a temperature of about 0° C. to about 120° C. In some embodiments, the temperature is about 60° C. to about 10° C. In some embodiments, the temperature is about 50° C. to about 20° C. In some embodiments, the crystallization comprises cooling a solution comprising Compound IIIa from about 50° C. to about 20° C.

In some embodiments, the crystallization (e.g., crystallization of Compound IIIa) occurs under a condition comprising a solvent such as a nitrile solvent (e.g., acetonitrile) and a suitable temperature such as a temperature of about 60° C. to about 10° C. (e.g., about 50° C. to about 20° C.).

In some embodiments, the process further comprises equilibrating Compound IIIa or a salt thereof prior to crystallization. In accordance with any embodiments described herein, Compound IIIa may represent a mixture of atropisomers including Compound III. The equilibration may be performed under an acidic or thermal condition.

In some embodiments, the equilibration is performed under an acidic condition (e.g., acidic equilibration).

In some embodiments, the acidic condition (e.g., the acidic condition for equilibrating Compound IIIa) comprises an acid. Suitable acids include, but are not limited to, hydrochloric acid, hydrobromic acid, sulfuric acid, carboxylic acids (e.g., acetic, pivalic, trifluoroacetic acid, etc.), phosphoric acid, and sulfonic acids (e.g., methanesulfonic acid, p-toluenesulfonic acid, etc.).

In some embodiments, the acid for acidic equilibration is a sulfonic acid. In some embodiments, the acid for acidic equilibration is methanesulfonic acid.

The acidic condition (e.g., the acidic condition for equilibrating Compound IIIa) may comprise a solvent. In some embodiments, the solvent is selected from ethers (e.g., tetrahydrofuran, 2-methyltetrahydrofuran, 1,4-dioxane, di-n-butyl ether, etc.), halogenated solvents (e.g., dichloromethane, 1,2-dichloroethane, etc.), nitriles (e.g., acetonitrile), and a combination of any of the foregoing with ethanol. In some embodiments, the solvent is ethanol.

In some embodiments, the acidic condition (e.g., the acidic condition for equilibrating Compound IIIa) comprises a temperature of about 20° C. to about 120° C. In some embodiments, the temperature is about 65° C. to about 85° C. In some embodiments, the temperature is about 75° C.

In some embodiments, the equilibration occurs under a condition comprising an acid such as a sulfonic acid (e.g., methanesulfonic acid), a solvent (e.g., ethanol), and a suitable temperature such as a temperature of about 65° C. to about 85° C. (e.g., about 75° C.).

Alternatively or additionally, the equilibration may be performed under a thermal condition (e.g., thermal equilibration). In some embodiments, the thermal equilibration is performed in a continuous manner by using a flow reactor.

In some embodiments, the thermal condition (e.g., the thermal condition for equilibrating Compound IIIa) comprises a solvent. Suitable solvents include, but are not limited to, nitriles (e.g., acetonitrile), alcohols (e.g., methanol, ethanol, 2-propanol, etc.), ethers (e.g., tetrahydrofuran, 2-methyltetrahydrofuran, tert-butyl methyl ether, etc.), ketones (e.g., acetone, 2-butanone, 4-methyl-2-pentanone, etc.), esters (e.g., ethyl acetate, isopropyl acetate, etc.), hydrocarbons (e.g., benzene, toluene, etc.), and halogenated solvents (e.g., dichloromethane, 1,2-dichloroethane, chlorobenzene, etc.).

In some embodiments, the solvent is a nitrile solvent. In some embodiments, the solvent is acetonitrile.

In some embodiments, the thermal condition (e.g., the thermal condition for equilibrating Compound IIIa) comprises a temperature of about 80° C. to about 200° C. In some embodiments, the temperature is about 140° C. to about 200° C. In some embodiments, the temperature is about 150° C. to about 195° C. In some embodiments, the temperature is about 170° C. to about 190° C. In some embodiments, the temperature is about 180° C. In some embodiments, the temperature is about 160° C.

In some embodiments, the equilibration (e.g., equilibrating Compound IIIa) occurs under a condition comprising a solvent such as a nitrile solvent (e.g., acetonitrile) and a suitable temperature such as a temperature of about 150° C. to about 195° C. (e.g., about 160° C.).

In some embodiments, the equilibration (e.g., equilibrating Compound IIIa) occurs under a condition comprising a solvent such as a nitrile solvent (e.g., acetonitrile) and a suitable temperature such as a temperature of about 170° C. to about 190° C. (e.g., about 180° C.).

In some embodiments, the process comprises equilibrating Compound IIIa under an acidic or thermal condition (e.g., an acidic or thermal condition as described herein), followed by crystallizing Compound IIIa from a solvent to provide Compound III as a single atropisomer.

### Synthesis of Compound III-1a

In some embodiments, provided is a process for preparing Compound III-1a

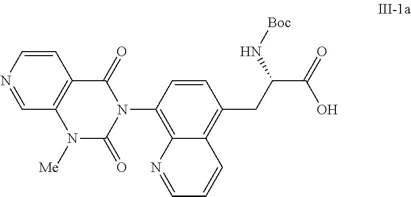 [SMILES: Cn1c(=O)n(-c2ccc(C[C@H](NC(=O)OC(C)(C)C)C(=O)O)c3cccnc23)c(=O)c2ccncc21]

or a salt thereof, comprising:
(a) contacting Compound IV-1

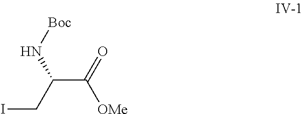 [SMILES: COC(=O)[C@H](CI)NC(=O)OC(C)(C)C]

or a salt thereof with zinc; and
(b) contacting a product from step (a) with a compound of Formula Va

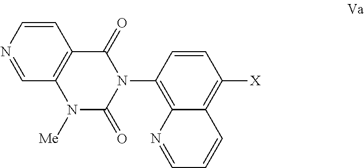

wherein X is Br or I,
or a salt thereof in the presence of a catalytic system to form Compound III-2a

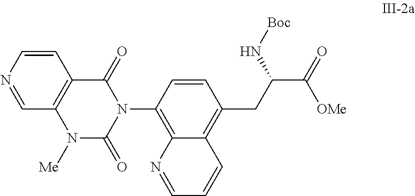 [SMILES: COC(=O)[C@H](Cc1ccc(-n2c(=O)c3ccncc3n(C)c2=O)c2ncccc12)NC(=O)OC(C)(C)C]

or a salt thereof.

In some embodiments, the reaction between Compound IV-1 or a salt thereof and zinc (step (a)) occurs in the presence of a solvent. Suitable solvents include, but are not limited to aromatic solvents (e.g., toluene, xylene, etc.), polar aprotic solvents (e.g., N,N-dimethylformamide, N,N-dimethylacetamide, N-methyl-2-pyrrolidone, dimethyl sulfoxide, etc.), ethers (e.g., tetrahydrofuran, 2-methyltetrahydrofuran, 1,4-dioxane, etc.), and nitriles (e.g., acetonitrile, benzonitrile, etc.), and any combination thereof.

In some embodiments, the solvent is a mixed solvent. In some embodiments, the solvent is a mixture of toluene and N,N-dimethylformamide.

In some embodiments, the reaction between Compound IV-1 or a salt thereof and zinc (step (a)) occurs in the presence of an activator. Suitable activators include, but are not limited to methanesulfonic acid, iodine, 1,2-dibromoethane, and silyl chlorides (e.g., trimethylsilyl chloride, etc.).

In some embodiments, the activator is a silyl chloride. In some embodiments, the activator is trimethylsilyl chloride.

In some embodiments, the reaction between Compound IV-1 or a salt thereof and zinc occurs at a temperature of about −20° C. to about 50° C. In some embodiments, the temperature is about 10° C. to about 30° C. In some embodiments, the temperature is about 20° C.

In some embodiments, the reaction between Compound IV-1 or a salt thereof and zinc occurs under a condition comprising a solvent (e.g. a mixture of toluene and N,N-Dimethylformamide), an activator such as a silyl chloride (e.g., trimethylsilyl chloride), and at a suitable temperature such as a temperature of about 10° C. to about 30° C. (e.g., about 20° C.).

In some embodiments, the reaction between Compound IV-1 or a salt thereof and zinc provides Compound IV-2

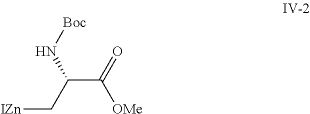

or a salt thereof.

In some embodiments, the process comprises contacting Compound IV-2 or a salt thereof with a compound of Formula Va or a salt thereof in the presence of a catalytic system to form Compound III-2a or a salt thereof.

The catalytic system (e.g., the catalytic system for providing Compound III-2a from Compound IV-2 and a compound of Formula Va) may comprise a catalyst. The catalyst may be a palladium catalyst or a nickel catalyst. The palladium catalyst can be a palladium(II) source (e.g., palladium(II) acetate, palladium(II) chloride, etc.) or a palladium(0) source (e.g., tetrakis(triphenylphosphine)palladium(0), etc.). The nickel catalyst can be a nickel(II) source (e.g., nickel(II) chloride, etc.) or a nickel(0) source (e.g., bis(cyclooctadiene)nickel(0)).

In some embodiments, the catalyst is a palladium(II) source. In some embodiments, the catalyst is palladium(II) acetate.

The catalytic system (e.g., the catalytic system for providing Compound III-2a from Compound IV-2 and a compound of Formula Va) may further comprise a ligand. Suitable ligands include, but are not limited to, monodentate phosphine ligands (e.g., triphenylphosphine, 2-dicyclohexylphosphino-2′,4′,6′-triisopropylbiphenyl, 2-dicyclohexylphosphino-2′,6′-dimethoxybiphenyl, etc.), bidentate phosphine ligands (e.g., 1,2-bis(diphenylphosphino)ethane, 2,2′-bis(diphenylphosphino)-1,1′-binaphthyl, 1,1′-bis(diphenylphosphino)ferrocene, etc.), bipyridine ligands (e.g., 4,4′-di-tert-butyl-2,2′-dipyridyl, 2,2′-dipyridine), 1,10-phenanthroline, and terpyridine ligands.

In some embodiments, the ligand is a monodentate phosphine ligand. In some embodiments, the ligand is 2-dicyclohexylphosphino-2′,4′,6′-triisopropylbiphenyl.

The catalytic system (e.g., the catalytic system for providing Compound III-2a from Compound IV-2 and a compound of Formula Va) may further comprise a solvent. Suitable solvents include, but are not limited to, aromatic solvents (e.g., toluene, xylene, etc.), polar aprotic solvents (e.g., N,N-dimethylformamide, N,N-dimethylacetamide, N-methyl-2-pyrrolidone, dimethyl sulfoxide, etc.), ethers (e.g., tetrahydrofuran, 2-methyltetrahydrofuran, 1,4-dioxane, etc.), and nitriles (e.g., acetonitrile, benzonitrile, etc.).

In some embodiments, the solvent is an aromatic solvent. In some embodiments, the solvent is toluene.

In some embodiments, the reaction between Compound IV-2 or a salt thereof and a compound of Formula Va or a salt thereof occurs at a temperature of about 20° C. to about 120° C. In some embodiments, the temperature is about 50° C. to about 70° C. In some embodiments, the temperature is about 60° C.

In some embodiments, the reaction between Compound IV-2 or a salt thereof and a compound of Formula Va or a salt thereof occurs at a suitable temperature such as a temperature of about 50° C. to about 70° C. (e.g., about 60° C.), and in the presence of a catalytic system comprising a catalyst such as a palladium catalyst (e.g., palladium(II) acetate), a ligand such as a monodentate phosphine ligand (e.g., 2-dicyclohexylphosphino-2′,4′,6′-triisopropylbiphenyl), and a solvent (e.g., a mixture of toluene and N,N-dimethylformamide).

In some embodiments, the process further comprises subjecting Compound III-2a

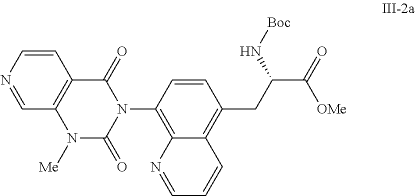 [SMILES: COC(=O)[C@H](Cc1ccc(-n2c(=O)c3ccncc3n(C)c2=O)c2ncccc12)NC(=O)OC(C)(C)C]

or a salt thereof to a hydrolysis condition to form Compound III-1a

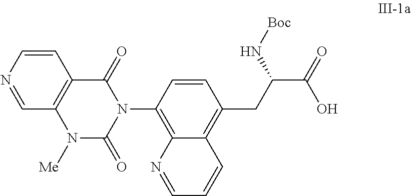 [SMILES: Cn1c(=O)n(-c2ccc(C[C@H](NC(=O)OC(C)(C)C)C(=O)O)c3cccnc23)c(=O)c2ccncc21]

or a salt thereof.

In some embodiments, the hydrolysis condition (e.g., the hydrolysis condition for proving Compound III-1a from Compound III-2a) comprises a base. Suitable bases include, but are not limited to, hydroxides (e.g., lithium hydroxide, sodium hydroxide, potassium hydroxide, ammonium hydroxide, etc.), carbonates (e.g., lithium carbonate, potassium carbonate, sodium carbonate, cesium carbonate, etc.), alkoxides (e.g., sodium methoxide, sodium tert-butoxide, potassium tert-butoxide, etc.), and phosphates (e.g., potassium phosphate, sodium phosphate, etc.).

In some embodiments, the base is a hydroxide. In some embodiments, the base is sodium hydroxide (e.g., aqueous sodium hydroxide).

In some embodiments, the hydrolysis condition (e.g., the hydrolysis condition for proving Compound III-1a from Compound III-2a) comprises a solvent. Suitable solvents include, but are not limited to, ethers (e.g., tetrahydrofuran, 2-methyltetrahydrofuran, tert-butyl methyl ether, etc.), nitriles (e.g., benzonitrile, etc.), alcohols (e.g., methanol, etc.), aromatic solvents (e.g., toluene, etc.), any combination thereof, and a combination of any of the foregoing with water.

In some embodiments, the solvent is a mixed solvent. In some embodiments, the solvent is a mixture of toluene and 2-methyltetrahydrofuran.

In some embodiments, the hydrolysis of Compound III-2a or a salt thereof occurs at a temperature of about −10° C. to about 80° C. In some embodiments, the temperature is about −10° C. to about 10° C. In some embodiments, the temperature is about 0° C.

In some embodiments, the hydrolysis of Compound III-2a occurs under a hydrolysis condition comprising a base such as a hydroxide (e.g., sodium hydroxide such as aqueous sodium hydroxide), a solvent such as a mixture of toluene and 2-methyltetrahydrofuran, and a suitable temperature such as a temperature of about −10° C. to about 10° C. (e.g., about 0° C.).

In some embodiments, Compound III-2a or a salt thereof is subject to hydrolysis without isolation and/or purification.

In some embodiments, the process further comprises converting Compound III-1a or a salt thereof to Compound III or a salt thereof according to a process described anywhere herein.

### Synthesis of Compound III

In some embodiments, provided is a process for preparing Compound III

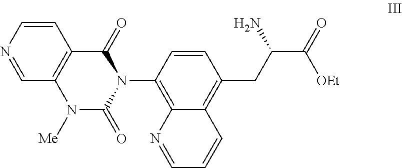

or a salt thereof, comprising:
(a) contacting Compound IV-3

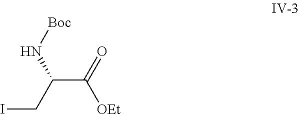 [SMILES: CCOC(=O)[C@H](CI)NC(=O)OC(C)(C)C]

or a salt thereof with zinc;
(b) contacting a product from step (a) with Compound V-I

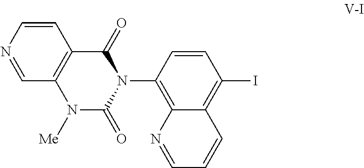

or a salt thereof in the presence of a catalytic system to form Compound III-3

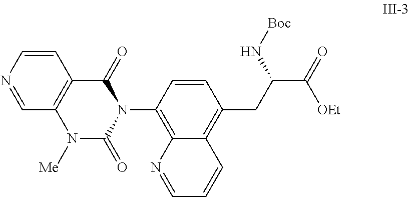

or a salt thereof; and
(c) subjecting Compound III-3 or a salt thereof to a deprotection condition to form Compound III or a salt thereof.

In some embodiments, the reaction between Compound IV-3 or a salt thereof and zinc occurs in the presence of an activator. Suitable activators include, but are not limited to, silyl chlorides (e.g., trimethylsilyl chloride, triethylsilyl chloride, etc.), methanesulfonic acid, iodine, and 1,2-dibromoethane.

In some embodiments, the activator is a silyl chloride. In some embodiments, the activator is trimethylsilyl chloride.

In some embodiments, the reaction between Compound IV-3 or a salt thereof and zinc occurs in the presence of a solvent. Suitable solvents include, but are not limited to, polar aprotic solvents (e.g., N,N-dimethylformamide, N,N-dimethylacetamide, N-methyl-2-pyrrolidone, dimethyl sulfoxide, etc.), ethers (e.g., tetrahydrofuran, 2-methyltetrahydrofuran, 1,4-dioxane, etc.), nitriles (e.g., acetonitrile, benzonitrile, etc.), aromatic solvents (e.g., toluene, etc.), and any combination thereof.

In some embodiments, the solvent is a mixture of N,N-dimethylformamide and toluene.

In some embodiments, the reaction between Compound IV-3 or a salt thereof and zinc occurs at a temperature of about −10° C. to about 50° C. In some embodiments, the temperature is about 10° C. to about 30° C. In some embodiments, the temperature is about 20° C.

In some embodiments, the reaction between Compound IV-3 or a salt thereof and zinc occurs in the presence of an activator (e.g., trimethylsilyl chloride), a solvent (e.g., a mixture of N,N-dimethylformamide and toluene), and at a suitable temperature such as a temperature of about 10° C. to about 30° C. (e.g., about 20° C.).

In some embodiments, the product from the reaction between Compound IV-3 or a salt thereof and zinc (step (a)) is Compound IV-4 or a salt thereof.

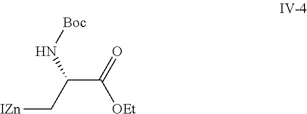

In some embodiments, Compound IV-4 or a salt thereof (e.g., the product from step (a)) is subject to the next step without isolation and/or purification.

In some embodiments, the process comprises contacting Compound IV-4 or a salt thereof (e.g., the product from step (a)) with Compound V-I or a salt thereof in the presence of a catalytic system to form Compound III-3.

The catalytic system (e.g., the catalytic system for providing Compound III-3 from Compound IV-4 and Compound V-1) may comprises a catalyst. The catalyst may be a palladium catalyst or a nickel catalyst. The palladium catalyst can be a palladium(II) source (e.g., palladium(II) acetate, palladium(II) chloride, etc.) or a palladium(0) source (e.g., tetrakis(triphenylphosphine)palladium(0), etc.). The nickel catalyst can be a nickel(II) source (e.g., nickel(II) chloride, etc.) or a nickel(0) source (e.g., bis(cyclooctadiene)nickel(0)).

In some embodiments, the catalyst is a palladium(II) source. In some embodiments, the catalyst is palladium(II) acetate.

The catalytic system (e.g., the catalytic system for providing Compound III-3 from Compound IV-4 and Compound V-I) may further comprise a ligand. Suitable ligands include, but are not limited to, monodentate phosphine ligands (e.g., triphenylphosphine, 2-dicyclohexylphosphino-2′,4′,6′-triisopropylbiphenyl, 2-dicyclohexylphosphino-2′,6′-dimethoxybiphenyl, etc.), bidentate phosphine ligands (e.g., 1,2-bis(diphenylphosphino)ethane, 2,2′-bis(diphenylphosphino)-1,1′-binaphthyl, 1,1′-bis(diphenylphosphino)ferrocene, etc.), bipyridine ligands (e.g., 4,4′-di-tert-butyl-2,2′-dipyridyl, 2,2′-dipyridine), 1,10-phenanthroline, and terpyridine ligands.

In some embodiments, the ligand is a monodentate phosphine ligand. In some embodiments, the ligand is 2-dicyclohexylphosphino-2′,4′,6′-triisopropylbiphenyl.

The catalytic system (e.g., the catalytic system for providing Compound III-3 from Compound IV-4 and Compound V-I) may further comprise a solvent. Suitable solvents include, but are not limited to, aromatic solvents (e.g., toluene, etc.), polar aprotic solvents (e.g., N,N-dimethylformamide, N,N-dimethylacetamide, N-methyl-2-pyrrolidone, dimethyl sulfoxide, etc.), ethers (e.g., tetrahydrofuran, 2-methyltetrahydrofuran, 1,4-dioxane, etc.), nitriles (e.g., acetonitrile, benzonitrile, etc.), and any combination thereof.

In some embodiments, the solvent is a mixed solvent. In some embodiments, the solvent is a mixture of toluene and N,N-dimethylformamide.

In some embodiments, the reaction between Compound IV-4 or a salt thereof and Compound V-I or a salt thereof occurs at a temperature of about 20° C. to about 120° C. In some embodiments, the temperature is about 50° C. to about 70° C. In some embodiments, the temperature is about 60° C.

In some embodiments, the reaction between Compound IV-4 or a salt thereof and Compound V-I or a salt thereof occurs at a suitable temperature such as a temperature of about 50° C. to about 70° C. (e.g., about 60° C.), and in the presence of a catalytic system comprising a catalyst such as a palladium catalyst (e.g., palladium(II) acetate), a ligand such as a monodentate phosphine ligand (e.g., 2-dicyclohexylphosphino-2′,4′,6′-triisopropylbiphenyl), and a solvent (e.g., a mixture of toluene and N,N-dimethylformamide).

In some embodiments, the process comprises subjecting Compound III-3 or a salt thereof to a deprotection condition to form Compound III or a salt thereof.

In some embodiments, the deprotection condition (e.g., the deprotection condition for providing Compound III from Compound III-3) comprises a deprotection reagent. Suitable deprotection reagents include, but are not limited to, mineral acids (e.g., hydrochloric acid, hydrobromic acid, sulfuric acid, phosphoric acid, etc.), carboxylic acids (e.g., acetic acid, pivalic acid, trifluoroacetic acid, etc.), sulfonic acids (e.g., ptoluenesulfonic acid, etc.), Lewis acids (e.g., boron trifluoride diethyl etherate, boron trifluoride THF complex, etc.), oxalyl chloride, and metal salts (e.g., iron(III) chloride, zinc(II) bromide, etc.).

In some embodiments, the deprotection reagent is an acid. In some embodiments, the deprotection reagent is phosphoric acid.

In some embodiments, the deprotection condition (e.g., the deprotection condition for providing Compound III from Compound III-3) comprises a solvent. Suitable solvents include, but are not limited to, ethers (e.g., tetrahydrofuran, tert-butyl methyl ether, etc.), polar aprotic solvents (e.g., N,N-dimethylformamide, N,N-dimethylacetamide, dimethyl sulfoxide, etc.), nitriles (e.g., acetonitrile), halogenated solvents (e.g., dichloromethane), and a combination of any of the foregoing with ethanol.

In some embodiments, the solvent is a mixed solvent. In some embodiments, the solvent is a mixture of ethanol and tetrahydrofuran.

In some embodiments, the deprotection condition (e.g., the deprotection condition for providing Compound III from Compound III-3) comprises a temperature of about 0° C. to about 50° C. In some embodiments, the temperature is about 10° C. to about 30° C. In some embodiments, the temperature is about 20° C.

In some embodiments, the deprotection condition (e.g., the deprotection condition for providing Compound III from Compound III-3) comprises a deprotection reagent such as an acid (e.g., phosphoric acid), a solvent (e.g., a mixture of ethanol and tetrahydrofuran), and a suitable temperature such as a temperature of about 10° C. to about 30° C. (e.g., about 20° C.).

### Synthesis of Compound III-2a

In some embodiments, provided is a process for preparing Compound III-2a

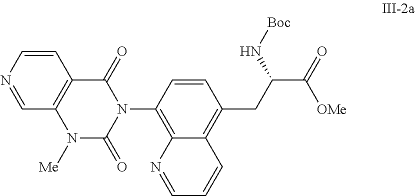 [SMILES: COC(=O)[C@H](Cc1ccc(-n2c(=O)c3ccncc3n(C)c2=O)c2ncccc12)NC(=O)OC(C)(C)C]

or a salt thereof, comprising:
(a) contacting Compound IV-6

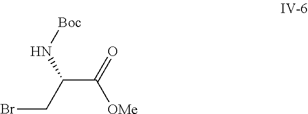 [SMILES: COC(=O)[C@H](CBr)NC(=O)OC(C)(C)C]

or a salt thereof with Compound Va-Br

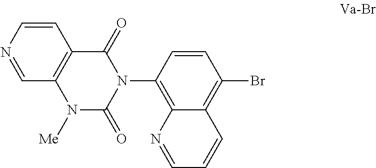 [SMILES: Cn1c(=O)n(-c2ccc(Br)c3cccnc23)c(=O)c2ccncc21]

or a salt thereof in the presence of a photosensitizer to provide Compound III-2a or a salt thereof.

Suitable photosensitizers (e.g., photosensitizers for providing Compound III-2a from Compound Va-Br and Compound IV-6) include, but are not limited to, iridium photocatalyst (e.g., Ir(dFCF₃ppy)₂(dtbbpy)PF₆, Ir[ppy]₂(dtbbpy)PF₆, Ir(FMeppy)₂(dtbbpy)PF₆, Ir(dFMeppy)₂(dtbbpy)PF₆, Ir(dFFppy)₂(dtbbpy)PF₆, Ir(dFCF₃ppy)₂(bpy)PF₆, Ir(dFCF₃ppy)₂(dCF₃bpy)PF₆), and donor-acceptor fluorophores photocatalyst (e.g., 1,2,3,5-tetrakis(carbazol-9-yl)-4,6-dicyanobenzene, 2,4,5,6-Tetrakis(9H-carbazol-9-yl) isophthalonitrile (4CzIPN, CAS 1416881-52-1), 2,4,6-tris(diphenylamino)-5-fluoroisophthalonitrile (3DPAIPN, CAS 2260543-73-3), 3,4,5,6-tetrakis(carbazol-9-yl)-1,2-dicyanobenzene (4CzPN, CAS 1416881-51-0), 2,3,5,6-tetrakis(carbazol-9-yl)-1,4-dicyanobenzene (4CzTPN, CAS 1416881-53-2), 2,4,5,6-tetrakis(diphenylamino)isophthalonitrile (4DPAIPN, CAS 1846598-27-3), etc.).

In some embodiments, the photosensitizer is an iridium photocatalyst. In some embodiments, the photosensitizer is Ir(dFCF₃ppy)₂(dtbbpy)PF₆.

In some embodiments, the reaction between Compound IV-6 or a salt thereof and Compound Va-Br or a salt thereof occurs in the presence of a co-catalyst. In some embodiments, the co-catalyst is a nickel co-catalyst. Suitable nickel co-catalysts include, but are not limited to, nickel(II) bromide ethylene glycol dimethyl ether, nickel(II) chloride hexahydrate, nickel(II) chloride ethylene glycol dimethyl ether, nickel(II) tetrafluoroborate, nickel(II) acetate, nickel(II) bromide trihydrate, nickel(II) acetylacetonate, and nickel(II) stearate.

In some embodiments, the co-catalyst is nickel(II) bromide ethylene glycol dimethyl ether.

In some embodiments, the reaction between Compound IV-6 or a salt thereof and Compound Va-Br or a salt thereof occurs in the presence of a ligand. Suitable ligands include, but are not limited to, bipyridine ligands (e.g., 4,4′-di-tert-butyl-2,2′-dipyridyl, 2,2′-dipyridine, etc.) and 1,10-phenanthroline.

In some embodiments, the ligand is a bipyridine ligand. In some embodiments, the ligand is 4,4′-di-tert-butyl-2,2′-dipyridyl.

In some embodiments, the reaction between Compound IV-6 or a salt thereof and Compound Va-Br or a salt thereof occurs in the presence of a reductant. Suitable reductants include, but are not limited to, silanes (e.g., tris(trimethylsilyl)silane, triphenylsilane, triethylsilane, dimethyl(phenyl)silane, N-(tert-butyl)-1,1,1,3,3,3-hexamethyl-2-(trimethylsilyl)trisilan-2-amine, N-isopropyl-1,1,1,3,3,3-hexamethyl-2-(trimethylsilyl)trisilan-2-amine, N-butyl-1,1,1,3,3,3-hexamethyl-2-(trimethylsilyl)trisilan-2-amine, tris(trimethylsilyl)silanol, etc.), and amines (e.g., triethylamine, triethanolamine, N,N-diisopropylethylamine, tripropylamine, diethylpropylamine, etc.).

In some embodiments, the reductant is a silane. In some embodiments, the reductant is tris(trimethylsilyl)silane.

In some embodiments, the reaction between Compound IV-6 or a salt thereof and Compound Va-Br or a salt thereof occurs in the presence of a base. Suitable bases include, but are not limited to, cyclic aliphatic amines (e.g., 2,2,6,6-tetramethylpiperidine, 1-methylpiperidine, pyrrolidine, piperidine, etc.), acetates (e.g., potassium acetate, lithium acetate, sodium acetate, ammonium acetate, cesium acetate, etc.), carbonates (e.g., sodium carbonate, potassium carbonate, cesium carbonate, etc.), quinuclidine-based bases (e.g., quinuclidine, 1,4-diazabicyclo[2.2.2]octane (DABCO), hydroxyquinuclidine, 3-acetoxyquinuclidine, etc.), guanidine bases (e.g., 1,1,3,3-tetramethylguanidine, etc.), hydroxides (e.g., lithium hydroxide, sodium hydroxide, potassium hydroxide, etc.), heterocyclic aromatic bases (e.g., 2,6-lutidine, etc.), and phosphates (e.g., tripotassium phosphate, trilithium phosphate, etc.).

In some embodiments, the base is a carbonate base. In some embodiments, the base is sodium carbonate.

In some embodiments, the reaction between Compound IV-6 or a salt thereof and Compound Va-Br or a salt thereof occurs in the absence of an additive.

In some embodiments, the reaction between Compound IV-6 or a salt thereof and Compound Va-Br or a salt thereof occurs in the presence of an additive. Suitable additives include, but are not limited to, phthalimide additives (e.g., phthalimide, 4-tert-butylphthalimide, 4-chloro-1,3-dioxo-1,3-dihydroisoindole, methyl 1,3-dioxoisoindoline-5-carboxylate, etc.) and succinimide additives (e.g., succinimide, 1,2,3,6-tetrahydrophthalimide, 2,2,3,3-tetramethylsuccinimide, (±)-α-methyl-α-phenylsuccinimide, bicyclo[2.2.1]heptane-2,3-dicarboximide, etc.).

In some embodiments, the reaction between Compound IV-6 or a salt thereof and Compound Va-Br or a salt thereof occurs in the presence of a solvent. Suitable solvents include, but are not limited to, ethers (e.g., 1,2-dimethoxyethane, cyclopentyl methyl ether, 1,4-dioxane, 2-methyltetrahydrofuran, dibutyl ether, tert-butyl methyl ether, tetrahydrofuran, etc.), nitriles (e.g., acetonitrile, etc.), polar aprotic solvents (e.g., dimethylacetamide, N,N′-dimethylpropyleneurea, 1,3-dimethyl-2-imidazolidinone, dimethyl sulfoxide, N-methyl-2-pyrrolidone, N,N-dimethylformamide, etc.), and alcohols (e.g., isopropyl alcohol, 1,1,1,3,3,3-hexafluoro-2-propanol, etc.).

In some embodiments, the solvent is an ether solvent. In some embodiments, the solvent is 1,2-dimethoxyethane.

In some embodiments, the reaction between Compound IV-6 or a salt thereof and Compound Va-Br or a salt thereof occurs at a temperature of about 0° C. to about 120° C. In some embodiments, the temperature is about 15° C. to about 35° C. In some embodiments, the temperature is about 25° C.

In some embodiments, the reaction between Compound IV-6 or a salt thereof and Compound Va-Br or a salt thereof occurs under a light having a wavelength of about 330 nm to about 500 nm. In some embodiments, the wavelength is about 450 nm.

In some embodiments, the reaction between Compound IV-6 or a salt thereof and Compound Va-Br or a salt thereof occurs in the presence of a photosensitizer such as an iridium photocatalyst (e.g., Ir(dFCF₃ppy)₂(dtbbpy)PF₆), a co-catalyst such as a nickel co-catalyst (e.g., nickel(II) bromide ethylene glycol dimethyl ether), a ligand such as a bipyridine ligand (e.g., 4,4′-di-tert-butyl-2,2′-bipyridine), a base such as a carbonate (e.g., sodium carbonate), a solvent such as an ether solvent (e.g., 1,2-dimethoxyethane), at a suitable temperature such as a temperature of about 15° C. to about 35° C. (e.g., about 25° C.), and under a light having a suitable wavelength such as a wavelength of about 330 nm to about 500 nm (e.g., about 450 nm).

In some embodiments, the process further comprises converting Compound III-2a or a salt thereof to Compound IIIa or Compound III or a salt thereof according to a process described anywhere herein.

### Synthesis of Compound III-3a

In some embodiments, provided is a process for preparing Compound III-3a

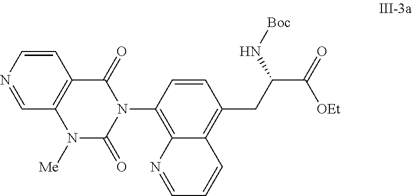 [SMILES: CCOC(=O)[C@H](Cc1ccc(-n2c(=O)c3ccncc3n(C)c2=O)c2ncccc12)NC(=O)OC(C)(C)C]

or a salt thereof, comprising:
contacting Compound Va-I

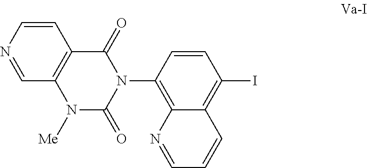 [SMILES: Cn1c(=O)n(-c2ccc(I)c3cccnc23)c(=O)c2ccncc21]

or a salt thereof with Compound IV-8

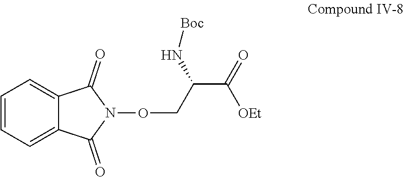 [SMILES: CCOC(=O)[C@H](CON1C(=O)c2ccccc2C1=O)NC(=O)OC(C)(C)C]

under an electrochemical coupling condition to provide Compound III-3a or a salt thereof.

In some embodiments, the electrochemical coupling condition (e.g., the electrochemical coupling condition for providing Compound III-3a from Compound Va-I and Compound IV-8) comprises a catalyst. In some embodiments, the catalyst is a nickel catalyst. Suitable nickel catalysts include, but are not limited to, nickel(II) chloride hexahydrate, nickel(II) chloride ethylene glycol dimethyl ether, nickel(II) bromide ethylene glycol dimethyl ether, nickel(II) chloride, nickel(II) iodide, nickel(II) tetrafluoroborate, nickel(II) acetate, nickel(II) bromide trihydrate, nickel(II) acetylacetonate, and nickel(II) stearate.

In some embodiments, the catalyst is nickel(II) chloride hexahydrate.

In some embodiments, the electrochemical coupling condition (e.g., the electrochemical coupling condition for providing Compound III-3a from Compound Va-I and Compound IV-8) comprises a ligand. Suitable ligands include, but are not limited to, bipyridine ligands (e.g., 2,2′-bipyridine, 4,4′-di-tert-butyl-2,2′-dipyridyl, 4,4′-dimethyl-2,2′-dipyridine, etc.), 1,10-phenanthroline, picolinimidamide hydrochloride, 2,6-bis(4,5-dihydrooxazol-2-yl)pyridine, and 2-(pyridin-2-yl)-4,5-dihydrooxazole.

In some embodiments, the ligand is a bipyridine ligand. In some embodiments, the ligand is 2,2′-bipyridine.

In some embodiments, the electrochemical coupling condition (e.g., the electrochemical coupling condition for providing Compound III-3a from Compound Va-I and Compound IV-8) comprises an additive. Suitable additives include, but are not limited to, silver salts (e.g., silver nitrate, etc.), acids (e.g., acetic acid, trifluoroacetic acid, etc.), and bases (e.g., sodium acetate, sodium bicarbonate, sodium carbonate, potassium phosphate monobasic, potassium phosphate dibasic, etc.).

In some embodiments, the additive is a silver salt. In some embodiments, the additive is silver nitrate.

In some embodiments, the electrochemical coupling condition (e.g., the electrochemical coupling condition for providing Compound III-3a from Compound Va-I and Compound IV-8) comprises a solvent. The solvent may be a polar aprotic solvent (e.g., N,N-dimethylacetamide, N,N′-dimethylpropyleneurea, 1,3-dimethyl-2-imidazolidinone, dimethyl sulfoxide, N-methyl-2-pyrrolidone, N,N-dimethylformamide, etc.).

In some embodiments, the solvent is N,N-dimethylacetamide.

In some embodiments, the electrochemical coupling condition (e.g., the electrochemical coupling condition for providing Compound III-3a from Compound Va-I and Compound IV-8) comprises a temperature of about 0° C. to about 70° C. In some embodiments, the temperature is about 15° C. to about 35° C. In some embodiments, the temperature is about 25° C.

In some embodiments, the electrochemical coupling condition (e.g., the electrochemical coupling condition for providing Compound III-3a from Compound Va-I and Compound IV-8) comprises a current of about 2 F/mol to about 10 F/mol. In some embodiments, the current is about 3.2 F/mol.

The electrochemical coupling rection (e.g., the reaction between Compound Va-I and Compound IV-8) may occur by using suitable electrodes known in the art. For example, the electrochemical coupling rection may occur by using a magnesium anode combined with a reticulated vitreous carbon cathode (Mg(+)/RVC(−)), a zinc anode combined with a reticulated vitreous carbon cathode (Zn(+)/RVC(−)), an aluminum anode combined with a reticulated vitreous carbon cathode (Al(+)/RVC(−)), a magnesium anode combined with a carbon cathode (Mg(+)/C(−)), a zinc anode combined with a carbon cathode (Zn(+)/C(−)), or an aluminum anode combined with a carbon cathode (Al(+)/C(−)).

In some embodiments, the electrochemical coupling occurs by using a magnesium anode combined with a reticulated vitreous carbon cathode (Mg(+)/RVC(−)).

In some embodiments, the electrochemical coupling condition (e.g., the electrochemical coupling condition for providing Compound III-3a from Compound Va-I and Compound IV-8) comprises a catalyst such as a nickel catalyst (e.g., nickel(II) chloride hexahydrate), a ligand such as a bipyridine ligand (e.g., 2,2′-bipyridine), an additive such as a silver salt (e.g., silver nitrate), a solvent such as a polar aprotic solvent (e.g., N,N-dimethylacetamide), a suitable temperature such as a temperature of about 15° C. to about 35° C. (e.g., about 25° C.), and under a suitable current such as a current of about 3.2 F/mol by using suitable electrodes such as a magnesium anode combined with a reticulated vitreous carbon cathode (Mg(+)/RVC(−)).

In some embodiments, the process further comprises converting Compound III-3a or a salt thereof to Compound IIIa or Compound III or a salt thereof according to a process described anywhere herein.

### Synthesis of Compound III-3

In some embodiments, provided is a process for preparing Compound III-3

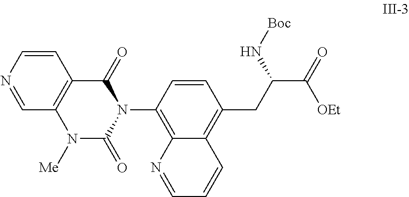

or a salt thereof, comprising:
(a) contacting Compound IV-5

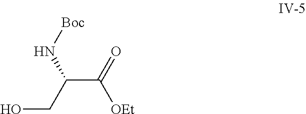 [SMILES: CCOC(=O)[C@H](CO)NC(=O)OC(C)(C)C]

or a salt thereof with a benzoxazolium salt activator;
(b1) contacting a product from step (a) with Compound V-1-Br

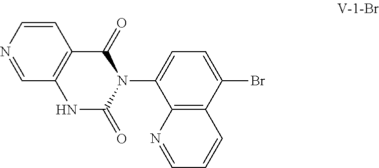

or a salt thereof in the presence of a photosensitizer to provide Compound III-4

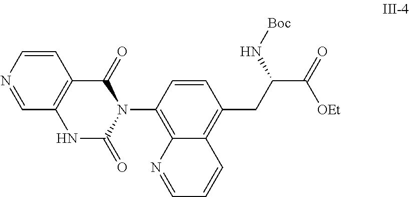

or a salt thereof; or
(b2) contacting the product from step (a) with Compound V-Br

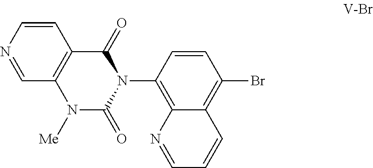

or a salt thereof in the presence of a photosensitizer to provide Compound III-3 or a salt thereof; and
(c) when step (b1) occurs, contacting Compound III-4 or a salt thereof with a methylation agent to form Compound III-3 or a salt thereof.

In some embodiments, the process comprises contacting Compound IV-5 or a salt thereof with a benzoxazolium salt activator. Suitable benzoxazolium salt activators include, but are not limited to, 5,7-di-tert-butyl-3-phenylbenzo[d]oxazol-3-ium tetrafluoroborate, 5,7-di-tert-butyl-3-phenylbenzo[d]oxazol-3-ium triflate, 5,7-di-tert-butyl-3-(4-(trifluoromethyl)phenyl)benzo[d]oxazol-3-ium tetrafluoroborate, 5,7-di-tert-butyl-3-(4-chlorophenyl)benzo[d]oxazol-3-ium tetrafluoroborate, 5,7-di-tert-butyl-3-(4-methoxyphenyl)benzo[d]oxazol-3-ium tetrafluoroborate, and 5,7-di-tert-butyl-3-(4-(trifluoromethoxy)phenyl)benzo[d]oxazol-3-ium tetrafluoroborate.

In some embodiments, the benzoxazolium salt activator is 5,7-di-tert-butyl-3-phenylbenzo[d]oxazol-3-ium tetrafluoroborate.

In some embodiments, the reaction between Compound IV-5 or a salt thereof and the benzoxazolium salt activator occurs in the presence of a base. Suitable bases include, but are not limited to, pyridine bases (e.g., pyridine, 2,6-dimethylpyridine, etc.), and amine bases (e.g., tetramethylpiperidine, etc.).

In some embodiments, the base is a pyridine base. In some embodiments, the base is pyridine.

In some embodiments, the reaction between Compound IV-5 or a salt thereof and the benzoxazolium salt activator occurs in the presence of a solvent. Suitable solvents include, but are not limited to, ethers (e.g., tert-butyl methyl ether, cyclopentyl methyl ether, 1,4-dioxane, 2-methyltetrahydrofuran, dibutyl ether, etc.), aromatic solvents (e.g., toluene, trifluorotoluene, etc.), and mixtures of (i) an aromatic solvent or an ether with (ii) a polar aprotic solvent (e.g., N,N-dimethylacetamide, N,N-dimethylformamide, N-methyl-2-pyrrolidone, dimethyl sulfoxide, etc.).

In some embodiments, the solvent is an ether solvent. In some embodiments, the solvent is tert-butyl methyl ether.

In some embodiments, the reaction between Compound IV-5 or a salt thereof and the benzoxazolium salt activator occurs at a temperature of about −20° C. to about 50° C. In some embodiments, the temperature is about 10° C. to about 30° C. In some embodiments, the temperature is about 20° C.

In some embodiments, the reaction between Compound IV-5 or a salt thereof and the benzoxazolium salt activator occurs in the presence of a benzoxazolium salt activator (e.g., 5,7-di-tert-butyl-3-phenylbenzo[d]oxazol-3-ium tetrafluoroborate), a base such as a pyridine base (e.g., pyridine), a solvent such as an ether solvent (e.g., tert-butyl methyl ether), and at a suitable temperature such as a temperature of about 10° C. to about 30° C. (e.g., about 20° C.).

In some embodiments, the product from the reaction between Compound IV-5 or a salt thereof and the benzoxazolium salt activator (e.g., the product from step (a)) is Compound IV-7 or a salt thereof.

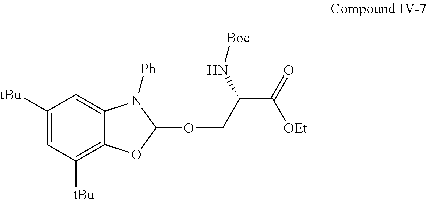 [SMILES: CCOC(=O)[C@H](COC1Oc2c(cc(C(C)(C)C)cc2C(C)(C)C)N1c1ccccc1)NC(=O)OC(C)(C)C]

In some embodiments, the process comprises contacting Compound IV-5 or a salt thereof with Compound V-1-Br or a salt thereof in the presence of a photosensitizer to provide Compound III-4 or a salt thereof.

In some embodiments, the process comprises contacting Compound IV-5 or a salt thereof with Compound V-Br or a salt thereof in the presence of a photosensitizer to provide Compound III-3 or a salt thereof.

Suitable photosensitizers (e.g., photosensitizers for providing Compound III-4 or Compound III-3) include, but are not limited to iridium photocatalyst (e.g., Ir[ppy]₂(dtbbpy)PF₆, Ir(FMeppy)₂(dtbbpy)PF₆, Ir(dFMeppy)₂(dtbbpy)PF₆, Ir(dFFppy)₂(dtbbpy)PF₆, Ir(dFCF₃ppy)₂(dtbbpy)PF₆, Ir(dFCF₃ppy)₂(dCF₃bpy)PF₆), and donor-acceptor fluorophores photocatalyst (e.g., 1,2,3,5-tetrakis(carbazol-9-yl)-4,6-dicyanobenzene, 2,4,5,6-Tetrakis(9H-carbazol-9-yl) isophthalonitrile (4CzIPN, CAS 1416881-52-1), 2,4,6-tris(diphenylamino)-5-fluoroisophthalonitrile (3DPAIPN, CAS 2260543-73-3), 3,4,5,6-tetrakis(carbazol-9-yl)-1,2-dicyanobenzene (4CzPN, CAS 1416881-51-0), 2,3,5,6-tetrakis(carbazol-9-yl)-1,4-dicyanobenzene (4CzTPN, CAS 1416881-53-2), 2,4,5,6-tetrakis(diphenylamino)isophthalonitrile (4DPAIPN, CAS 1846598-27-3), etc.).

In some embodiments, the photosensitizer is a donor-acceptor fluorophores photocatalyst. In some embodiments, the photosensitizer is 4CzIPN (CAS 1416881-52-1).

In some embodiments, the reaction between Compound IV-5 or a salt thereof and Compound V-1-Br or V-Br or a salt thereof occurs in the presence of a co-catalyst. In some embodiments, the co-catalyst is a nickel co-catalyst. Suitable nickel co-catalysts include, but are not limited to, nickel(II) bromide ethylene glycol dimethyl ether, nickel(II) chloride hexahydrate, nickel(II) chloride ethylene glycol dimethyl ether, nickel(II) tetrafluoroborate, nickel(II) acetate, nickel(II) bromide trihydrate, nickel(II) acetylacetonate, and nickel(II) stearate.

In some embodiments, the co-catalyst is nickel(II) bromide ethylene glycol dimethyl ether.

In some embodiments, the reaction between Compound IV-5 or a salt thereof and Compound V-1-Br or V-Br or a salt thereof occurs in the presence of a ligand. Suitable ligands include, but are not limited to, bipyridine ligands (e.g., 4,4′-dimethyl-2,2′-bipyridine, 4,4′-di-tert-butyl-2,2′-dipyridyl, 2,2′-dipyridine, etc.) and 1,10-phenanthroline.

In some embodiments, the ligand is a bipyridine ligand. In some embodiments, the ligand is 4,4′-dimethyl-2,2′-bipyridine.

In some embodiments, the reaction between Compound IV-5 or a salt thereof and Compound V-1-Br or V-Br or a salt thereof occurs in the presence of a base. Suitable bases include, but are not limited to, cyclic aliphatic amines (e.g., 2,2,6,6-tetramethylpiperidine, 1-methylpiperidine, pyrrolidine, piperidine, etc.), acetates (e.g., potassium acetate, lithium acetate, sodium acetate, ammonium acetate, cesium acetate, etc.), carbonates (e.g., cesium carbonate, potassium carbonate, cesium carbonate, etc.), quinuclidine-based bases (e.g., quinuclidine, 1,4-diazabicyclo[2.2.2]octane (DABCO), hydroxyquinuclidine, 3-acetoxyquinuclidine, etc.), and guanidine bases (e.g., 1,1,3,3-tetramethylguanidine, etc.).

In some embodiments, the base is a cyclic aliphatic amine. In some embodiments, the base is 2,2,6,6-tetramethylpiperidine.

In some embodiments, the reaction between Compound IV-5 or a salt thereof and Compound V-1-Br or V-Br or a salt thereof occurs in the presence of an additive. Suitable additives include, but are not limited to, phthalimide additives (e.g., phthalimide, 4-tert-butylphthalimide, 4-chloro-1,3-dioxo-1,3-dihydroisoindole, methyl 1,3-dioxoisoindoline-5-carboxylate, etc.) and succinimide additives (e.g., succinimide, 1,2,3,6-tetrahydrophthalimide, 2,2,3,3-tetramethylsuccinimide, (±)-α-methyl-α-phenylsuccinimide, bicyclo[2.2.1]heptane-2,3-dicarboximide, etc.).

In some embodiments, the additive is a phthalimide additive. In some embodiments, the additive is phthalimide. In some embodiments, the additive is succinimide.

In some embodiments, the reaction between Compound IV-5 or a salt thereof and Compound V-1-Br or V-Br or a salt thereof occurs in the presence of a solvent. Suitable solvents include, but are not limited to, ethers (e.g., tert-butyl methyl ether, cyclopentyl methyl ether, 1,4-dioxane, 2-methyltetrahydrofuran, tetrahydrofuran, dibutyl ether, etc.), polar aprotic solvents (e.g., N,N-dimethylacetamide, N,N-dimethylformamide, N,N′-dimethylpropyleneurea, 1,3-dimethyl-2-imidazolidinone, dimethyl sulfoxide, N-methyl-2-pyrrolidone, etc.), ketones (e.g., acetone, etc.), aromatic solvents (e.g., toluene, etc.), nitriles (e.g., acetonitrile, etc.), and any combination thereof.

In some embodiments, the solvent is a mixed solvent. In some embodiments, the solvent is a mixture of tert-butyl methyl ether and dimethyl sulfoxide.

In some embodiments, the reaction between Compound IV-5 or a salt thereof and Compound V-1-Br or V-Br or a salt thereof occurs at a temperature of about 0° C. to about 120° C. In some embodiments, the temperature is about 15° C. to about 35° C. In some embodiments, the temperature is about 25° C.

In some embodiments, the reaction between Compound IV-5 or a salt thereof and Compound V-1-Br or V-Br or a salt thereof occurs under a light having a wavelength of about 330 nm to about 500 nm. In some embodiments, the wavelength is about 450 nm.

In some embodiments, the reaction between Compound IV-5 or a salt thereof and Compound V-1-Br or V-Br or a salt thereof occurs in the presence of a photosensitizer such as a donor-acceptor fluorophores photocatalyst (e.g., 4CzIPN (CAS 1416881-52-1)), a co-catalyst such as a nickel co-catalyst (e.g., nickel(II) bromide ethylene glycol dimethyl ether), a ligand such as a bipyridine ligand (e.g., 4,4′-dimethyl-2,2′-bipyridine), a base such as a cyclic aliphatic amine base (e.g., 2,2,6,6-tetramethylpiperidine), an additive such as phthalimide, a solvent such as a mixture of tert-butyl methyl ether and N,N-dimethylacetamide, at a suitable temperature such as a temperature of about 15° C. to about 35° C. (e.g., about 25° C.), and under a light having a suitable wavelength such as a wavelength of about 330 nm to about 500 nm (e.g., about 450 nm).

In some embodiments, the reaction between Compound IV-5 and Compound V-1-Br (step (b1) occurs and provides Compound III-4 or a salt thereof.

In some embodiments, the reaction between Compound IV-5 and Compound V-Br (step (b2) occurs and provides Compound III-3 or a salt thereof.

In some embodiments, the reaction between Compound IV-5 and Compound V-1-Br (step (b1) occurs and provides Compound III-4 or a salt thereof, and the process further comprises contacting Compound III-4 or a salt thereof with a methylation agent to form Compound III-3 or a salt thereof.

Suitable methylation agents (e.g., the methylation agent used to provide Compound III-3 from Compound III-4) include, but are not limited to, methyl iodide, methyl bromide, methyl chloride, methyl triflate, diazomethane, dimethyl sulfate, and dimethyl carbonate.

In some embodiments, the methylation agent is methyl iodide.

In some embodiments, the methylation reaction (e.g., the methylation reaction of Compound III-4) occurs in the presence of a base. Suitable bases include, but are not limited to, amines (e.g., N,N-diisopropylethylamine, triethylamine, tripropylamine, tributylamine, 4-methylmorpholine, 1,8-diazabicyclo[5.4.0]undec-7-ene, etc.), basic aromatic compounds (e.g., pyridine, 2,6-di-tert-butylpyridine, etc.), carbonates (e.g., lithium carbonate, sodium carbonate, potassium carbonate, cesium carbonate, etc.), alkoxides (e.g., sodium methoxide, sodium tert-butoxide, lithium tert-butoxide, etc.), phosphates (e.g., sodium phosphate, potassium phosphate, etc.), and amides (e.g., lithium bis(trimethylsilyl)amide, potassium bis(trimethylsilyl)amide, lithium diisopropylamide, etc.).

In some embodiments, the base is a carbonate. In some embodiments, the base is potassium carbonate.

In some embodiments, the methylation reaction (e.g., the methylation reaction of Compound III-4) occurs in the presence of a solvent. Suitable solvents include, but are not limited to, ethers (e.g., tetrahydrofuran, 2-methyltetrahydrofuran, etc.), polar aprotic solvents (e.g., N,N-dimethylformamide, N,N-dimethylacetamide, dimethyl sulfoxide, N-methyl-2-pyrrolidone, etc.), nitriles (e.g., acetonitrile, etc.), and halogenated solvents (e.g., dichloromethane, etc.).

In some embodiments, the solvent is a polar aprotic solvent. In some embodiments, the solvent is N-methyl-2-pyrrolidone.

In some embodiments, the methylation reaction (e.g., the methylation reaction of Compound III-4) occurs at a temperature of about −10° C. to about 60° C. In some embodiments, the temperature is about 10° C. to about 30° C. In some embodiments, the temperature is about 20° C.

In some embodiments, the methylation reaction (e.g., the methylation reaction of Compound III-4 with a methylation agent such as methyl iodide) occurs in the presence of a base such as a carbonate (e.g., potassium carbonate), a solvent such as an polar aprotic solvent (e.g., N-methyl-2-pyrrolidone), and at a suitable temperature such as a temperature of about 10° C. to about 30° C. (e.g., about 20° C.).

In some embodiments, the process further comprises converting Compound III-3 or a salt thereof to Compound III or a salt thereof according to a process described anywhere herein.

### Synthesis of Compound III Via Asymmetric Cyclization (Synthesis of Compound III-4)

Also provided is the synthesis of Compound III or a salt thereof via asymmetric cyclization.

For example, Compound III-4

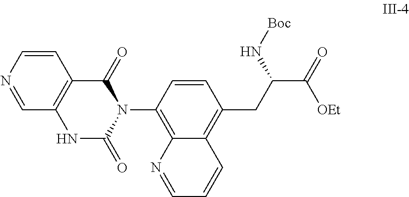

or a salt thereof may be prepared by a process comprising one or more or all of the following steps:
(a) contacting Compound IV-4

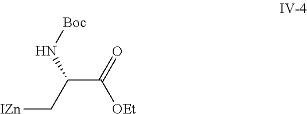

or a salt thereof with Compound VII-1

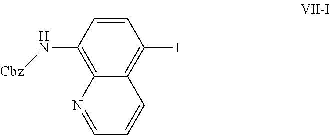 [SMILES: O=C(Nc1ccc(I)c2cccnc12)OCc1ccccc1]

or a salt thereof to form Compound X-2

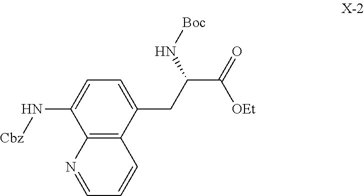 [SMILES: CCOC(=O)[C@H](Cc1ccc(NC(=O)OCc2ccccc2)c2ncccc12)NC(=O)OC(C)(C)C]

or a salt thereof;
(b) subjecting Compound X-2 or a salt thereof to a deprotection condition to form Compound X-3

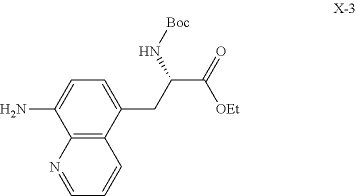 [SMILES: CCOC(=O)[C@H](Cc1ccc(N)c2ncccc12)NC(=O)OC(C)(C)C]

or a salt thereof;
(c) contacting Compound X-3 or a salt thereof with Compound VI

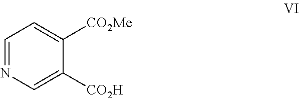 [SMILES: COC(=O)c1ccncc1C(=O)O]

or a salt thereof to form Compound III-5

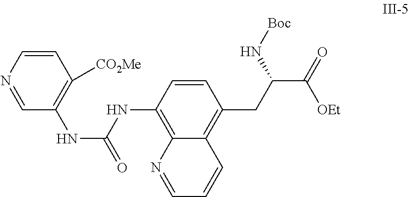 [SMILES: CCOC(=O)[C@H](Cc1ccc(NC(=O)Nc2cnccc2C(=O)OC)c2ncccc12)NC(=O)OC(C)(C)C]

or a salt thereof; and
(d) subjecting Compound III-5 or a salt thereof to a cyclization condition to form Compound III-4 or a salt thereof.

In some embodiments, provided is a process for preparing Compound III-4

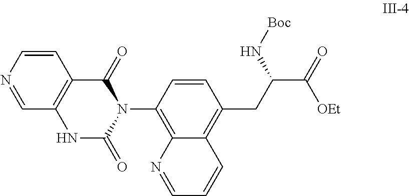

or a salt thereof, comprising:
subjecting Compound III-5

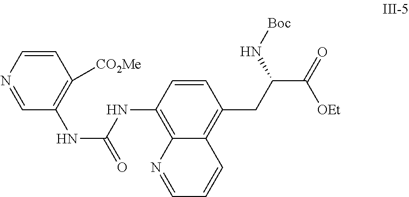 [SMILES: CCOC(=O)[C@H](Cc1ccc(NC(=O)Nc2cnccc2C(=O)OC)c2ncccc12)NC(=O)OC(C)(C)C]

or a salt thereof into a cyclization condition to form Compound III-4 or a salt thereof.

In some embodiments, Compound X-3 or a salt thereof is prepared according to a process described anywhere herein, and Compound III-4 is prepared by a process comprising: (c) contacting Compound X-3 or a salt thereof with Compound VI or a salt thereof to form Compound III-5 or a salt thereof; and (d) subjecting Compound III-5 or a salt thereof to a cyclization condition to form Compound 111-4 or a salt thereof.

In some embodiments, the process further comprises converting Compound III-4 or a salt thereof to Compound III-3 or a salt thereof according to a process described anywhere herein.

In some embodiments, the process further comprises converting Compound III-4 or a salt thereof to Compound III or a salt thereof according to a process described anywhere herein.

In some embodiments, the process comprises contacting Compound IV-4 or a salt thereof with Compound VII-1 or a salt thereof to form Compound X-2 or a salt thereof.

In some embodiments, the reaction (e.g., the reaction between Compound IV-4 and Compound VII-1) occurs in the presence of a catalyst. The catalyst may be a palladium catalyst or a nickel catalyst. The palladium catalyst can be a palladium(II) source (e.g., palladium(II) chloride, etc.) or a palladium(O) source (e.g., tris(dibenzylideneacetone)dipalladium(O), tetrakis(triphenylphosphine)palladium(O), etc.). The nickel catalyst can be a nickel(II) source (e.g., nickel(II) chloride, etc.) or a nickel(O) source (e.g., bis(cyclooctadiene)nickel(O)).

In some embodiments, the catalyst is a palladium(O) source. In some embodiments, the catalyst is tris(dibenzylideneacetone)dipalladium(O).

In some embodiments, the reaction (e.g., the reaction between Compound IV-4 and Compound VII-1) occurs in the presence of a ligand. Suitable ligands include, but are not limited to, monodentate phosphine ligands (e.g., triphenylphosphine, 2-dicyclohexylphosphino-2′,4′,6′-triisopropylbiphenyl, 2-dicyclohexylphosphino-2′,6′-dimethoxybiphenyl, etc.), bidentate phosphine ligands (e.g., 1,2-bis(diphenylphosphino)ethane, 2,2′-bis(diphenylphosphino)-1,1′-binaphthyl, 1,1′-bis(diphenylphosphino)ferrocene, etc.), bipyridine ligands (e.g., 4,4′-di-tert-butyl-2,2′-dipyridyl, 2,2′-dipyridine), 1,10-phenanthroline, and terpyridine ligands.

In some embodiments, the ligand is a monodentate phosphine ligand. In some embodiments, the ligand is 2-dicyclohexylphosphino-2′,6′-dimethoxybiphenyl.

In some embodiments, the reaction (e.g., the reaction between Compound IV-4 and Compound VII-1) occurs in the presence of a solvent. Suitable solvents include, but are not limited to, polar aprotic solvents (e.g., N,N-dimethylformamide, N,N-dimethylacetamide, N-methyl-2-pyrrolidone, N-butyl-2-pyrrolidone, dimethyl sulfoxide, etc.), ethers (e.g., tetrahydrofuran, 2-methyltetrahydrofuran, 1,4-dioxane, etc.), and nitriles (e.g., acetonitrile, benzonitrile, etc.).

In some embodiments, the solvent is a polar aprotic solvent. In some embodiments, the solvent is N,N-dimethylformamide.

In some embodiments, the reaction (e.g., the reaction between Compound IV-4 and Compound VII-1) occurs at a temperature of about 20° C. to about 120° C. In some embodiments, the temperature is about 45° C. to about 65° C. In some embodiments, the temperature is about 55° C.

In some embodiments, the reaction (e.g., the reaction between Compound IV-4 and Compound VII-1) occurs in the presence of a catalyst such as a palladium(0) source (e.g., tris(dibenzylideneacetone)dipalladium(0)), a ligand such as a monodentate phosphine ligand (e.g., 2-dicyclohexylphosphino-2′,6′-dimethoxybiphenyl), a solvent such as a polar aprotic solvent (e.g., N,N-dimethylformamide), and at a suitable temperature such as a temperature of about 45° C. to about 65° C. (e.g., about 55° C.).

In some embodiments, the process comprises subjecting Compound X-2 or a salt thereof to a deprotection condition to form Compound X-3 or a salt thereof.

In some embodiments, the deprotection condition (e.g., the condition for providing Compound X-3 from Compound X-2) comprises a catalyst. Suitable catalysts include, but are not limited to, solid-supported transition metals (e.g., platinum on carbon, palladium on carbon), Raney Nickel, transition metal catalysts (e.g., (SP-4)-(η²,η²-cycloocta-1,5-diene)(pyridine)(tricyclohexylphosphine)iridium(1+) hexafluoridophosphate(1−), (SP-4)-chloridotris(triphenylphosphene)rhodium, etc.), a palladium(II) source (e.g., palladium(II) chloride, etc.), a palladium(0) source (e.g., tetrakis(triphenylphosphine)palladium(0), etc.), a nickel(II) source (e.g., nickel(II) chloride, etc.), and a nickel(0) source (e.g., bis(cyclooctadiene)nickel(0), etc.).

In some embodiments, the catalyst is a palladium(II) source. In some embodiments, the catalyst is palladium(II) chloride.

In some embodiments, the deprotection condition (e.g., the deprotection condition for providing Compound X-3 from Compound X-2) comprises a ligand. Suitable ligands include, but are not limited to, monodentate phosphine ligands (e.g., triphenylphosphine, 2-dicyclohexylphosphino-2′,4′,6′-triisopropylbiphenyl, etc.), bidentate phosphine ligands (e.g., 1,2-bis(diphenylphosphino)ethane, 2,2′-bis(diphenylphosphino)-1,1′-binaphthyl, 1,1′-bis(diphenylphosphino)ferrocene, etc.), bipyridine ligands (e.g., 4,4′-di-tert-butyl-2,2′-dipyridyl, 2,2′-dipyridine), 1,10-phenanthroline, and terpyridine ligands.

In some embodiments, the deprotection condition (e.g., the deprotection condition for providing Compound X-3 from Compound X-2) does not comprise a ligand.

In some embodiments, the deprotection condition (e.g., the deprotection condition for providing Compound X-3 from Compound X-2) comprises a hydrogen source such as hydrogen gas, an organosilane (e.g., triethylsilane, etc.), formic acid, or a formate (e.g., sodium formate, tetrabutylammonium formate, etc).

In some embodiments, the hydrogen source is an organosilane. In some embodiments, the hydrogen source is triethylsilane.

In some embodiments, the deprotection condition (e.g., the deprotection condition for providing Compound X-3 from Compound X-2) comprises a base such as an amine base such as triethylamine, tripropylamine, and tributylamine. In some embodiments, the base is triethylamine.

In some embodiments, the deprotection condition (e.g., the deprotection condition for providing Compound X-3 from Compound X-2) comprises a solvent. Suitable solvents include, but are not limited to, alcohols (e.g., methanol, etc.), halogenated solvents (e.g., dichloromethane, etc.), polar aprotic solvents (e.g., N,N-dimethylacetamide, N-methyl-2-pyrrolidone, N-butyl-2-pyrrolidone, dimethyl sulfoxide, etc.), ethers (e.g., tetrahydrofuran, 2-methyltetrahydrofuran, 1,4-dioxane, etc.), and nitriles (e.g., acetonitrile, benzonitrile, etc.), and any combination thereof.

In some embodiments, the solvent is a mixed solvent. In some embodiments, the solvent is a mixture of methanol and dichloromethane.

In some embodiments, the deprotection condition (e.g., the deprotection condition for providing Compound X-3 from Compound X-2) comprises a temperature of about 0° C. to about 60° C. In some embodiments, the temperature is about 10° C. to about 30° C. In some embodiments, the temperature is about 20° C.

In some embodiments, the deprotection condition (e.g., the deprotection condition for providing Compound X-3 from Compound X-2) comprises a catalyst such as a palladium(II) source (e.g., palladium(II) chloride), a hydrogen source such as an organosilane (e.g., triethylsilane), a base such as an amine base (e.g., triethylamine), a solvent such as a mixture of methanol and dichloromethane, and at a suitable temperature such as a temperature of about 10° C. to about 30° C. (e.g., about 20° C.).

In some embodiments, the process comprises contacting Compound X-3 or a salt thereof with Compound VI or a salt thereof to form Compound III-5 or a salt thereof.

In some embodiments, the reaction (e.g., the reaction between Compound X-3 and Compound VI) occurs in the presence of a base. Suitable bases include, but are not limited to, amines (e.g., triethylamine, N,N-diisopropylethylamine, tripropylamine, tributylamine, 4-methylmorpholine, 1,4-diazabicylo[2.2.2]-octane, 1,8-diazabicyclo[5.4.0]undec-7-ene, etc.) and basic aromatic compounds (e.g., pyridine, 2,6-lutidine, imidazole, etc.).

In some embodiments, the base is an amine base. In some embodiments, the base is triethylamine.

In some embodiments, the reaction (e.g., the reaction between Compound X-3 and Compound VI) occurs in the presence of an azide source (e.g., diphenylphosphoryl azide (DPPA), trimethylsilyl azide, sodium azide, lithium azide, 4-carboxybenzenesulfonazide, 4-acetamidobenenesulfonyl azide, etc.) or deoxo-fluor.

In some embodiments, the reaction (e.g., the reaction between Compound X-3 and Compound VI) occurs in the presence of an azide source. In some embodiments, the azide source is diphenylphosphoryl azide (DPPA).

In some embodiments, the reaction (e.g., the reaction between Compound X-3 and Compound VI) occurs in the presence of a solvent. Suitable solvents include, but are not limited to, ethers (e.g., tetrahydrofuran, 2-methyltetrahydrofuran, 1,4-dioxane, etc.), aromatic solvents (e.g., benzene, toluene, trifluorotoluene, etc.), nitriles (e.g., acetonitrile, benzonitrile, etc.), and halogenated solvents (e.g., dichloromethane, 1,2-dichloroethane, chlorobenzene, etc.).

In some embodiments, the solvent is an aromatic solvent. In some embodiments, the solvent is toluene.

In some embodiments, the reaction (e.g., the reaction between Compound X-3 and Compound VI) occurs at a temperature of about −10° C. to about 120° C. In some embodiments, the temperature is about 50° C. to about 70° C. In some embodiments, the temperature is about 60° C.

In some embodiments, the reaction (e.g., the reaction between Compound X-3 and Compound VI) occurs in the presence of a base such as an amine base (e.g., triethylamine), an azide source such as diphenylphosphoryl azide (DPPA), a solvent such as an aromatic solvent (e.g., toluene), and at a suitable temperature such as a temperature of about 50° C. to about 70° C. (e.g., about 60° C.).

In some embodiments, the process comprises subjecting Compound III-5 or a salt thereof to a cyclization condition to form Compound III-4 or a salt thereof.

In some embodiments, the process comprises subjecting Compound III-5 or a salt thereof to a cyclization condition to form Compound III-4 or a salt thereof.

In some embodiments, the cyclization condition (e.g., the cyclization condition for providing Compound III-4 from Compound III-5) comprises an organocatalyst. Suitable organocatalysts include, but are not limited to, squaramides (e.g., 3-[[3,5-bis(trifluoromethyl)phenyl]amino]-4-[(8a,9s)-cinchonan-9-ylamino]-3-cyclobutene-1,2-dione (CAS 1256245-86-9), 3-[[3,5-bis(trifluoromethyl)phenyl]amino]-4-[[(8a,9S)-6′-methoxycinchonan-9-yl]amino]-3-cyclobutene-1,2-dione (CAS 1256245-84-7), 3-[[3,5-bis(trifluoromethyl)phenyl]amino]-4-[[(8a,9S)-10,11-dihydrocinchonan9-yl]amino]-3-cyclobutene-1,2-dione (CAS 1407166-62-4), 3-((3,5-bis(trifluoromethyl)phenyl)amino)-4-(((1S,2S)-1,2-diphenyl-2-(piperidin-1-yl)ethyl)amino)cyclobut-3-ene-1,2-dione (CAS 1943732-17-9), 3-[[3,5-bis(trifluoromethyl)phenyl]amino]-4-[[(1S,2S)-2-(1-piperidinyl)cyclohexyl]amino]-3-Cyclobutene-1,2-dione (CAS 1312991-15-3), etc.), chiral guanidines (e.g., (2R,6R)-2,6-di-tert-butyl-2,3,5,6-tetrahydro-1H-imidazo[1,2-a]imidazole (CAS 1502026-83-6), etc.), and chiral phase transfer catalysts.

In some embodiments, the organocatalyst is a squaramide organocatalyst. In some embodiments, the organocatalyst is CAS 1256245-86-9.

In some embodiments, the cyclization condition (e.g., the cyclization condition for providing Compound III-4 from Compound III-5) comprises a base. Suitable bases include, but are not limited to, carbonates (e.g., lithium carbonate, sodium carbonate, potassium carbonate, cesium carbonate, etc.), alkoxides (e.g., sodium methoxide, sodium tert-butoxide, lithium tert-butoxide, etc.), phosphates (e.g., sodium phosphate, potassium phosphate, etc.), amines (e.g., triethylamine, N,N-diisopropylethylamine, tripropylamine, tributylamine, 4-methylmorpholine, 1,8-diazabicyclo(5.4.0)undec-7-ene, etc.), basic aromatic compounds (e.g., pyridine, 2,6-di-tert-butylpyridine, etc.), and amides (e.g., lithium bis(trimethylsilyl)amide, potassium bis(trimethylsilyl)amide, lithium diisopropylamide, etc.).

In some embodiments, the base is a carbonate. In some embodiments, the base is cesium carbonate.

In some embodiments, the cyclization condition (e.g., the cyclization condition for providing Compound III-4 from Compound III-5) comprises a solvent. Suitable solvents include, but are not limited to, ethers (e.g., tetrahydrofuran, 2-methyltetrahydrofuran, etc.), polar aprotic solvents (e.g., N,N-dimethylformamide, N,N-dimethylacetamide, N-methyl-2-pyrrolidone, dimethyl sulfoxide, etc.), nitriles (e.g., acetonitrile), and halogenated solvents (e.g., dichloromethane).

In some embodiments, the solvent is a polar aprotic solvent. In some embodiments, the solvent is N,N-dimethylformamide.

In some embodiments, the cyclization condition (e.g., the cyclization condition for providing Compound III-4 from Compound III-5) comprises a temperature of about 0° C. to about 50° C. In some embodiments, the temperature is about 10° C. to about 30° C. In some embodiments, the temperature is about 20° C.

In some embodiments, the cyclization condition (e.g., the cyclization condition for providing Compound III-4 from Compound III-5) comprises an organocatalyst such as a squaramide organocatalyst (e.g., CAS 1256245-86-9), a base such as a carbonate (e.g., cesium carbonate), a solvent such as a polar aprotic solvent (e.g., N,N-dimethylformamide), and a suitable temperature of about 10° C. to about 30° C. (e.g., about 20° C.).

### Synthesis of Compound III Via Erlenmeyer Reaction

Also provided is the synthesis of Compound III or a salt thereof via Erlenmeyer reaction.

For example, Compound IIIa

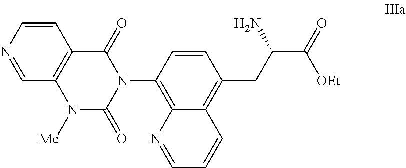 [SMILES: CCOC(=O)[C@@H](N)Cc1ccc(-n2c(=O)c3ccncc3n(C)c2=O)c2ncccc12]

or a salt thereof may be prepared by a process comprising one or more or all of the following steps:
(a) subjecting Compound Va-I

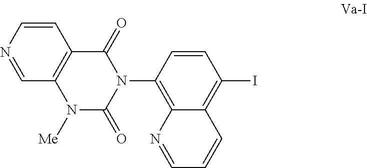 [SMILES: Cn1c(=O)n(-c2ccc(I)c3cccnc23)c(=O)c2ccncc21]

or a salt thereof to a reductive carbonylation condition to form Compound V-4a

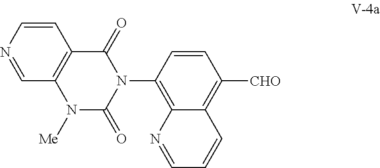 [SMILES: Cn1c(=O)n(-c2ccc(C=O)c3cccnc23)c(=O)c2ccncc21]

or a salt thereof;
(b) contacting Compound V-4a or a salt thereof with N-acetylglycine to form Compound III-7a

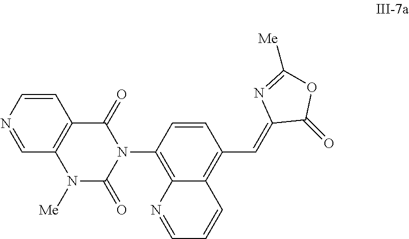 [SMILES: CC1=N/C(=C\c2ccc(-n3c(=O)c4ccncc4n(C)c3=O)c3ncccc23)C(=O)O1]

or a salt thereof;
(c) subjecting Compound III-7a or a salt thereof to a ring opening condition to form a compound of Formula III-8a

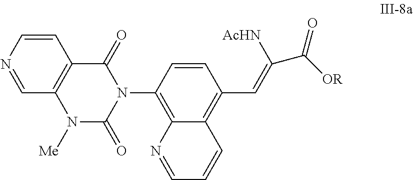

wherein R is a C₁₋₄ alkyl,
or a salt thereof;
(d) subjecting a compound of Formula III-8a or a salt thereof to an asymmetric hydrogenation condition to form a compound of Formula III-9a

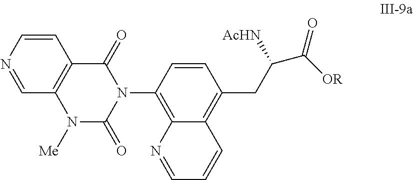

wherein R is a C₁₋₄ alkyl,
or a salt thereof; and
(e) converting a compound of Formula III-9a or a salt thereof to Compound IIIa or a salt thereof.

In some embodiments, R is methyl or ethyl. In some embodiments, R is ethyl.

In some embodiments, the process further comprises: (f) converting Compound IIIa

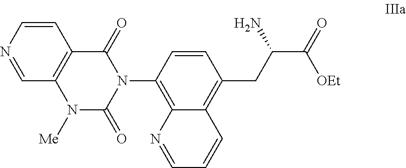 [SMILES: CCOC(=O)[C@@H](N)Cc1ccc(-n2c(=O)c3ccncc3n(C)c2=O)c2ncccc12]

or a salt thereof to Compound III

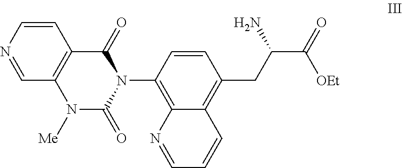

or a salt thereof according to a process described anywhere herein.

In some embodiments, the process comprises subjecting Compound Va-I or a salt thereof to a reductive carbonylation condition to form Compound V-4a or a salt thereof.

In some embodiments, R is a C₁₋₄ alkyl. In some embodiments, R is methyl or ethyl.

In some embodiments, the reductive carbonylation condition (e.g., the carbonylation condition for providing form Compound V-4a from Compound Va-I) comprises a carbonylation source (e.g., carbon monoxide, molybdenum hexacarbonyl, etc.) and a hydride source (e.g., hydrogen gas, triethylsilane, sodium formate, etc.).

In some embodiments, syngas (a mixture of carbon monoxide and hydrogen gas) serves as both carbonylation source and hydride source.

In some embodiments, the reductive carbonylation condition (e.g., the carbonylation condition for providing form Compound V-4a from Compound Va-I) comprises a base, such as an amine base (e.g., N,N,N′,N′-Tetramethylethylenediamine (TMEDA), triethylamine, N,N-diisopropylethylamine, tributylamine, tripropylamine, etc.).

In some embodiments, the base is N,N,N′,N′-Tetramethylethylenediamine (TMEDA).

In some embodiments, the reductive carbonylation condition (e.g., the carbonylation condition for providing Compound V-4a from Compound Va-I) comprises a catalyst such as a palladium catalyst. For example, the catalyst may be a palladium(II) source (e.g., palladium(II) acetate, palladium(II) chloride, etc.) or a palladium(0) source (e.g., tetrakis(triphenylphosphine)palladium(0), etc.).

In some embodiments, the catalyst is a palladium(II) source. In some embodiments, the catalyst is palladium(II) acetate.

In some embodiments, the reductive carbonylation condition (e.g., the carbonylation condition for providing Compound V-4a from Compound Va-I) comprises a ligand, such as a monodentate or bidentate phosphine ligand. Suitable ligands include, but are not limited to, di(1-adamantyl)-n-butylphosphine (cataCXium A), 1,1′-binaphthyl-2,2′-diphemyl phosphine (rac-BINAP), dicyclohexyl (2′,6′-dimethoxy[1,1′-biphenyl]-2-yl)phosphine (SPhos), 4,5-bis(diphenylphosphino)-9,9-dimethylxanthene (XantPhos), 2-(dicyclohexylphosphino)-3,6-dimethoxy-2′-4′-6′-tri-i-propyl-1,1′-biphenyl (BrettPhos), 2-dicyclohexylphosphino-2′-(N,N-dimethylamino)-biphenyl (DavePhos), P^{t}Bu₃, and JosiPhos ligands.

In some embodiments, the ligand is di(1-adamantyl)-n-butylphosphine (cataCXium A).

In some embodiments, the reductive carbonylation condition (e.g., the carbonylation condition for providing Compound V-4a from Compound Va-I) comprises a drying agent such as a molecular sieve (e.g., 3 Å molecular sieves).

In some embodiments, the reductive carbonylation condition (e.g., the carbonylation condition for providing Compound V-4a from Compound Va-I) comprises a solvent. Suitable solvents include, but are not limited to, aromatic solvents (e.g., toluene, xylene, etc.), polar aprotic solvents (e.g., N,N-dimethylacetamide, N,N-dimethylformamide, N-methyl-2-pyrrolidone, dimethyl sulfoxide, etc.), ethers (e.g., tetrahydrofuran, 2-methyltetrahydrofuran, 1,4-dioxane, etc.), nitriles (acetonitrile, benzonitrile, etc.), and any combination thereof.

In some embodiments, the solvent is a mixed solvent. In some embodiments, the solvent is a mixture of toluene and N,N-dimethylacetamide.

In some embodiments, the reductive carbonylation condition (e.g., the carbonylation condition for providing Compound V-4a from Compound Va-I) comprises a temperature of about 20° C. to about 120° C. In some embodiments, the temperature is about 75° C. to about 95° C. In some embodiments, the temperature is about 85° C.

In some embodiments, the reductive carbonylation condition (e.g., the carbonylation condition for providing Compound V-4a from Compound Va-I) comprises an elevated pressure. In some embodiments, the pressure is about 15 psig to about 200 psig. In some embodiments, the pressure is about 140 psig to about 160 psig. In some embodiments, the pressure is about 150 psig.

In some embodiments, the reductive carbonylation condition (e.g., the carbonylation condition for providing Compound V-4a from Compound Va-I) comprises a carbonylation source and a hydride source such as syngas, a catalyst such as a palladium (II) source (e.g., palladium(II) acetate), a ligand such as a monodentate or bidentate phosphine ligand (e.g., di(1-adamantyl)-n-butylphosphine (cataCXium A)), a drying agent such as a 3 Å molecular sieve, a solvent such as a mixture of toluene and N,N-dimethylacetamide, a suitable temperature such as a temperature of about 75° C. to about 95° C. (e.g., about 85° C.), and an elevated pressure such as a pressure of about 140 psig to about 160 psig (e.g., about 150 psig).

In some embodiments, the process comprises contacting Compound V-4a or a salt thereof with N-acetylglycine to form Compound III-7a or a salt thereof.

In some embodiments, the reaction (e.g., the reaction for proving Compound III-7a from Compound V-4a) occurs in the presence of a base. Suitable base include, but are not limited to, carbonates (e.g., lithium carbonate, sodium carbonate, potassium carbonate, etc.), phosphates (e.g., tripotassium phosphate, dipotassium phosphate, trisodium phosphate, trilithium phosphate, etc.), alkoxides (e.g., sodium methoxide, sodium tert-butoxide, potassium tert-butoxide, etc.), amides (e.g., lithium bis(trimethylsilyl)amide, etc.), and amines (e.g., triethylamine, N,N-diisopropylethylamine, tributylamine, tripropylamine, etc.).

In some embodiments, the base is a phosphate base. In some embodiments, the base is tripotassium phosphate.

In some embodiments, the reaction (e.g., the reaction for proving Compound III-7a from Compound V-4a) occurs in the presence of a solvent. The solvent may be acetic anhydride or a mixture of acetic anhydride and another solvent such as an ether (e.g., cyclopentyl methyl ether, 1,4-dioxane, 2-methyltetrahydrofuran, dibutyl ether, tert-butyl, methyl ether, tetrahydrofuran etc.), a nitrile (e.g., acetonitrile, etc.), a polar aprotic solvent (e.g., dimethylacetamide, N,N′-dimethylpropyleneurea, 1,3-dimethyl-2-imidazolidinone, dimethyl sulfoxide, N-methyl-2-pyrrolidone, N,N-dimethylformamide, etc.), or an aromatic solvent (e.g., toluene, etc.).

In some embodiments, the solvent is acetic anhydride.

In some embodiments, the reaction (e.g., the reaction for proving Compound III-7a from Compound V-4a) occurs at a temperature of about 20° C. to about 120° C. In some embodiments, the temperature is about 75° C. to about 95° C. In some embodiments, the temperature is about 85° C.

In some embodiments, the reaction (e.g., the reaction for proving Compound III-7a from Compound V-4a) occurs in the presence of a base such as a phosphate base (e.g., tripotassium phosphate), a solvent such as acetic anhydride, and at a suitable temperature such as a temperature of about 75° C. to about 95° C. (e.g., about 85° C.).

In some embodiments, the process comprises subjecting Compound III-7a or a salt thereof to a ring opening condition to form a compound of Formula III-8a.

In some embodiments, the ring opening condition (e.g., the condition for converting Compound III-7a to a compound of Formula III-8a) comprises a base. Suitable bases include, but are not limited to, carbonates (e.g., lithium carbonate, sodium carbonate, potassium carbonate, etc.), phosphates (e.g., tripotassium phosphate, trisodium phosphate, trilithium phosphate, etc.), alkoxides (e.g., sodium methoxide, sodium tert-butoxide, potassium tert-butoxide, etc.), and acetates (e.g., sodium acetate, potassium acetate, lithium acetate, tetrabutylammonium acetate, etc.).

In some embodiments, the base is an acetate base. In some embodiments, the base is sodium acetate.

In some embodiments, the ring opening condition (e.g., the condition for converting Compound III-7a to a compound of Formula III-8a) comprises a solvent. The solvent may be ethanol or methanol. Additionally or alternatively, the solvent may be a mixture of (i) ethanol or methanol and (ii) another solvent such as an ether (e.g., cyclopentyl methyl ether, 1,4-dioxane, 2-methyltetrahydrofuran, dibutyl ether, tertbutyl methyl ether, tetrahydrofuran, etc.), a nitrile (e.g., acetonitrile, etc.), a polar aprotic solvent (e.g., dimethylacetamide, N,N′-dimethylpropyleneurea, 1,3-dimethyl-2-imidazolidinone, dimethyl sulfoxide, N-methyl-2-pyrrolidone, N,N-dimethylformamide, etc.), an aromatic solvent (e.g., toluene, etc.).

In some embodiments, the solvent is ethanol or methanol.

In some embodiments, the ring opening condition (e.g., the condition for converting Compound III-7a to a compound of Formula III-8a) comprises a temperature of about 20° C. to about 120° C. In some embodiments, the temperature is about 50° C. to about 70° C. In some embodiments, the temperature is about 60° C.

In some embodiments, the ring opening condition (e.g., the condition for converting Compound III-7a to a compound of Formula III-8a) comprises a base such as an acetate base (e.g., sodium acetate), a solvent such as ethanol or methanol, and a suitable temperature such as a temperature of about 50° C. to about 70° C. (e.g., about 60° C.).

In some embodiments, the process comprises subjecting a compound of Formula III-8a or a salt thereof to an asymmetric hydrogenation condition to form a compound of Formula III-9a or a salt thereof.

In some embodiments, the asymmetric hydrogenation condition (e.g., the condition for converting a Compound of Formula III-8a to a compound of Formula III-9a) comprises a catalyst. Suitable catalysts include, but are not limited to, rhodium(I) sources (e.g., bis(1,5-cyclooctadiene)rhodium(I) trifluoromethanesulfonate, bis(1,5-cyclooctadiene)rhodium(I) tetrafluoroborate, tris(triphenylphosphine)rhodium(I) chloride, etc.), ruthenium(II) sources (e.g., bis(2-methylallyl) (1,5-cyclooctadiene)ruthenium(II), etc.), and nickel(II) sources (e.g., nickel(II) acetate tetrahydrate, etc.).

In some embodiments, the catalyst is a rhodium(I) source. In some embodiments, the catalyst is bis(1,5-cyclooctadiene)rhodium(I) trifluoromethanesulfonate.

In some embodiments, the asymmetric hydrogenation condition (e.g., the condition for converting a Compound of Formula III-8a to a compound of Formula III-9a) comprises a ligand, such as a chiral phosphine ligand. Suitable phosphine ligand ligands include, but are not limited to, (1R)-1-(diphenylphosphino)2-[(1R)-1-[(diphenylphosphino)methylamino]ethyl]ferrocene, 2,2′-bis(diphenylphosphino)-1,1′-binaphthyl, and 1,2-bis[2,5-dimethylphospholano]benzene, 2,2′-di-tert-butyl-2,3,2′,3′-tetrahydro-1H,1′H-(1,1′)biisophosphindolyl.

In some embodiments, the ligand is (1R)-1-(diphenylphosphino)2-[(1R)-1-[(diphenylphosphino)methylamino]ethyl]ferrocene.

In some embodiments, the asymmetric hydrogenation condition (e.g., the condition for converting a Compound of Formula III-8a to a compound of Formula III-9a) comprises a hydrogen source. Suitable hydrogen sources include, but are not limited to, hydrogen gas, formic acid, sodium formate, and tetrabutylammonium formate.

In some embodiments, the hydrogen source is hydrogen gas.

In some embodiments, the asymmetric hydrogenation condition (e.g., the condition for converting a Compound of Formula III-8a to a compound of Formula III-9a) comprises a solvent. Suitable solvents include, but are not limited to, alcohols (e.g., methanol, ethanol, 2-propanol, etc.), aromatic solvents (e.g., toluene, etc.), polar aprotic solvents (e.g., N,N-dimethylformamide, N,N-dimethylacetamide, N-methyl-2-pyrrolidone, dimethyl sulfoxide, etc.), ethers (e.g., tetrahydrofuran, 2-methyltetrahydrofuran, 1,4-dioxane, etc.), and nitriles (e.g., acetonitrile, benzonitrile, etc.).

In some embodiments, the solvent is an alcohol. In some embodiments, the solvent is methanol.

In some embodiments, the asymmetric hydrogenation condition (e.g., the condition for converting a Compound of Formula III-8a to a compound of Formula III-9a) comprises a temperature of about 20° C. to about 120° C. In some embodiments, the temperature is about 40° C. to about 60° C. In some embodiments, the temperature is about 50° C.

In some embodiments, the asymmetric hydrogenation condition (e.g., the condition for converting a Compound of Formula III-8a to a compound of Formula III-9a) comprises a pressure of about 10 psig to about 100 psig. In some embodiments, the pressure is about 40 psig.

In some embodiments, the asymmetric hydrogenation condition (e.g., the condition for converting a Compound of Formula III-8a to a compound of Formula III-9a) comprises a catalyst such as a rhodium(I) source (e.g., bis(1,5-cyclooctadiene)rhodium(I) trifluoromethanesulfonate), a ligand such as a chiral phosphine ligand (e.g., (1R)-1-(diphenylphosphino)2-[(1R)-1-[(diphenylphosphino)methylamino]ethyl]ferrocene), a hydrogen source such as hydrogen gas, a solvent such as an alcohol solvent (e.g., methanol), a suitable temperature such as a temperature of about 40° C. to about 60° C. (e.g., about 50° C.), and a suitable pressure such as a pressure of about 40 psig.

In some embodiments, the process comprises converting a compound of Formula III-9a or a salt thereof to Compound IIIa or a salt thereof.

In some embodiments, the reaction (e.g., the reaction for providing Compound IIIa from a compound of Formula III-9a) occurs in the presence of an acid. Suitable acids include, but are not limited to, sulfonic acids (e.g., methanesulfonic acid, p-toluenesulfonic acid, etc.), mineral acids (e.g., hydrochloric acid, hydrobromic acid, sulfuric acid, phosphoric acid, etc.), and carboxylic acids (e.g., acetic acid, pivalic acid, trifluoroacetic acid, etc.).

In some embodiments, the acid is a sulfonic acid. In some embodiments, the acid is methanesulfonic acid.

In some embodiments, the reaction (e.g., the reaction for providing Compound IIIa from a compound of Formula III-9a) occurs in the presence of a solvent. In some embodiments, the solvent is ethanol. In some embodiments, the solvent is selected from ethers (e.g., tetrahydrofuran, 2-methyltetrahydrofuran, tert-butyl methyl ether, etc.), esters (e.g., ethyl acetate, isopropyl acetate, etc.), nitriles (e.g., acetonitrile, etc.), halogenated solvents (e.g., dichloromethane, 1,2-dichloroethane, etc.), and a combination of any of the foregoing with ethanol.

In some embodiments, the solvent is ethanol.

In some embodiments, the reaction (e.g., the reaction for providing Compound IIIa from a compound of Formula III-9a) occurs at a temperature of about 30° C. to about 120° C. In some embodiments, the temperature is about 65° C. to about 85° C. In some embodiments, the temperature is about 75° C.

In some embodiments, the reaction (e.g., the reaction for providing Compound IIIa from a compound of Formula III-9a) occurs in the presence of an acid such as a sulfonic acid (e.g., methanesulfonic acid), a solvent such as ethanol, and at a suitable temperature such as a temperature of about 65° C. to about 85° C. (e.g., about 75° C.).

### Synthesis of Compound III Via Heck Reaction

Also provided is the synthesis of Compound III or a salt thereof via Heck reaction.

For example, Compound III-8a

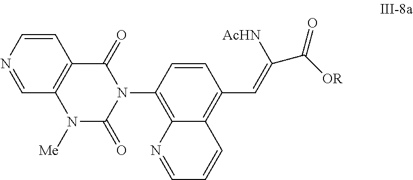

wherein R is a C₁₋₄ alkyl, In some embodiments, R is methyl or ethyl. In some embodiments, R is ethyl.
or a salt thereof may be prepared by a process comprising one or more or all of the following steps:
(a) subjecting an alkyl serinate:

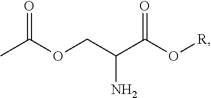

wherein R is a C₁₋₄ alkyl,
or a salt thereof to an acylation condition to form a compound of Formula IV-9

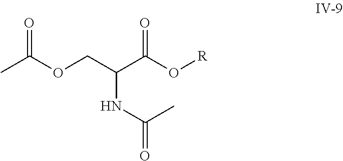

wherein R is a C₁₋₄ alkyl,
or a salt thereof;
(b) subjecting a compound of Formula IV-9 or a salt thereof to a dehydration condition to form a compound of Formula IV-10

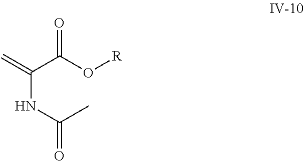

wherein R is a C₁₋₄ alkyl,
or a salt thereof; and
(c) contacting a compound of Formula IV-10 or a salt thereof with Compound Va-I

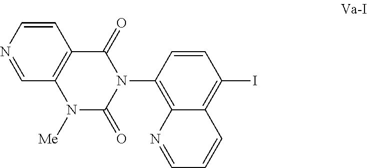 [SMILES: Cn1c(=O)n(-c2ccc(I)c3cccnc23)c(=O)c2ccncc21]

or a salt thereof to form Compound III-8a or a salt thereof.

In some embodiments, the process further comprises: (d) converting III-8a

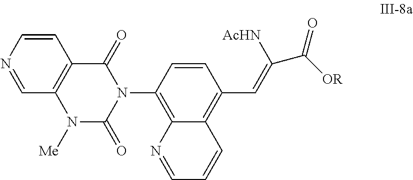

wherein R is a C₁₋₄ alkyl,
or a salt thereof to Compound IIIa

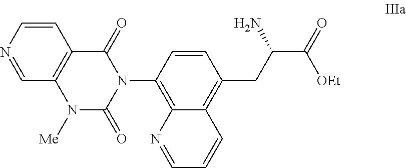 [SMILES: CCOC(=O)[C@@H](N)Cc1ccc(-n2c(=O)c3ccncc3n(C)c2=O)c2ncccc12]

or a salt thereof according to a process described anywhere herein.

In some embodiments, the process further comprises: (e) converting Compound IIIa

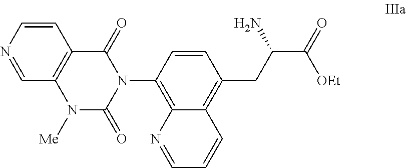 [SMILES: CCOC(=O)[C@@H](N)Cc1ccc(-n2c(=O)c3ccncc3n(C)c2=O)c2ncccc12]

or a salt thereof to Compound III

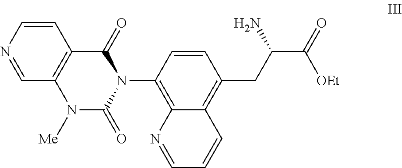

or a salt thereof according to a process described anywhere herein.

In some embodiments, R is a C₁₋₄ alkyl. In some embodiments, R is methyl or ethyl.

In some embodiments, the process comprises subjecting an alkyl serinate (e.g., methyl serinate) or a salt thereof to an acylation condition to form a compound of Formula IV-9 or a salt thereof.

In some embodiments, the acylation condition (e.g., the condition for providing a compound of Formula IV-9 from alkyl serinate) comprises an acyl source (e.g., an acetyl source) and an optional coupling reagent. The acyl source may be acetic anhydride, acetyl chloride, or acetic acid; and the coupling reagent, when present, may be 1,1′-carbonyldiimidazole.

In some embodiments, the acyl source is acetic anhydride.

In some embodiments, the acylation condition (e.g., the condition for providing a compound of Formula IV-9 from alkyl serinate) comprises a base. Suitable bases include, but are not limited to, amines (e.g., triethylamine, N,N-diisopropylethylamine, 4-methylmorpholine, tributylamine, 1,8-diazabicyclo[5.4.0]undec-7-ene, 1,5-diazabicyclo[4.3.0]non-5-ene, etc.), carbonates (e.g., lithium carbonate, sodium carbonate, potassium carbonate, cesium carbonate, etc.), alkoxides (e.g., sodium methoxide, sodium tert-butoxide, lithium tert-butoxide, etc.), and phosphates (e.g., sodium phosphate, potassium phosphate, etc.).

In some embodiments, the base is an amine base. In some embodiments, the base is triethylamine.

In some embodiments, the acylation condition (e.g., the condition for providing a compound of Formula IV-9 from alkyl serinate) comprises a catalyst. The catalyst can be a nucleophilic compound, such as a N-containing heterocycle. Suitable catalysts include, but are not limited to, 4-(dimethylamino)pyridine, 1-methylimidazole, pyridine, and 4-methylpyridine.

In some embodiments, the catalyst is 4-(dimethylamino)pyridine.

In some embodiments, the acylation condition (e.g., the condition for providing a compound of Formula IV-9 from alkyl serinate) comprises a solvent. Suitable solvents include, but are not limited to, esters (e.g., ethyl acetate, isopropyl acetate, etc.), ethers (e.g., tetrahydrofuran, 2-methyltetrahydrofuran, tert-butyl methyl ether, etc.), halogenated solvents (e.g., dichloromethane, 1,2-dichloroethane, chlorobenzene, etc.), hydrocarbons (e.g., benzene, toluene, etc.), and polar aprotic solvents (e.g., N,N-dimethylformamide, N,N-dimethylacetamide, N-methyl-2-pyrrolidone, dimethyl sulfoxide, etc.).

In some embodiments, the solvent is a halogenated solvent. In some embodiments, the solvent is dichloromethane.

In some embodiments, the acylation condition (e.g., the condition for providing a compound of Formula IV-9 from alkyl serinate) comprises a temperature of about −20° C. to about 50° C. In some embodiments, the temperature is about −5° C. to about 15° C. In some embodiments, the temperature is about 5° C.

In some embodiments, the acylation condition (e.g., the condition for providing a compound of Formula IV-9 from alkyl serinate) comprises an acyl source (e.g., an acetyl source) such as acetic anhydride, a base such as an amine base (e.g., triethylamine), a catalyst such as a N-containing heterocycle (4-(dimethylamino)pyridine), a solvent such as a halogenated solvent (e.g., dichloromethane), and a suitable temperature such a temperature of about −5° C. to about 15° C. (e.g., about 5° C.).

In some embodiments, the process comprises subjecting a compound of Formula IV-9 or a salt thereof to a dehydration condition to form compound of Formula IV-10 or a salt thereof.

In some embodiments, the dehydration condition (e.g., the condition for providing a compound of Formula IV-10 from a compound of Formula IV-9) comprises a base. Suitable bases include, but are not limited to, amines (e.g., triethylamine, N,N-diisopropylethylamine, tributylamine, 1,8-diazabicyclo[5.4.0]undec-7-ene, 1,5-diazabicyclo[4.3.0]non-5-ene, etc.), heterocyclic bases (e.g., pyridine), carbonates (e.g., sodium carbonate, lithium carbonate, potassium carbonate), acetates (e.g., lithium acetate, sodium acetate, potassium acetate, tetrabutylammonium acetate, etc.), phosphates such as phosphate tribasic (e.g., potassium phosphate tribasic, etc.), alkoxides (e.g., sodium methoxide, sodium tert-butoxide, potassium tert-butoxide, etc.), lithium bis(trimethylsilyl)amide, and hydroxides (e.g., potassium hydroxide, sodium hydroxide, etc.).

In some embodiments, the base is an amine base. In some embodiments, the base is triethylamine.

In some embodiments, the dehydration condition (e.g., the condition for providing a compound of Formula IV-10 from a compound of Formula IV-9) comprises a solvent. Suitable solvents include, but are not limited to, ethers (e.g., tetrahydrofuran, 2-methyltetrahydrofuran, tert-butyl methyl ether, etc.), hydrocarbons (e.g., toluene, etc.), halogenated solvents (e.g., 1,2-dichloroethane, chlorobenzene, etc.), polar aprotic solvents (e.g., N,N-dimethylformamide, N,N-dimethylacetamide, N-methyl-2-pyrrolidone, dimethyl sulfoxide, etc.), nitriles (e.g., acetonitrile, benzonitrile, etc.), hydrocarbons (e.g., benzene, toluene, etc.), and halogenated solvents (e.g., dichloromethane, 1,2-dichloroethane, etc.).

In some embodiments, the solvent is a polar aprotic solvent. In some embodiments, the solvent is N,N-dimethylformamide.

In some embodiments, the dehydration condition (e.g., the condition for providing a compound of Formula IV-10 from a compound of Formula IV-9) comprises a temperature of about −30° C. to about 120° C. In some embodiments, the temperature is about 80° C. to about 100° C. In some embodiments, the temperature is about 90° C.

In some embodiments, the dehydration condition (e.g., the condition for providing a compound of Formula IV-10 from a compound of Formula IV-9) comprises a base such as an amine base (e.g., triethylamine), a solvent such as a polar aprotic solvent (e.g., N,N-dimethylformamide), a suitable temperature such as a temperature of about 80° C. to about 100° C. (e.g., about 90° C.).

In some embodiments, the compound of Formula IV-10 or a salt thereof obtained from step (b) is subject to the next step (e.g., step (c)) without isolation and/or purification.

In some embodiments, the process comprises contacting a compound of Formula IV-10 or a salt thereof with Compound Va-I or a salt thereof to form a compound of Formula III-8a or a salt thereof.

In some embodiments, the reaction (e.g., the reaction between a compound of Formula IV-10 and Compound Va-I) occurs in the presence of a catalyst such as a palladium catalyst. The palladium catalyst can be a palladium(II) source (e.g., palladium(II) acetate, palladium(II) chloride, etc.) or a palladium(0) source (e.g., tetrakis(triphenylphosphine)palladium(0), etc.).

In some embodiments, the catalyst is a palladium(II) source. In some embodiments, the catalyst is palladium(II) acetate.

In some embodiments, the reaction (e.g., the reaction between a compound of Formula IV-10 and Compound Va-I) occurs in the presence of a ligand. Suitable ligands include, but are not limited to, phosphine ligands (e.g., 2,2′-bis(diphenylphosphino)-1,1′-binaphthyl, etc.) and N-heterocyclic carbene ligands (e.g., 1,3-bis(1-adamantyl)imidazolium tetrafluoroborate, etc.).

In some embodiments, the reaction (e.g., the reaction between a compound of Formula IV-10 and Compound Va-I) occurs in the absence of a ligand.

In some embodiments, the reaction (e.g., the reaction between a compound of Formula IV-10 and Compound Va-I) occurs in the presence of a base. Suitable bases include, but are not limited to, carbonates (e.g., sodium carbonate, lithium carbonate, potassium carbonate, etc.), acetates (e.g., lithium carbonate, sodium carbonate, potassium carbonate, tetrabutylammonium carbonate, etc.), phosphates such as phosphate tribasic (e.g., potassium phosphate tribasic, etc.), alkoxides (e.g., sodium methoxide, sodium tert-butoxide, potassium tert-butoxide, etc.), lithium bis(trimethylsilyl)amide, hydroxides (e.g., potassium hydroxide, sodium hydroxide, etc.), and amines (e.g., triethylamine, N,N-diisopropylethylamine, tributylamine, etc.).

In some embodiments, the base is an amine base. In some embodiments, the base is triethylamine.

In some embodiments, the reaction (e.g., the reaction between a compound of Formula IV-10 and Compound Va-I) occurs in the presence of an additive such as tetrabutylammonium salts (e.g., tetrabutylammonium iodide, tetrabutylammonium bromide, tetrabutylammonium chloride, etc.) or lithium salts (e.g., lithium iodide, lithium bromide, lithium chloride, etc.).

In some embodiments, the additive is a tetrabutylammonium salt. In some embodiments, the additive is tetrabutylammonium chloride.

In some embodiments, the reaction (e.g., the reaction between a compound of Formula IV-10 and Compound Va-I) occurs in the presence of a solvent. Suitable solvents include, but are not limited to, polar aprotic solvents (e.g., N,N-dimethylformamide, N,N-dimethylacetamide, N-methyl-2-pyrrolidone, dimethyl sulfoxide, etc.), ethers (e.g., tetrahydrofuran, 2-methyltetrahydrofuran, 1,4-dioxane, etc.), nitriles (e.g., acetonitrile, benzonitrile, etc.), hydrocarbons (e.g., benzene, toluene, etc.), alcohols (e.g., methanol, ethanol, 2-propanol, 2-butanol, etc.), and halogenated solvents (e.g., dichloromethane, 1,2-dichloroethane, etc.).

In some embodiments, the solvent is a polar aprotic solvent. In some embodiments, the solvent is N,N-dimethylformamide.

In some embodiments, the reaction (e.g., the reaction between a compound of Formula IV-10 and Compound Va-I) occurs in the presence of a temperature of about 20° C. to about 120° C. In some embodiments, the temperature is about 80° C. to about 100° C. In some embodiments, the temperature is about 90° C.

In some embodiments, the reaction (e.g., the reaction between a compound of Formula IV-10 and Compound Va-I) occurs in the presence of a catalyst such as a palladium(II) source (e.g., palladium(II) acetate), a base such as an amine base (e.g., triethylamine), an additive such as a tetrabutylammonium salt (e.g., tetrabutylammonium chloride), a solvent such as a polar aprotic solvent (e.g., N,N-dimethylformamide), and a suitable temperature such as a temperature of about 80° C. to about 100° C. (e.g., about 90° C.).

### Synthesis of Compound III Via Asymmetric Cyclization

Also provided is the synthesis of Compound III or a salt thereof via asymmetric cyclization.

For example, Compound III

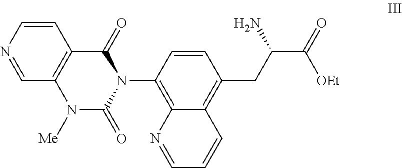

or a salt thereof may be prepared by a process comprising one or more or all of the following steps:
(a) subjecting a compound of Formula X-1

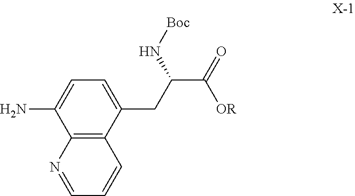

wherein R is a C₁₋₄ alkyl,
or a salt thereof to an isocyanation condition to form a compound of Formula X-2

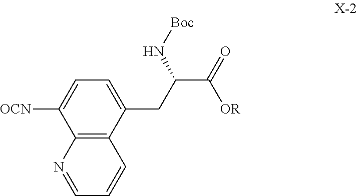

wherein R is a C₁₋₄ alkyl,
or a salt thereof;
(b) contacting a compound of Formula X-2 or a salt thereof with Compound VI-1

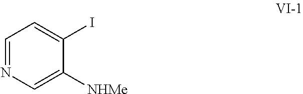

or a salt thereof to form a compound of Formula III-10

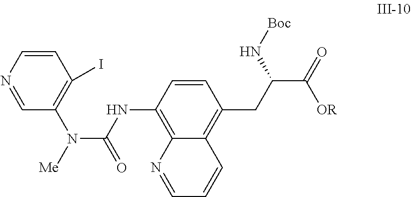

wherein R is a C₁₋₄ alkyl,
or a salt thereof;
(c) subjecting Compound III-10 or a salt thereof to a cyclization condition to form a compound of Formula III-11

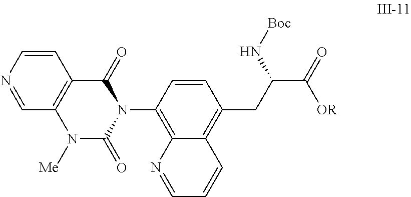

wherein R is a C₁₋₄ alkyl,
or a salt thereof.

In some embodiments, R is a C₁₋₄ alkyl. In some embodiments, R is methyl or ethyl.

In some embodiments, R is methyl and a compound of Formula III-11 is Compound III-2

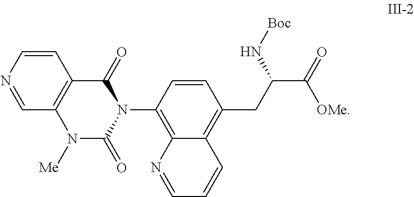

In some embodiments, R is ethyl and a compound of Formula III-11 is Compound III-3

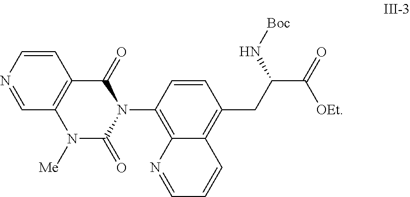

In some embodiments, the process further comprises: (d) converting Compound III-2

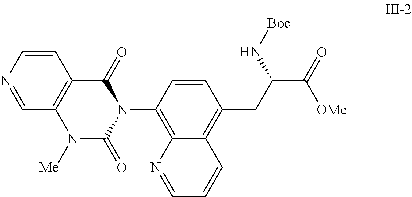

or a salt thereof to Compound III

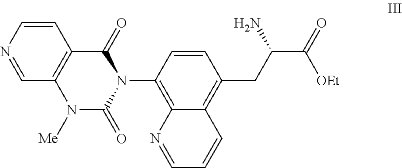

or a salt thereof.

In some embodiments, the reaction (e.g., the reaction for providing Compound III from III-2) occurs in the presence of an acid. In some embodiments, the acid is a sulfonic acid (e.g., methanesulfonic acid).

In some embodiments, the reaction (e.g., the reaction for providing Compound III from III-2) occurs in the presence of a solvent. In some embodiments, the solvent is an alcohol (e.g., ethanol).

In some embodiments, the process comprises subjecting a compound of Formula X-1 or a salt thereof to an isocyanation condition to form a compound of Formula X-2 or a salt thereof.

In some embodiments, the isocyanation condition (e.g., the condition for providing a compound of Formula X-2 from a compound of Formula X-1) comprises an isocyanation reagent, such as 1,1′-carbonyldiimidazole, phosgene, triphosgene, or carbon monoxide.

In some embodiments, the isocyanation reagent is 1,1′-carbonyldiimidazole.

In some embodiments, the isocyanation condition (e.g., the condition for providing a compound of Formula X-2 from a compound of Formula X-1) comprises a base. Suitable bases include, but are not limited to, amines (e.g., N,N-diisopropylethylamine, triethylamine, tributylamine, 4-methylmorpholine, etc.), carbonates (e.g., sodium carbonate, lithium carbonate, potassium carbonate, etc.), acetates (e.g., lithium acetate, sodium acetate, potassium acetate, tetrabutylammonium acetate, etc.), phosphates such as phosphate tribasic (e.g., potassium phosphate tribasic, etc.), alkoxides (e.g., sodium methoxide, sodium tert-butoxide, potassium tert-butoxide, etc.), lithium bis(trimethylsilyl)amide, and hydroxides (e.g., potassium hydroxide, sodium hydroxide, etc.).

In some embodiments, the base is an amine base. In some embodiments, the base is N,N-diisopropylethylamine.

In some embodiments, the isocyanation condition (e.g., the condition for providing a compound of Formula X-2 from a compound of Formula X-1) comprises a solvent. Suitable solvents include, but are not limited to, polar aprotic solvents (e.g., N,N-dimethylacetamide, N-methyl-2-pyrrolidone, dimethyl sulfoxide, etc.), ethers (e.g., tetrahydrofuran, 2-methyltetrahydrofuran, 1,4-dioxane, etc.), nitriles (e.g., acetonitrile, benzonitrile, etc.), hydrocarbons (e.g., benzene, toluene, etc.), esters (e.g., ethyl acetate, isopropyl acetate, etc.), and halogenated solvents (e.g., dichloromethane, 1,2-dichloroethane, etc.).

In some embodiments, the solvent is a halogenated solvent. In some embodiments, the solvent is dichloromethane.

In some embodiments, the isocyanation condition (e.g., the condition for providing a compound of Formula X-2 from a compound of Formula X-1) comprises a temperature of about −20° C. to about 50° C. In some embodiments, the temperature is about 10° C. to about 30° C. In some embodiments, the temperature is about 20° C.

In some embodiments, the isocyanation condition (e.g., the condition for providing a compound of Formula X-2 from a compound of Formula X-1) comprises an isocyanation reagent such as 1,1′-carbonyldiimidazole, a base such as an amine base (e.g., N,N-diisopropylethylamine), a solvent such as a halogenated solvent (e.g., dichloromethane), and a suitable temperature such as a temperature of about 10° C. to about 30° C. (e.g., about 20° C.).

In some embodiments, the process comprises contacting a compound of Formula X-2 or a salt thereof with Compound VI-1 or a salt thereof to form a compound of Formula III-10 or a salt thereof.

In some embodiments, the reaction (e.g., the reaction between a compound of Formula X-2 and Compound VI-1) occurs in the presence of a base. Suitable bases include, but are not limited to, basic aromatic compounds (e.g., pyridine, etc.), amines (e.g., triethylamine, tributylamine, 4-methylmorpholine, etc.), carbonates (e.g., sodium carbonate, lithium carbonate, potassium carbonate, etc.), acetates (e.g., lithium acetate, sodium acetate, potassium acetate, tetrabutylammonium acetate, etc.), phosphates such as phosphate tribasic (e.g., potassium phosphate tribasic, etc.), alkoxides (e.g., sodium methoxide, sodium tert-butoxide, potassium tert-butoxide, etc.), lithium bis(trimethylsilyl)amide, and hydroxides (e.g., potassium hydroxide, sodium hydroxide, etc.).

In some embodiments, the base is a basic aromatic compound. In some embodiments, the base is pyridine.

In some embodiments, the reaction (e.g., the reaction between a compound of Formula X-2 and Compound VI-1) occurs in the presence of a solvent. The solvent may be a polar aprotic solvent (e.g., N,N-dimethylacetamide, N-methyl-2-pyrrolidone, dimethyl sulfoxide, etc.), an ether solvent (e.g., tetrahydrofuran, 2-methyltetrahydrofuran, 1,4-dioxane, etc.), a nitrile solvent (e.g., acetonitrile, benzonitrile, etc.), or a hydrocarbon solvent (e.g., benzene, toluene, etc.). In some embodiments, pyridine (e.g., the pyridine base) is used as a solvent.

In some embodiments, the reaction (e.g., the reaction between a compound of Formula X-2 and Compound VI-1) occurs at a temperature of about 20° C. to about 120° C. In some embodiments, the temperature is about 90° C. to about 110° C. In some embodiments, the temperature is about 100° C.

In some embodiments, the reaction (e.g., the reaction between a compound of Formula X-2 and Compound VI-1) occurs in the presence of a base such as a basic aromatic compound (e.g., pyridine), a solvent (e.g., pyridine), and at a suitable temperature such as a temperature of about 90° C. to about 110° C. (e.g., about 100° C.).

In some embodiments, the process comprises subjecting a compound of Formula III-10 or a salt thereof to a cyclization condition to form a compound of Formula III-11 or a salt thereof.

In some embodiments, the cyclization condition (e.g., the condition for providing a compound of Formula III-11 from a compound of Formula III-10) comprises a catalyst such as a palladium catalyst. The palladium catalyst can be a palladium(II) source (e.g., allyl(cyclopentadienyl)palladium(II), palladium(II) chloride, etc.) or a palladium(0) source (e.g., tetrakis(triphenylphosphine)palladium(0), etc.).

In some embodiments, the catalyst is a palladium(II) source. In some embodiments, the catalyst is allyl(cyclopentadienyl)palladium(II).

In some embodiments, the cyclization condition (e.g., the condition for providing a compound of Formula III-11 from a compound of Formula III-10) comprises a ligand. Suitable ligands include, but are not limited to, chiral phosphine ligands such as Josiphos ligands or 2,2′-p-tolylphosphino-1,1′-binaphthyl.

In some embodiments, the ligand is Josiphos ligand. In some embodiments, the ligand is Josiphos ligand SL-J013-1 (CAS 187733-50-2).

In some embodiments, the cyclization condition (e.g., the condition for providing a compound of Formula III-11 from a compound of Formula III-10) comprises a base. Suitable bases include, but are not limited to, amines (e.g., diisopropylethylamine, triethylamine, tripropylamine, tributylamine, etc.), carbonates (e.g., sodium carbonate, lithium carbonate, potassium carbonate, etc.), phosphates such as phosphate tribasic (e.g., potassium phosphate tribasic, etc.), alkoxides (e.g., sodium methoxide, sodium tert-butoxide, potassium tert-butoxide, etc.), lithium bis(trimethylsilyl)amide, and hydroxides (e.g., potassium hydroxide, sodium hydroxide, etc.).

In some embodiments, the base is an amine base. In some embodiments, the base is diisopropylethylamine.

In some embodiments, the cyclization condition (e.g., the condition for providing a compound of Formula III-11 from a compound of Formula III-10) comprises a carbonyl source such as carbon monoxide.

In some embodiments, the cyclization condition (e.g., the condition for providing a compound of Formula III-11 from a compound of Formula III-10) comprises a solvent. Suitable solvents include, but are not limited to, polar aprotic solvents (e.g., N,N-dimethylformamide, N,N-dimethylacetamide, N-methyl-2-pyrrolidone, dimethyl sulfoxide, etc.), ethers (e.g., tetrahydrofuran, 2-methyltetrahydrofuran, 1,4-dioxane, etc.), nitriles (e.g., acetonitrile, benzonitrile, etc.), and hydrocarbons (e.g., benzene, toluene, etc.).

In some embodiments, the solvent is a polar aprotic solvent. In some embodiments, the solvent is N,N-dimethylformamide.

In some embodiments, the cyclization condition (e.g., the condition for providing a compound of Formula III-11 from a compound of Formula III-10) comprises a temperature of about 20° C. to about 120° C. In some embodiments, the temperature is about 60° C. to about 80° C. In some embodiments, the temperature is about 70° C.

In some embodiments, the cyclization condition (e.g., the condition for providing a compound of Formula III-11 from a compound of Formula III-10) comprises a pressure of about 15 psig to about 500 psig. In some embodiments, the pressure is about 450 psig.

In some embodiments, the cyclization condition (e.g., the condition for providing a compound of Formula III-11 from a compound of Formula III-10) comprises a catalyst such as a palladium(II) source (e.g., allyl(cyclopentadienyl)palladium(II)), a ligand such as a Josiphos ligand (e.g., Josiphos ligand SL-J013-1), a base such as an amine base (e.g., diisopropylethylamine), a solvent such as a polar aprotic solvent (e.g., N,N-dimethylformamide), a suitable temperature such as a temperature of about 60° C. to about 80° C. (e.g., about 70° C.), and a suitable pressure such as about 450 psig.

Synthesis of a compound of Formula V or Va
Synthesis of a compound of Formula Va

### Cyclization Route A

A compound of Formula Va or a salt thereof may be prepared via a cyclization reaction.

In some embodiments, provided is a process for preparing a compound of Formula Va

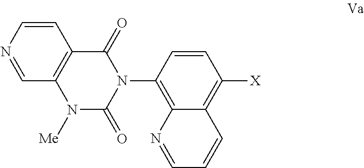

wherein X is Br or I,
or a salt thereof, comprising:
(a) contacting Compound VI

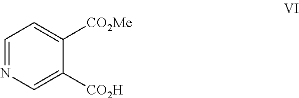 [SMILES: COC(=O)c1ccncc1C(=O)O]

or a salt thereof with a compound of Formula VII

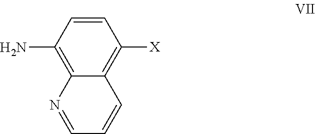

wherein X is Br or I,
or a salt thereof in the presence of an azide source or a deoxo-fluor to form a compound of Formula V-3

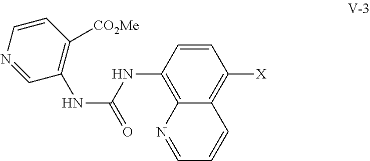

wherein X is Br or I,
or a salt thereof;
(b) subjecting the compound of Formula V-3 or a salt thereof into a cyclization condition to provide a compound of Formula V-1

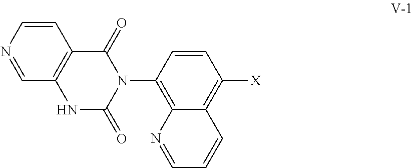

wherein X is Br or I,
or a salt thereof; and
(c) contacting the compound of Formula V-1 or a salt thereof with a methylation agent to form the compound of Formula Va.

In some embodiments, the process comprises contacting Compound VI or a salt thereof with a compound of Formula VII or a salt thereof in the presence of an azide source or a deoxo-fluor to form a compound of Formula V-3 or a salt thereof.

In some embodiments, the reaction between Compound VI or a salt thereof and a compound of Formula VII or a salt thereof occurs in the presence of an azide source. Suitable azide source include, but are not limited to, diphenylphosphoryl azide (DPPA), trimethylsilyl azide, sodium azide, lithium azide, carboxybenzenesulfonazide, and acetamidobenenesulfonyl.

In some embodiments, the azide source is diphenylphosphoryl azide (DPPA).

In some embodiments, the reaction between Compound VI or a salt thereof and a compound of Formula VII or a salt thereof occurs in the presence of a base. Suitable bases include, but are not limited to, amines (e.g., N,N-diisopropylethylamine, triethylamine, tripropylamine, tributylamine, 4-methylmorpholine, 1,4-diazabicylo[2.2.2]-octane, 1,8-diazabicyclo[5.4.0]undec-7-ene, etc.), basic aromatic compounds (e.g., pyridine, 2,6-lutidine, imidazole, etc.), carbonates (e.g., lithium carbonate, sodium carbonate, potassium carbonate, cesium carbonate, etc.), bicarbonates (e.g., lithium bicarbonate, sodium bicarbonate, potassium bicarbonate, etc.), and phosphates (e.g., sodium phosphate, potassium phosphate, etc.).

In some embodiments, the base is an amine base. In some embodiments, the base is triethylamine.

In some embodiments, the reaction between Compound VI or a salt thereof and a compound of Formula VII or a salt thereof occurs in the presence of a solvent. Suitable solvents include, but are not limited to, ethers (e.g., tetrahydrofuran, 2-methyltetrahydrofuran, 1,4-dioxane, etc.), aromatic solvents (e.g., benzene, toluene, etc.), nitriles (e.g., acetonitrile, benzonitrile, etc.), halogenated solvents (e.g., dichloromethane, 1,2-dichloroethane, chlorobenzene, trifluorotoluene, etc.).

In some embodiments, the solvent is a halogenated solvent. In some embodiments, the solvent is trifluorotoluene.

In some embodiments, the reaction between Compound VI or a salt thereof and a compound of Formula VII or a salt thereof occurs at a temperature of about −10° C. to about 120° C. In some embodiments, the temperature is about 50° C. to about 70° C. In some embodiments, the temperature is about 60° C.

In some embodiments, the reaction between Compound VI or a salt thereof and a compound of Formula VII or a salt thereof occurs in the presence of an azide source (e.g., diphenylphosphoryl azide (DPPA)), a base such as an amine base (e.g., triethylamine), a solvent such as a halogenated solvent (e.g., trifluorotoluene), and at a suitable temperature such as a temperature of about 50° C. to about 70° C. (e.g., about 60° C.).

In some embodiments, the process comprises subjecting the compound of Formula V-3 or a salt thereof into a cyclization condition to provide a compound of Formula V-1 or a salt thereof.

In some embodiments, the cyclization condition (e.g., the cyclization condition for providing a compound of Formula V-1 from a compound of Formula V-3) comprise a base. Suitable bases include, but are not limited to, amines (e.g., N,N-diisopropylethylamine, triethylamine, tripropylamine, tributylamine, 4-methylmorpholine, 1,8-diazabicyclo[5.4.0]undec-7-ene, etc.), basic aromatic compounds (e.g., pyridine, 2,6-di-tert-butylpyridine, etc.), carbonates (e.g., lithium carbonate, sodium carbonate, potassium carbonate, cesium carbonate, etc.), alkoxides (e.g., sodium methoxide, sodium tert-butoxide, lithium tert-butoxide, etc.), phosphates (e.g., sodium phosphate, potassium phosphate, etc.), and amides (e.g., lithium bis(trimethylsilyl)amide, potassium bis(trimethylsilyl)amide, lithium diisopropylamide, etc.).

In some embodiments, the base is an amine base. In some embodiments, the base is 1,8-diazabicyclo[5.4.0]undec-7-ene.

In some embodiments, the cyclization condition (e.g., the cyclization condition for providing a compound of Formula V-1 from a compound of Formula V-3) comprise a solvent. Suitable solvents include, but are not limited to, ethers (e.g., tetrahydrofuran, 2-methyltetrahydrofuran, etc.), polar aprotic solvents (e.g., N,N-dimethylformamide, N,N-dimethylacetamide, dimethyl sulfoxide, N-methyl-2-pyrrolidone, etc.), nitriles (e.g., acetonitrile, etc.), and halogenated solvents (e.g., dichloromethane, etc.).

In some embodiments, the solvent is a polar aprotic solvent. In some embodiments, the solvent is N-methyl-2-pyrrolidone.

In some embodiments, the cyclization condition (e.g., the cyclization condition for providing a compound of Formula V-1 from a compound of Formula V-3) comprises a temperature of about −10° C. to about 60° C. In some embodiments, the temperature is about 10° C. to about 30° C. In some embodiments, the temperature is about 20° C.

In some embodiments, the cyclization condition (e.g., the cyclization condition for providing a compound of Formula V-1 from a compound of Formula V-3) comprises a base such as an amine base (e.g., 1,8-diazabicyclo[5.4.0]undec-7-ene), a solvent such as a polar aprotic solvent (e.g., N-methyl-2-pyrrolidone), and a suitable temperature such as a temperature of about 10° C. to about 30° C. (e.g., about 20° C.).

In some embodiments, the methylation agent (e.g., the methylation agent used to provide a compound of Formula Va from a compound of Formula V-1) includes, but is not limited to, methyl iodide, methyl bromide, methyl chloride, methyl triflate, diazomethane, dimethyl sulfate, and dimethyl carbonate.

In some embodiments, the methylation agent is methyl iodide.

In some embodiments, the methylation reaction (e.g., the methylation reaction of a compound of Formula V-1) occurs in the presence of a base. Suitable bases include, but are not limited to, amines (e.g., N,N-diisopropylethylamine, triethylamine, tripropylamine, tributylamine, 4-methylmorpholine, 1,8-diazabicyclo[5.4.0]undec-7-ene, etc.), basic aromatic compounds (e.g., pyridine, 2,6-di-tert-butylpyridine, etc.), carbonates (e.g., lithium carbonate, sodium carbonate, potassium carbonate, cesium carbonate, etc.), alkoxides (e.g., sodium methoxide, sodium tert-butoxide, lithium tert-butoxide, etc.), phosphates (e.g., sodium phosphate, potassium phosphate, etc.), and amides (e.g., lithium bis(trimethylsilyl)amide, potassium bis(trimethylsilyl)amide, lithium diisopropylamide, etc.).

In some embodiments, the base is an amine base. In some embodiments, the base is 1,8-diazabicyclo[5.4.0]undec-7-ene.

In some embodiments, the methylation reaction (e.g., the methylation reaction of a compound of Formula V-1) occurs in the presence of a solvent. Suitable solvents include, but are not limited to, ethers (e.g., tetrahydrofuran, 2-methyltetrahydrofuran, etc.), polar aprotic solvents (e.g., N,N-dimethylformamide, N,N-dimethylacetamide, dimethyl sulfoxide, N-methyl-2-pyrrolidone, etc.), nitriles (e.g., acetonitrile, etc.), and halogenated solvents (e.g., dichloromethane, etc.).

In some embodiments, the solvent is a polar aprotic solvent. In some embodiments, the solvent is N-methyl-2-pyrrolidone.

In some embodiments, the methylation reaction (e.g., the methylation reaction of a compound of Formula V-1) occurs at a temperature of about −10° C. to about 60° C. In some embodiments, the temperature is about 10° C. to about 30° C. In some embodiments, the temperature is about 20° C.

In some embodiments, the methylation reaction (e.g., the methylation reaction of a compound of Formula V-1 with a methylation agent such as methyl iodide) occurs in the presence of a base such as an amine base (e.g., 1,8-diazabicyclo[5.4.0]undec-7-ene), a solvent such as an polar aprotic solvent (e.g., N-methyl-2-pyrrolidone), and at a suitable temperature such as a temperature of about 10° C. to about 30° C. (e.g., about 20° C.).

In some embodiments, the compound of Formula V-1 (e.g., the product obtained from step (b)) or a salt thereof is subject to the methylation reaction (step (c)) without isolation.

In some embodiments, X is Br. In some embodiments, X is I.

In some embodiments, Compound VI or a salt thereof is prepared from pyridine-3,4-dicarboxylic acid through an anhydride intermediate that is furo[3,4-c]pyridine-1,3-dione.

In some embodiments, provided is a process for preparing Compound VI

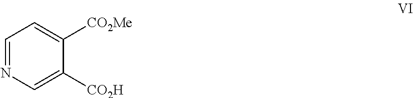 [SMILES: COC(=O)c1ccncc1C(=O)O]

or a salt thereof, comprising
(a) converting pyridine-3,4-dicarboxylic acid into an anhydride intermediate that is furo[3,4-c]pyridine-1,3-dione; and
(b) subjecting the anhydride intermediate into a ring opening condition to form Compound VI or a salt thereof.

In some embodiments, the conversion of pyridine-3,4-dicarboxylic acid to the anhydride intermediate occurs in the presence of an anhydride forming reagent. Suitable anhydride forming reagents include, but are not limited to acetic anhydride, di-tert-butyl dicarbonate, oxalyl chloride, 1,1′-carbonyldiimidazole, oxalyl chloride, phosgene, triphosgene, alkyl chloroformates (e.g., ethyl chloroformate, isobutyl chloroformate, etc.), carbodiimides (e.g., dicyclohexylcarbodiimide, diisopropylcarbodiimide, 1-ethyl-3-(3-dimethylaminopropyl)carbodiimide), propanephosphonic acid anhydride (T3P), and peptide coupling reagents (e.g., HATU, HBTU, TATU, TBTU, HCTU, BOP, PyBOP, COMU, etc.).

In some embodiments, the anhydride forming reagent is acetic anhydride.

In some embodiments, the conversion of pyridine-3,4-dicarboxylic acid to the anhydride intermediate occurs in the presence of a catalyst. Suitable catalysts include but are not limited to magnesium salts (e.g., magnesium iodide, magnesium bromide, magnesium chloride, etc.).

In some embodiments, the conversion of pyridine-3,4-dicarboxylic acid to the anhydride intermediate occurs in the absence of a catalyst.

In some embodiments, the conversion of pyridine-3,4-dicarboxylic acid to the anhydride intermediate occurs in the presence of a solvent. Suitable solvents include, but are not limited to, halogenated solvents (e.g., dichloromethane, 1,2-dichloroethane, chlorobenzene, chloroform, etc.), and ethers (e.g., tetrahydrofuran, 2-methyltetrahydrofuran, 1,4-dioxane, etc.).

In some embodiments, the conversion of pyridine-3,4-dicarboxylic acid to the anhydride intermediate occurs in the absence of a solvent.

In some embodiments, the conversion of pyridine-3,4-dicarboxylic acid to the anhydride intermediate occurs at a temperature of about 20° C. to about 120° C. In some embodiments, the temperature is about 95° C. to about 115° C. In some embodiments, the temperature is about 105° C.

In some embodiments, the conversion of pyridine-3,4-dicarboxylic acid to the anhydride intermediate occurs in the presence of a suitable reagent such as acetic anhydride, and at a suitable temperature such as a temperature of about 95° C. to about 115° C. (e.g., about 105° C.).

In some embodiments, the ring opening condition (e.g., the ring opening condition for proving a salt of Compound VI) comprises a ring opening reagent. Suitable ring opening reagents include, but are not limited to, acetyl chloride, reagents capable of generating acids in situ (chlorosilanes such as trimethylsilyl chloride and methanol), and acids (e.g., hydrochloric acid, hydrobromic acid, hydroiodic acid, sulfuric acid, phosphoric acid, etc.).

In some embodiments, the ring opening reagent is acetyl chloride.

In some embodiments, the ring opening condition (e.g., the ring opening condition for proving a salt of Compound VI) comprises a base. Suitable bases include, but are not limited to, carbonates (e.g., lithium carbonate, sodium carbonate, potassium carbonate, cesium carbonate, etc.), bicarbonates (e.g., lithium bicarbonate, sodium bicarbonate, potassium bicarbonate, etc.), phosphates (e.g., sodium phosphate, potassium phosphate, etc.), and amines (e.g., triethylamine, N,N-diisopropylethylamine, etc.).

In some embodiments, the ring opening condition (e.g., the ring opening condition for proving a salt of Compound VI) does not comprise a base.

In some embodiments, the ring opening condition (e.g., the ring opening condition for proving a salt of Compound VI) comprises a solvent. Suitable solvents include, but are not limited to, water, methanol, and a combination thereof.

In some embodiments, the solvent is methanol.

In some embodiments, the ring opening condition (e.g., the ring opening condition for proving a salt of Compound VI) comprises a temperature of about −10° C. to about 80° C. In some embodiments, the temperature is about 0° C. to about 20° C. In some embodiments, the temperature is about 10° C.

In some embodiments, the ring opening condition (e.g., the ring opening condition for proving a salt of Compound VI) comprises a ring opening reagent (e.g., acetyl chloride), a solvent (e.g., methanol), and a suitable temperature such as a temperature of about 0° C. to about 20° C. (e.g., about 10° C.).

In some embodiments, the ring opening reaction (step (b)) provides a salt of Compound VI. In some embodiments, the ring opening reaction (step (b)) provides Compound VI HCl salt.

In some embodiments, the process further comprises free basing the salt of Compound VI (e.g., the salt of Compound VI obtained from the ring opening reaction (step (b)). In some embodiments, the salt of Compound VI (e.g., the salt of Compound VI obtained from the ring opening reaction (step (b)) is Compound VI HCl salt.

In some embodiments, the free basing step (e.g., free basing the salt of Compound VI) occurs in the presence of a base. Suitable bases include, but are not limited to, amines (e.g., triethylamine, N,N-diisopropylethylamine, tripropylamine, tributylamine, 1,4-diazabicylo[2.2.2]-octane, 1,8-diazabicyclo[5.4.0]undec-7-ene, etc.), carbonates (e.g., lithium carbonate, sodium carbonate, potassium carbonate, cesium carbonate, etc.), bicarbonates (e.g., lithium bicarbonate, sodium bicarbonate, potassium bicarbonate, cesium bicarbonate, etc.), phosphates (e.g., sodium phosphate, dipotassium hydrogen phosphate, disodium phosphate, potassium phosphate, cesium phosphate, etc.), basic aromatic compounds (e.g., pyridine, 2,6-lutidine, imidazole, etc.), and hydroxides (e.g., sodium hydroxide, potassium hydroxide, etc.).

In some embodiments, the base is a phosphate. In some embodiments, the base is dipotassium hydrogen phosphate.

In some embodiments, the free basing step (e.g., free basing the salt of Compound VI) occurs in the presence of a solvent. In some embodiments, the solvent is water. In some embodiments, the solvent is selected from ethers (e.g., tetrahydrofuran, 2-methyltetrahydrofuran, tert-butyl methyl ether, etc.), ketones (e.g., acetone, 2-butanone, 4-methyl-2-pentanone, etc.), esters (e.g., ethyl acetate, isopropyl acetate, methyl acetate, etc.), nitriles (e.g., acetonitrile), halogenated solvents (e.g., dichloromethane, 1,2-dichloroethane, chlorobenzene, etc.), and a combination of any of the foregoing with water.

In some embodiments, the solvent is water.

In some embodiments, the free basing step (e.g., free basing the salt of Compound VI) occurs at a temperature of about −10° C. to about 60° C. In some embodiments, the temperature is about 10° C. to about 30° C. In some embodiments, the temperature is about 20° C.

In some embodiments, the free basing step (e.g., free basing the salt of Compound VI) occurs in the presence of a base such as a phosphate (e.g., dipotassium hydrogen phosphate), a solvent such as water, and at a suitable temperature such as a temperature of about 10° C. to about 30° C. (e.g., about 20° C.).

In some embodiments, the compound of Formula VII can be prepared by contacting quinolin-8-amine with an iodination agent or a bromination agent.

In some embodiments, X is iodo, and the compound of Formula VII is Compound VII-I

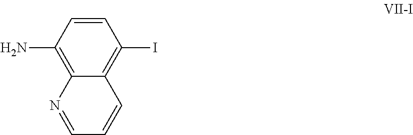 [SMILES: Nc1ccc(I)c2cccnc12]

In some embodiments, provided is a process for preparing Compound VII-I

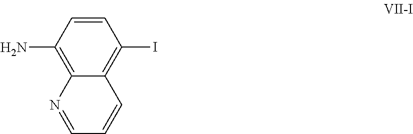 [SMILES: Nc1ccc(I)c2cccnc12]

or a salt thereof, comprising contacting quinolin-8-amine with an iodination agent to form Compound VII-I or a salt thereof.

Suitable iodination agents (e.g., iodination agents for proving Compound VII-I) include, but are not limited to, iodide, N-iodosuccinimide, iodine monochloride, and 1,3-diiodo-5,5-dimethylhydantoin.

In some embodiments, the iodination agent is iodine.

In some embodiments, the reaction between quinolin-8-amine with an iodination agent occurs in the presence of an additive such as tert-butyl hydroperoxide in water.

In some embodiments, the reaction between quinolin-8-amine with an iodination agent occurs in the presence of a solvent. Suitable solvents include, but are not limited to, pyridine, ethers (e.g., tetrahydrofuran, 2-methyltetrahydrofuran, tert-butyl methyl ether, 1,4-dioxane, etc.), esters (e.g., ethyl acetate, methyl acetate, isopropyl acetate, etc.), halogenated solvents (e.g., dichloromethane, 1,2-dichloroethane, etc.), and nitriles (e.g., acetonitrile, etc.), and any combination thereof.

In some embodiments, the solvent is a mixed solvent. In some embodiments, the solvent is a mixture of isopropyl acetate and pyridine.

In some embodiments, the reaction between quinolin-8-amine and an iodination agent occurs at a temperature of about −10° C. to about 60° C. In some embodiments, the temperature is about 15° C. to about 35° C. In some embodiments, the temperature is about 25° C.

In some embodiments, the reaction between quinolin-8-amine with an iodination agent (e.g., iodine) occurs in the presence of an additive (e.g., tert-butyl hydroperoxide in water), a solvent (e.g., a mixture of isopropyl acetate and pyridine), and at a suitable temperature such as a temperature of about 15° C. to about 35° C. (e.g., about 25° C.).

### Cyclization Route B

Additionally or alternatively, Compound Va-Br

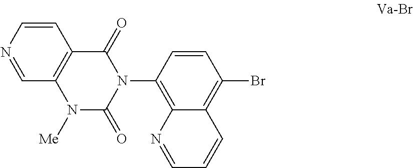 [SMILES: Cn1c(=O)n(-c2ccc(Br)c3cccnc23)c(=O)c2ccncc21]

or a salt thereof may be prepared by a process comprising one or more or all of the following steps:
(a) coupling isonicotinic acid or a salt thereof with Compound VII-Br

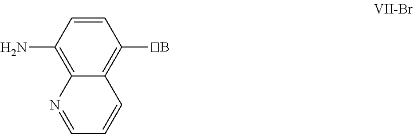

or a salt thereof in the presence of a coupling agent to form Compound XI

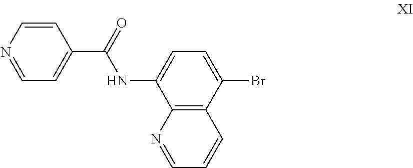 [SMILES: O=C(Nc1ccc(Br)c2cccnc12)c1ccncc1]

or a salt thereof;
(b) converting Compound XI or a salt thereof to Compound V-1a-Br

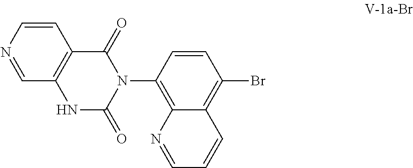 [SMILES: O=c1[nH]c2cnccc2c(=O)n1-c1ccc(Br)c2cccnc12]

or a salt thereof;
(c) contacting Compound V-1a-Br or a salt thereof with a methylation agent to form Compound Va-Br or a salt thereof.

In some embodiments, the process comprises coupling isonicotinic acid or a salt thereof with Compound VII-Br or a salt thereof in the presence of a coupling agent to form Compound XI or a salt thereof.

In some embodiments, the coupling agent (e.g., coupling agents used to provide Compound XI from isonicotinic acid and Compound VII-Br) include, but are not limited to, acid halide forming agents (e.g., thionyl chloride, oxalyl chloride, phosgene, triphosgene, etc.), 1,1′-carbonyldiimidazole (CDI), alkyl chloroformates (e.g., ethyl chloroformate, isobutyl chloroformate, etc.), carbodiimides (e.g., N,N′-dicyclohexylcarbodiimide (DCC), N,N′-diisopropylcarbodiimide (DIC), 1-ethyl-3-(3-dimethylaminopropyl)carbodiimide (EDC), etc.), propanephosphonic acid anhydride (T3P), and peptide coupling reagents (e.g., hexafluorophosphate azabenzotriazole tetramethyl uronium (HATU), hexafluorophosphate benzotriazole tetramethyl uronium (HBTU), 2-(3h-[1,2,3]triazolo[4,5-b]pyridin-3-yl)-1,1,3,3-tetramethyluronium tetrafluoroborate (TATU), 2-(1H-benzotriazole-1-yl)-1,1,3,3-tetramethyluronium tetrafluoroborate (TBTU), 0-(6-Chlorobenzotriazol-1-yl)-N,N,N′,N′-tetramethyluronium hexafluorophosphate (HCTU), benzotriazol-1-yloxytris(dimethylamino)phosphonium hexafluorophosphate (BOP), benzotriazol-1-yloxytripyrrolidinophosphonium hexafluorophosphate (PyBOP), [[(Z)-(1-cyano-2-ethoxy-2-oxoethylidene)amino]oxy-morpholin-4-ylmethylidene]-dimethylazanium hexafluorophosphate (COMU), etc.).

In some embodiments, the coupling agent is an acid halide forming agent. In some embodiments, the coupling agent is oxalyl chloride.

In some embodiments, the coupling agent is an acid halide forming agent (e.g., oxalyl chloride), and isonicotinic acid is converted to isonicotinoyl chloride by the coupling agent prior to reacting with Compound VII-Br.

In some embodiments, the reaction (e.g., the reaction for providing Compound XI from isonicotinic acid and Compound VII-Br) occurs in the presence of a catalyst. Suitable catalysts include, but are not limited to, amides (e.g., N,N-dimethylformamide, dimethylacetamide, N-methyl-2-pyrrolidone, etc.), 4-(dimethylamino)pyridine, 1-hydroxybenzotriazole (HOBt), and 1-hydroxy-7-azabenzotriazole (HOAt).

In some embodiments, the catalyst is an amine catalyst. In some embodiments, the catalyst is N,N-dimethylformamide.

In some embodiments, the reaction (e.g., the reaction for providing Compound XI from isonicotinic acid and Compound VII-Br) occurs in the presence of a base. Suitable bases include, but are not limited to, amines (e.g., N,N-diisopropylethylamine, triethylamine, tripropylamine, tributylamine, 4-methylmorpholine, etc.), basic aromatic compounds (e.g., pyridine, 2,6-lutidine, imidazole, etc.), carbonates (e.g., lithium carbonate, sodium carbonate, potassium carbonate, cesium carbonate, etc.), bicarbonates (e.g., lithium bicarbonate, sodium bicarbonate, potassium bicarbonate, etc.), and phosphates (e.g., sodium phosphate, potassium phosphate, etc.).

In some embodiments, the base is an amine base. In some embodiments, the base is N,N-diisopropylethylamine.

In some embodiments, the reaction (e.g., the reaction for providing Compound XI from isonicotinic acid and Compound VII-Br) occurs in the presence of a solvent. Suitable solvents include, but are not limited to, esters (e.g. ethyl acetate, isopropyl acetate, etc.), ethers (e.g., tetrahydrofuran, 2-methyltetrahydrofuran, tert-butyl methyl ether, etc.), halogenated solvents (e.g., dichloromethane, 1,2-dichloroethane, chlorobenzene, etc.), hydrocarbons (e.g., benzene, toluene, n-heptane, etc.), nitriles (e.g., acetonitrile, benzonitrile, etc.), and polar aprotic solvents (e.g., N,N-dimethylformamide, N,N-dimethylacetamide, N-methyl-2-pyrrolidone, etc.).

In some embodiments, the solvent is a halogenated solvent. In some embodiments, the solvent is dichloromethane.

In some embodiments, the reaction (e.g., the reaction for providing Compound XI from isonicotinic acid and Compound VII-Br) occurs at a temperature of about −15° C. to about 80° C. In some embodiments, the temperature is about 5° C. to about 25° C. In some embodiments, the temperature is about 15° C.

In some embodiments, the reaction (e.g., the reaction for providing Compound XI from isonicotinic acid and Compound VII-Br) occurs in the presence of a coupling agent such as an acid halide forming agent (e.g., oxalyl chloride), a catalyst such as an amine catalyst (e.g., N,N-dimethylformamide), a base such as an amine base (N,N-diisopropylethylamine), a solvent such as a halogenated solvent (e.g., dichloromethane), and at a suitable temperature such as a temperature of about 5° C. to about 25° C. (e.g., about 15° C.).

In some embodiments, the reaction provides Compound XI. In some embodiments, the reaction provides a salt of Compound XI (e.g., a HCl salt of Compound XI).

In some embodiments, the process comprises converting Compound XI or a salt thereof to Compound V-1a-Br or a salt thereof.

The reaction (e.g., the reaction for proving Compound V-1a-Br from Compound XI) may occur in the presence of a catalyst. The catalyst may be a copper catalyst such as copper(II) acetate (e.g., copper(II) acetate monohydrate, anhydrous copper(II) acetate, etc.), copper(II) chloride, or copper(II) bromide.

In some embodiments, the catalyst is copper(II) acetate monohydrate.

In some embodiments, the reaction (e.g., the reaction for proving Compound V-1a-Br from Compound XI) occurs in the presence of a cyanate. The cyanate may be sodium cyanate, potassium cyanate, or silver cyanate.

In some embodiments, the cyanate is potassium cyanate.

In some embodiments, the reaction (e.g., the reaction for proving Compound V-1a-Br from Compound XI) occurs in the presence of a base. Suitable bases include, but are not limited to, carbonates (e.g., potassium carbonate, sodium carbonate, lithium carbonate, etc.), acetates (e.g., potassium acetate, sodium acetate, lithium acetate, etc.), and propionates (e.g., potassium propionate, lithium propionate, etc.).

In some embodiments, the reaction (e.g., the reaction for proving Compound V-1a-Br from Compound XI) occurs in the absence of a base.

In some embodiments, the reaction (e.g., the reaction for proving Compound V-1a-Br from Compound XI) occurs in the presence of a solvent. The solvent may be a polar aprotic solvent such as N,N-dimethylformamide, N,N-dimethylacetamide, N-methyl-2-pyrrolidone, or dimethyl sulfoxide.

In some embodiments, the solvent is N,N-dimethylformamide.

In some embodiments, the reaction (e.g., the reaction for proving Compound V-1a-Br from Compound XI) occurs at a temperature of about 20° C. to about 120° C. In some embodiments, the temperature is about 70° C. to about 90° C. In some embodiments, the temperature is about 80° C.

In some embodiments, the reaction (e.g., the reaction for proving Compound V-1a-Br from Compound XI) occurs in the presence of a catalyst such as a copper catalyst (e.g., copper(II) acetate monohydrate), a cyanate such as potassium cyanate, a solvent such as a polar aprotic solvent (e.g., N,N-dimethylformamide), and at a suitable temperature such as a temperature of about 70° C. to about 90° C. (e.g., about 80° C.).

In some embodiments, the process comprises contacting Compound V-1a-Br or a salt thereof with a methylation agent to form Compound Va-Br or a salt thereof.

Suitable methylation agents (e.g., methylation agents for providing Compound Va-Br from Compound V-1a-Br) include, but are not limited to, methyl iodide, methyl bromide, methyl chloride, methyl triflate, diazomethane, dimethyl sulfate, and dimethyl carbonate.

In some embodiments, the methylation agent is methyl iodide.

In some embodiments, the reaction (e.g., the reaction for providing Compound Va-Br from Compound V-1a-Br) occurs in the presence of a base. Suitable bases include, but are not limited to, carbonates (e.g., lithium carbonate, sodium carbonate, potassium carbonate, cesium carbonate, etc.), alkoxides (e.g., sodium methoxide, sodium tert-butoxide, lithium tert-butoxide, etc.), phosphates (e.g., sodium phosphate, potassium phosphate, etc.), amines (e.g., triethylamine, N,N-diisopropylethylamine, tripropylamine, tributylamine, 4-methylmorpholine, 1,8-diazabicyclo[5.4.0]undec-7-ene, etc.), basic aromatic compounds (e.g., pyridine, 2,6-di-tert-butylpyridine, etc.), and amides (e.g., lithium bis(trimethylsilyl)amide, potassium bis(trimethylsilyl)amide, lithium diisopropylamide, etc.).

In some embodiments, the base is an amine base. In some embodiments, the base is 1,8-diazabicyclo[5.4.0]undec-7-ene.

In some embodiments, the reaction (e.g., the reaction for providing Compound Va-Br from Compound V-1a-Br) occurs in the presence of a solvent. Suitable solvents include, but are not limited to, ethers (e.g., tetrahydrofuran, 2-methyltetrahydrofuran, etc.), polar aprotic solvents (e.g., N-methyl-2-pyrrolidone, N,N-dimethylformamide, N,N-dimethylacetamide, dimethyl sulfoxide, etc.), nitriles (e.g., acetonitrile), and halogenated solvents (e.g., dichloromethane, etc.).

In some embodiments, the solvent is a polar aprotic solvent. In some embodiments, the solvent is N-methyl-2-pyrrolidone.

In some embodiments, the reaction (e.g., the reaction for providing Compound Va-Br from Compound V-1a-Br) occurs at a temperature of about −10° C. to about 60° C. In some embodiments, the temperature is about 10° C. to about 30° C. In some embodiments, the temperature is about 20° C.

In some embodiments, the reaction (e.g., the reaction for providing Compound Va-Br from Compound V-1a-Br) occurs in the presence of a methylation agent such as methyl iodide, a base such as an amine base (e.g., 1,8-diazabicyclo[5.4.0]undec-7-ene), a solvent such as a polar aprotic solvent (e.g., N-methyl-2-pyrrolidone), and at a suitable temperature such as a temperature of about 10° C. to about 30° C. (e.g., about 20° C.).

The process may further comprise free basing a salt of Compound XI (e.g., a HCl salt of Compound XI) prior to subjecting to the next step (e.g., prior to converting to Compound V-1a-Br).

The free basing step (e.g., free basing a salt such as a HCl salt of Compound XI) can be performed in the presence of a base. Suitable bases include, but are not limited to, amines (e.g., triethylamine, N,N-diisopropylethylamine, tripropylamine, tributylamine, 1,4-diazabicylo[2.2.2]-octane, 1,8-diazabicyclo[5.4.0]undec-7-ene, etc.), carbonates (e.g., lithium carbonate, sodium carbonate, potassium carbonate, cesium carbonate, etc.), bicarbonates (e.g., lithium bicarbonate, sodium bicarbonate, potassium bicarbonate, cesium bicarbonate, etc.), phosphates (e.g., sodium phosphate, disodium phosphate, potassium phosphate, dipotassium phosphate, cesium phosphate, etc.), basic aromatic compounds (e.g., pyridine, 2,6-lutidine, imidazole, etc.), and hydroxides (e.g., sodium hydroxide, potassium hydroxide, etc.).

In some embodiments, the base is a bicarbonate. In some embodiments, the base is potassium bicarbonate.

In some embodiments, the free basing step (e.g., free basing a salt such as a HCl salt of Compound XI) is performed in the presence of a solvent. Suitable solvents include, but are not limited to, ethers (e.g., tetrahydrofuran, 2-methyltetrahydrofuran, tert-butyl methyl ether, etc.), ketones (e.g., acetone, 2-butanone, 4-methyl-2-pentanone, etc.), esters (e.g., ethyl acetate, isopropyl acetate, methyl acetate, etc.), nitriles (e.g., acetonitrile, etc.), halogenated solvents (e.g., 1,2-dichloroethane, chlorobenzene, etc.) or a combinate of any of the foregoing with water.

In some embodiments, the solvent is a mixed solvent. In some embodiments, the solvent is a mixture of dichloromethane and water.

In some embodiments, the free basing step (e.g., free basing a salt such as a HCl salt of Compound XI) is performed at a temperature of about 0° C. to about 120° C. In some embodiments, the temperature is about 10° C. to about 30° C. In some embodiments, the temperature is about 20° C.

In some embodiments, the free basing step (e.g., free basing a salt such as a HCl salt of Compound XI) is performed in the presence of a base such as a bicarbonate (e.g., potassium bicarbonate), a solvent such as a mixture of dichloromethane and water, and at a suitable temperature such as a temperature of about 10° C. to about 30° C. (e.g., about 20° C.).

Synthesis of a compound of Formula V

Also provided is the synthesis of a compound of Formula V (including Compound V-I and Compound V-Br) or a salt thereof.

A compound of Formula V-1 or a salt thereof may be prepared via an asymmetric cyclization reaction.

### Asymmetric Cyclization Route A

For example, in some embodiments, provided is a process for preparing a compound of Formula V-1

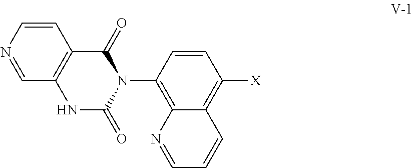

wherein R is a C₁₋₃ alkyl and X is Br or I,
or a salt thereof, comprising:
subjecting a compound of Formula V-2

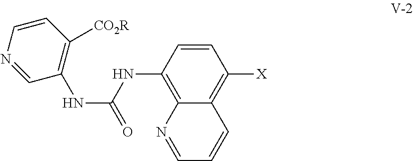

wherein R is a C₁₋₃ alkyl and X is Br or I,
or a salt thereof into a cyclization condition to form a compound of Formula V-1 or a salt thereof.

In some embodiments, R is methyl, ethyl, or isopropyl. In some embodiments, X is Br or I.

In some embodiments, R is methyl and X is I.

In some embodiments, R is methyl and X is Br.

In some embodiments, R is ethyl and X is I.

In some embodiments, R is ethyl and X is Br.

In some embodiments, R is isopropyl and X is I.

In some embodiments, R is isopropyl and X is Br.

In some embodiments, the cyclization condition (e.g., the cyclization condition for providing a compound of Formula V-1 from a compound of Formula V-2) comprises an organocatalyst. Suitable organocatalysts include, but are not limited to, squaramides (e.g., 3-[[3,5-bis(trifluoromethyl)phenyl]amino]-4-[[(8a,9S)-6′-methoxycinchonan-9-yl]amino]-3-cyclobutene-1,2-dione (CAS 1256245-84-7), 3-[[3,5-bis(trifluoroMethyl)phenyl]amino]-4-[(8a,9S)-cinchonan-9-ylamino]-3-cyclobutene-1,2-dione (CAS 1256245-86-9), 3-[[3,5-bis(trifluoromethyl)phenyl]amino]-4-[[(8a,9S)-10,11-dihydrocinchonan9-yl]amino]-3-cyclobutene-1,2-dione (CAS 1407166-62-4), 3-((3,5-bis(trifluoromethyl)phenyl)amino)-4-(((1S,2S)-1,2-diphenyl-2-(piperidin-1-yl)ethyl)amino)cyclobut-3-ene-1,2-dione (CAS 1943732-17-9), 3-[[3,5-bis(trifluoromethyl)phenyl]amino]-4-[[(1S,2S)-2-(1-piperidinyl)cyclohexyl]amino]-3-cyclobutene-1,2-dione (CAS 1312991-15-3), etc.), chiral guanidines (e.g., (2R,6R)-2,6-di-tert-butyl-2,3,5,6-tetrahydro-1H-imidazo[1,2-a]imidazole (CAS 1502026-83-6), etc.), and chiral phase transfer catalysts.

In some embodiments, the organocatalyst is a squaramide organocatalyst. In some embodiments, the organocatalyst is CAS 1256245-84-7.

In some embodiments, the cyclization condition (e.g., the cyclization condition for providing a compound of Formula V-1 from a compound of Formula V-2) comprises a base. Suitable bases include, but are not limited to, carbonates (e.g., lithium carbonate, sodium carbonate, potassium carbonate, cesium carbonate, etc.), alkoxides (e.g., sodium methoxide, sodium tert-butoxide, lithium tert-butoxide, etc.), phosphates (e.g., sodium phosphate, potassium phosphate, etc.), amines (e.g., triethylamine, N,N-diisopropylethylamine, tripropylamine, tributylamine, 4-methylmorpholine, 1,8-diazabicyclo(5.4.0)undec-7-ene, etc.), basic aromatic compounds (e.g., pyridine, 2,6-di-tert-butylpyridine, etc.), and amides (e.g., lithium bis(trimethylsilyl)amide, potassium bis(trimethylsilyl)amide, lithium diisopropylamide, etc.).

In some embodiments, the base is a carbonate or an amine base. In some embodiments, the base is potassium carbonate or 1,8-diazabicyclo(5.4.0)undec-7-ene.

In some embodiments, the cyclization condition (e.g., the cyclization condition for providing a compound of Formula V-1 from a compound of Formula V-2) comprises a solvent. Suitable solvents include, but are not limited to, ethers (e.g., tetrahydrofuran, 2-methyltetrahydrofuran, etc.), polar aprotic solvents (e.g., N,N-dimethylformamide, N,N-dimethylacetamide, N-methyl-2-pyrrolidone, dimethyl sulfoxide, etc.), nitriles (e.g., acetonitrile), and halogenated solvents (e.g., dichloromethane).

In some embodiments, the solvent is a polar aprotic solvent. In some embodiments, the solvent is N-methyl-2-pyrrolidone.

In some embodiments, the cyclization condition (e.g., the cyclization condition for providing a compound of Formula V-1 from a compound of Formula V-2) comprises a temperature of about 0° C. to about 50° C. In some embodiments, the temperature is about 10° C. to about 30° C. In some embodiments, the temperature is about 20° C.

In some embodiments, the cyclization condition (e.g., the cyclization condition for providing a compound of Formula V-1 from a compound of Formula V-2) comprises an organocatalyst such as a squaramide organocatalyst (e.g., CAS 1256245-84-7), a base such as a carbonate (e.g., cesium carbonate) or an amine base (e.g., 1,8-diazabicyclo(5.4.0)undec-7-ene), a solvent such as a polar aprotic solvent (e.g., N-methyl-2-pyrrolidone), and a suitable temperature of about 10° C. to about 30° C. (e.g., about 20° C.).

Additionally or alternatively, a compound of Formula V-1-Br may be prepared by a process comprising one or more or all of the following steps:
(a) subjecting a compound of Formula V-2-Br

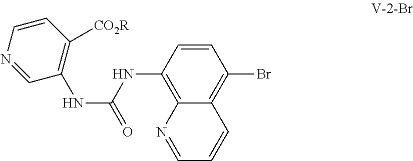

wherein R is (+)-menthyl or (−)-1-phenylethyl,
or a salt thereof into a cyclization condition to form Compound V-1-Br

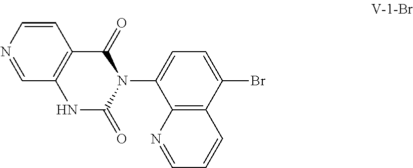

or a salt thereof.

In some embodiments, R is (+)-menthyl. In some embodiments, R is (−)-1-phenylethyl.

In some embodiments, the cyclization condition (e.g., the cyclization condition for providing a compound of Formula V-1-Br from a compound of Formula V-2-Br) comprises an organocatalyst. Suitable organocatalysts include, but are not limited to, squaramides (e.g., 3-[[3,5-bis(trifluoromethyl)phenyl]amino]-4-[[(8a,9S)-6′-methoxycinchonan-9-yl]amino]-3-cyclobutene-1,2-dione (CAS 1256245-84-7), 3-[[3,5-bis(trifluoromethyl)phenyl]amino]-4-[[(8a,9S)-10,11-dihydrocinchonan9-yl]amino]-3-cyclobutene-1,2-dione (CAS 1407166-62-4), 3-((3,5-bis(trifluoromethyl)phenyl)amino)-4-(((1S,2S)-1,2-diphenyl-2-(piperidin-1-yl)ethyl)amino)cyclobut-3-ene-1,2-dione (CAS 1943732-17-9), 3-[[3,5-bis(trifluoromethyl)phenyl]amino]-4-[[(1S,2S)-2-(1-piperidinyl)cyclohexyl]amino]-3-cyclobutene-1,2-dione (CAS 1312991-15-3), etc.), chiral guanidines (e.g., (2R,6R)-2,6-di-tert-butyl-2,3,5,6-tetrahydro-1H-imidazo[1,2-a]imidazole (CAS 1502026-83-6), etc.), and chiral phase transfer catalysts.

In some embodiments, the cyclization condition (e.g., the cyclization condition for providing a compound of Formula V-1-Br from a compound of Formula V-2-Br) does not comprise an organocatalyst.

In some embodiments, the cyclization condition (e.g., the cyclization condition for providing a compound of Formula V-1-Br from a compound of Formula V-2-Br) comprises a base. Suitable bases include, but are not limited to, carbonates (e.g., lithium carbonate, sodium carbonate, potassium carbonate, cesium carbonate, etc.), alkoxides (e.g., sodium methoxide, sodium tert-butoxide, lithium tert-butoxide, etc.), phosphates (e.g., sodium phosphate, potassium phosphate, etc.), amines (e.g., triethylamine, N,N-diisopropylethylamine, tripropylamine, tributylamine, 4-methylmorpholine, 1,8-diazabicyclo[5.4.0]undec-7-ene, etc.), aromatic compounds (e.g., pyridine, 2,6-di-tert-butylpyridine, etc.), and amides (e.g., lithium bis(trimethylsilyl)amide, potassium bis(trimethylsilyl)amide, lithium diisopropylamide, etc.).

In some embodiments, the base is a carbonate. In some embodiments, the base is potassium carbonate.

In some embodiments, the cyclization condition (e.g., the cyclization condition for providing a compound of Formula V-1-Br from a compound of Formula V-2-Br) comprises a solvent. Suitable solvents include, but are not limited to, ethers (e.g., tetrahydrofuran, 2-methyltetrahydrofuran, etc.), polar aprotic solvents (e.g., N-methyl-2-pyrrolidone, N,N-dimethylformamide, N,N-dimethylacetamide, dimethyl sulfoxide, etc.), nitriles (e.g., acetonitrile, etc.), halogenated solvents (e.g., dichloromethane, etc.), aromatics (e.g., benzene, toluene, trifluorotoluene, etc.), nitriles (e.g., acetonitrile, benzonitrile, etc.), and halogenated solvents (e.g., dichloromethane, 1,2-dichloroethane, chlorobenzene, etc.).

In some embodiments, the solvent is a polar aprotic solvent. In some embodiments, the solvent is N-methyl-2-pyrrolidone.

In some embodiments, the cyclization condition (e.g., the cyclization condition for providing a compound of Formula V-1-Br from a compound of Formula V-2-Br) comprises a temperature of about −10° C. to about 60° C. In some embodiments, the temperature is about 10° C. to about 30° C. In some embodiments, the temperature is about 20° C.

In some embodiments, the cyclization condition (e.g., the cyclization condition for providing a compound of Formula V-1-Br from a compound of Formula V-2-Br) comprises a base such as a carbonate (e.g., potassium carbonate), a solvent such as a polar aprotic solvents (e.g., N-methyl-2-pyrrolidone), and at a suitable temperature such as a temperature of about 10° C. to about 30° C. (e.g., about 20° C.).

In some embodiments, the process further comprises converting a compound of Formula V-1 or a salt thereof to a compound of Formula V or a salt thereof according to a process described anywhere herein.

For example, the process may comprise contacting a compound of Formula V-1

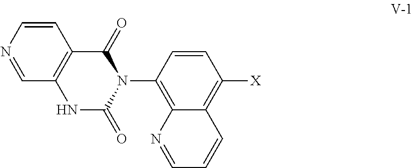

wherein X is Br or I,
or a salt thereof with a methylation agent to form a compound of Formula V

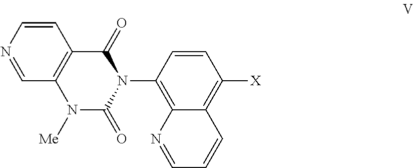

wherein X is Br or I,
or a salt thereof.

In some embodiments, X is Br. In some embodiments, X is I.

Suitable methylation agents (e.g., the methylation agent for providing a compound of Formula V from a compound of Formula V-1) include, but are not limited to, methyl bromide, methyl chloride, methyl triflate, diazomethane, dimethyl sulfate, and dimethyl carbonate.

In some embodiments, the methylation agent is methyl iodide.

In some embodiments, the reaction (e.g., the reaction for proving a compound of Formula V from a compound of Formula V-1) occurs in the presence of a base. Suitable bases include, but are not limited to, carbonates (e.g., lithium carbonate, sodium carbonate, potassium carbonate, cesium carbonate, etc.), alkoxides (e.g., sodium methoxide, sodium tert-butoxide, lithium tert-butoxide, etc.), amines (e.g., triethylamine, N,N-diisopropylethylamine, tripropylamine, tributylamine, 4-methylmorpholine, 1,8-diazabicyclo(5.4.0)undec-7-ene, etc.), aromatic compounds (e.g., pyridine, 2,6-di-tert-butylpyridine, etc.), and amides (e.g., lithium bis(trimethylsilyl)amide, potassium bis(trimethylsilyl)amide, lithium diisopropylamide, etc.).

In some embodiments, the base is an amine base. In some embodiments, the base is 1,8-diazabicyclo(5.4.0)undec-7-ene.

In some embodiments, the reaction (e.g., the reaction for proving a compound of Formula V from a compound of Formula V-1) occurs in the presence of a solvent. Suitable solvents include, but are not limited to, ethers (e.g., tetrahydrofuran, 2-methyltetrahydrofuran, etc.), polar aprotic solvents (e.g., N,N-dimethylformamide, N,N-dimethylacetamide, N-methyl-2-pyrrolidone, dimethyl sulfoxide, etc.), nitriles (e.g., acetonitrile, etc.), halogenated solvents (e.g., dichloromethane, etc.).

In some embodiments, the solvent is a polar aprotic solvent. In some embodiments, the solvent is N-methyl-2-pyrrolidone.

In some embodiments, the reaction (e.g., the reaction for proving a compound of Formula V from a compound of Formula V-1) occurs at a temperature of about −10° C. to about 60° C. In some embodiments, the temperature is about 10° C. to about 30° C. In some embodiments, the temperature is about 20° C.

In some embodiments, the reaction (e.g., the reaction for proving a compound of Formula V from a compound of Formula V-1) occurs in the presence of a methylation agent such as methyl iodide, a base such as an amine base (e.g., 1,8-diazabicyclo(5.4.0)undec-7-ene), a solvent such as a polar aprotic solvents (e.g., N-methyl-2-pyrrolidone), and at a suitable temperature such as a temperature of about 10° C. to about 30° C. (e.g., about 20° C.).

In some embodiments, a compound of Formula V-2 is a compound of Formula V-2-Br

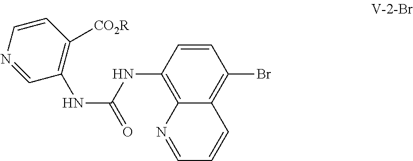

wherein R is (+)-menthyl, (−)-1-phenylethyl, or a C₁₋₃ alkyl.

In some embodiments, provided is a process for preparing a compound of Formula V-2-Br

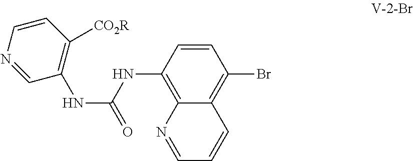

wherein R is (+)-menthyl, (−)-1-phenylethyl, or a C₁₋₃ alkyl,
or a salt thereof, comprising:
(a) subjecting Compound VI-2

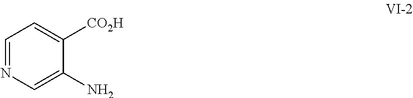

or a salt thereof to a cyclization condition to form Compound VI-3

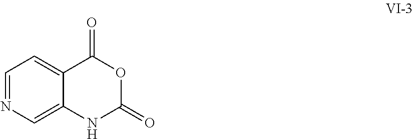 [SMILES: O=c1[nH]c2cnccc2c(=O)o1]

or a salt thereof;
(b) contacting Compound VI-3 or a salt thereof with an alcohol to form a compound of Formula VI-4

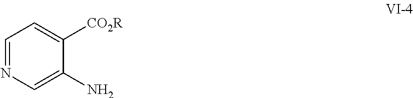

wherein R is (+)-menthyl, (−)-1-phenylethyl, or a C₁₋₃ alkyl,
or a salt thereof;
(c) contacting a compound of Formula VI-4 or a salt thereof with phenyl chloroformate to form a compound of Formula VI-5

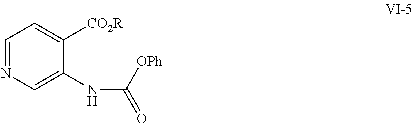

wherein R is (+)-menthyl, (−)-1-phenylethyl, or a C₁₋₃ alkyl,
or a salt thereof; and
(d) contacting a compound of Formula VI-5 or a salt thereof with Compound VII-Br

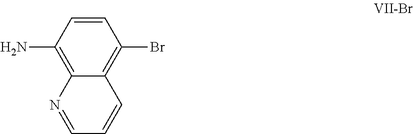 [SMILES: Nc1ccc(Br)c2cccnc12]

or a salt thereof to form a compound of Formula V-2-Br or a salt thereof.

In some embodiments, R is (+)-menthyl or (−)-1-phenylethyl. In some embodiments, R is (+)-menthyl. In some embodiments, R is (−)-1-phenylethyl.

In some embodiments, R is a C₁₋₃ alkyl. In some embodiments, R is ethyl. In some embodiments, R is isopropyl.

In some embodiments, the process comprises subjecting Compound VI-2 or a salt thereof to a cyclization condition to form Compound VI-3 or a salt thereof.

The cyclization condition (e.g., the cyclization condition for providing Compound VI-3 from Compound VI-2) may comprise a carbonyl (or thionyl) source such as 1,1′-carbonyldiimidazole, carbon monoxide, phosgene, triphosgene, or thionyl chloride.

In some embodiments, the carbonyl source is 1,1′-carbonyldiimidazole.

In some embodiments, the cyclization condition (e.g., the cyclization condition for providing Compound VI-3 from Compound VI-2) comprises a solvent. Suitable solvents include, but are not limited to, ethers (e.g., tetrahydrofuran, 2-methyltetrahydrofuran, 1,4-dioxane, etc.), aromatic solvents (e.g., benzene, toluene, trifluorotoluene, etc.), nitriles (e.g., acetonitrile, benzonitrile, etc.), halogenated solvents (e.g., dichloromethane, 1,2-dichloroethane, chlorobenzene, etc.), and polar aprotic solvents (e.g., N,N-dimethylformamide, N,N-dimethylacetamide, dimethyl sulfoxide, etc.).

In some embodiments, the solvent is an ether solvent. In some embodiments, the solvent is tetrahydrofuran.

In some embodiments, the cyclization condition (e.g., the cyclization condition for providing Compound VI-3 from Compound VI-2) comprises a temperature of about 0° C. to about 50° C. In some embodiments, the temperature is about 10° C. to about 30° C. In some embodiments, the temperature is about 20° C.

In some embodiments, the cyclization condition (e.g., the cyclization condition for providing Compound VI-3 from Compound VI-2) comprises a carbonyl (or thionyl) source such as 1,1′-carbonyldiimidazole, a solvent such as an ether solver (e.g., tetrahydrofuran), and a suitable temperature such as a temperature of about 10° C. to about 30° C. (e.g., about 20° C.).

In some embodiments, the process comprises contacting Compound VI-3 or a salt thereof with an alcohol to form Compound VI-4 or a salt thereof.

The alcohol (e.g., the alcohol for providing Compound VI-4 from Compound VI-3) may be a chiral alcohol that is optically pure, such as (+)-menthol or (−)-1-phenylethanol. Additionally or alternatively, the alcohol may be ethanol or isopropanol.

In some embodiments, the reaction (e.g., the reaction for providing Compound VI-4 from Compound VI-3) occurs in the presence of a solvent. Suitable solvents include, but are not limited to, ethers (e.g., tetrahydrofuran, 2-methyltetrahydrofuran, 1,4-dioxane, etc.), aromatic solvents (e.g., benzene, trifluorotoluene, etc.), nitriles (e.g., acetonitrile, benzonitrile, etc.), halogenated solvents (e.g., dichloromethane, 1,2-dichloroethane, chlorobenzene, etc.), and polar aprotic solvents (e.g., N,N-dimethylformamide, N,N-dimethylacetamide, dimethyl sulfoxide, etc.).

In some embodiments, the solvent is an ether solvent. In some embodiments, the solvent is tetrahydrofuran.

In some embodiments, the reaction (e.g., the reaction for providing Compound VI-4 from Compound VI-3) occurs at a temperature of about 10° C. to about 100° C. In some embodiments, the temperature is about 40° C. to about 60° C. In some embodiments, the temperature is about 50° C.

In some embodiments, the reaction (e.g., the reaction for providing Compound VI-4 from Compound VI-3) occurs in the presence of an alcohol (e.g., (+)-menthol, (−)-1-phenylethanol, ethanol, or isopropanol), a solvent such as an ether solver (e.g., tetrahydrofuran), and at a suitable temperature such as a temperature of about 40° C. to about 60° C. (e.g., about 50° C.).

In some embodiments, the process comprises contacting Compound VI-4 or a salt thereof with phenyl chloroformate to form a compound of Formula VI-5 or a salt thereof.

In some embodiments, the reaction (e.g., the reaction between Compound VI-4 and phenyl chloroformate) occurs in the presence of a base. Suitable bases include, but are not limited to, carbonates (e.g., lithium carbonate, sodium carbonate, potassium carbonate, cesium carbonate, etc.), alkoxides (e.g., sodium methoxide, sodium tert-butoxide, lithium tert-butoxide, etc.), phosphates (e.g., sodium phosphate, potassium phosphate, etc.) amines (e.g., triethylamine, N,N-diisopropylethylamine, tripropylamine, tributylamine, 4-methylmorpholine, 1,8-diazabicyclo[5.4.0]undec-7-ene, etc.), basic aromatic compounds (e.g., pyridine, 2,6-lutidine, 2,6-di-tert-butylpyridine, etc.), amides (e.g., lithium bis(trimethylsilyl)amide, potassium bis(trimethylsilyl)amide, lithium diisopropylamide, etc.).

In some embodiments, the base is a basic aromatic compound. In some embodiments, the base is pyridine.

In some embodiments, the reaction (e.g., the reaction between Compound VI-4 and phenyl chloroformate) occurs in the presence of a solvent. Suitable solvents include, but are not limited to, ethers (e.g., tetrahydrofuran, 2-methyltetrahydrofuran, 1,4-dioxane, etc.), aromatic solvents (e.g., benzene, toluene, trifluorotoluene, etc.), nitriles (e.g., acetonitrile, benzonitrile, etc.), halogenated solvents (e.g., dichloromethane, 1,2-dichloroethane, chlorobenzene, etc.), and polar aprotic solvents (e.g., N,N-dimethylformamide, N,N-dimethylacetamide, dimethyl sulfoxide, etc.).

In some embodiments, the solvent is an ether solvent. In some embodiments, the solvent is tetrahydrofuran.

In some embodiments, the reaction (e.g., the reaction between Compound VI-4 and phenyl chloroformate) occurs at a temperature of about 0° C. to about 60° C. In some embodiments, the temperature is about 30° C. to about 50° C. In some embodiments, the temperature is about 40° C.

In some embodiments, the reaction (e.g., the reaction between Compound VI-4 and phenyl chloroformate) occurs in the presence of a base such as basic aromatic compound (e.g., pyridine), a solvent such as an ether solvent (e.g., tetrahydrofuran), and at a suitable temperature such as a temperature of about 30° C. to about 50° C. (e.g., about 40° C.).

In some embodiments, the process comprises contacting a compound of Formula VI-5 or a salt thereof with Compound VII-Br or a salt thereof to form a compound of Formula V-2-Br or a salt thereof.

In some embodiments, the reaction (e.g., the reaction between a compound of Formula VI-5 and Compound VII-Br) occurs in the presence of a base. Suitable bases include, but are not limited to, carbonates (e.g., lithium carbonate, sodium carbonate, potassium carbonate, cesium carbonate, etc.), alkoxides (e.g., sodium methoxide, sodium tert-butoxide, lithium tert-butoxide, etc.), phosphates (e.g., sodium phosphate, potassium phosphate, etc.), amines (e.g., N,N-diisopropylethylamine, triethylamine, tripropylamine, tributylamine, 4-methylmorpholine, 1,8-diazabicyclo[5.4.0]undec-7-ene, etc.), basic aromatic compounds (e.g., pyridine, 2,6-di-tert-butylpyridine, etc.), and amides (e.g., lithium bis(trimethylsilyl)amide, potassium bis(trimethylsilyl)amide, lithium diisopropylamide, etc.).

In some embodiments, the base is an amine base. In some embodiments, the base is triethylamine.

In some embodiments, the reaction (e.g., the reaction between a compound of Formula VI-5 and Compound VII-Br) occurs in the presence of a solvent. Suitable solvents include, but are not limited to, ethers (e.g., tetrahydrofuran, 2-methyltetrahydrofuran, etc.), polar aprotic solvents (e.g., N,N-dimethylformamide, N,N-dimethylacetamide, dimethyl sulfoxide, etc.), nitriles (e.g., acetonitrile, benzonitrile, etc.), halogenated solvents (e.g., dichloromethane, 1,2-dichloroethane, chlorobenzene, etc.), and aromatic solvents (e.g., benzene, toluene, trifluorotoluene, etc.).

In some embodiments, the solvent is an aromatic solvent. In some embodiments, the solvent is trifluorotoluene.

In some embodiments, the reaction (e.g., the reaction between a compound of Formula VI-5 and Compound VII-Br) occurs at a temperature of about 20° C. to about 100° C. In some embodiments, the temperature is about 60° C. to about 80° C. In some embodiments, the temperature is about 70° C.

In some embodiments, the reaction (e.g., the reaction between a compound of Formula VI-5 and Compound VII-Br) occurs in the presence of a base such as an amine base (e.g., triethylamine), a solvent such as an aromatic solvent (e.g., trifluorotoluene), and at a suitable temperature such as a temperature of about 60° C. to about 80° C. (e.g., about 70° C.).

### Asymmetric Cyclization Route B

Additionally or alternatively, Compound V-I

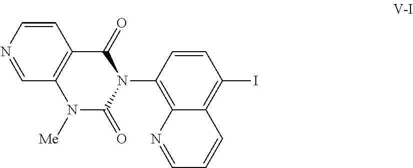

or a salt thereof may be prepared by a process comprising one or more or all of the following steps:
(a) converting pyridine-3,4-dicarboxylic acid into an anhydride intermediate that is furo[3,4-c]pyridine-1,3-dione;
(b) contacting the anhydride intermediate with an chiral auxiliary to form a compound of Formula VI-6

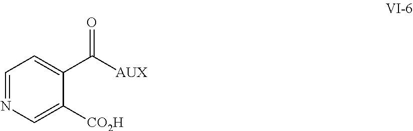

wherein AUX is a residue from the chiral auxiliary,
or a salt thereof;
(b) contacting a compound of Formula VI-6 or a salt thereof with Compound VII-I

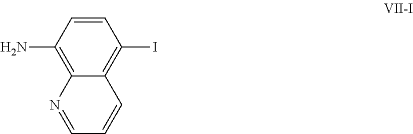 [SMILES: Nc1ccc(I)c2cccnc12]

or a salt thereof to form a compound of Formula V-5

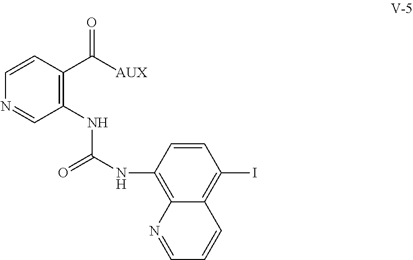

wherein AUX is a residue from the chiral auxiliary,
or a salt thereof;
(c) subjecting a compound of Formula V-5 or a salt thereof to a cyclization condition to form Compound V-1-I

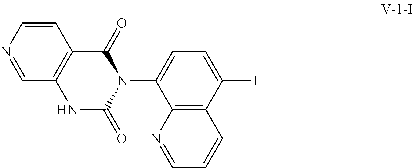

or a salt thereof;
(d) contacting Compound V-1-I or a salt thereof with a methylation agent to form Compound V-I

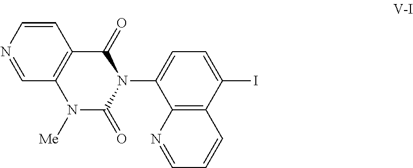

or a salt thereof.

In some embodiments, the process comprises converting pyridine-3,4-dicarboxylic acid into an anhydride intermediate that is furo[3,4-c]pyridine-1,3-dione according to a process described anywhere herein.

In some embodiments, the process comprises contacting the anhydride intermediate (i.e., furo[3,4-c]pyridine-1,3-dione) with an chiral auxiliary to form a compound of Formula VI-6 or a salt thereof.

As used here, term “chiral auxiliary” refers to a chemical compound or unit that is temporarily incorporated into an organic compound in order to control the stereochemical outcome of the synthesis.

Suitable chiral auxiliaries (e.g., chiral auxiliaries for reacting with furo[3,4-c]pyridine-1,3-dione) include, but are not limited to, (tert-butoxycarbonyl)-D-threoninate esters (e.g., methyl (tert-butoxycarbonyl)-D-threoninate, isopropyl (tert-butoxycarbonyl)-D-threoninate, tert-butyl (tert-butoxycarbonyl)-D-threoninate, diphenylmethyl (tert-butoxycarbonyl)-D-threoninate, benzyl (tert-butoxycarbonyl)-D-threoninate, 1-phenylethyl (tert-butoxycarbonyl)-D-threoninate, etc.), methyl (methoxycarbonyl)-D-threoninate, and chiral oxazolidinones (e.g., (S)-(−)-4-isopropyl-2-oxazolidinone, (S)-(+)-4-phenyl-2-oxazolidinone, (S)-4-benzyl-2-oxazolidinone, (4S)-(−)-4-isopropyl-5,5-diphenyl-2-oxazolidinone), (S)-4-benzylthiazolidine-2-thione, (1S)-(−)-2,10-camphorsultam, (R)-1-(anthracen-9-yl)-2,2,2-trifluoroethan-1-ol, (R)-2-methyl-1-phenylpropan-1-ol, etc.).

In some embodiments, the chiral auxiliary is a (tert-butoxycarbonyl)-D-threoninate ester. In some embodiments, the chiral auxiliary is methyl (tert-butoxycarbonyl)-D-threoninate, isopropyl (tert-butoxycarbonyl)-D-threoninate, tert-butyl (tert-butoxycarbonyl)-D-threoninate, or diphenylmethyl (tert-butoxycarbonyl)-D-threoninate.

In some embodiments, a compound of Formula VI-6 is a compound of Formula VI-7

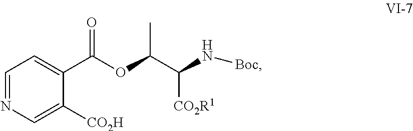

wherein R¹ is C₁₋₆ alkyl or diphenylmethyl; and
a compound of Formula V-5 is a compound of Formula V-6

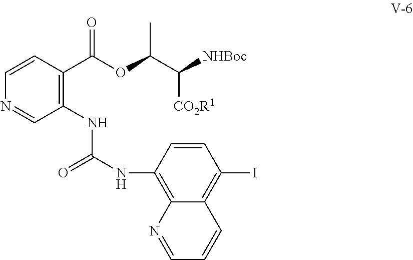

wherein R¹ is C₁₋₆ alkyl or diphenylmethyl.

In some embodiments, R¹ is methyl, isopropyl, tert-butyl, or diphenylmethyl.

In some embodiments, the reaction (e.g., the reaction between furo[3,4-c]pyridine-1,3-dione and the chiral auxiliary) occurs in the presence of a solvent. Suitable solvents include, but are not limited to, ethers (e.g., tetrahydrofuran, 2-methyltetrahydrofuran, tert-butyl methyl ether, 1,4-dioxane, etc.), esters (e.g., ethyl acetate, isopropyl acetate, etc.), hydrocarbons (e.g., benzene, toluene, etc.), nitriles (e.g., acetonitrile, benzonitrile, etc.), halogenated solvents (e.g., dichloromethane, 1,2-dichloroethane, etc.), polar aprotic solvents (e.g., N,N-dimethylformamide, N,N-dimethylacetamide, N-methyl-2-pyrrolidone, dimethyl sulfoxide).

In some embodiments, the chiral auxiliary is methyl (tert-butoxycarbonyl)-D-threoninate or isopropyl (tert-butoxycarbonyl)-D-threoninate, and the solvent is a halogenated solvent. In some embodiments, the chiral auxiliary is methyl (tert-butoxycarbonyl)-D-threoninate or isopropyl (tert-butoxycarbonyl)-D-threoninate, and the solvent is dichloromethane.

In some embodiments, the chiral auxiliary is tert-butyl (tert-butoxycarbonyl)-D-threoninate, and the solvent is a hydrocarbon solvent. In some embodiments, the chiral auxiliary is tert-butyl (tert-butoxycarbonyl)-D-threoninate, and the solvent is toluene.

In some embodiments, the chiral auxiliary is diphenylmethyl (tert-butoxycarbonyl)-D-threoninate, and the solvent is an ether. In some embodiments, the chiral auxiliary is diphenylmethyl (tert-butoxycarbonyl)-D-threoninate, and the solvent is tetrahydrofuran.

In some embodiments, the reaction (e.g., the reaction between furo[3,4-c]pyridine-1,3-dione and the chiral auxiliary) occurs in the presence of a base. Suitable bases include, but are not limited to, amines (e.g., N,N-diisopropylethylamine, triethylamine, tripropylamine, tributylamine, 4-methylmorpholine, 1,8-diazabicyclo[5.4.0]undec-7-ene, etc.), basic aromatic compounds (e.g., pyridine, 2,6-di-tert-butylpyridine, etc.), amides (e.g., lithium bis(trimethylsilyl)amide, potassium bis(trimethylsilyl)amide, lithium diisopropylamide, etc.), carbonates (e.g., lithium carbonate, sodium carbonate, cesium carbonate, etc.), alkoxides (e.g., sodium methoxide, sodium tert-butoxide, lithium tert-butoxide, etc.), and phosphates (e.g., sodium phosphate, potassium phosphate, etc.).

In some embodiments, the reaction (e.g., the reaction between furo[3,4-c]pyridine-1,3-dione and the chiral auxiliary) occurs in the absence of a base.

In some embodiments, the chiral auxiliary is methyl (tert-butoxycarbonyl)-D-threoninate, isopropyl (tert-butoxycarbonyl)-D-threoninate, or tert-butyl (tert-butoxycarbonyl)-D-threoninate, and the reaction (e.g., the reaction between furo[3,4-c]pyridine-1,3-dione and the chiral auxiliary) occurs in the absence of a base.

In some embodiments, the chiral auxiliary is diphenylmethyl (tert-butoxycarbonyl)-D-threoninate, and the reaction (e.g., the reaction between furo[3,4-c]pyridine-1,3-dione and the chiral auxiliary) occurs in the presence of an amine base. In some embodiments, the base is triethylamine.

In some embodiments, the reaction (e.g., the reaction between furo[3,4-c]pyridine-1,3-dione and the chiral auxiliary) occurs in the presence of a catalyst, such as 4-(dimethylamino)pyridine, N-methylimidazole, pyridine, or 1,8-diazabicyclo[5.4.0]undec-7-ene.

In some embodiments, the reaction (e.g., the reaction between furo[3,4-c]pyridine-1,3-dione and the chiral auxiliary) occurs in the absence of a catalyst,

In some embodiments, the chiral auxiliary is methyl (tert-butoxycarbonyl)-D-threoninate, isopropyl (tert-butoxycarbonyl)-D-threoninate, or tert-butyl (tert-butoxycarbonyl)-D-threoninate, and the reaction (e.g., the reaction between furo[3,4-c]pyridine-1,3-dione and the chiral auxiliary) occurs in the absence of a catalyst.

In some embodiments, the chiral auxiliary is diphenylmethyl (tert-butoxycarbonyl)-D-threoninate, and the reaction (e.g., the reaction between furo[3,4-c]pyridine-1,3-dione and the chiral auxiliary) occurs in the presence of a catalyst that is 4-(dimethylamino)pyridine.

In some embodiments, the reaction (e.g., the reaction between furo[3,4-c]pyridine-1,3-dione and the chiral auxiliary) occurs at a temperature of about 0° C. to about 80° C. In some embodiments, the temperature is about 30° C. to about 50° C. In some embodiments, the temperature is about 40° C.

In some embodiments, the reaction (e.g., the reaction between furo[3,4-c]pyridine-1,3-dione and the chiral auxiliary) occurs in the presence of a chiral auxiliary such as methyl (tert-butoxycarbonyl)-D-threoninate or isopropyl (tert-butoxycarbonyl)-D-threoninate, a solvent such as a halogenated solvent (e.g. dichloromethane), and at a suitable temperature such as a temperature of about 30° C. to about 50° C. (e.g., about 40° C.).

In some embodiments, the reaction (e.g., the reaction between furo[3,4-c]pyridine-1,3-dione and the chiral auxiliary) occurs in the presence of a chiral auxiliary such as tert-butyl (tert-butoxycarbonyl)-D-threoninate, a solvent such as a hydrocarbon solvent (e.g., toluene), and at a suitable temperature such as a temperature of about 30° C. to about 50° C. (e.g., about 40° C.).

In some embodiments, the reaction (e.g., the reaction between furo[3,4-c]pyridine-1,3-dione and the chiral auxiliary) occurs in the presence of a chiral auxiliary such as diphenylmethyl (tert-butoxycarbonyl)-D-threoninate, a solvent such as an ether (e.g., tetrahydrofuran), a base such as an amine base (e.g., triethylamine), a catalyst such as 4-(dimethylamino)pyridine, and at a suitable temperature such as a temperature of about 30° C. to about 50° C. (e.g., about 40° C.).

In some embodiments, the process comprises contacting a compound of Formula VI-6 or a salt thereof with Compound VII-I or a salt thereof to form a compound of Formula V-5 or a salt thereof.

In some embodiments, the compound of Formula VI-6 is a compound of Formula VI-7, the compound of Formula V-5 is a compound of Formula V-6, and the process comprises contacting a compound of Formula VI-7 or a salt thereof with Compound VII-I or a salt thereof to form a compound of Formula V-6 or a salt thereof.

In some embodiments, the reaction (e.g., the reaction between a compound of Formula VI-7 and Compound VII-I) occurs in the presence of a base. Suitable bases include, but are not limited to, amines (e.g., N,N-diisopropylethylamine, triethylamine, tripropylamine, tributylamine, 4-methylmorpholine, etc.), and basic aromatic compounds (e.g., pyridine, 2,6-lutidine, imidazole, etc.).

In some embodiments, the base is an amine base. In some embodiments, the base is triethylamine.

In some embodiments, the reaction (e.g., the reaction between a compound of Formula VI-7 and Compound VII-I) occurs in the presence of an azide source, such as diphenylphosphoryl azide (DPPA), trimethylsilyl azide, sodium azide, or lithium azide.

In some embodiments, the azide source is diphenylphosphoryl azide (DPPA).

In some embodiments, the reaction (e.g., the reaction between a compound of Formula VI-7 and Compound VII-I) occurs in the presence of a solvent. Suitable solvents include, but are not limited to, ethers (e.g., tetrahydrofuran, 2-methyltetrahydrofuran, 1,4-dioxane, etc.), aromatic solvents (e.g., benzene, toluene, trifluorotoluene, etc.), nitriles (e.g., acetonitrile, benzonitrile, etc.), and halogenated solvents (e.g., dichloromethane, 1,2-dichloethane, chlorobenzene, etc.).

In some embodiments, the solvent is an aromatic solvent. In some embodiments, the solvent is trifluorotoluene.

In some embodiments, the reaction (e.g., the reaction between a compound of Formula VI-7 and Compound VII-I) occurs at a temperature of about −10° C. to about 120° C. In some embodiments, the temperature is about 50° C. to about 70° C. In some embodiments, the temperature is about 60° C.

In some embodiments, the reaction (e.g., the reaction between a compound of Formula VI-7 and Compound VII-I) occurs in the presence of a base such as an amine base (e.g., triethylamine), an azide source such as diphenylphosphoryl azide (DPPA), a solvent such as an aromatic solvent (e.g., trifluorotoluene), and at a suitable temperature such as a temperature of about 50° C. to about 70° C. (e.g., about 60° C.).

In some embodiments, the process comprises subjecting a compound of Formula V-5 or a salt thereof to a cyclization condition to form Compound V-1-I or a salt thereof.

In some embodiments, the compound of Formula V-5 is a compound of Formula V-6, and process comprises subjecting a compound of Formula V-6 or a salt thereof to a cyclization condition to form Compound V-1-I or a salt thereof.

In some embodiments, the cyclization condition (e.g., the cyclization condition for providing Compound V-1-I from a compound of Formula V-6) comprises a base. Suitable bases include, but are not limited to, carbonates (e.g., lithium carbonate, sodium carbonate, potassium carbonate, cesium carbonate, etc.), alkoxides (e.g., sodium methoxide, sodium tert-butoxide, lithium tert-butoxide, etc.), phosphates (e.g., sodium phosphate, potassium phosphate, etc.), amines (e.g., triethylamine, N,N-diisopropylethylamine, tripropylamine, tributylamine, 4-methylmorpholine, 1,8-diazabicyclo[5.4.0]undec-7-ene, etc.), basic aromatic compounds (e.g., pyridine, 2,6-di-tert-butylpyridine, etc.), and amides (e.g., lithium bis(trimethylsilyl)amide, potassium bis(trimethylsilyl)amide, lithium diisopropylamide, etc.).

In some embodiments, the base is a carbonate. In some embodiments, the base is potassium carbonate.

In some embodiments, the cyclization condition (e.g., the cyclization condition for providing Compound V-1-I from a compound of Formula V-6) comprises a solvent. Suitable solvents include, but are not limited to, ethers (e.g., tetrahydrofuran, 2-methyltetrahydrofuran, etc.), polar aprotic solvents (e.g., N,N-dimethylformamide, N,N-dimethylacetamide, N-methyl-2-pyrrolidone, dimethyl sulfoxide, etc.), nitriles (e.g., acetonitrile, etc.), and halogenated solvents (e.g., dichloromethane).

In some embodiments, the solvent is a polar aprotic solvent. In some embodiments, the solvent is N-methyl-2-pyrrolidone.

In some embodiments, the cyclization condition (e.g., the cyclization condition for providing Compound V-1-I from a compound of Formula V-6) comprises a temperature of about −10° C. to about 60° C. In some embodiments, the temperature is about 10° C. to about 30° C. In some embodiments, the temperature is about 20° C.

In some embodiments, the cyclization condition (e.g., the cyclization condition for providing Compound V-1-I from a compound of Formula V-6) comprises a base such as a carbonate (e.g., potassium carbonate), a solvent such as a polar aprotic solvent (e.g., N-methyl-2-pyrrolidone), and a suitable temperature such as a temperature of about 10° C. to about 30° C. (e.g., about 20° C.).

In some embodiments, the process comprises contacting Compound V-1-I or a salt thereof with a methylation agent to form Compound of V-I or a salt thereof.

In some embodiments, Compound V-1-I (e.g., Compound V-1-I obtained from step (c)) is subject to the methylation step without isolation and/or purification.

The methylation agent (e.g., the methylation agent for providing Compound of V-I from Compound V-1-I) may be methyl iodide, methyl bromide, methyl chloride, methyl triflate, diazomethane, dimethyl sulfate, or dimethyl carbonate.

In some embodiments, the methylation agent is methyl iodide.

In some embodiments, the reaction (e.g., the reaction for providing Compound of V-I from Compound V-1-I) occurs at a temperature of about −10° C. to about 60° C. In some embodiments, the temperature is about 10° C. to about 30° C. In some embodiments, the temperature is about 20° C.

### Synthesis of Compound II and Synthetic Intermediates

Also provided are processes for preparing Compound II or a salt thereof (or a precursor for preparing Compound II or a salt thereof). In some embodiments, the Compound II or a salt thereof (or a precursor for preparing Compound II or a salt thereof) prepared according to a process described herein may be used for preparing Compound I or a salt thereof.

### Route A

In some embodiments, provided is a process for preparing Compound II

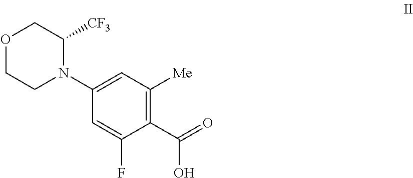 [SMILES: Cc1cc(N2CCOC[C@@H]2C(F)(F)F)cc(F)c1C(=O)O]

or a salt thereof, comprising:
(a) contacting Compound VIII

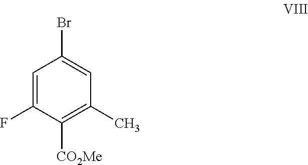

or a salt thereof with Compound IX

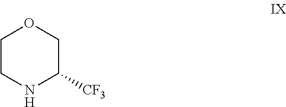

or a salt thereof in the presence of a catalytic system to provide Compound II-2

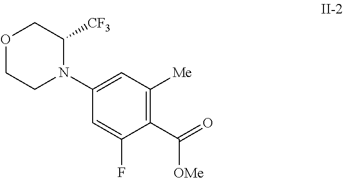 [SMILES: COC(=O)c1c(C)cc(N2CCOC[C@@H]2C(F)(F)F)cc1F]

or a salt thereof; and
(b) subjecting Compound II-2 or a salt thereof into a hydrolysis condition to form Compound II or a salt thereof.

In some embodiments, the process comprises contacting Compound VIII or a salt thereof with Compound IX or a salt thereof in the presence of a catalytic system to provide Compound II-2 or a salt thereof.

In some embodiments, the catalytic system (e.g., the catalytic system for providing Compound II-2 from Compound VIII) comprises a catalyst. In some embodiments, the catalyst is a palladium catalyst. Suitable palladium catalysts include, but are not limited to, a palladium(II) source (e.g., palladium(II) acetate, palladium(II) chloride, etc.) and a palladium(0) source (e.g., tetrakis(triphenylphosphine)palladium(0), etc.).

In some embodiments, the catalyst is a palladium(II) source. In some embodiments, the catalyst is palladium(II) acetate.

In some embodiments, the catalytic system (e.g., the catalytic system for providing Compound II-2 from Compound VIII) comprises a ligand. In some embodiments, the ligand is a monodentate or bidentate phosphine ligand. Exemplary phosphine ligands include, but are not limited to, 2-dicyclohexylphosphino-2′-6′-diisopropoxybiphenyl (RuPhos), 2,2′-bis(diphenylphosphino)-1,1′-binaphthyl (rac-BINAP), (2-dicyclohexylphosphino-2′,6′-dimethoxybiphenyl) [2-(2′-amino-1,1′-biphenyl)]methanesulfonate (SPhos G3), [(4,5-Bis(diphenylphosphino)-9,9-dimethylxanthene)-2-(2′-amino-1,1′-biphenyl)]methanesulfonate (XantPhos G3), [(2-di-cyclohexylphosphino-3,6-dimethoxy-2′,4′,6′-triisopropyl-1,1′-biphenyl)-2-(2′-amino-1,1′-biphenyl)]methanesulfonate (BrettPhos G3), methanesulfonato 2-dicyclohexylphosphino-2-(N,N-dimethylamino)biphenyl(2′-amino-1,1′-biphenyl-2-yl) (DavePhos G3), tri-tert-butylphosphine (P^{t}Bus), and {(R)-1-[(Sp)-2-(dicyclohexylphosphino)ferrocenyl]ethyldi-tert-butylphosphine}[2-(2′-amino-1,1′-biphenyl)]methanesulfonate (JosiPhos G3).

In some embodiments, the ligand is 2-dicyclohexylphosphino-2′-6′-diisopropoxybiphenyl (RuPhos).

In some embodiments, the catalytic system (e.g., the catalytic system for providing Compound II-2 from Compound VIII) comprises a base. Suitable bases include, but are not limited to, amines (e.g., triethylamine, N,N-diisopropylethylamine, tripropylamine, tributylamine, etc.), carbonates (e.g., lithium carbonate, sodium carbonate, potassium carbonate, cesium carbonate, etc.), alkoxides (e.g., sodium methoxide, sodium ethoxide, sodium tert-butoxide, sodium tert-pentoxide, lithium tert-butoxide, potassium ethoxide, potassium tert-butoxide, potassium tert-pentoxide, etc.), lithium bis(trimethylsilyl)amide, hydroxides (e.g., potassium hydroxide, sodium hydroxide, etc.), amides (e.g., lithium bis(trimethylsilyl)amide, potassium bis(trimethylsilyl)amide, lithium diisopropylamide, etc.), and any combination thereof.

In some embodiments, the base is a combination of an carbonate and an amine base. In some embodiments, the base is a combination of cesium carbonate and tripropylamine.

In some embodiments, the catalytic system (e.g., the catalytic system for providing Compound II-2 from Compound VIII) comprises a phase transfer catalyst. Suitable phase transfer catalysts include, but are not limited to, tetrabutylammonium salts (e.g., tetrabutylammonium iodide, tetrabutylammonium bromide, tetrabutylammonium chloride, tetrabutylammonium hydrogen sulfate, etc.), potassium tetrafluoroborate, potassium hexafluorophosphate, lithium bromide, benzyltributylammonium bromide, and phosphonium salts (e.g., tetrabutylphosphonium chloride, etc.).

In some embodiments, the catalytic system (e.g., the catalytic system for providing Compound II-2) does not comprise a phase transfer catalyst.

In some embodiments, the catalytic system (e.g., the catalytic system for providing Compound II-2 from Compound VIII) comprises a solvent. Suitable solvents include, but are not limited to, ethers (e.g., tetrahydrofuran, 2-methyltetrahydrofuran, 1,4-dioxane, etc.), esters (e.g., isopropyl acetate, etc.), aromatics (e.g., toluene, trifluorotoluene, etc.), polar aprotic solvents (e.g., N-methyl-2-pyrrolidone, N,N-dimethylformamide, N,N-dimethylacetamide, dimethyl sulfoxide, sulfolane, etc.), halogenated (e.g., dichloromethane, 1,2-dichloroethane, etc.), hydrocarbons (e.g., n-heptane, pentane, etc.), any combination thereof, and a combinations of any of the foregoing with water.

In some embodiments, the solvent is a mixed solvent. In some embodiments, the solvent is a mixture of toluene and N-methyl-2-pyrrolidone.

In some embodiments, the reaction between Compound VIII or a salt thereof and Compound IX or a salt thereof occurs at a temperature of about 20° C. to about 120° C. In some embodiments, the temperature is about 85° C. to about 105° C. In some embodiments, the temperature is about 95° C.

In some embodiments, the reaction between Compound VIII or a salt thereof and Compound IX or a salt thereof occurs at a suitable temperature such as a temperature of about 85° C. to about 105° C. (e.g., about 95° C.), and in the presence of a catalytic system comprising a catalyst such as a palladium(II) source (e.g., palladium(II) acetate), a ligand such as a monodentate or bidentate phosphine ligand (e.g., 2-dicyclohexylphosphino-2′-6′-diisopropoxybiphenyl (RuPhos), a base such as a mixture of a carbonate (e.g., cesium carbonate) and an amine base (e.g., tripropylamine), and a solvent such as a mixture of toluene and N-methyl-2-pyrrolidone.

In some embodiments, the process further comprises subjecting Compound II-2 or a salt thereof into a hydrolysis condition to form Compound II or a salt thereof.

In some embodiments, the hydrolysis condition (e.g., the hydrolysis condition for providing Compound II from Compound II-2) comprises a base. Suitable bases include, but are not limited to, hydroxides (e.g., lithium hydroxide, sodium hydroxide, potassium hydroxide, ammonium hydroxide, etc.), carbonates (e.g., lithium carbonate, potassium carbonate, sodium carbonate, cesium carbonate, etc.), alkoxides (e.g., sodium methoxide, sodium tert-butoxide, potassium tert-butoxide, etc.), and phosphates (e.g., potassium phosphate, sodium phosphate, etc.).

In some embodiments, the base is a hydroxide. In some embodiments, the base is sodium hydroxide.

In some embodiments, the hydrolysis condition (e.g., the hydrolysis condition for providing Compound II from Compound II-2) comprises a phase transfer catalyst. Exemplary phase transfer catalysts include, but are not limited to, tetrabutylammonium salts (e.g., tetrabutylammonium iodide, tetrabutylammonium bromide, tetrabutylammonium chloride, tetrabutylammonium hydrogen sulfate, etc.), potassium tetrafluoroborate, potassium hexafluorophosphate, lithium bromide, benzyltributylammonium bromide, and phosphonium salts (e.g., tetrabutylphosphonium chloride, etc.).

In some embodiments, the hydrolysis condition (e.g., the hydrolysis condition for providing Compound II from Compound II-2) does not comprise a phase transfer catalyst.

In some embodiments, the hydrolysis condition (e.g., the hydrolysis condition for providing Compound II from Compound II-2) comprises a solvent, Suitable solvents include, but are not limited to, alcohols (e.g., methanol, etc.), ether (e.g., tetrahydrofuran, 2-methyltetrahydrofuran, tert-butyl methyl ether, 1,4-dioxane, etc.), ketones (e.g., acetone, 2-butanone, etc.), polar aprotic solvents (e.g., N,N-dimethylformamide, N,N-dimethylacetamide, N-methyl-2-pyrrolidone, dimethyl sulfoxide, etc.), nitriles (e.g., acetonitrile), aromatic solvents (e.g., benzene, toluene, etc.), and a combination of any of the foregoing with water.

In some embodiments, the solvent is a mixed solvent. In some embodiments, the solvent is a mixture of water, toluene, and methanol.

In some embodiments, the hydrolysis condition (e.g., the hydrolysis condition for providing Compound II from Compound II-2) comprises a temperature of about 20° C. to about 120° C. In some embodiments, the temperature is about 50° C. to about 70° C. In some embodiments, the temperature is about 60° C.

In some embodiments, the hydrolysis condition (e.g., the hydrolysis condition for providing Compound II from Compound II-2) comprises a base such as a hydroxide (e.g., sodium hydroxide), a solvent such as a mixture of water, toluene, and methanol, and a suitable temperature such as a temperature of about 50° C. to about 70° C. (e.g., about 60° C.).

In some embodiments, Compound VIII or a salt thereof is prepared by contacting 4-bromo-2-fluoro-6-methylbenzoic acid or a salt thereof with a methylation agent.

Suitable methylation agent (e.g., methylation agent for providing Compound VIII) include, but are not limited to, methyl iodide, methyl bromide, methyl chloride, methyl triflate, diazomethane, dimethyl sulfate, and dimethyl carbonate.

In some embodiments, the methylation agent is methyl iodide.

In some embodiments, the methylation reaction (e.g., the methylation reaction for providing Compound VIII) occurs in the presence of a base. Suitable bases include, but are not limited to, amines (e.g., N,N-diisopropylethylamine, tripropylamine, tributylamine, 4-methylmorpholine, etc.), basic aromatic compounds (e.g., pyridine, 2,6-lutidine, imidazole, etc.), carbonates (e.g., potassium carbonate, lithium carbonate, sodium carbonate, cesium carbonate, etc.), bicarbonates (e.g., lithium bicarbonate, sodium bicarbonate, potassium bicarbonate, etc.), and phosphates (e.g., sodium phosphate, potassium phosphate, etc.).

In some embodiments, the base is a carbonate. In some embodiments, the base is potassium carbonate.

In some embodiments, the methylation reaction (e.g., the methylation reaction for providing Compound VIII) occurs in the presence of a solvent. Suitable solvents include, but are not limited to, ethers (e.g., tetrahydrofuran, 2-methyltetrahydrofuran, tert-butyl methyl ether, etc.), polar aprotic solvents (e.g., N,N-dimethylformamide, N,N-dimethylacetamide, N-methyl-2-pyrrolidone, dimethyl sulfoxide, sulfolane, 1,4-dioxane, etc.), aromatic solvents (e.g., trifluorotoluene), sulfolane, halogenated solvents (e.g., dichloromethane, 1,2-dichloroethane, etc.), hydrocarbons (e.g., n-heptane, pentane), and ketones (e.g., acetone, 2-butanone, etc.).

In some embodiments, the solvent is a polar aprotic solvent. In some embodiments, the solvent is N,N-dimethylformamide.

In some embodiments, the methylation reaction (e.g., the methylation reaction for providing Compound VIII) occurs at a temperature of about −10° C. to about 60° C. In some embodiments, the temperature is about 5° C. to about 25° C. In some embodiments, the temperature is about 15° C.

In some embodiments, the methylation reaction (e.g., the methylation reaction using methyl iodide for providing Compound VIII) occurs in the presence of a base such as a carbonate (e.g., potassium carbonate), and a solvent such as a polar aprotic solvents (e.g., N,N-dimethylformamide).

In some embodiments, the process further comprises recrystallizing Compound IX or a salt thereof. In some embodiments, the process further comprises recrystallizing Compound IX HCl salt. The recrystallization may be performed using a solvent such as nitriles (e.g., acetonitrile, benzonitrile, etc.), ethers (e.g., tetrahydrofuran, 2-methyltetrahydrofuran, tert-butyl methyl ether, etc.), alcohols (e.g., ethanol, 2-propanol, etc.), esters (e.g., ethyl acetate, isopropyl acetate, etc.), ketones (e.g., acetone, 2-butanone, 4-methyl-2-pentanone, etc.), or halogenated solvents (e.g., dichloromethane, 1,2-dichloroethane, etc.).

In some embodiments, the recrystallization is performed using a nitrile solvent (e.g., acetonitrile).

The recrystallization may be performed at a temperature of about 20° C. to about 80° C., such as a temperature of about 35° C. to about 55° C. In some embodiments, the recrystallization is performed at a temperature of about 45° C.

In some embodiments, the process further comprises recrystallizing Compound IX or a salt thereof (e.g., Compound IX HCl salt) from a solvent such as a nitrile solvent (e.g., acetonitrile) at a suitable temperature such as a temperature of about 35° C. to about 55° C. (e.g., about 45° C.).

### Route B

Also provided is a process for preparing Compound II,

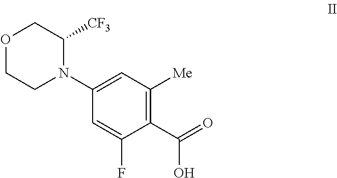 [SMILES: Cc1cc(N2CCOC[C@@H]2C(F)(F)F)cc(F)c1C(=O)O]

or a salt thereof, comprising:
(a) contacting Compound VIII

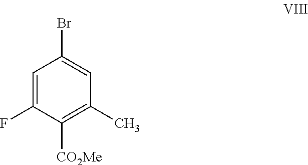

or a salt thereof with morpholin-3-one in the presence of a catalytic system to form Compound II-3

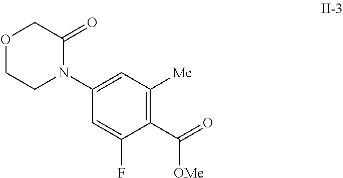 [SMILES: COC(=O)c1c(C)cc(N2CCOCC2=O)cc1F]

or a salt thereof;
(b) contacting Compound II-3 with a trifluoromethylation agent;
(c) subjecting a product from step (b) into an elimination condition to form Compound II-4

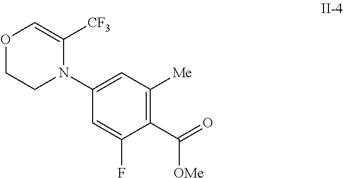 [SMILES: COC(=O)c1c(C)cc(N2CCOC=C2C(F)(F)F)cc1F]

or a salt thereof;
(d) subjecting Compound II-4 into an asymmetric hydrogenation condition to form Compound II-2

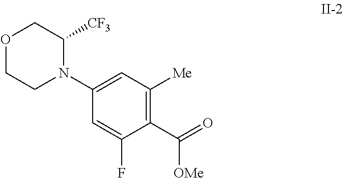 [SMILES: COC(=O)c1c(C)cc(N2CCOC[C@@H]2C(F)(F)F)cc1F]

or a salt thereof.

In some embodiments, the process comprises contacting Compound VIII or a salt thereof with morpholin-3-one in the presence of a catalytic system to form Compound II-3 or a salt thereof.

In some embodiments, the catalytic system (e.g., the catalytic system for providing Compound II-3 from Compound VIII) comprises a catalyst. In some embodiments, the catalyst is a copper catalyst (e.g., Cu(I) or Cu(II) salts). Suitable copper catalysts include, but are not limited to, CuI₂, CuBr, CuBr₂, CuCl, CuCl₂, Cu(OAc)₂, Cu₂O, CuO, and CuSO₄.

In some embodiments, the catalyst is CuI.

In some embodiments, the catalytic system (e.g., the catalytic system for providing Compound II-3 from Compound VIII) comprising a copper catalyst further comprises a ligand. Suitable ligands include, but are not limited to, diamines (e.g., trans-N,N′-bis-methyl-1,2-cyclohexanediamine, trans-N,N-dimethylcyclohexane-1,2-diamine, trans-cyclohexane-1,2-diamine, N,N′-dimethylethylenediamine, N,N-dimethylethylenediamine, ethylenediamine, N,N,N′,N′-tetramethyl ethylenediamine, N-cyclohexyl-N-methylethane-1,2-diamine, dimethyl-1,2-diphenylethane-1,2-diamine, etc.), 1,10-phenanthroline, [(2,6-dimethylphenyl)amino](oxo)acetic acid (DMPAO), and α-amino acids (e.g., L-hydroxyproline, etc.).

In some embodiments, the ligand is a diamine ligand. In some embodiments, the ligand is trans-N,N′-bis-methyl-1,2-cyclohexanediamine.

In some embodiments, the catalytic system (e.g., the catalytic system for providing Compound II-3 from Compound VIII) comprising a copper catalyst further comprises a base, such as an inorganic or organic base (e.g. triethylamine, pyridine, sodium carbonate, sodium bicarbonate, potassium bicarbonate, tripotassium phosphate, sodium tert-butoxide, cesium carbonate, sodium methoxide, potassium hydroxide, sodium hydroxide, etc.).

In some embodiments, the base is a carbonate. In some embodiments, the base is potassium carbonate.

In some embodiments, the catalytic system (e.g., the catalytic system for providing Compound II-3 from Compound VIII) comprising a copper catalyst further comprises a solvent. Suitable solvents include, but are not limited to, toluene, dioxane, N,N-dimethylformamide, pyridine, dimethyl sulfoxide, nitrobenzene, glycol, diglyme, acetonitrile, water, and ethanol.

In some embodiments, the solvent is toluene.

In some embodiments, the reaction between Compound VIII and morpholin-3-one occurs at a temperature of about −50° C. to about 150° C. In some embodiments, the temperature is about 90° C. to about 110° C. In some embodiments, the temperature is about 100° C.

In some embodiments, the reaction between Compound VIII and morpholin-3-one occurs at suitable temperature such as a temperature of about 90° C. to about 110° C. (e.g., about 100° C.), and in the presence of a catalytic system comprising a catalyst such as a copper catalyst (e.g., CuI), a ligand such as a diamine ligand (e.g., trans-N,N′-bis-methyl-1,2-cyclohexanediamine), a base such as a carbonate (e.g., potassium carbonate), and a solvent such as toluene.

In some embodiments, the catalytic system (e.g., the catalytic system for providing Compound II-3 from Compound VIII) comprises a catalyst. In some embodiments, the catalyst is a palladium catalyst. Suitable palladium catalysts include, but are not limited to, palladium(II) sources (e.g., XantPhos Pd G4, palladium(II) chloride, etc.) and palladium(0) source (e.g., tetrakis(triphenylphosphine)palladium(0), etc.).

In some embodiments, the catalyst is a XantPhos Pd G4 (9-{[5-(diphenylphosphanyl)-9,9-dimethyl-9H-xanthen-4-yl]diphenyl-lambda-phosphanyl}-8-methyl-8-aza-9-palladatricyclo[8.4.0.0(2),]tetradeca-1(14),2,4,6,10,12-hexaen-9-yl methanesulfonate).

In some embodiments, the catalytic system (e.g., the catalytic system for providing Compound II-3 from Compound VIII) comprising a palladium catalyst further comprises a ligand. Suitable ligands include, but are not limited to, XantPhos, monodentate phosphine ligands (e.g., triphenylphosphine, 2-dicyclohexylphosphino-2′,6′-dimethoxybiphenyl, etc.), and bidentate phosphine ligands (e.g., 1,2-bis(diphenylphosphino)ethane, 2,2′-bis(diphenylphosphino)-1,1′-binaphthyl, 1,1′-bis(diphenylphosphino)ferrocene, etc.).

In some embodiments, the ligand is XantPhos (e.g., from the XantPhos Pd G4 catalyst).

In some embodiments, the catalytic system (e.g., the catalytic system for providing Compound II-3 from Compound VIII) comprising a palladium catalyst further comprises a base, such as an inorganic or organic base (e.g. triethylamine, pyridine, sodium carbonate, sodium bicarbonate, cesium bicarbonate, tripotassium phosphate, sodium tert-butoxide, cesium carbonate, sodium methoxide, potassium hydroxide, sodium hydroxide, etc.).

In some embodiments, the base is a carbonate. In some embodiments, the base is cesium carbonate.

In some embodiments, the catalytic system (e.g., the catalytic system for providing Compound II-3 from Compound VIII) comprising a palladium catalyst further comprises a solvent. Suitable solvents include, but are not limited to, ethers (e.g. tetrahydrofuran, methyl tert-butyl ether, dioxane, cyclopentyl methyl ether, etc.), aromatics (e.g. toluene, etc.), alcohols (e.g., methanol, ethanol, etc.), amide (N,N-dimethylformamide, N-methyl-2-pyrrolidone, dimethylacetamide, etc.), and ester (e.g. isopropyl acetate, ethyl acetate, etc.).

In some embodiments, the solvent is an aromatic solvent. In some embodiments, the solvent is toluene.

In some embodiments, the reaction between Compound VIII and morpholin-3-one occurs at a temperature of about 20° C. to about 150° C. In some embodiments, the temperature is about 75° C. to about 95° C. In some embodiments, the temperature is about 85° C.

In some embodiments, the reaction between Compound VIII and morpholin-3-one occurs at a suitable such as a temperature of about 75° C. to about 95° C. (e.g., about 85° C.), and in the presence of a catalytic system comprising a catalyst such as a palladium catalyst (e.g., XantPhos Pd G4), a ligand such as XantPhos ((from the XantPhos Pd G4 catalyst), a base such as a carbonate (e.g., cesium carbonate), and a solvent such as toluene.

In some embodiments, the process comprises contacting Compound II-3 with a trifluoromethylation agent. Suitable trifluoromethylation agents include, but are not limited to, trifluoroacetophenone, trifluoromethyllithium, (trifluoromethyl)trimethylsilane, and (trifluoromethyl)triethylsilane.

In some embodiments, the trifluoromethylation agent is (trifluoromethyl)trimethylsilane.

In some embodiments, the trifluoromethylation reaction (e.g., the trifluoromethylation of Compound II-3) occurs in the presence of an initiator. Suitable initiators include, but are not limited to, fluoride sources (e.g., cesium fluoride, tetrabutylammonium fluoride, tetrabutylammonium difluorotriphenylsilicate, potassium fluoride, sodium fluoride, lithium fluoride, etc.), carboxylates (e.g., potassium ethylhexanoate, sodium ethylhexanoate, etc.), and alkoxides (e.g., potassium tert-butoxide, sodium tert-butoxide, etc.).

In some embodiments, the initiator is a fluoride source. In some embodiments, the initiator is cesium fluoride.

In some embodiments, the trifluoromethylation reaction (e.g., the trifluoromethylation of Compound II-3) occurs in the presence of a solvent. Suitable solvents include, but are not limited to, ethers (e.g., tetrahydrofuran, 2-methyltetrahydrofuran, tert-butyl methyl ether, 1,4-dioxane, cyclopentyl methyl ether, etc.) and aromatic solvents (e.g., toluene, etc.).

In some embodiments, the solvent is an ether solvent. In some embodiments, the solvent is 2-methyltetrahydrofuran.

In some embodiments, the trifluoromethylation reaction (e.g., the trifluoromethylation of Compound II-3) occurs at a temperature of about −78° C. to about 50° C. In some embodiments, the temperature is about −15° C. to about 15° C.

In some embodiments, the trifluoromethylation reaction (e.g., the trifluoromethylation of Compound II-3) occurs in the presence of a trifluoromethylation agent (e.g., (trifluoromethyl)trimethylsilane), a initiator such as a fluoride source (e.g., cesium fluoride), a solvent such as an ether solvent (e.g., 2-methyltetrahydrofuran), and at a suitable temperature such as a temperature of about −15° C. to about 15° C.

In some embodiments, the process comprises subjecting a product from step (b) into an elimination condition to form Compound II-4 or a salt thereof.

In some embodiments, the elimination condition (e.g., the elimination condition for proving Compound II-4) comprises an acid. Suitable acids include, but are not limited to, hydrogen chloride solutions (e.g., hydrogen chloride solution in dioxane, hydrogen chloride solution in cyclopentyl methyl ether, hydrogen chloride solution in ethanol, hydrogen chloride solution in 2-propanol, etc.), mineral acids (e.g., hydroiodic acid, hydrobromic acid, sulfuric acid, phosphoric acid, etc.), sulfonic acids (e.g., p-toluenesulfonic acid, etc.), and carboxylic acids (e.g., acetic acid, pivalic acid, trifluoroacetic acid, etc.).

In some embodiments, the acid is a hydrogen chloride solution. In some embodiments, the acid is hydrogen chloride solution in dioxane.

In some embodiments, the elimination condition (e.g., the elimination condition for proving Compound II-4) comprises a solvent. Suitable solvents include, but are not limited to, ethers (e.g., tetrahydrofuran, 2-methyltetrahydrofuran, tert-butyl methyl ether, cyclopentyl methyl ether, etc.), aromatic solvents (e.g., toluene, etc.), alcohols (e.g., methanol, ethanol, 2-propanol, etc.), polar aprotic solvents (e.g., N,N-dimethylformamide, N,N-dimethylacetamide, N-methyl-2-pyrrolidone, etc.).

In some embodiments, the solvent is an ether solvent. In some embodiments, the solvent is 2-methyltetrahydrofuran.

In some embodiments, the elimination condition (e.g., the elimination condition for proving Compound II-4) comprises a temperature of about 0° C. to about 50° C. In some embodiments, the temperature is about 5° C. to about 25° C. In some embodiments, the temperature is about 15° C.

In some embodiments, the elimination condition (e.g., the elimination condition for proving Compound II-4) comprises an acid such as a hydrogen chloride solution (e.g., hydrogen chloride solution in dioxane), a solvent such as an ether solvent (e.g., 2-methyltetrahydrofuran), and a suitable temperature such as a temperature of about 5° C. to about 25° C. (e.g., about 15° C.).

In some embodiments, the process comprises subjecting Compound II-4 or a salt thereof into an asymmetric hydrogenation condition to form Compound II-2 or a salt thereof.

In some embodiments, the asymmetric hydrogenation condition (e.g., the asymmetric hydrogenation condition for proving Compound II-2) comprises a catalyst. Suitable catalysts include, but are not limited to, rhodium catalysts (e.g., bis(1,5-cyclooctadiene)rhodium(I) tetrafluoroborate, etc.), iridium catalysts (e.g., cyclooctadiene iridium chloride dimer, etc.), ruthenium catalysts (e.g., bis(2-methylallyl)(1,5-cyclooctadiene)ruthenium(II), etc.), nickel catalysts (e.g., nickel(II) acetate, nickel(II) nitrate, etc.), and palladium catalysts (e.g., palladium(II) trifluoroacetate, palladium(II) acetate, etc.).

In some embodiments, the catalyst is a rhodium catalyst. In some embodiments, the catalyst is bis(1,5-cyclooctadiene)rhodium(I) tetrafluoroborate.

In some embodiments, the asymmetric hydrogenation condition (e.g., the asymmetric hydrogenation condition for proving Compound II-2) comprises a ligand. Suitable ligands include, but are not limited to, (R)-(+)-1-[(R)-2-(2′-dicyclohexylphosphinophenyl)ferrocenyl]ethylbis(3,5-trifluoromethylphenyl)phosphine (SL-W008-1), (1S)-1-[(1R)-1-[Bis[3,5-bis(trifluoromethyl)phenyl]phosphino]ethyl]-2-[2-(dicyclohexylphosphino)phenyl]ferrocene, (1Sp)-1-[Bis(4-methoxy-3,5-dimethylphenyl)phosphino]-2-[(1R)-1-(dicyclohexylphosphino)ethyl]ferrocene, (1R,1′R)-1,1′-Bis[bis[3,5-bis(trifluoromethyl)phenyl]phosphino]-2,2′-bis[(R)-(dimethylamino)phenylmethyl]ferrocene, 1,2-Bis((2R,5R)-2,5-diisopropylphospholan-1-yl)ethane, 1,1′-[(5aR,8aR,14aR)-5a,6,7,8,8a,9-Hexahydro-5H-[1]benzopyrano[3,2-d]xanthene-1,13-diyl]bis[1,1-diphenyl-phosphine], (R)-5,5′-Bis(diphenylphosphino)-4,4′-bibenzo[d][1,3]dioxole, (S)-(−)-2,2′-Bis[di(3,5-di-t-butylphenyl)phosphino]-6,6′-dimethoxy-1,1′-biphenyl, 1-{(2R)-1-[(11bR)-8,9,10,11,12,13,14,15-Octahydrodinaphtho[2,1-d:1′,2′-f][1,3,2]dioxaphosphepin-4-yloxy]propan-2-yl}-3-phenylurea, and N-[(11bS)-Dinaphtho[2,1-d:1′,2′-f][1,3,2]dioxaphosphepin-4-yl]-1,1,1-trifluoromethanesulfonamide.

In some embodiments, the ligand is (R)-(+)-1-[(R)-2-(2′-dicyclohexylphosphinophenyl)ferrocenyl]ethylbis(3,5-trifluoromethylphenyl)phosphine (SL-W008-1).

In some embodiments, the asymmetric hydrogenation condition (e.g., the asymmetric hydrogenation condition for proving Compound II-2) comprises a hydrogen source. Suitable hydrogen source includes, but is not limited to, hydrogen gas, formic acid, sodium formate, tetrabutylammonium formate, tetrahydroxydiboron, triethylsilane, phenylsilane, and hydrazine.

In some embodiments, the hydrogen source is hydrogen gas.

In some embodiments, the asymmetric hydrogenation condition (e.g., the asymmetric hydrogenation condition for proving Compound II-2) comprises an additive. Suitable additives include, but are not limited to, acids (e.g., acetic acid, trifluoroacetic acid, p-toluenesulfonic acid, methanesulfonic acid, hydrochloric acid, formic acid, etc.), bases (e.g., triethylamine, N,N-diisopropylethylamine, pyridine, potassium bicarbonate, sodium bicarbonate, etc.), and iodine and iodide salts (e.g., sodium iodide, cesium iodide, potassium iodide, tetrabutylammonium iodide, etc.).

In some embodiments, the asymmetric hydrogenation condition (e.g., the asymmetric hydrogenation condition for proving Compound II-2) does not comprise an additive.

In some embodiments, the asymmetric hydrogenation condition (e.g., the asymmetric hydrogenation condition for proving Compound II-2) comprises a solvent. Suitable solvents include, but are not limited to, ethers (e.g., tetrahydrofuran, tert-butyl methyl ether, 1,4-dioxane, cyclopentyl methyl ether, etc.), aromatic solvents (e.g., toluene, etc.), alcohols (e.g., methanol, ethanol, 2-propanol, 2,2,2-trifluoroethanol, etc.), polar aprotic solvents (e.g., N,N-dimethylformamide, N,N-dimethylacetamide, N-methyl-2-pyrrolidone, etc.), and ester solvents (e.g., ethyl acetate, isopropyl acetate, etc.).

In some embodiments, the solvent is an alcohol. In some embodiments, the solvent is 2,2,2-trifluoroethanol.

In some embodiments, the asymmetric hydrogenation condition (e.g., the asymmetric hydrogenation condition for proving Compound II-2) comprises a temperature of about 20° C. to about 100° C. In some embodiments, the temperature is about 50° C. to about 70° C. In some embodiments, the temperature is about 60° C.

In some embodiments, the asymmetric hydrogenation condition (e.g., the asymmetric hydrogenation condition for proving Compound II-2) comprises an elevated pressure. In some embodiments, the pressure is about 15 psi to about 200 psi. In some embodiments, the pressure is about 120 psi.

In some embodiments, the asymmetric hydrogenation condition (e.g., the asymmetric hydrogenation condition for proving Compound II-2) comprises a hydrogen source such as hydrogen gas, a catalyst such as a rhodium catalyst (e.g., bis(1,5-cyclooctadiene)rhodium(I) tetrafluoroborate), a ligand such as SL-W008-1, a solvent such as an alcohol solvent (e.g., 2,2,2-trifluoroethanol), a suitable temperature such as a temperature of about 50° C. to about 70° C. (e.g., about 60° C.), and an elevated pressure such as about 120 psi.

In some embodiments, the process further comprises converting Compound II-2 or a salt thereof to Compound II or a salt thereof according to a process described anywhere herein.

### Synthesis of Compound IX and Derivatives

Also provided are processes for preparing Compound IX or a salt thereof.

### Chiral Resolution Route

For example, Compound IX or a salt thereof may be prepared via chiral resolution.

In some embodiments, provided herein is a process preparing Compound IX

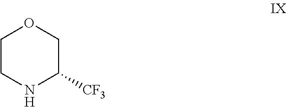

or a salt thereof, comprising
(a) optionally, free basing a salt of Compound IXa; and

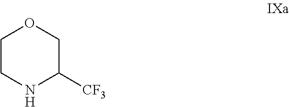

(b) contacting Compound IXa free base with a chiral acid that is optically pure to form Compound IX chiral acid salt.

In some embodiments, the process comprises free basing a salt of Compound IXa. In some embodiments, the process comprises free basing Compound IXa HCl salt.

In some embodiments, free basing a salt of Compound IXa is performed in the presence of a base. Suitable bases include, but are not limited to, carbonates (e.g., lithium carbonate, sodium carbonate, potassium carbonate, cesium carbonate, etc), bicarbonates (e.g., lithium bicarbonate, sodium bicarbonate, potassium bicarbonate, etc.), phosphates (e.g., sodium phosphate, potassium phosphate, etc.), and hydroxides (e.g., sodium hydroxide, potassium hydroxide, etc.).

In some embodiments, the base is a carbonate. In some embodiments, the base is cesium carbonate.

In some embodiments, free basing a salt of Compound IXa is performed in the presence of a solvent. Suitable solvents include, but are not limited to, ethers (e.g., 2-methyltetrahydrofuran, tert-butyl methyl ether, etc.), ketones (e.g., acetone, 2-butanone, 4-methyl-2-pentanone, etc.), esters (e.g., ethyl acetate, isopropyl acetate, methyl acetate, etc.), nitriles (e.g., acetonitrile, etc.), halogenated solvents (e.g., dichloromethane, 1,2-dichloroethane, chlorobenzene, etc.), or a combination of any of the foregoing with water.

In some embodiments, the solvent is a halogenated solvent. In some embodiments, the solvent is dichloromethane.

In some embodiments, free basing a salt of Compound IXa is performed at a temperature of about 0° C. to about 80° C. In some embodiments, the temperature is about 10° C. to about 30° C. In some embodiments, the temperature is about 20° C.

In some embodiments, free basing a salt of Compound IXa is performed in the presence of a base such as a carbonate (e.g., cesium carbonate), a solvent such as a halogenated solvent (e.g., dichloromethane), and at a suitable temperature such as a temperature of about 10° C. to about 30° C. (e.g., about 20° C.).

In some embodiments, the process comprises contacting Compound IXa free base with a chiral acid that is optically pure to form Compound IX chiral acid salt.

Suitable chiral acids include, but are not limited to, (+)-Camphorsulfonic acid, (−)-camphorsulfonic acid, L-(−)-malic acid, N-acetyl-L-leucine, (+)-mandelic acid, and dibenzoyl-L-tartaric acid.

In some embodiments, the chiral acid is (+)-Camphorsulfonic acid. In some embodiments, the Compound IX chiral acid salt is Compound IX (+)-Camphorsulfonic acid salt.

In some embodiments, the contacting (e.g., the contacting of Compound IXa free base and a chiral acid) occurs in the presence of a solvent. Suitable solvents include, but are not limited to, ethers (e.g., tetrahydrofuran, 2-methyltetrahydrofuran, tert-butyl methyl ether, etc.), ketones (e.g., methyl ethyl ketone, acetone, 2-butanone, 4-methyl-2-pentanone, etc.), esters (e.g., ethyl acetate, isopropyl acetate, methyl acetate, etc.), nitriles (e.g., acetonitrile, etc.), halogenated solvents (e.g., dichloromethane, 1,2-dichloroethane, chlorobenzene, etc.), and alcohols (e.g., 2-propanol, 2-butanol, etc.).

In some embodiments, the solvent is a ketone. In some embodiments, the solvent is methyl ethyl ketone.

In some embodiments, the contacting (e.g., the contacting of Compound IXa free base and a chiral acid) occurs at a temperature of about 0° C. to about 80° C. In some embodiments, the temperature is about 10° C. to about 30° C. In some embodiments, the temperature is about 20° C.

In some embodiments, the contacting (e.g., the contacting of Compound IXa free base and a chiral acid such as (+)-Camphorsulfonic acid) occurs in the presence of a solvent such as a ketone solvent (e.g., methyl ethyl ketone) at a suitable temperature such as a temperature of about 10° C. to about 30° C. (e.g., about 20° C.).

In some embodiments, Compound IXa or a salt thereof is prepared by a process compressing:
(a) subjecting Compound IX-2-Bn

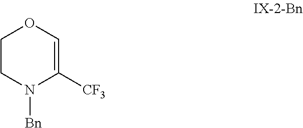 [SMILES: FC(F)(F)C1=COCCN1Cc1ccccc1]

or a salt thereof to a hydrogenation condition to form Compound IX-3a-Bn

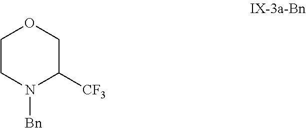 [SMILES: FC(F)(F)C1COCCN1Cc1ccccc1]

or a salt thereof;
(b) subjecting Compound IX-3a-Bn to a deprotection condition; and
(c) subjecting the reaction mixture from step (b) to an acidic condition to form Compound IXa

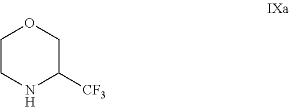

or a salt thereof.

In some embodiments, the hydrogenation condition (e.g., the condition for providing Compound IX-3a-Bn from Compound IX-2-Bn) comprises a catalyst. Suitable catalysts include, but are not limited to, solid-supported transition metals (e.g., palladium on carbon, platinum on carbon, etc.), Raney Nickel, and transition metal catalysts (e.g., (SP-4)-(η²,η²-cycloocta-1,5-diene)(pyridine)(tricyclohexylphosphine)iridium(1+) hexafluoridophosphate(1−), (SP-4)-chloridotris(triphenylphosphene)rhodium), a palladium(II) source such as palladium(II) chloride, a palladium(0) source such as tetrakis(triphenylphosphine)palladium(0), a nickel(II) source such as nickel(II) chloride, or a nickel(0) source such as bis(cyclooctadiene)nickel(0), etc.).

In some embodiments, the catalyst is solid-supported transition metals. In some embodiments, the catalyst is palladium on carbon.

In some embodiments, the hydrogenation condition (e.g., the condition for providing Compound IX-3a-Bn from Compound IX-2-Bn) comprises a reductant. The reductant may be a hydrogen source such as hydrogen gas, formic acid, sodium formate, or tetrabutylammonium formate.

In some embodiments, the reductant is hydrogen gas.

In some embodiments, the hydrogenation condition (e.g., the condition for providing Compound IX-3a-Bn from Compound IX-2-Bn) comprises a solvent. The solvent may be alcohols, such as methanol, ethanol, 2-propanol, tert-butanol, or tert-amyl alcohol.

In some embodiments, the solvent is ethanol.

In some embodiments, the hydrogenation condition (e.g., the condition for providing Compound IX-3a-Bn from Compound IX-2-Bn) comprises a temperature of about 0° C. to about 60° C. In some embodiments, the temperature is about 10° C. to about 30° C. In some embodiments, the temperature is about 20° C.

In some embodiments, the hydrogenation condition (e.g., the condition for providing Compound IX-3a-Bn from Compound IX-2-Bn) comprises a catalyst such as a solid-supported transition metal catalyst (e.g., palladium on carbon), a reductant such as hydrogen gas, a solvent such as ethanol, and at a suitable temperature such as a temperature of about 10° C. to about 30° C. (e.g., about 20° C.).

In some embodiments, the obtained Compound IX-3a-Bn is subject to the next step without isolation and/or purification.

In some embodiments, the deprotection condition (e.g., the condition for step (b)) comprises a catalyst. Suitable catalysts include, but are not limited to, solid-supported transition metals (e.g., palladium on carbon, platinum on carbon, etc.), Raney Nickel, and transition metal catalysts (e.g., (SP-4)-(η²,η²-cycloocta-1,5-diene)(pyridine)(tricyclohexylphosphine)iridium(1+) hexafluoridophosphate(1−), (SP-4)-chloridotris(triphenylphosphene)rhodium), a palladium(II) source such as palladium(II) chloride, a palladium(0) source such as tetrakis(triphenylphosphine)palladium(0), a nickel(II) source such as nickel(II) chloride, or a nickel(0) source such as bis(cyclooctadiene)nickel(0), etc.).

In some embodiments, the catalyst is solid-supported transition metals. In some embodiments, the catalyst is palladium on carbon.

In some embodiments, the deprotection condition (e.g., the condition for step (b)) comprises a reductant. The reductant may be a hydrogen source such as hydrogen gas, formic acid, sodium formate, or tetrabutylammonium formate.

In some embodiments, the reductant is hydrogen gas.

In some embodiments, the deprotection (e.g., the condition for step (b)) comprises a solvent. The solvent may be alcohols, such as methanol, ethanol, 2-propanol, tert-butanol, or tert-amyl alcohol.

In some embodiments, the solvent is ethanol.

In some embodiments, the deprotection condition (e.g., the condition for step (b)) comprises a temperature of about 0° C. to about 60° C. In some embodiments, the temperature is about 10° C. to about 30° C. In some embodiments, the temperature is about 20° C.

In some embodiments, the deprotection condition (e.g., the condition for step (b)) comprises a catalyst such as a solid-supported transition metal catalyst (e.g., palladium on carbon), a reductant such as hydrogen gas, a solvent such as methanol, and at a suitable temperature such as a temperature of about 10° C. to about 30° C. (e.g., about 20° C.).

The acid used in the acidic condition (e.g., the condition for step (c)) may be hydrochloric acid, p-toluenesulfonic acid, or oxalic acid.

In some embodiments, the acid is hydrochloric acid, and the salt of Compound IXa is Compound IXa HCl salt.

In some embodiments, the acidic condition (e.g., the condition for step (c)) comprises a solvent. The solvent may be alcohols, such as methanol, ethanol, 2-propanol, tert-butanol, or tert-amyl alcohol.

In some embodiments, the solvent is methanol.

In some embodiments, the acidic condition (e.g., the condition for step (c)) comprises a temperature of about 0° C. to about 60° C. In some embodiments, the temperature is about 10° C. to about 30° C. In some embodiments, the temperature is about 20° C.

In some embodiments, the acidic condition (e.g., the condition for step (c)) comprises an acid such as hydrochloric acid, a solvent such as methanol, and at a suitable temperature such as a temperature of about 10° C. to about 30° C. (e.g., about 20° C.).

Additionally or alternatively, Compound IX-3a-Bn

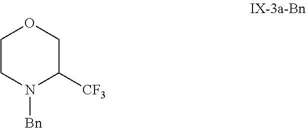 [SMILES: FC(F)(F)C1COCCN1Cc1ccccc1]

or a salt thereof may be prepared by a process comprising:
subjecting Compound IX-2-Bn

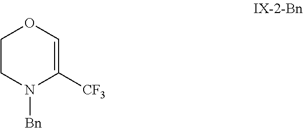 [SMILES: FC(F)(F)C1=COCCN1Cc1ccccc1]

or a salt thereof to a hydrogenation condition to form Compound IX-3a-Bn or a salt thereof.

In some embodiments, the hydrogenation condition (e.g., the condition for providing Compound IX-3a-Bn from IX-2-Bn) comprises a catalyst such as an iron catalyst. The catalyst may be an iron(III) source such as iron(III) chloride or tris(acetylacetonato)iron(III).

In some embodiments, the catalyst is tris(acetylacetonato)iron(III).

In some embodiments, the hydrogenation condition (e.g., the condition for providing Compound IX-3a-Bn from IX-2-Bn) comprises an additive that is thiophenol.

In some embodiments, the hydrogenation condition (e.g., the condition for providing Compound IX-3a-Bn from IX-2-Bn) comprises a hydrogen source such as an organosilane compound. The organosilane may be diphenylsilane, triethylsilane, or phenylsilane.

In some embodiments, the hydrogen source is phenylsilane.

In some embodiments, the hydrogenation condition (e.g., the condition for providing Compound IX-3a-Bn from IX-2-Bn) comprises a solvent. Suitable solvents include, but are not limited to, alcohols (e.g., methanol, ethanol, 2-propanol, etc.), ethers (e.g., 2-methyltetrahydrofuran, tert-butyl methyl ether, etc.), and polar aprotic solvents (e.g., N,N-dimethylformamide, N,N-dimethylacetamide, dimethyl sulfoxide, sulfolane, etc.).

In some embodiments, the solvent is an alcohol. In some embodiments, the solvent is ethanol.

In some embodiments, the hydrogenation condition (e.g., the condition for providing Compound IX-3a-Bn from IX-2-Bn) comprises a temperature of about 0° C. to about 60° C. In some embodiments, the temperature is about 10° C. to about 30° C. In some embodiments, the temperature is about 20° C.

In some embodiments, the hydrogenation condition (e.g., the condition for providing Compound IX-3a-Bn from IX-2-Bn) comprises a catalyst such as an iron catalyst (e.g., tris(acetylacetonato)iron(III)), an additive that is thiophenol, a hydrogen source such as an organosilane compound (e.g., phenylsilane), a solvent such as an alcohol (e.g., ethanol), and a suitable temperature such as a temperature of about 10° C. to about 30° C. (e.g., about 20° C.).

Additionally or alternatively, Compound IX-3a-Bn

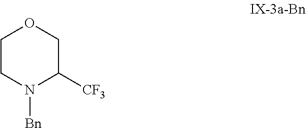 [SMILES: FC(F)(F)C1COCCN1Cc1ccccc1]

or a salt thereof may be prepared by a process comprising:
subjecting Compound IX-4a

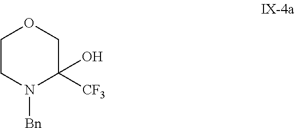 [SMILES: OC1(C(F)(F)F)COCCN1Cc1ccccc1]

or a salt thereof to a reductive condition to form Compound IX-3a-Bn or a salt thereof.

In some embodiments, the reductive condition (e.g., the condition for providing Compound IX-3a-Bn from Compound IX-4a) comprises a Lewis acid. Suitable Lewis acids include, but are not limited to, boron trifluoride complexes (e.g., boron trifluoride dietherate, boron trifluoride tetrahydrofuran, etc.), aluminum salts (e.g., aluminum trichloride, etc.), zinc salts (e.g., zinc(II) chloride, etc.), and scandium salts (e.g., scandium(III) triflate, etc.).

In some embodiments, the Lewis acid is a boron trifluoride complex. In some embodiments, the Lewis acid is boron trifluoride dietherate.

In some embodiments, the reductive condition (e.g., the condition for providing Compound IX-3a-Bn from Compound IX-4a) comprises a reductant. The reductant may be sodium borohydride, sodium triacetoxyborohydride, or sodium cyanoborohydride.

In some embodiments, the reductant is sodium borohydride.

In some embodiments, the reductive condition (e.g., the condition for providing Compound IX-3a-Bn from Compound IX-4a) comprises a solvent. Suitable solvents include, but are not limited to, ethers (e.g., tetrahydrofuran, 2-methyltetrahydrofuran, tert-butyl methyl ether, etc), esters (e.g., ethyl acetate, isopropyl acetate, methyl acetate, etc.), nitriles (e.g., acetonitrile, etc.), halogenated solvents (e.g., dichloromethane, 1,2-dichloroethane, chlorobenzene, etc.), and polar aprotic solvents (e.g., N,N-dimethylformamide, N,N-dimethylacetamide, N-methyl-2-pyrrolidone, etc.).

In some embodiments, the solvent is an ether solvent. In some embodiments, the solvent is tetrahydrofuran.

In some embodiments, the reductive condition (e.g., the condition for providing Compound IX-3a-Bn from Compound IX-4a) comprises a temperature of about −30° C. to about 50° C. In some embodiments, the temperature is about −10° C. to about 30° C. In some embodiments, the temperature is about 0° C. to about 20° C.

In some embodiments, the reductive condition (e.g., the condition for providing Compound IX-3a-Bn from Compound IX-4a) comprises a Lewis acid such as a boron trifluoride complex (e.g., boron trifluoride dietherate), a reductant such as sodium borohydride, a solvent such as an ether solvent (e.g., tetrahydrofuran), and a suitable temperature such as a temperature of about −10° C. to about 30° C. (e.g., about 0° C. to about 20° C.).

### Asymmetric Hydrogenation Route

Additionally or alternatively, Compound IX or a salt thereof may be prepared via asymmetric hydrogenation.

In some embodiments, provided is a process for preparing Compound IX

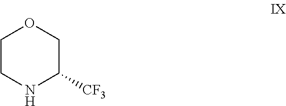

or a salt thereof, comprising:
(a) contacting a compound of Formula IX-1

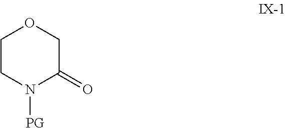

wherein PG is benzyl or

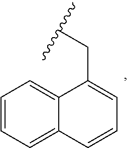 [SMILES: *Cc1cccc2ccccc12]

or a salt thereof with a trifluoromethylation agent to form a compound of Formula IX-2

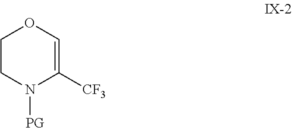

wherein PG is benzyl or

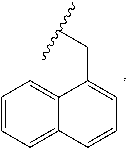 [SMILES: *Cc1cccc2ccccc12]

or a salt thereof;
(b) subjecting the compound of Formula IX-2 or a salt thereof to an asymmetric hydrogenation condition to form a compound of Formula IX-3

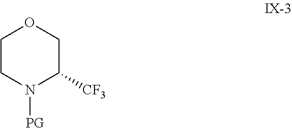

wherein PG is benzyl or

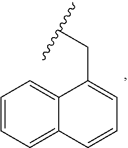 [SMILES: *Cc1cccc2ccccc12]

or a salt thereof; and
(c) subjecting the compound of formula IX-3 or a salt thereof to a deprotection condition to form Compound IX or a salt thereof, wherein step (c) occurs in the presence of a chiral acid.

In some embodiments, PG is benzyl. In some embodiments, PG is

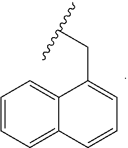 [SMILES: *Cc1cccc2ccccc12]

In some embodiments, the process comprises contacting a compound of Formula IX-1 or a salt thereof with a trifluoromethylation agent. Suitable trifluoromethylation agents include, but are not limited to, trifluoromethyl silanes (e.g., (trifluoromethyl)trimethylsilane, (trifluoromethyl)triethylsilane, (trifluoromethyl)n-propyldimethylsilane, (trifluoromethyl)triisopropylsilane, (trifluoromethyl)t-butyldimethylsilane, etc.) and fluoroform.

In some embodiments, the trifluoromethylation agent is a trifluoromethyl silane compound. In some embodiments, the trifluoromethylation agent is (trifluoromethyl)trimethylsilane.

In some embodiments, the reaction between a compound of Formula IX-1 or a salt thereof and a trifluoromethylation agent occurs in the presence of a base. Suitable bases include, but are not limited to, fluorides (e.g., lithium fluoride, potassium fluoride, cesium fluoride, etc.), tetrabutylammonium salts (e.g., tetrabutylammonium fluoride, tetrabutylammonium acetate, etc.), tetramethylammonium salts (e.g., tetramethylammonium fluoride, tetramethylammonium acetate, etc.), tert-butoxides (e.g., sodium tert-butoxide, potassium tert-butoxide, etc.), acetates (e.g., lithium acetate, sodium acetate, potassium acetate, etc.), carbonates (e.g., potassium carbonate, cesium carbonate, etc.), phosphates (e.g., potassium phosphate, lithium phosphate, cesium phosphate, etc.), and amides (e.g., potassium bis(trimethylsilyl)amide, lithium diisopropylamide, etc.).

In some embodiments, the base is a tert-butoxide base. In some embodiments, the base is potassium tert-butoxide.

In some embodiments, the reaction between a compound of Formula IX-1 or a salt thereof and a trifluoromethylation agent occurs in the presence of a solvent. Suitable solvents include, but are not limited to, aromatic solvents (e.g., toluene), ethers (e.g., tetrahydrofuran, 2-methyltetrahydrofuran, 1,4-dioxane, etc.), chlorinated (e.g., dichloromethane, 1,2-dichloroethane, trifluorotoluene, etc.), polar aprotic (e.g., N,N-dimethylformamide, N,N-dimethylacetamide, N-methyl-2-pyrrolidone, dimethyl sulfoxide, etc.), and nitriles (e.g., acetonitrile).

In some embodiments, the solvent is an aromatic solvent. In some embodiments, the solvent is toluene.

In some embodiments, the reaction between a compound of Formula IX-1 or a salt thereof and a trifluoromethylation agent occurs at a temperature of about −20° C. to about 50° C. In some embodiments, the temperature is about −10° C. to about 10° C. In some embodiments, the temperature is about 0° C.

In some embodiments, the reaction between a compound of Formula IX-1 or a salt thereof and a trifluoromethylation agent (e.g., a trifluoromethyl silane compound such as (trifluoromethyl)trimethylsilane) occurs in the presence of a base such as a tert-butoxide base (e.g., potassium tert-butoxide), a solvent such as an aromatic solvent (e.g., toluene), and at a suitable temperature such as a temperature of about −10° C. to about 10° C. (e.g., about 0° C.).

In some embodiments, the process comprises subjecting the compound of Formula IX-2 or a salt thereof to an asymmetric hydrogenation condition.

In some embodiments, the asymmetric hydrogenation condition (e.g., the asymmetric hydrogenation condition for proving a compound of Formula IX-3) comprises a catalyst. Suitable catalysts include, but are not limited to, ruthenium catalyst (e.g., (1,5-cyclooctadiene)bis(2-methylallyl)ruthenium, etc.), rhodium catalyst (e.g., bis(1,5-cyclooctadiene)rhodium(I) tetrafluoroborate, etc.), iridium catalyst (e.g., bis(1,5-cyclooctadiene)diiridium(I) dichloride, etc.), nickel catalyst (e.g., nickel(II) acetate, nickel(II) acetate tetrahydrate, nickel(II) trifluoromethanesulfonate, etc.), and palladium catalyst (e.g., palladium on carbon, etc.).

In some embodiments, the catalyst is a nickel catalyst. In some embodiments, the catalyst is nickel(II) acetate.

In some embodiments, the asymmetric hydrogenation condition (e.g., the asymmetric hydrogenation condition for proving a compound of Formula IX-3) comprises a ligand. Suitable ligands include, but are not limited to, Josiphos ligands (e.g., (S)-1-[(Rp)-2-(diphenylphosphino)ferrocenyl]-ethyldi-tert-butylphosphine (CAS 277306-29-3), (S)-(+)-1-[(R)-2-(diphenylphosphino)ferrocenyl]ethyldicyclohexylphosphine (CAS 162291-02-3), (S,S)-1-(dicyclohexylphosphino)-2-[1-(diphenylphosphino)ethyl]ferrocene (CAS 162291-01-2), (S)-1-[(RP)-2-(dicyclohexylphosphino)ferrocenyl]ethyldicyclohexylphosphine (CAS 246231-77-6), (S)-1-{(R)-2-[bis(4-methoxy-3,5-dimethylphenyl)phosphino]ferrocenyl}ethyldicyclo (CAS 849923-88-2), etc.), BPE ligands (e.g., (−)-1,2-bis((2R,5R)-2,5-diphenylphospholano)ethane, 1,2-bis[(2R,5R)-2,5-dimethyl-1-phospholanyl]ethane, etc.), DUPHOS ligands (e.g., (−)-1,2-bis[(2R,5R)-2,5-dimethylphospholano]benzene, (+)-1,2-bis[(2R,5R)-2,5-diisopropylphospholano]benzene, etc.), SEGPHO ligands (e.g., (R)-(+)-5,5′-bis(diphenylphosphino)-4,4′-bi-1,3-benzodioxole, etc.), SKP ligands (e.g., (+)-1,13-bis(diphenyl)phosphino-(5aR,8aR,14aR)-5a,6,7,8,8a,9-hexahydro-5H-[1]benzopyrano[3,2-d]xanthene, etc.).

In some embodiments, the ligand is a Josiphos ligand. In some embodiments, the ligand is CAS 277306-29-3.

In some embodiments, the asymmetric hydrogenation condition (e.g., the asymmetric hydrogenation condition for proving a compound of Formula IX-3) comprises a hydrogen source. Suitable hydrogen source includes, but is not limited to, hydrogen gas and hydrogen precursors (e.g., formic acid, sodium formate, or tetrabutylammonium formate).

In some embodiments, the hydrogen source is hydrogen gas.

In some embodiments, the asymmetric hydrogenation condition (e.g., the asymmetric hydrogenation condition for proving a compound of Formula IX-3) comprises an additive such acids (e.g., acetic acid, etc.).

In some embodiments, the asymmetric hydrogenation condition (e.g., the asymmetric hydrogenation condition for proving a compound of Formula IX-3) does not comprise an additive.

In some embodiments, the asymmetric hydrogenation condition (e.g., the asymmetric hydrogenation condition for proving a compound of Formula IX-3) comprises a solvent. Suitable solvents include, but are not limited to, alcohol (e.g., methanol, ethanol, 2,2,2-trifluoroethanol, etc.) and ether (e.g., cyclopentyl methyl ether, 1,4-dioxane, 2-methyltetrahydrofuran, dibutyl ether, tert-butyl methyl ether, etc.).

In some embodiments, the solvent is an alcohol. In some embodiments, the solvent is 2,2,2-trifluoroethanol.

In some embodiments, the asymmetric hydrogenation condition (e.g., the asymmetric hydrogenation condition for proving a compound of Formula IX-3) comprises a temperature of about 20° C. to about 120° C. In some embodiments, the temperature is about 50° C. to about 70° C. In some embodiments, the temperature is about 60° C.

In some embodiments, the asymmetric hydrogenation condition (e.g., the asymmetric hydrogenation condition for proving a compound of Formula IX-3) comprises a hydrogen source such as hydrogen gas, a catalyst such as a nickel catalyst (e.g., nickel(II) acetate), a ligand such as a Josiphos ligand (e.g., CAS 277306-29-3), a solvent such as an alcohol solvent (e.g., 2,2,2-trifluoroethanol), and a suitable temperature such as a temperature of about 50° C. to about 70° C. (e.g., about 60° C.).

In some embodiments, the process comprises subjecting the compound of formula IX-3 or a salt thereof to a deprotection condition to form Compound IX or a salt thereof.

In some embodiments, the deprotection condition (e.g., the deprotection condition for providing Compound IX) comprises an acid. Suitable acids include, but not limited to, (+)-Camphorsulfonic acid, p-toluenesulfonic acid, oxalic acid, and hydrogen chloride.

In some embodiments, the acid is a chiral acid. In some embodiments, the acid is (+)-Camphorsulfonic acid.

In some embodiments, the deprotection condition (e.g., the deprotection condition for providing Compound IX) comprises a catalyst. Suitable catalysts include, but are not limited to, solid-supported transition metals (e.g., platinum on carbon, palladium on carbon, etc), Raney Nickel, transition metal catalysts (e.g., (SP-4)-(η²,η²-cycloocta-1,5-diene)(pyridine)(tricyclohexylphosphine)iridium(1+) hexafluoridophosphate(1−), (SP-4)-chloridotris(triphenylphosphene)rhodium, etc.), a palladium(II) source (e.g., palladium(II) chloride, etc.), a palladium(0) source (e.g., tetrakis(triphenylphosphine)palladium(0), etc.), a nickel(II) source (e.g., nickel(II) chloride, etc.), and a nickel(0) source (e.g., bis(cyclooctadiene)nickel(0), etc.).

In some embodiments, the catalyst is a solid-supported transition metal. In some embodiments, the catalyst is palladium on carbon.

In some embodiments, the deprotection condition (e.g., the deprotection condition for providing Compound IX) comprises a hydrogen source such as hydrogen gas, an organosilane (e.g., triethylsilane, etc.), formic acid, a formate (e.g., sodium formate, tetrabutylammonium formate, etc).

In some embodiments, the hydrogen source is hydrogen gas.

In some embodiments, the deprotection condition (e.g., the deprotection condition for providing Compound IX) comprises a solvent. Suitable solvents include, but are not limited to, alcohols (e.g., methanol, ethanol, 2-propanol, tert-butanol, t-amyl alcohol, etc.), esters (e.g., isopropyl acetate, etc.), halogenated solvents (e.g., dichloromethane, 1,2-dichloroethane, chlorobenzene, etc.), hydrocarbons (e.g., toluene, heptane, etc.).

In some embodiments, the solvent is an alcohol solvent. In some embodiments, the solvent is methanol.

In some embodiments, the deprotection condition (e.g., the deprotection condition for providing Compound IX) comprises a temperature of about 0° C. to about 60° C. In some embodiments, the temperature is about 10° C. to about 30° C. In some embodiments, the temperature is about 20° C.

In some embodiments, the deprotection condition (e.g., the deprotection condition for providing Compound IX) comprises an acid such as a chiral acid (e.g., (+)-Camphorsulfonic acid), a catalyst such as palladium on carbon, a reductant such as a hydrogen source (e.g., hydrogen gas), a solvent such as an alcohol solvent (e.g., methanol), and at a suitable temperature such as a temperature of about 10° C. to about 30° C. (e.g., about 20° C.).

In some embodiments, the product from step (c) is Compound IX (+)-camphorsulfonic acid salt.

In some embodiments, the compound of Formula IX-1 is Compound IX-1-Np.

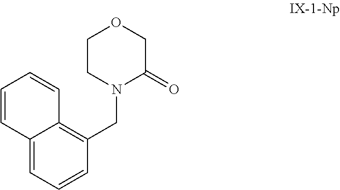 [SMILES: O=C1COCCN1Cc1cccc2ccccc12]

In some embodiments, the process further comprises preparing Compound IX-1-Np by contacting morpholin-3-one with a naphthalenylmethyl precursor. The naphthalenylmethyl precursor may be 1-(chloromethyl)naphthalene, 1-(bromomethyl)naphthalene, 1-(iodomethyl)naphthalene, or 1-(hydroxymethyl)naphthalenes activated as sulfonates or phosphates.

In some embodiments, the naphthalenylmethyl precursor is 1-(chloromethyl)naphthalene.

In some embodiments, the reaction between morpholin-3-one and the naphthalenylmethyl precursor occurs in the presence of a base. Suitable bases include, but are not limited to, hydroxides (e.g., lithium hydroxide, sodium hydroxide, potassium hydroxide, cesium hydroxide, etc.), alkoxides (e.g., lithium tert-butoxide, sodium tert-butoxide, potassium tert-butoxide, etc.), n-butyllithium, hydrides (e.g., sodium hydride, potassium hydride), and amides (e.g., lithium bis(trimethylsilyl)amide, potassium bis(trimethylsilyl)amide, lithium diisopropylamide, etc.).

In some embodiments, the base is an amide base. In some embodiments, the base is lithium bis(trimethylsilyl)amide.

In some embodiments, the reaction between morpholin-3-one and the naphthalenylmethyl precursor occurs in the presence of a catalyst, Suitable catalysts include, but are not limited to, sodium iodide and tetrabutylammonium salts (e.g., tetrabutylammonium iodide, tetrabutylammonium bromide, tetrabutylammonium chloride, etc.).

In some embodiments, the catalyst is sodium iodide.

In some embodiments, the reaction between morpholin-3-one and the naphthalenylmethyl precursor occurs in the presence of a solvent. Suitable solvents include, but are not limited to, hydrocarbons (e.g., toluene, benzene, etc.), ethers (e.g., tetrahydrofuran, 2-methyltetrahydrofuran, 1,4-dioxane, etc.), and polar aprotic solvents (e.g., N,N-dimethylacetamide, N-methyl-2-pyrrolidone, N-butyl-2-pyrrolidone, dimethyl sulfoxide, etc.).

In some embodiments, the solvent is an ether solvent. In some embodiments, the solvent is tetrahydrofuran.

In some embodiments, the reaction between morpholin-3-one and the naphthalenylmethyl precursor occurs at a temperature of about −20° C. to about 60° C. In some embodiments, the temperature is about −10° C. to about 50° C. In some embodiments, the temperature is about 0° C. to about 40° C.

In some embodiments, the reaction between morpholin-3-one and the naphthalenylmethyl precursor (e.g., 1-(chloromethyl)naphthalene) occurs in the presence of a base such as an amide base (e.g., lithium bis(trimethylsilyl)amide), a catalyst such as sodium iodide, a solvent such as an ether solvent (e.g., tetrahydrofuran), and at a suitable temperature such as a temperature of about −10° C. to about 50° C. (e.g., about 0° C. to about 40° C.).

In some embodiments, the compound of Formula IX-1 is Compound IX-1-Bn.

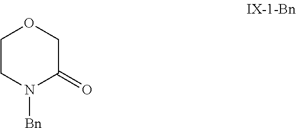 [SMILES: O=C1COCCN1Cc1ccccc1]

In some embodiments, the process further comprises preparing Compound IX-1-Bn by contacting morpholin-3-one with a benzyl precursor. The benzyl precursor may be benzyl chloride, benzyl bromide, benzyl iodide, benzyl tosylate, or benzyl mesylate.

In some embodiments, the benzyl precursor is benzyl bromide.

In some embodiments, the reaction between morpholin-3-one and the benzyl precursor occurs in the presence of a base. Suitable bases include, but are not limited to, hydroxides (e.g., lithium hydroxide, sodium hydroxide, potassium hydroxide, cesium hydroxide, etc.), alkoxides (e.g., lithium tert-butoxide, sodium tert-butoxide, potassium tert-butoxide, etc.), n-butyllithium, hydrides (e.g., sodium hydride, potassium hydride), and amides (e.g., lithium bis(trimethylsilyl)amide, potassium bis(trimethylsilyl)amide, lithium diisopropylamide, etc.).

In some embodiments, the base is a hydride base. In some embodiments, the base is sodium hydride.

In some embodiments, the reaction between morpholin-3-one and the benzyl precursor occurs in the presence of a catalyst. The catalyst may be a tetrabutylammonium salt, such as tetrabutylammonium iodide, tetrabutylammonium bromide, or tetrabutylammonium chloride.

In some embodiments, the reaction between morpholin-3-one and the benzyl precursor occurs in the absence of a catalyst.

In some embodiments, the reaction between morpholin-3-one and the benzyl precursor occurs in the presence of a solvent. Suitable solvents include, but are not limited to, hydrocarbons (e.g., toluene, benzene, etc.), ethers (e.g., tetrahydrofuran, 2-methyltetrahydrofuran, 1,4-dioxane, etc.), and polar aprotic solvents (e.g., N,N-dimethylacetamide, N-methyl-2-pyrrolidone, N-butyl-2-pyrrolidone, dimethyl sulfoxide, etc.).

In some embodiments, the solvent is an ether solvent. In some embodiments, the solvent is tetrahydrofuran.

In some embodiments, the reaction between morpholin-3-one and the benzyl precursor occurs at a temperature of about −20° C. to about 60° C. In some embodiments, the temperature is about −0° C. to about 20° C. In some embodiments, the temperature is about 10° C.

In some embodiments, the reaction between morpholin-3-one and the benzyl precursor (e.g., benzyl bromide) occurs in the presence of a base such as a hydride base (e.g., sodium hydride), a solvent such as an ether solvent (e.g., tetrahydrofuran), and at a suitable temperature such as a temperature of about 0° C. to about 20° C. (e.g., about 10° C.).

### Mitsunobu Cyclization Route

Additionally or alternatively, Compound IX or a salt thereof may be prepared via a compound of Formula IX-5a or a salt thereof by using Mitsunobu cyclization.

In some embodiments, provided is a process for preparing a compound of Formula IX-5a

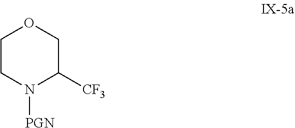

or a salt thereof, wherein PGN is a nitrogen protecting group;
the process comprises one or more or all of the following steps:
(a) contacting Compound XIII-1

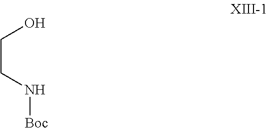 [SMILES: CC(C)(C)OC(=O)NCCO]

or a salt thereof with Compound XIII-2

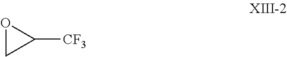

or a salt thereof to provide Compound XII-1

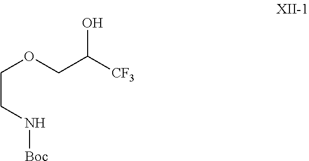 [SMILES: CC(C)(C)OC(=O)NCCOCC(O)C(F)(F)F]

or a salt thereof;
(b) subjecting Compound XII-1 or a salt thereof to a deprotection condition to provide Compound XII-2

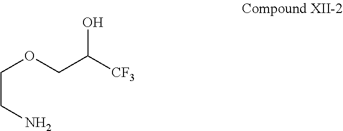 [SMILES: NCCOCC(O)C(F)(F)F]

or a salt thereof;
(c) converting Compound XII-2 or a salt thereof to a compound of Formula XII-3

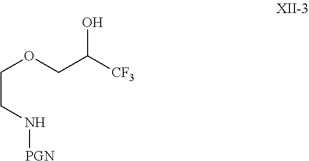

or a salt thereof, wherein PGN is a nitrogen protecting group; and
(d) subjecting a compound of Formula XII-3 or a salt thereof to a cyclization condition to provide a compound of Formula IX-5a.

The nitrogen protecting group (PGN) (e.g., the PGN group of Formula IX-5a) can be a sulfonyl group, acyl group, or carbamate group. For example, the PGN may be p-toluenesulfonyl, 4-nitrobenzenesulfonyl, methanesulfonyl, benzenesulfonyl, triflouromethylsulfonyl, acetyl, trifluoroacetyl, benzoyl, pivaloyl, benzyloxycarbonyl, or tert-butyloxycarbonyl.

In some embodiments, PGN is a sulfonyl group. In some embodiments, PGN is p-toluenesulfonyl or 4-nitrobenzenesulfonyl.

In some embodiments, the process comprises contacting Compound XIII-1 or a salt thereof with Compound XIII-2 or a salt thereof to provide Compound XII-1 or a salt thereof.

In some embodiments, the reaction (e.g., the reaction between Compound XIII-1 and Compound XIII-2) occurs in the presence of a base. Suitable bases include, but are not limited to, hydroxides (e.g., lithium hydroxide, sodium hydroxide, potassium hydroxide, cesium hydroxide, etc.), alkoxides (e.g., alkali salts of methanol, ethanol, tert-butanol, etc.), carbonates (e.g., potassium carbonate, cesium carbonate, etc.), alkyllithium reagents (e.g., n-butyllithium, t-butyllithium, etc.), hydrides (e.g., sodium hydride, potassium hydride, etc.), and amides (e.g., lithium bis(trimethylsilyl)amide, potassium bis(trimethylsilyl)amide, lithium diisopropylamide, etc.).

In some embodiments, the base is a hydride. In some embodiments, the base is sodium hydride.

In some embodiments, the reaction (e.g., the reaction between Compound XIII-1 and Compound XIII-2) occurs in the presence of a catalyst. In some embodiments, the reaction (e.g., the reaction between Compound XIII-1 and Compound XIII-2) occurs in the presence of a catalyst and in the absence of a base. Suitable catalysts include, but are not limited to, Lewis acids (e.g., boron trifluoride etherate, aluminum triflate, trimethylsilyl triflate, titanium tetrachloride, cobalt salen, zeolites, etc.) and protic acids (e.g., sulfuric acid, trifluoroacetic acid, p-toluenesulfonic acid, non-aqueous solutions of hydrochloric acid, etc.).

In some embodiments, the reaction (e.g., the reaction between Compound XIII-1 and Compound XIII-2) occurs in the absence of a catalyst.

In some embodiments, the reaction (e.g., the reaction between Compound XIII-1 and Compound XIII-2) occurs in the presence of a solvent. Suitable solvents include, but are not limited to, hydrocarbons (e.g., toluene, benzene, etc.), ethers (e.g., tetrahydrofuran, 2-methyltetrahydrofuran, 1,4-dioxane, etc.), and polar aprotic solvents (e.g., N,N-dimethylacetamide, N-methyl-2-pyrrolidone, N-butyl-2-pyrrolidone, dimethyl sulfoxide, etc.).

In some embodiments, the solvent is an ether. In some embodiments, the solvent is tetrahydrofuran.

In some embodiments, the reaction (e.g., the reaction between Compound XIII-1 and Compound XIII-2) occurs at a temperature of about −60° C. to about 60° C. In some embodiments, the temperature is about −10° C. to about 10° C. In some embodiments, the temperature is about 0° C.

In some embodiments, the reaction (e.g., the reaction between Compound XIII-1 and Compound XIII-2) occurs in the presence of a base such as a hydride (e.g., sodium hydride), a solvent such as an ether (e.g., tetrahydrofuran), and at a suitable temperature such as a temperature of about −10° C. to about 10° C. (e.g., about 0° C.).

In some embodiments, Compound XII-1 is obtained as a racemic mixture. In some embodiments, Compound XII-1 is obtained as a single enantiomer (e.g., a single (S)-enantiomer).

In some embodiments, the process comprises subjecting Compound XII-1 or a salt thereof to a deprotection condition to provide Compound XII-2 or a salt thereof.

In some embodiments, the deprotection condition (e.g., the deprotection condition for providing Compound XII-2 from Compound XII-1) comprises a deprotection reagent. Suitable deprotection reagents include, but are not limited to, hydrogen chloride in an anhydrous solvent (e.g., hydrogen chloride in dioxane, etc.), mineral acids (e.g., hydrochloric acid, hydrobromic acid, sulfuric acid, etc.), carboxylic acids (e.g., acetic acid, pivalic acid, trifluoroacetic acid, etc.), sulfonic acids (e.g., p-toluenesulfonic acid, etc.), oxalyl chloride, Lewis acids (e.g., boron trifluoride diethyl etherate, boron trifluoride THF complex, aluminum trichloride, silicon tetrachloride, etc.), and metal salts (e.g., iron(III) chloride, zinc(II) bromide, etc.).

In some embodiments, the deprotection reagent is hydrogen chloride in an anhydrous solvent. In some embodiments, the deprotection reagent is hydrogen chloride in dioxane.

In some embodiments, the deprotection condition (e.g., the deprotection condition for providing Compound XII-2 from Compound XII-1) comprises a solvent. Suitable solvents include, but are not limited to, ethers (e.g., tetrahydrofuran, 2-methyltetrahydrofuran, tert-butyl methyl ether, etc.), polar aprotic solvents (e.g., N,N-dimethylformamide, N,N-dimethylacetamide, dimethyl sulfoxide, etc.), nitriles (e.g., acetonitrile), halogenated solvents (e.g., dichloromethane), alcohols (e.g., methanol, ethanol, etc.), and any combination thereof.

In some embodiments, the solvent is an ether. In some embodiments, the solvent is tetrahydrofuran.

In some embodiments, the deprotection condition (e.g., the deprotection condition for providing Compound XII-2 from Compound XII-1) comprises a temperature of about −40° C. to about 40° C. In some embodiments, the temperature is about −10° C. to about 30° C. In some embodiments, the temperature is about 0° C. to about 20° C.

In some embodiments, the deprotection condition (e.g., the deprotection condition for providing Compound XII-2 from Compound XII-1) comprises a deprotection reagent such as hydrogen chloride in an anhydrous solvent (e.g., hydrogen chloride in dioxane), a solvent such as an ether (e.g., tetrahydrofuran), and a suitable temperature such as a temperature of about −10° C. to about 30° C. (e.g., about 0° C. to about 20° C.).

In some embodiments, the process comprises converting Compound XII-2 or a salt thereof to a compound of Formula XII-3 or a salt thereof.

In some embodiments, the reaction (e.g., the reaction for providing a compound of Formula XII-3 from Compound XII-2) occurs in the presence of a protecting group source. The protecting group source may be a sulfonyl source such as sulfonyl chlorides (e.g., p-toluenesulfonyl chloride, 4-nitrobenzenesulfonyl chloride, etc.), sulfonyl bromides, sulfonyl fluorides, sulfonyl anhydrides, or N-phenyltriflimides. Additionally or alternatively, the protecting group source may be an acyl source such as acyl chlorides or acyl anhydrides. The protecting group source may also be a carbamate source such as benzyl chloroformate or di-tert-butyl dicarbonate.

In some embodiments, the protecting group source is a sulfonyl source. In some embodiments, the protecting group source is p-toluenesulfonyl chloride or 4-nitrobenzenesulfonyl chloride.

In some embodiments, the reaction (e.g., the reaction for providing a compound of Formula XII-3 from Compound XII-2) occurs in the presence of a base. Suitable bases include, but are not limited to, aromatic amines (e.g., pyridine, picoline isomers, lutidine isomers, collidine isomers, indole, isoindole, quinolone, isoquinoline, etc.) and amines (e.g., N-methylmorpholine, N-ethylmorpholine, tri-n-propylamine, N,N-diisopropylethylamine, trimethylamine, triethylamine, tri-n-butylamine, 1,8-diazabicyclo[5.4.0]undec-7-ene, 1,4-diazabicyclo[2.2.2]octane, etc.).

In some embodiments, the base is an amine base. In some embodiments, the base is triethylamine.

In some embodiments, the reaction (e.g., the reaction for providing a compound of Formula XII-3 from Compound XII-2) occurs in the presence of a catalyst. Suitable catalysts include, but are not limited to, N-methylimidazole, 4-dimethylaminopyridine, 4-pyrrolidinopyridine, 4-piperidinopyridine, and 9-azajulolidine.

In some embodiments, the reaction (e.g., the reaction for providing a compound of Formula XII-3 from Compound XII-2) occurs in the absence of a catalyst.

In some embodiments, the reaction (e.g., the reaction for providing a compound of Formula XII-3 from Compound XII-2) occurs in the presence of a solvent. Suitable solvents include, but are not limited to, esters (e.g., ethyl acetate, isopropyl acetate, etc.), ethers (e.g., tetrahydrofuran, 2-methyltetrahydrofuran, methyl tert-butyl ether, cyclopentyl methyl ether, etc.), hydrocarbons (e.g., toluene, n-heptane, etc.), polar aprotic solvents (e.g., N,N-dimethylformamide, N,N-dimethylacetamide, N-methyl-2-pyrrolidinone, dimethyl sulfoxide, etc.), halogenated solvents (e.g., dichloromethane, chloroform, 1,2-dichloroethane, chlorobenzene, etc.), and nitriles (e.g., acetonitrile).

In some embodiments, the solvent is a halogenated solvent. In some embodiments, the solvent is dichloromethane.

In some embodiments, the reaction (e.g., the reaction for providing a compound of Formula XII-3 from Compound XII-2) occurs at a temperature of about −40° C. to about 80° C. In some embodiments, the temperature is about −10° C. to about 30° C. In some embodiments, the temperature is about 0° C. to about 20° C.

In some embodiments, the reaction (e.g., the reaction for providing a compound of Formula XII-3 from Compound XII-2) occurs in the presence of a protecting group source such as a sulfonyl source (e.g., p-toluenesulfonyl chloride or 4-nitrobenzenesulfonyl chloride), a base such as an amine base (e.g., triethylamine), a solvent such as a halogenated solvent (e.g., dichloromethane), and at a suitable temperature such as a temperature of about −10° C. to about 30° C. (e.g., about 0° C. to about 20° C.).

In some embodiments, the process comprises subjecting a compound of Formula XII-3 or a salt thereof to a cyclization condition to provide a compound of Formula IX-5a or a salt thereof.

In some embodiments, the cyclization condition (e.g., the cyclization condition for providing a compound of Formula IX-5a from a compound of Formula XII-3) comprises a reductive activator. Suitable reductive activators include, but are not limited to, arylphosphines (e.g., triphenylphosphine, 4-(diphenylphosphino)benzoic acid, 1,2-bis(diphenylphosphino)ethane, 4-(dimethylamino)phenyldiphenylphosphine, etc.), alkylphosphines (e.g., trimethylphosphine, tricyclohexylphosphine, tributylphosphine, etc.), and resin-bound analogues thereof.

In some embodiments, the reductive activator is an arylphosphine. In some embodiments, the reductive activator is triphenylphosphine.

In some embodiments, the cyclization condition (e.g., the cyclization condition for providing a compound of Formula IX-5a from a compound of Formula XII-3) comprises a reagent such as azodicarboxylates (e.g., diisopropyl azodicarboxylate, diethyl azodicarboxylate, di-2-methoxyethyl azodicarboxylate, di-p-chlorobenzyl azodicarboxylate, di-tert-butylazodicarboxylate, etc.), azo-type reagents (e.g., 1,1-(azodicarbonyl)dipiperidine, 4,4′-azopyridine, ethyl 2-(3,4-dichlorophenyl)azocarboxylate, etc.), or phosphonium ylides (e.g., (cyanomethylene)tributylphosphorane, (cyanomethylene)trimethylphosphorane, etc.).

In some embodiments, the cyclization condition (e.g., the cyclization condition for providing a compound of Formula IX-5a from a compound of Formula XII-3) comprises diisopropyl azodicarboxylate.

In some embodiments, the cyclization condition (e.g., the cyclization condition for providing a compound of Formula IX-5a from a compound of Formula XII-3) comprises a catalyst such as (2-hydroxybenzyl)diphenylphosphine oxide.

In some embodiments, the cyclization condition (e.g., the cyclization condition for providing a compound of Formula IX-5a from a compound of Formula XII-3) does not comprise a catalyst.

In some embodiments, the cyclization condition (e.g., the cyclization condition for providing a compound of Formula IX-5a from a compound of Formula XII-3) comprises a solvent. Suitable solvents include, but are not limited to, esters (e.g., ethyl acetate, isopropyl acetate, etc.), ethers (e.g., tetrahydrofuran, 2-methyltetrahydrofuran, methyl tert-butyl ether, cyclopentyl methyl ether, etc.), hydrocarbons (e.g., toluene, n-heptane, etc.), polar aprotic solvents (e.g., N,N-dimethylformamide, N,N-dimethylacetamide, N-methyl-2-pyrrolidinone, dimethyl sulfoxide, etc.), halogenated solvents (e.g., dichloromethane, chloroform, 1,2-dichloroethane, chlorobenzene, etc.), and nitriles (e.g., acetonitrile, etc.).

In some embodiments, the solvent is a halogenated solvent. In some embodiments, the solvent is dichloromethane.

In some embodiments, the cyclization condition (e.g., the cyclization condition for providing a compound of Formula IX-5a from a compound of Formula XII-3) comprises a temperature of about −30° C. to about 100° C. In some embodiments, the temperature is about −10° C. to about 30° C. In some embodiments, the temperature is about 0° C. to about 20° C.

In some embodiments, the cyclization condition (e.g., the cyclization condition for providing a compound of Formula IX-5a or a salt thereof from a compound of Formula XII-3 or a salt thereof) comprises a reductive activator (e.g., such as triphenylphosphine), a reagent (e.g., such as diisopropyl azodicarboxylate), a halogenated solvent (e.g., such as dichloromethane), and a suitable temperature such as a temperature of about −10° C. to about 30° C. (e.g., about 0° C. to about 20° C.).

Additionally or alternatively, the process may comprise subjecting a compound of Formula XII-3 or a salt thereof to a cyclization condition to provide a compound of Formula IX-5a or a salt thereof, wherein the cyclization condition comprises (i) a sulfonylating reagent (e.g., sulfonyl chlorides, sulfonyl anhydrides, N-phenyltriflimide, etc.), an acylating reagent (e.g., acyl chlorides, anhydrides, etc.), a phosphorylating reagent (e.g., diphenylphosphoryl chloride, propanephosphonic acid anhydride, etc.), or an alkoxycarbonylating reagent (e.g., methyl chloroformate, di-tert-butyl decarbonate, etc.); and (ii) an organic or inorganic organic base (e.g., pyridine, triethylamine, 1,8-diazabicyclo[5.4.0]undec-7-ene, cesium carbonate, potassium phosphate, sodium tert-butoxide, sodium hydroxide, etc.); and wherein PGN is an acyl group (e.g., acetyl, trifluoroacetyl, benzoyl, pivaloyl, etc.) or carbamate group (e.g., benzyloxycarbonyl, or tert-butyloxycarbonyl, etc.).

The process may further comprise converting a compound of Formula IX-5a or a salt thereof to Compound IXa or Compound IX or a salt thereof according to a process described anywhere herein.

### Synthesis of Compound X and Derivatives

Also provided is a process for preparing Compound X

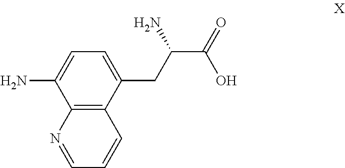 [SMILES: Nc1ccc(C[C@H](N)C(=O)O)c2cccnc12]

or a salt thereof, comprising:
contacting quinolin-8-amine with an ammonia source and a pyruvate source to form Compound X or a salt thereof.

Suitable ammonia source includes, but is not limited to, ammonium hydroxide, ammonium halide salts (e.g., ammonium chloride, ammonium bromide, etc.), ammonium carboxylate salts (e.g., ammonium formate, ammonium acetate, etc.), and ammonium phosphate.

In some embodiments, the ammonia source is an ammonium halide salt. In some embodiments, the ammonia source is ammonium chloride.

Suitable pyruvate source includes, but is not limited to, pyruvic acid and metal pyruvate salts (e.g., lithium pyruvate, sodium pyruvate, potassium pyruvate, etc.).

In some embodiments, the pyruvate source is a metal pyruvate salt. In some embodiments, the pyruvate source is sodium pyruvate.

In some embodiments, the reaction for providing Compound X occurs in the presence of a catalyst. In some embodiments, the catalyst is an enzyme. Suitable catalysts include, but are not limited to, tyrosine phenol lyase (e.g., C. freundii UniProt ID P31013 with additional mutations such as T124D/M288S/R381N/F448C, T124D/M288S/F448C, etc.), tryptophan synthase, and tryptophanase.

In some embodiments, the catalyst is tyrosine phenol lyase (C. freundii UniProt ID P31013 with additional mutations T124D/M288S/R381N/F448C).

In some embodiments, the reaction for providing Compound X occurs in the presence of a cofactor that is pyridoxal phosphate.

In some embodiments, the reaction for providing Compound X occurs in the absence of an additive.

In some embodiments, the reaction for providing Compound X occurs in the presence of an additive. Suitable additives include, but are not limited to, reducing agents (e.g., beta-mercaptoethanol, dithiothreitol, tris(2-carboxyethyl)phosphine, cysteine, glutathione, ascorbic acid, etc.), anti-foaming agents (e.g., poly(dialkylsiloxanes), polyalkylene glycols, etc.), and any combination thereof.

In some embodiments, the additive is a reducing agent. In some embodiments, the additive is beta-mercaptoethanol.

In some embodiments, the reaction for providing Compound X occurs in the presence of a buffer. Suitable buffers include, but are not limited to, potassium phosphate buffer, tris(hydroxymethyl)aminomethane buffer, 2-(N-morpholino)ethanesulfonic acid buffer, N-(2-acetamido)iminodiacetic acid buffer, 2,2′-(piperazine-1,4-diyl)di(ethane-1-sulfonic acid) buffer, 3-(morpholin-4-yl)propane-1-sulfonic acid buffer. The buffers may have a pH of about 5.0 to about 10.0 (e.g., about 8.0) and a concentration of about 0 M to about 4 M (e.g., about 0.1 M).

In some embodiments, the buffer is potassium phosphate, pH 8, 100 mM.

In some embodiments, the reaction for providing Compound X occurs at a temperature of about 0° C. to about 100° C. In some embodiments, the temperature is about 10° C. to about 30° C. In some embodiments, the temperature is about 20° C.

In some embodiments, the reaction between quinolin-8-amine, an ammonia source (e.g., ammonium chloride), and a pyruvate source (e.g., sodium pyruvate) occurs in the presence of an enzyme catalyst (e.g., tyrosine phenol lyase (C. freundii UniProt ID P31013 with additional mutations T124D/M288S/R381N/F448C)), a cofactor that is pyridoxal phosphate, an additive such as a reducing agent (e.g., beta-mercaptoethanol), a buffer such as potassium phosphate buffer (e.g., potassium phosphate, pH 8, 100 mM), and at a suitable temperature such as a temperature of about 10° C. to about 30° C. (e.g., about 20° C.).

In some embodiments, provided is a process for preparing Compound X-1

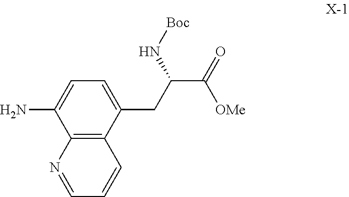 [SMILES: COC(=O)[C@H](Cc1ccc(N)c2ncccc12)NC(=O)OC(C)(C)C]

or a salt thereof, comprising:
(a) contacting Compound IV-6

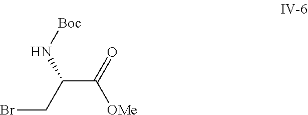 [SMILES: COC(=O)[C@H](CBr)NC(=O)OC(C)(C)C]

or a salt thereof with Compound VII-Br

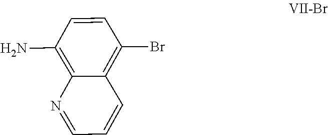 [SMILES: Nc1ccc(Br)c2cccnc12]

or a salt thereof in the presence of a photosensitizer to provide Compound X-1 or a salt thereof.

Suitable photosensitizers (e.g., photosensitizers for providing Compound X-1 from Compound VII-Br and Compound IV-6) include, but are not limited to, iridium photocatalyst (e.g., Ir(dFCF₃ppy)₂(dtbbpy)PF₆, Ir(ppy)₂(dtbbpy)PF₆, Ir(FMeppy)₂(dtbbpy)PF₆, Ir(dFMeppy)₂(dtbbpy)PF₆, Ir(dFFppy)₂(dtbbpy)PF₆, Ir(dFCF₃ppy)₂(bpy)PF₆, Ir(dFCF₃ppy)₂(dCF₃bpy)PF₆), and donor-acceptor fluorophores photocatalyst (e.g., 1,2,3,5-tetrakis(carbazol-9-yl)-4,6-dicyanobenzene, 2,4,5,6-Tetrakis(9H-carbazol-9-yl) isophthalonitrile (4CzIPN, CAS 1416881-52-1), 2,4,6-tris(diphenylamino)-5-fluoroisophthalonitrile (3DPAIPN, CAS 2260543-73-3), 3,4,5,6-tetrakis(carbazol-9-yl)-1,2-dicyanobenzene (4CzPN, CAS 1416881-51-0), 2,3,5,6-tetrakis(carbazol-9-yl)-1,4-dicyanobenzene (4CzTPN, CAS 1416881-53-2), 2,4,5,6-tetrakis(diphenylamino)isophthalonitrile (4DPAIPN, CAS 1846598-27-3), etc.).

In some embodiments, the photosensitizer is an iridium photocatalyst. In some embodiments, the photosensitizer is Ir(dFCF₃ppy)₂(bpy)PF₆.

In some embodiments, the reaction between Compound IV-6 or a salt thereof and Compound VII-Br or a salt thereof occurs in the presence of a co-catalyst. In some embodiments, the co-catalyst is a nickel co-catalyst. Suitable nickel co-catalysts include, but are not limited to, nickel(II) bromide ethylene glycol dimethyl ether, nickel(II) chloride hexahydrate, nickel(II) chloride ethylene glycol dimethyl ether, nickel(II) tetrafluoroborate, nickel(II) acetate, nickel(II) bromide trihydrate, nickel(II) acetylacetonate, and nickel(II) stearate.

In some embodiments, the co-catalyst is nickel(II) bromide ethylene glycol dimethyl ether.

In some embodiments, the reaction between Compound IV-6 or a salt thereof and Compound VII-Br or a salt thereof occurs in the presence of a ligand. Suitable ligands include, but are not limited to, bipyridine ligands (e.g., 4,4′-di-tert-butyl-2,2′-dipyridyl, 2,2′-dipyridine, etc.) and 1,10-phenanthroline.

In some embodiments, the ligand is a bipyridine ligand. In some embodiments, the ligand is 4,4′-di-tert-butyl-2,2′-dipyridyl.

In some embodiments, the reaction between Compound IV-6 or a salt thereof and Compound VII-Br or a salt thereof occurs in the presence of a reductant. Suitable reductants include, but are not limited to, silanes (e.g., tris(trimethylsilyl)silane, triphenyl silane, triethyl silane, dimethyl(phenyl)silane, N-(tert-butyl)-1,1,1,3,3,3-hexamethyl-2-(trimethylsilyl)trisilan-2-amine, N-isopropyl-1,1,1,3,3,3-hexamethyl-2-(trimethylsilyl)trisilan-2-amine, N-butyl-1,1,1,3,3,3-hexamethyl-2-(trimethylsilyl)trisilan-2-amine, tris(trimethylsilyl)silanol, etc.), amines (e.g., triethylamine, triethanolamine, N,N-diisopropylethylamine, tripropylamine, diethylpropylamine, etc.), and benzophenones (e.g., benzophenone, (4-methoxyphenyl)[4-(trifluoromethyl)phenyl]methanone, etc.).

In some embodiments, the reductant is a silane. In some embodiments, the reductant is tris(trimethylsilyl)silane.

In some embodiments, the reaction between Compound IV-6 or a salt thereof and Compound VII-Br or a salt thereof occurs in the presence of a base. Suitable bases include, but are not limited to, cyclic aliphatic amines (e.g., 2,2,6,6-tetramethylpiperidine, 1-methylpiperidine, pyrrolidine, piperidine, etc.), acetates (e.g., potassium acetate, lithium acetate, sodium acetate, ammonium acetate, cesium acetate, etc.), carbonates (e.g., sodium carbonate, potassium carbonate, cesium carbonate, etc.), quinuclidine-based bases (e.g., quinuclidine, 1,4-diazabicyclo[2.2.2]octane (DABCO), hydroxyquinuclidine, 3-acetoxyquinuclidine, etc.), guanidine bases (e.g., 1,1,3,3-tetramethylguanidine, etc.), hydroxides (e.g., lithium hydroxide, sodium hydroxide, potassium hydroxide, etc.), heterocyclic aromatic bases (e.g., 2,6-lutidine, etc.), and phosphates (e.g., tripotassium phosphate, trilithium phosphate, etc.).

In some embodiments, the base is a carbonate base. In some embodiments, the base is sodium carbonate.

In some embodiments, the reaction between Compound IV-6 or a salt thereof and Compound Va-Br or a salt thereof occurs in the absence of an additive.

In some embodiments, the reaction between Compound IV-6 or a salt thereof and Compound Va-Br or a salt thereof occurs in the presence of an additive. Suitable additives include, but are not limited to, phthalimide additives (e.g., phthalimide, 4-tert-butylphthalimide, 4-chloro-1,3-dioxo-1,3-dihydroisoindole, methyl 1,3-dioxoisoindoline-5-carboxylate, etc.) and succinimide additives (e.g., succinimide, 1,2,3,6-tetrahydrophthalimide, 2,2,3,3-tetramethylsuccinimide, (±)-α-methyl-α-phenylsuccinimide, bicyclo[2.2.1]heptane-2,3-dicarboximide, etc.).

In some embodiments, the reaction between Compound IV-6 or a salt thereof and Compound Va-Br or a salt thereof occurs in the presence of a solvent. Suitable solvents include, but are not limited to, ethers (e.g., 1,2-dimethoxyethane, cyclopentyl methyl ether, 1,4-dioxane, 2-methyltetrahydrofuran, dibutyl ether, tert-butyl methyl ether, tetrahydrofuran, etc.), nitriles (e.g., acetonitrile, etc.), polar aprotic solvents (e.g., dimethylacetamide, N,N′-dimethylpropyleneurea, 1,3-dimethyl-2-imidazolidinone, dimethyl sulfoxide, N-methyl-2-pyrrolidone, N,N-dimethylformamide, etc.), and alcohols (e.g., isopropyl alcohol, 1,1,1,3,3,3-hexafluoro-2-propanol, etc.).

In some embodiments, the solvent is an ether solvent. In some embodiments, the solvent is 1,2-dimethoxyethane.

In some embodiments, the reaction between Compound IV-6 or a salt thereof and Compound VII-Br or a salt thereof occurs at a temperature of about 0° C. to about 120° C. In some embodiments, the temperature is about 15° C. to about 35° C. In some embodiments, the temperature is about 25° C.

In some embodiments, the reaction between Compound IV-6 or a salt thereof and Compound VII-Br or a salt thereof occurs under a light having a wavelength of about 330 nm to about 500 nm. In some embodiments, the wavelength is about 450 nm.

In some embodiments, the reaction between Compound IV-6 or a salt thereof and Compound VII-Br or a salt thereof occurs in the presence of a photosensitizer such as an iridium photocatalyst (e.g., Ir(dFCF₃ppy)₂(bpy)PF₆), a co-catalyst such as a nickel co-catalyst (e.g., nickel(II) bromide ethylene glycol dimethyl ether), a ligand such as a bipyridine ligand (e.g., 4,4′-di-tert-butyl-2,2′-bipyridine), a base such as a carbonate (e.g., sodium carbonate), a solvent such as an ether solvent (e.g., 1,2-dimethoxyethane), at a suitable temperature such as a temperature of about 15° C. to about 35° C. (e.g., about 25° C.), and under a light having a suitable wavelength such as a wavelength of about 330 nm to about 500 nm (e.g., about 450 nm).

Also provided is a process for preparing Compound X-3

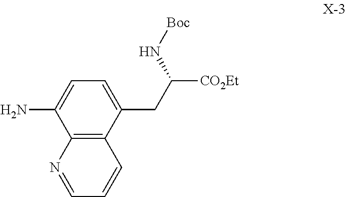 [SMILES: CCOC(=O)[C@H](Cc1ccc(N)c2ncccc12)NC(=O)OC(C)(C)C]

or a salt thereof, comprising:
(a) contacting Compound IV-4

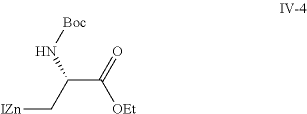

or a salt thereof with Compound VII-2

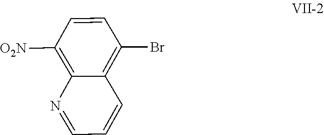 [SMILES: O=[N+]([O-])c1ccc(Br)c2cccnc12]

or a salt thereof in the presence of a catalytic system to provide Compound X-4

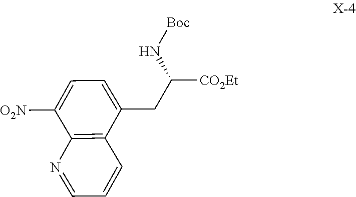 [SMILES: CCOC(=O)[C@H](Cc1ccc([N+](=O)[O-])c2ncccc12)NC(=O)OC(C)(C)C]

or a salt thereof; and
(b) subjecting Compound X-4 or a salt thereof to a reductive condition to provide Compound X-3 or a salt thereof.

In some embodiments, the process comprises contacting Compound IV-4 or a salt thereof with Compound VII-2 or a salt thereof in the presence of a catalytic system to provide Compound X-4 or a salt thereof.

The catalytic system (e.g., the catalytic system for providing Compound X-4 from Compound IV-4 and Compound VII-2) may comprise a catalyst. The catalyst may be a palladium catalyst or a nickel catalyst. The palladium catalyst can be a palladium(II) source (e.g., palladium(II) chloride, etc.) or a palladium(0) source (e.g., tetrakis(triphenylphosphine)palladium(0), tris(dibenzylideneacetone)dipalladium(0), etc.). The nickel catalyst can be a nickel(II) source (e.g., nickel(II) chloride, etc.) or a nickel(0) source (e.g., bis(cyclooctadiene)nickel(0), etc.).

In some embodiments, the catalyst is a palladium(0) source. In some embodiments, the catalyst is tris(dibenzylideneacetone)dipalladium(0).

The catalytic system (e.g., the catalytic system for providing Compound X-4 from Compound IV-4 and Compound VII-2) may further comprise a ligand. Suitable ligands include, but are not limited to, monodentate phosphine ligands (e.g., triphenylphosphine, 2-dicyclohexylphosphino-2′,4′,6′-triisopropylbiphenyl, 2-dicyclohexylphosphino-2′,6′-dimethoxybiphenyl, etc.), bidentate phosphine ligands (e.g., 1,2-bis(diphenylphosphino)ethane, 2,2′-bis(diphenylphosphino)-1,1′-binaphthyl, 1,1′-bis(diphenylphosphino)ferrocene, etc.), bipyridine ligands (e.g., 4,4′-di-tert-butyl-2,2′-dipyridyl, 2,2′-dipyridine), 1,10-phenanthroline, and terpyridine ligands.

In some embodiments, the ligand is a monodentate phosphine ligand. In some embodiments, the ligand is 2-dicyclohexylphosphino-2′,6′-dimethoxybiphenyl.

The catalytic system (e.g., the catalytic system for providing Compound X-4 from Compound IV-4 and Compound VII-2) may further comprise a solvent. Suitable solvents include, but are not limited to, polar aprotic solvents (e.g., N,N-dimethylformamide, N,N-dimethylacetamide, N-methyl-2-pyrrolidone, N-butyl-2-pyrrolidone, dimethyl sulfoxide, etc.), ethers (e.g., tetrahydrofuran, 2-methyltetrahydrofuran, 1,4-dioxane, etc.), and nitriles (e.g., acetonitrile, benzonitrile, etc.).

In some embodiments, the solvent is a polar aprotic solvent. In some embodiments, the solvent is N,N-dimethylformamide.

In some embodiments, the reaction between Compound IV-4 or a salt thereof and Compound VII-2 or a salt thereof occurs at a temperature of about 20° C. to about 120° C. In some embodiments, the temperature is about 45° C. to about 65° C. In some embodiments, the temperature is about 55° C.

In some embodiments, the reaction between Compound IV-4 or a salt thereof and Compound VII-2 or a salt thereof occurs at a suitable temperature such as a temperature of about 45° C. to about 65° C. (e.g., about 55° C.), and in the presence of a catalytic system comprising a catalyst such as a palladium catalyst (e.g., tris(dibenzylideneacetone)dipalladium(0)), a ligand such as a monodentate phosphine ligand (e.g., 2-dicyclohexylphosphino-2′,6′-dimethoxybiphenyl), and a solvent such as a polar aprotic solvent (e.g., N,N-dimethylformamide).

In some embodiments, Compound IV-4 or a salt thereof is prepared according to a process described anywhere herein.

In some embodiments, the process comprises subjecting Compound X-4 or a salt thereof to a reductive condition to provide Compound X-3 or a salt thereof.

In some embodiments, the reductive condition (e.g., the condition for providing Compound X-3 from Compound X-4) comprises a reducing agent. The reducing agent may be a reductant such as zinc (e.g., zinc powder, etc.), tin (e.g., tin(II) chloride, etc.), or iron (e.g., iron powder, etc.) in combination with an acid or an acidic salt (e.g., hydrochloric acid, acetic acid, ammonium chloride, etc.). Additionally or alternatively, the reducing agent may be a transition metal source such as a palladium source (e.g., palladium (II) hydroxide, etc.), a platinum source (e.g., platinum (IV) oxide, etc.), or a rhodium source, in combination with a hydrogen source, such as hydrogen gas, an organosilane (e.g., triethylsilane, etc.), or a formate salt.

In some embodiments, the reducing agent is a reductant in combination with an acid or an acidic salt. In some embodiments, the reducing agent is zinc powder in combination with ammonium chloride.

In some embodiments, the reductive condition (e.g., the condition for providing Compound X-3 from Compound X-4) comprises a solvent. Suitable solvents include, but are not limited to, polar aprotic solvents (e.g., N,N-dimethylacetamide, N-methyl-2-pyrrolidone, dimethyl sulfoxide, etc.), ethers (e.g., tetrahydrofuran, 2-methyltetrahydrofuran, 1,4-dioxane, etc), nitriles (e.g., acetonitrile, benzonitrile, etc.), and a mixed solvent such as a mixture of alcohol (e.g., ethanol, etc.) and water.

In some embodiments, the solvent is a mixture of alcohol and water. In some embodiments, the solvent is a mixture of ethanol and water.

In some embodiments, the reductive condition (e.g., the condition for providing Compound X-3 from Compound X-4) comprises a temperature of about 0° C. to about 60° C. In some embodiments, the temperature is about 10° C. to about 30° C. In some embodiments, the temperature is about 20° C.

In some embodiments, the reductive condition (e.g., the condition for providing Compound X-3 from Compound X-4) comprises a reducing agent such as a reductant in combination with an acid or an acidic salt (e.g., zinc powder in combination with ammonium chloride), a solvent such as a mixture of alcohol and water (e.g., a mixture of ethanol and water), and a suitable temperature such as a temperature of about 10° C. to about 30° C. (e.g., about 20° C.).

### Model Studies

Also provided is a process for preparing Compound XX-1

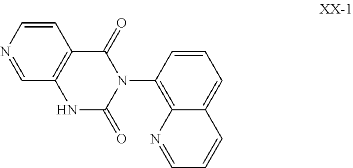 [SMILES: O=c1[nH]c2cnccc2c(=O)n1-c1cccc2cccnc12]

or a salt thereof, comprising:
(a) contacting Compound XX-2

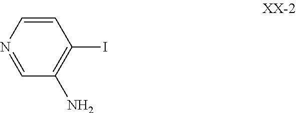

or a salt thereof with Compound XX-3

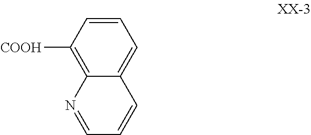 [SMILES: O=C(O)c1cccc2cccnc12]

or a salt thereof to form Compound XX-4

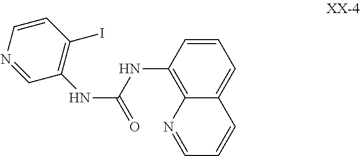 [SMILES: O=C(Nc1cnccc1I)Nc1cccc2cccnc12]

or a salt thereof;
(b) subjecting Compound XX-4 to a cyclization condition to form Compound XX-1 or a salt thereof.

In some embodiments, the reaction (e.g., the reaction between Compound XX-2 and Compound XX-3) occurs in the presence of a base. Suitable bases include, but are not limited to, amines (e.g., triethylamine, N,N-diisopropylethylamine, tripropylamine, tributylamine, 4-methylmorpholine, 1,4-diazabicyclo[2.2.2]-octane, 1,8-diazabicyclo[5.4.0]undec-7-ene, etc.), and basic aromatic compounds (e.g., pyridine, 2,6-lutidine, imidazole, etc.).

In some embodiments, the base is an amine base. In some embodiments, the base is triethylamine.

In some embodiments, the reaction (e.g., the reaction between Compound XX-2 and Compound XX-3) occurs in the presence of an azide source or deoxo-fluor. The azide source may be trimethylsilyl azide, sodium azide, lithium azide, carboxybenzenesulfonazide azide, acetamidobenenesulfonyl azide, or diphenylphosphoryl azide (DPPA).

In some embodiments, the reaction (e.g., the reaction between Compound XX-2 and Compound XX-3) occurs in the presence of an azide source. In some embodiments, the azide source is diphenylphosphoryl azide (DPPA).

In some embodiments, the reaction (e.g., the reaction between Compound XX-2 and Compound XX-3) occurs in the presence of a solvent. Suitable solvents include, but are not limited to, ethers (e.g., tetrahydrofuran, 2-methyltetrahydrofuran, 1,4-dioxane), aromatic solvents (e.g., benzene, toluene, etc.), nitriles (e.g., acetonitrile, benzonitrile, etc.), and halogenated solvents (e.g., dichloromethane, 1,2-dichloroethane, chlorobenzene, etc.).

In some embodiments, the solvent is an aromatic solvent. In some embodiments, the solvent is toluene.

In some embodiments, the reaction (e.g., the reaction between Compound XX-2 and Compound XX-3) occurs at a temperature of about −10° C. to about 120° C. In some embodiments, the temperature is about 70° C. to about 90° C. In some embodiments, the temperature is about 80° C.

In some embodiments, the reaction (e.g., the reaction between Compound XX-2 and Compound XX-3) occurs in the presence of a base such as an amine base (e.g., triethylamine), an azide source such as diphenylphosphoryl azide (DPPA), a solvent such as an aromatic solvent (e.g., toluene), and at a suitable temperature such as a temperature of about 70° C. to about 90° C. (e.g., about 80° C.).

In some embodiments, the process comprises subjecting Compound XX-4 to a cyclization condition to form Compound XX-1 or a salt thereof.

The cyclization condition (e.g., the cyclization condition for providing Compound XX-1 from Compound XX-4) may comprise a catalyst such as a palladium catalyst. The catalyst may be a palladium(II) source (e.g., palladium(II) chloride, etc.) or a palladium(0) source (e.g., tetrakis(triphenylphosphine)palladium(0), tris(dibenzylideneacetone)dipalladium(0), etc.).

In some embodiments, the catalyst is a palladium(0) source. In some embodiments, the catalyst is tris(dibenzylideneacetone)dipalladium(0).

In some embodiments, the cyclization condition (e.g., the cyclization condition for providing Compound XX-1 from Compound XX-4) comprises a ligand. The ligand may be monodentate phosphine ligands (e.g., triphenylphosphine, 2-dicyclohexylphosphino-2′,6′-dimethoxybiphenyl, etc.) or bidentate phosphine ligands (e.g., (S)-(−)-2,2′-p-tolyl-phosphino-1,1′-binaphthyl, 1,2-bis(diphenylphosphino)ethane, 2,2′-bis(diphenylphosphino)-1,1′-binaphthyl, 1,1′-bis(diphenylphosphino)ferrocene, etc.).

In some embodiments, the ligand is a bidentate phosphine ligand. In some embodiments, the ligand is (S)-(−)-2,2′-p-tolyl-phosphino-1,1′-binaphthyl.

In some embodiments, the cyclization condition (e.g., the cyclization condition for providing Compound XX-1 from Compound XX-4) comprises a base. Suitable bases include, but are not limited to, amines (e.g., diisopropylethylamine, triethylamine, tripropylamine, tributylamine, etc.), carbonates (e.g., lithium, sodium, potassium, etc.), phosphate tribasic (e.g., potassium phosphate tribasic, etc.), alkoxides (e.g., sodium methoxide, sodium tert-butoxide, potassium tert-butoxide, etc.), lithium bis(trimethylsilyl)amide, and hydroxides (e.g., potassium hydroxide, sodium hydroxide, etc.).

In some embodiments, the base is an amine base. In some embodiments, the base is diisopropylethylamine.

In some embodiments, the cyclization condition (e.g., the cyclization condition for providing Compound XX-1 from Compound XX-4) comprises a carbonyl source such as carbon monoxide.

In some embodiments, the cyclization condition (e.g., the cyclization condition for providing Compound XX-1 from Compound XX-4) comprises a solvent. Suitable solvents include, but are not limited to, polar aprotic solvents (e.g., N,N-dimethylformamide, N,N-dimethylacetamide, N-methyl-2-pyrrolidone, dimethyl sulfoxide, etc.), ethers (e.g., tetrahydrofuran, 2-methyltetrahydrofuran, 1,4-dioxane, etc.), nitriles (e.g., acetonitrile, benzonitrile, etc.), and hydrocarbons (e.g., benzene, toluene, etc.).

In some embodiments, the solvent is a polar aprotic solvent. In some embodiments, the solvent is N,N-dimethylformamide.

In some embodiments, the cyclization condition (e.g., the cyclization condition for providing Compound XX-1 from Compound XX-4) comprises a temperature of about 20° C. to about 120° C. In some embodiments, the temperature is about 55° C. to about 75° C. In some embodiments, the temperature is about 65° C.

In some embodiments, the cyclization condition (e.g., the cyclization condition for providing Compound XX-1 from Compound XX-4) comprises a catalyst such as a palladium(O) source (e.g., tris(dibenzylideneacetone)dipalladium(0)), a ligand such as a bidentate phosphine ligand (e.g., (S)-(−)-2,2′-p-tolyl-phosphino-1,1′-binaphthyl), a base such as an amine base (e.g., diisopropylethylamine), a carbonyl source such as carbon monoxide, a solvent such as a polar aprotic solvent (e.g., N,N-dimethylformamide), and at a suitable temperature such as a temperature of about 55° C. to about 75° C. (e.g., about 65° C.).

Also provided is a process of for preparing Compound XX-5

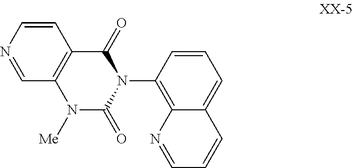

or a salt thereof, comprising:
(a) contacting Compound XX-6

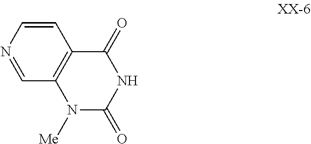 [SMILES: Cn1c(=O)[nH]c(=O)c2ccncc21]

with a tetrabutylammonium base to form Compound XX-6 tetrabutylammonium salt.
(b) contacting Compound XX-6 tetrabutylammonium salt with Compound XX-7

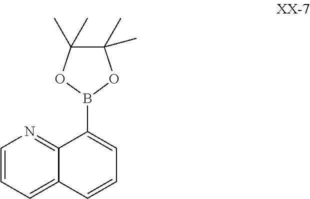 [SMILES: CC1(C)OB(c2cccc3cccnc23)OC1(C)C]

or a salt thereof to form Compound XX-5 or a salt thereof.

In some embodiments, the process comprises contacting Compound XX-6 with a tetrabutylammonium base to form Compound XX-6 tetrabutylammonium salt.

The tetrabutylammonium base may be tetrabutylammonium hydroxide (TBAOH), tetrabutylammonium alkoxides (e.g., TBAOMe, etc.), tetrabutylammonium carbonate, tetrabutylammonium bicarbonate, or tetrabutylammonium phosphate.

In some embodiments, the tetrabutylammonium base is tetrabutylammonium hydroxide (TBAOH).

In some embodiments, the reaction (e.g., the reaction for providing Compound XX-6 tetrabutylammonium salt) occurs in the presence of a solvent. Suitable solvents include, but are not limited to, water, alcohols (e.g., methanol, ethanol, etc.), polar aprotic solvents (e.g., N,N-dimethylacetamide, N-methyl-2-pyrrolidone, dimethyl sulfoxide, etc.), ethers (e.g., tetrahydrofuran, 2-methyltetrahydrofuran, 1,4-dioxane, etc.), nitriles (e.g., acetonitrile, benzonitrile, etc.), and hydrocarbons (e.g., benzene, toluene, etc.).

In some embodiments, the solvent is water.

In some embodiments, the reaction (e.g., the reaction for providing Compound XX-6 tetrabutylammonium salt) occurs at a temperature of about 0° C. to about 100° C. In some embodiments, the temperature is about 10° C. to about 30° C. In some embodiments, the temperature is about 20° C.

In some embodiments, the reaction (e.g., the reaction for providing Compound XX-6 tetrabutylammonium salt) occurs in the presence of a tetrabutylammonium base such as tetrabutylammonium hydroxide (TBAOH), a solvent such as water, and at a suitable temperature such as a temperature of about 10° C. to about 30° C. (e.g., about 20° C.).

In some embodiments, the process comprises contacting Compound XX-6 tetrabutylammonium salt with Compound XX-7 or a salt thereof to form Compound XX-5 or a salt thereof.

In some embodiments, the reaction (e.g., the reaction between Compound XX-6 tetrabutylammonium salt and Compound XX-7) occurs in the presence of a catalyst such as a copper catalyst. The copper catalyst may be a copper (II) source (e.g., Cu(OAc)₂, Cu(OTf)₂, CuCl₂) or a copper(I) source (e.g., CuI, CuCl, [Cu(CH₃CN)₄]PF₆, copper(I) thiophene-2-carboxylate (CuTC), etc.).

In some embodiments, the catalyst is a copper(I) source. In some embodiments, the catalyst is CuTC.

In some embodiments, the reaction (e.g., the reaction between Compound XX-6 tetrabutylammonium salt and Compound XX-7) occurs in the presence of a ligand. The ligand may be bidentate or monodentate P- or N-type ligands, such as (S)-4-benzyl-2-(isoquinolin-1-yl)-4,5-dihydrooxazole (Bn-isoquinox-S), 2,2′-methylenebis[(4S)-4-phenyl-2-oxazoline]((S)-PhBOX), 2-[(4R)-4-(1,1-dimethylethyl)-4,5-dihydro-2-oxazolyl]pyridine ((R)-tBuPyOx), (−)-1,2-Bis((2R,5R)-2,5-diphenylphospholano)ethane ((R,R)-Ph-BPE), 2-[(4S)-4-(1,1-dimethylethyl)-4,5-dihydro-2-oxazolyl]quinoline ((S)-t-Bu-Quinox), or (2S,3S)-(−)-2,3-Bis(diphenylphosphino)butane ((S,S)-Chiraphos).

In some embodiments, the ligand is (S)-4-benzyl-2-(isoquinolin-1-yl)-4,5-dihydrooxazole (Bn-isoquinox-S).

In some embodiments, the reaction (e.g., the reaction between Compound XX-6 tetrabutylammonium salt and Compound XX-7) occurs in the presence of a base. Suitable bases include, but are not limited to, amines (e.g., diisopropylethylamine, triethylamine, tripropylamine, tributylamine, etc.), carbonates (e.g., lithium carbonate, sodium carbonate, potassium carbonate, etc.), phosphate tribasic (e.g., potassium phosphate tribasic, etc.), alkoxides (e.g., sodium methoxide, sodium tert-butoxide, potassium tert-butoxide, etc.), lithium bis(trimethylsilyl)amide, hydroxides (e.g., potassium hydroxide, sodium hydroxide, etc.).

In some embodiments, the base is an amine base. In some embodiments, the base is diisopropylethylamine.

In some embodiments, the reaction (e.g., the reaction between Compound XX-6 tetrabutylammonium salt and Compound XX-7) occurs in the presence of a Lewis acid. The Lewis acids may be transition metal salts (e.g., Sc(OTf)₃, Ti(OMe)₄, FeCl₃, ZnCl₂, etc.), lanthanide salts (e.g., Ce(SO₄)₂, Yb(OTf)₃, etc.), post-transition metal salt (e.g., AlCl₃, etc.), boron reagents (e.g., BF₃, etc.), or alkali/alkaline metal salts (e.g., LiClO₄, Mg(OTf)₂, etc.).

In some embodiments, the Lewis acid is a transition metal salt. In some embodiments, the Lewis acid is Sc(OTf)₃.

In some embodiments, the reaction (e.g., the reaction between Compound XX-6 tetrabutylammonium salt and Compound XX-7) occurs in the presence of an oxidant such as oxygen gas (including air), NaOCl, KMnO₄, MnO₂, NaBrO₃, TEMPO (2,2,6,6-tetramethylpiperidine-1-oxyl), PIDA (phenyliodine(III) diacetate), V₂O₅, or SO₃Pyr (sulfur trioxide pyridine complex).

In some embodiments, the oxidant is air.

In some embodiments, the reaction (e.g., the reaction between Compound XX-6 tetrabutylammonium salt and Compound XX-7) occurs in the presence of a solvent. Suitable solvents include, but are not limited to, polar aprotic solvents (e.g., N,N-dimethylformamide, N,N-dimethylacetamide, N-methyl-2-pyrrolidone, dimethyl sulfoxide, etc.), ethers (e.g., tetrahydrofuran, 2-methyltetrahydrofuran, 1,4-dioxane, etc.), nitriles (e.g., acetonitrile, benzonitrile, etc.), and hydrocarbons (e.g., benzene, toluene, etc.).

In some embodiments, the solvent is a polar aprotic solvent. In some embodiments, the solvent is N,N-dimethylformamide.

In some embodiments, the reaction (e.g., the reaction between Compound XX-6 tetrabutylammonium salt and Compound XX-7) occurs at a temperature of about 40° C. to about 100° C. In some embodiments, the temperature is about 60° C. to about 80° C. In some embodiments, the temperature is about 70° C.

In some embodiments, the reaction (e.g., the reaction between Compound XX-6 tetrabutylammonium salt and Compound XX-7) occurs in the presence of a catalyst such as a copper(I) source (e.g., CuTC), a ligand such as (S)-4-benzyl-2-(isoquinolin-1-yl)-4,5-dihydrooxazole (Bn-isoquinox-S), a base such as an amine base (e.g., diisopropylethylamine), a Lewis acid such as a transition metal salt (e.g., Sc(OTf)₃), an oxidant such as air, a solvent such as a polar aprotic solvent (e.g., N,N-dimethylformamide), and at a suitable temperature such as a temperature of about 60° C. to about 80° C. (e.g., about 70° C.).

### Compounds

In certain embodiments, the disclosure provides intermediate compounds that are useful in the processes described herein.

It is to be understood that processes used for the preparation of a stereochemically enriched compound as described herein may be used for preparing a mixture of stereoisomers (including a racemic mixture). Additionally or alternatively, processes used for the preparation of a mixture of stereoisomers (including a racemic mixture) as described herein may be used for preparing a stereochemically enriched compound.

It can be appreciated that the wedged bolded or dashed bond is used to indicate absolute stereochemistry. Where the composition is identified as stereochemically enriched, it is intended that the composition comprises more than 50% of a single stereoisomer, or at least about 55%, or at least about 60%, or at least about 65%, or at least about 70%, or at least about 75%, or at least about 80%, or at least about 85%, or at least about 90%, or at least about 95%, or at least about 97%, or about 99% of a single stereoisomer.

Provided herein is Compound I or a salt thereof.

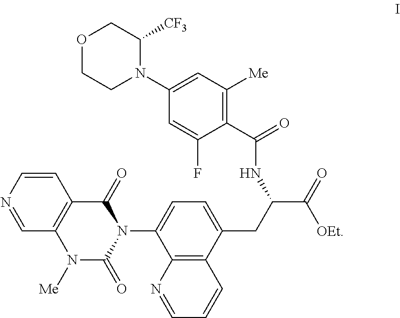

In some embodiments, provided is Compound I Mono-Malonate.

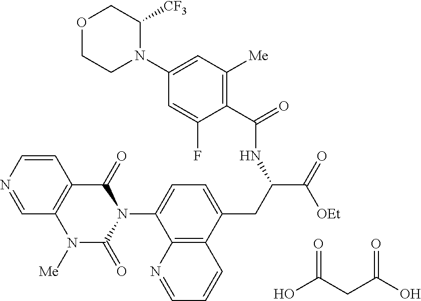

In some embodiments, provided is Compound II or a salt thereof.

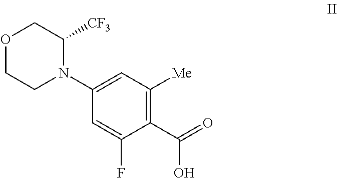 [SMILES: Cc1cc(N2CCOC[C@@H]2C(F)(F)F)cc(F)c1C(=O)O]

In some embodiments, provided is Compound II-1 or a salt thereof.

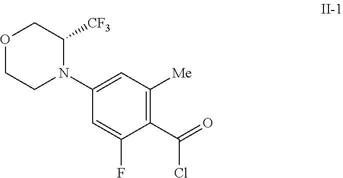 [SMILES: Cc1cc(N2CCOC[C@@H]2C(F)(F)F)cc(F)c1C(=O)Cl]

In some embodiments, provided is Compound II-2 or a salt thereof.

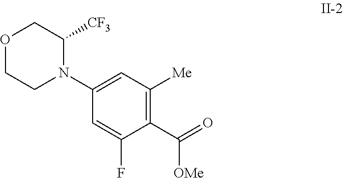 [SMILES: COC(=O)c1c(C)cc(N2CCOC[C@@H]2C(F)(F)F)cc1F]

In some embodiments, provided is Compound II-3 or a salt thereof.

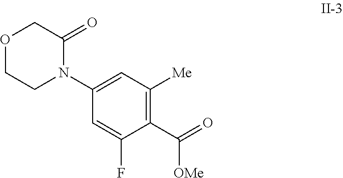 [SMILES: COC(=O)c1c(C)cc(N2CCOCC2=O)cc1F]

In some embodiments, provided is Compound II-4 or a salt thereof.

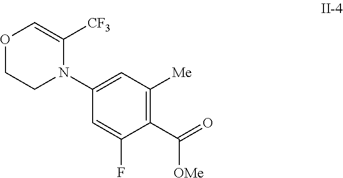 [SMILES: COC(=O)c1c(C)cc(N2CCOC=C2C(F)(F)F)cc1F]

In some embodiments, provided is Compound IIIa or a salt thereof.

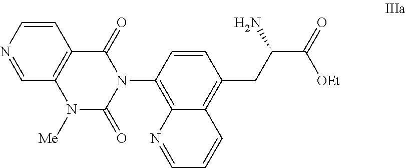 [SMILES: CCOC(=O)[C@@H](N)Cc1ccc(-n2c(=O)c3ccncc3n(C)c2=O)c2ncccc12]

In some embodiments, provided is a single atropisomer of Compound IIIa. In some embodiments, the single atropisomer of Compound IIIa is Compound III.

In some embodiments, provided is Compound III or a salt thereof.

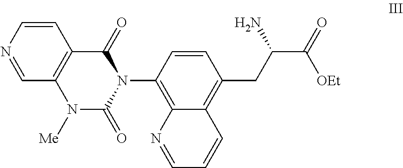

In some embodiments, provided is Compound III-1a or a salt thereof.

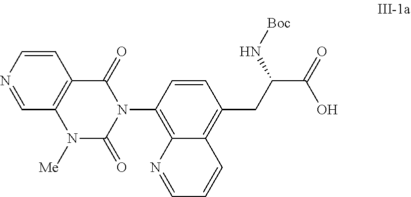 [SMILES: Cn1c(=O)n(-c2ccc(C[C@H](NC(=O)OC(C)(C)C)C(=O)O)c3cccnc23)c(=O)c2ccncc21]

In some embodiments, provided is Compound III-2a or a salt thereof.

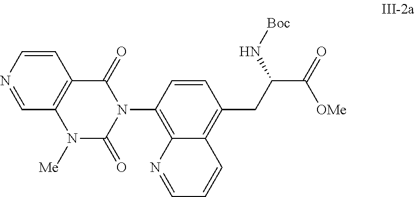 [SMILES: COC(=O)[C@H](Cc1ccc(-n2c(=O)c3ccncc3n(C)c2=O)c2ncccc12)NC(=O)OC(C)(C)C]

In some embodiments, provided is Compound III-3a or a salt thereof.

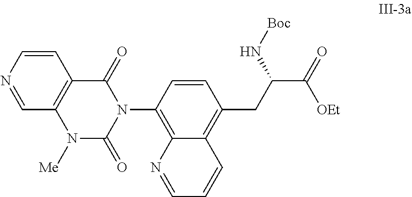 [SMILES: CCOC(=O)[C@H](Cc1ccc(-n2c(=O)c3ccncc3n(C)c2=O)c2ncccc12)NC(=O)OC(C)(C)C]

In some embodiments, provided is a single atropisomer of Compound III-3a. In some embodiments, the single atropisomer of Compound III-3a is Compound III-3.

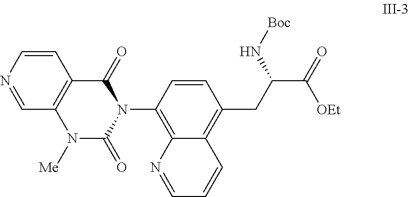

In some embodiments, provided is Compound III-4a or a salt thereof.

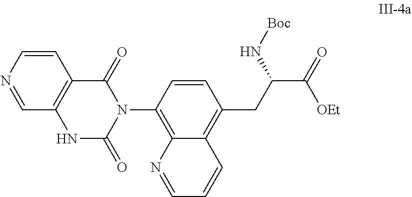 [SMILES: CCOC(=O)[C@H](Cc1ccc(-n2c(=O)[nH]c3cnccc3c2=O)c2ncccc12)NC(=O)OC(C)(C)C]

In some embodiments, provided is a single atropisomer of Compound III-4a. In some embodiments, the single atropisomer of Compound III-4a is Compound III-4.

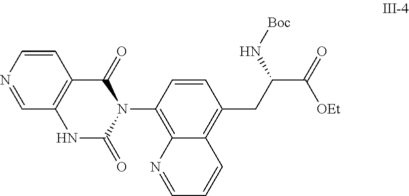

In some embodiments, provided is Compound III-5 or a salt thereof.

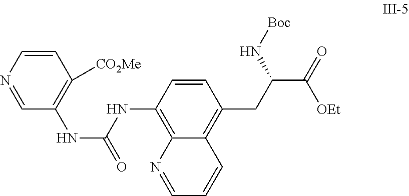 [SMILES: CCOC(=O)[C@H](Cc1ccc(NC(=O)Nc2cnccc2C(=O)OC)c2ncccc12)NC(=O)OC(C)(C)C]

In some embodiments, provided is Compound III-6a or a salt thereof.

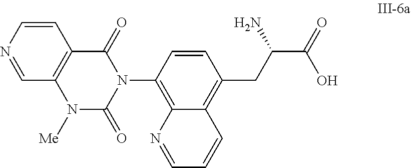 [SMILES: Cn1c(=O)n(-c2ccc(C[C@H](N)C(=O)O)c3cccnc23)c(=O)c2ccncc21]

In some embodiments, provided is a single atropisomer of Compound III-6a. In some embodiments, the single atropisomer of Compound III-6a is Compound III-6.

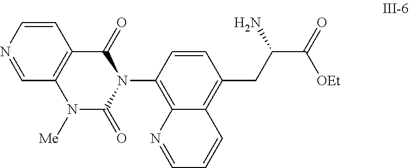

Also provided is a compound of Formula Va:

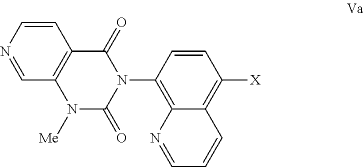

or a salt thereof, wherein X is Br or I.

In some embodiments, provided is a single atropisomer of a compound of Formula Va. In some embodiments, the single atropisomer of a compound of Formula Va is a compound of Formula V:

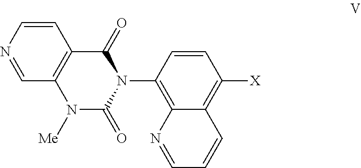

or a salt thereof, wherein X is Br or I.

In some embodiments, X of Formula Va is an iodo group. In some embodiments, provided is Compound Va-I or a salt thereof.

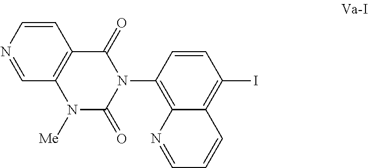 [SMILES: Cn1c(=O)n(-c2ccc(I)c3cccnc23)c(=O)c2ccncc21]

In some embodiments, X of Formula Va is a bromo group. In some embodiments, provided is Compound Va-Br or a salt thereof.

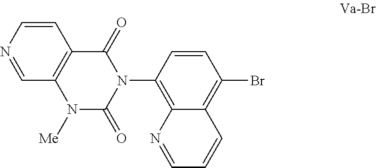 [SMILES: Cn1c(=O)n(-c2ccc(Br)c3cccnc23)c(=O)c2ccncc21]

In some embodiments, X of Formula V is an iodo group. In some embodiments, provided is Compound V-I or a salt thereof.

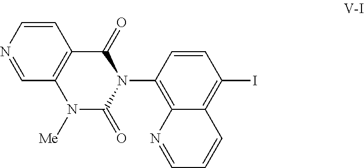

In some embodiments, X of Formula V is a bromo group. In some embodiments, provided is Compound V-Br or a salt thereof.

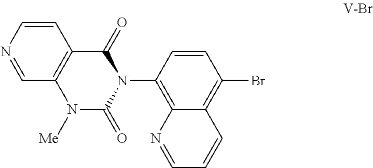

In some embodiments, provided is Compound V-1a:

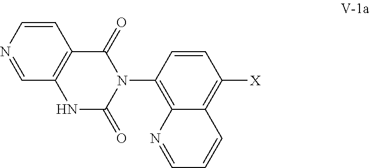

or a salt thereof, wherein X is Br or I.

In some embodiments, provided is a single atropisomer of a compound of Formula V-1a. In some embodiments, the single atropisomer of a compound of Formula V-1a is a compound of Formula V-1:

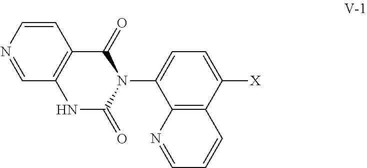

or a salt thereof, wherein X is Br or I.

In some embodiments, X of Formula V-1a is an iodo group. In some embodiments, provided is Compound V-1a-I or a salt thereof.

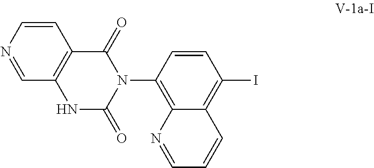 [SMILES: O=c1[nH]c2cnccc2c(=O)n1-c1ccc(I)c2cccnc12]

In some embodiments, X of Formula V-1a is a bromo group. In some embodiments, provided is Compound V-1a-Br or a salt thereof.

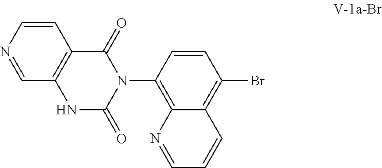 [SMILES: O=c1[nH]c2cnccc2c(=O)n1-c1ccc(Br)c2cccnc12]

In some embodiments, X of Formula V-1 is an iodo group. In some embodiments, provided is Compound V-1-I or a salt thereof.

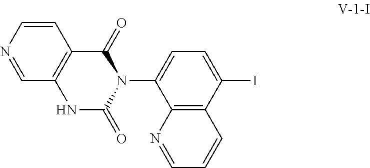

In some embodiments, X of Formula V-1 is a bromo group. In some embodiments, provided is Compound V-1-Br or a salt thereof.

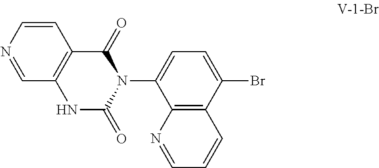

In some embodiments, provided is a compound of Formula V-2:

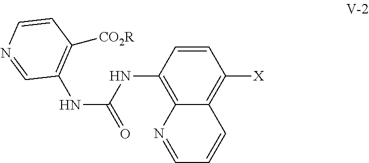

or a salt thereof, wherein
X is Br or I; and
R is a C₁₋₃ alkyl.

In some embodiments, R of Formula V-2 is a methyl group. In some embodiments, the compound of Formula V-2 is a compound of Formula V-3. In some embodiments, provided is a compound of Formula V-3:

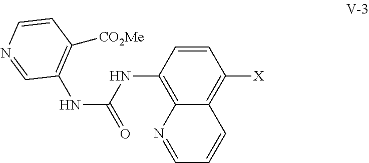

or a salt thereof, wherein X is Br or I.

In some embodiments, X of Formula V-3 is an iodo group. In some embodiments, provided is Compound V-3-I or a salt thereof.

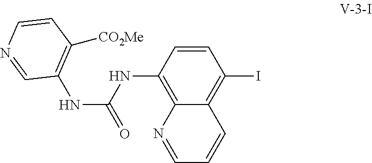 [SMILES: COC(=O)c1ccncc1NC(=O)Nc1ccc(I)c2cccnc12]

In some embodiments, X of Formula V-3 is a bromo group. In some embodiments, provided is Compound V-3-Br or a salt thereof.

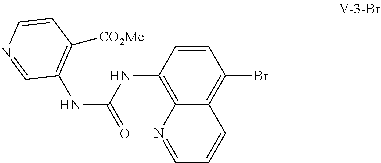 [SMILES: COC(=O)c1ccncc1NC(=O)Nc1ccc(Br)c2cccnc12]

In some embodiments, provided is Compound IXa or a salt thereof.

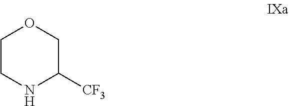

In some embodiments, provided is a single stereoisomer of Compound IXa. In some embodiments, the single stereoisomer of Compound IXa is Compound IX.

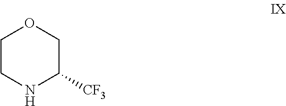

In some embodiments, provided is a compound of Formula IX-1:

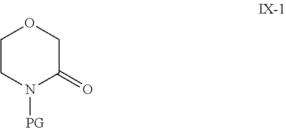

or a salt thereof, wherein PG is benzyl or

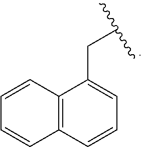 [SMILES: *Cc1cccc2ccccc12 ; "."]

In some embodiments, PG of Formula IX-1 is benzyl. In some embodiments, provided is Compound IX-1-Bn or a salt thereof.

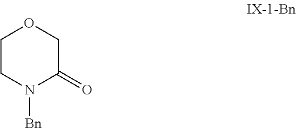 [SMILES: O=C1COCCN1Cc1ccccc1]

In some embodiments, PG of Formula IX-1 is

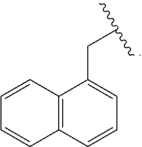 [SMILES: *Cc1cccc2ccccc12 ; "."]

In some embodiments, provided is Compound IX-1-Np or a salt thereof.

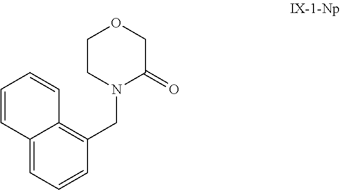 [SMILES: O=C1COCCN1Cc1cccc2ccccc12]

In some embodiments, provided is a compound of Formula IX-2:

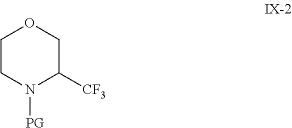

or a salt thereof, wherein PG is benzyl or

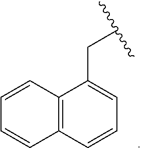 [SMILES: *Cc1cccc2ccccc12 ; "."]

In some embodiments, PG of Formula IX-2 is benzyl. In some embodiments, provided is Compound IX-2-Bn or a salt thereof.

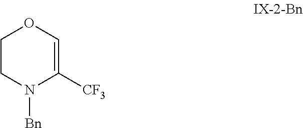 [SMILES: FC(F)(F)C1=COCCN1Cc1ccccc1]

In some embodiments, PG of Formula IX-2 is

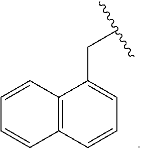 [SMILES: *Cc1cccc2ccccc12 ; "."]

In some embodiments, provided is Compound IX-2-Np or a salt thereof.

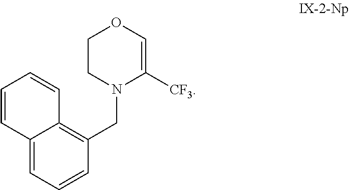 [SMILES: FC(F)(F)C1=COCCN1Cc1cccc2ccccc12]

In some embodiments, provided is Compound IX-3a:

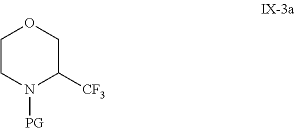

or a salt thereof, wherein PG is benzyl or

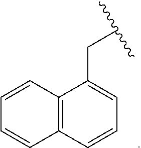 [SMILES: *Cc1cccc2ccccc12 ; "."]

In some embodiments, provided is a single stereoisomer of Compound IX-3a. In some embodiments, the single stereoisomer of Compound IX-3a is Compound IX-3:

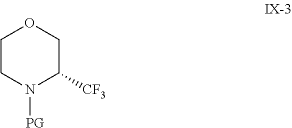

or a salt thereof, wherein PG is benzyl or

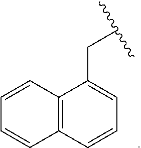 [SMILES: *Cc1cccc2ccccc12 ; "."]

In some embodiments, PG of Formula IX-3a is benzyl. In some embodiments, provided is Compound IX-3a-Bn or a salt thereof.

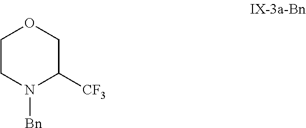 [SMILES: FC(F)(F)C1COCCN1Cc1ccccc1]

In some embodiments, PG of Formula IX-3a is

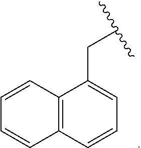 [SMILES: *Cc1cccc2ccccc12 ; "."]

In some embodiments, provided is Compound IX-3a-Np or a salt thereof.

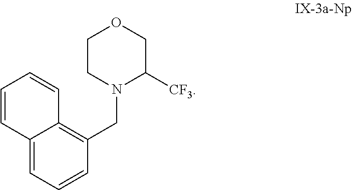 [SMILES: FC(F)(F)C1COCCN1Cc1cccc2ccccc12]

In some embodiments, PG of Formula IX-3 is benzyl. In some embodiments, provided is Compound IX-3-Bn or a salt thereof.

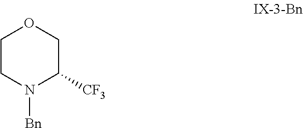 [SMILES: FC(F)(F)[C@H]1COCCN1Cc1ccccc1]

In some embodiments, PG of Formula IX-3 is

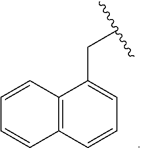 [SMILES: *Cc1cccc2ccccc12 ; "."]

In some embodiments, provided is Compound IX-3-Np or a salt thereof.

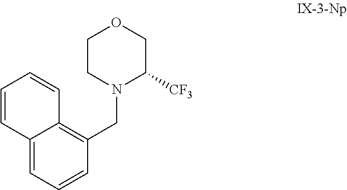 [SMILES: FC(F)(F)[C@H]1COCCN1Cc1cccc2ccccc12]

In some embodiments, provided is a compound of Formula IX-5a

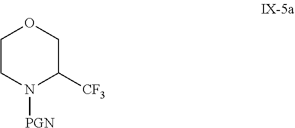

or a salt thereof, wherein PGN is a nitrogen protecting group.

In some embodiments, PGN is p-toluenesulfonyl, 4-nitrobenzenesulfonyl, methanesulfonyl, benzenesulfonyl, triflouromethylsulfonyl, acetyl, trifluoroacetyl, benzoyl, pivaloyl, benzyloxycarbonyl, or tert-butyloxycarbonyl.

In some embodiments, PGN is a sulfonyl group. In some embodiments, PGN is p-toluenesulfonyl or 4-nitrobenzenesulfonyl.

In some embodiments, PGN of Formula IX-5a is p-toluenesulfonyl (Ts). In some embodiments, provided is Compound IX-5a-Ts or a salt thereof.

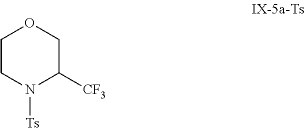 [SMILES: Cc1ccc(S(=O)(=O)N2CCOCC2C(F)(F)F)cc1]

In some embodiments, PGN of Formula IX-5a is 4-nitrobenzenesulfonyl (Ns). In some embodiments, provided is Compound IX-5a-Ns or a salt thereof.

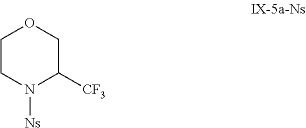

In some embodiments, provided is Compound X or a salt thereof.

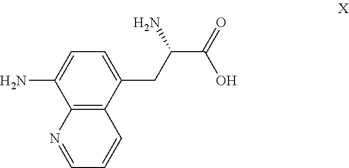 [SMILES: Nc1ccc(C[C@H](N)C(=O)O)c2cccnc12]

In some embodiments, provided is Compound X-3 or a salt thereof.

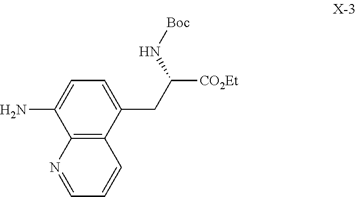 [SMILES: CCOC(=O)[C@H](Cc1ccc(N)c2ncccc12)NC(=O)OC(C)(C)C]

In some embodiments, provided is Compound X-4 or a salt thereof.

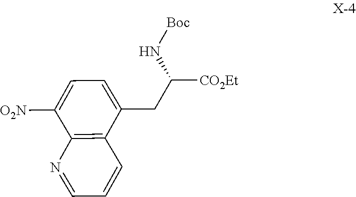 [SMILES: CCOC(=O)[C@H](Cc1ccc([N+](=O)[O-])c2ncccc12)NC(=O)OC(C)(C)C]

It is appreciated that certain features described herein, which are, for clarity, described in the context of separate embodiments, may also be provided in combination in a single embodiment. Conversely, various features described herein, which are, for brevity, described in the context of a single embodiment, may also be provided separately or in any suitable subcombination.

### EXAMPLES

The compounds of the disclosure may be prepared using methods disclosed herein and routine modifications thereof which will be apparent given the disclosure herein and methods well known in the art. Conventional and well-known synthetic methods may be used in addition to the teachings herein. The synthesis of compounds described herein may be accomplished as described in the following examples. If available, reagents may be purchased commercially, e.g., from Sigma Aldrich or other chemical suppliers. Unless otherwise noted, the starting materials for the following reactions may be obtained from commercial sources.

### Example 1: Synthesis of Compound I from Compound II and Compound III

Compound I can be prepared from Compound II and Compound III according to the following schemes.

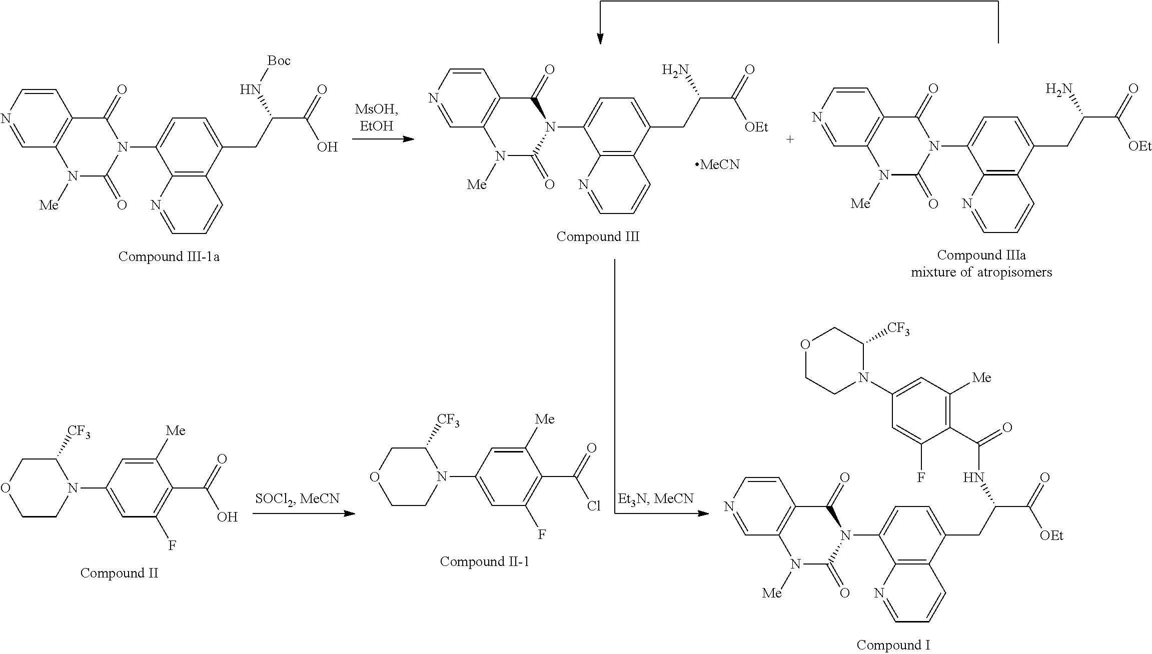

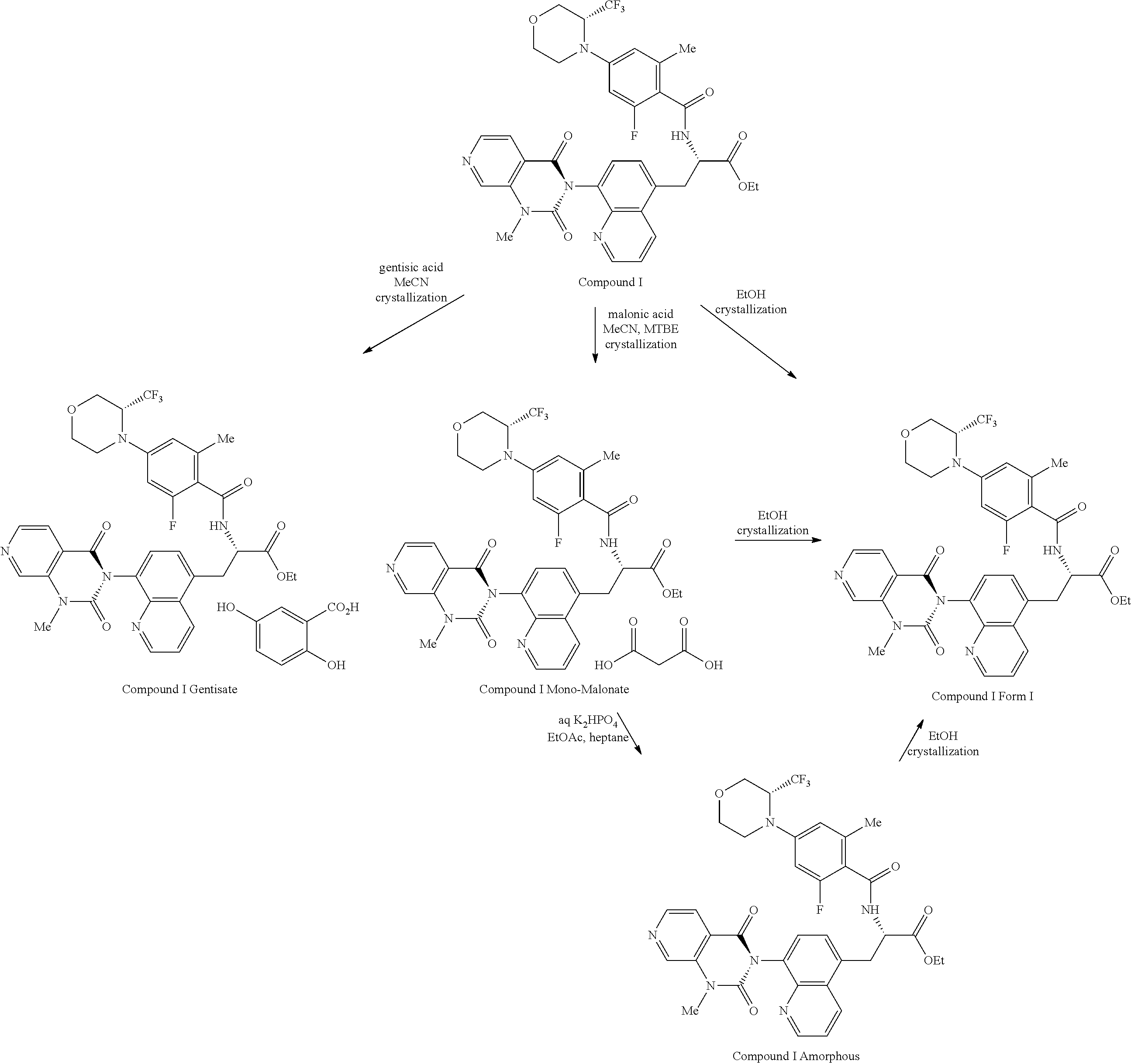

### Conversion of Compound III-1a to Compound IIIa to Compound III

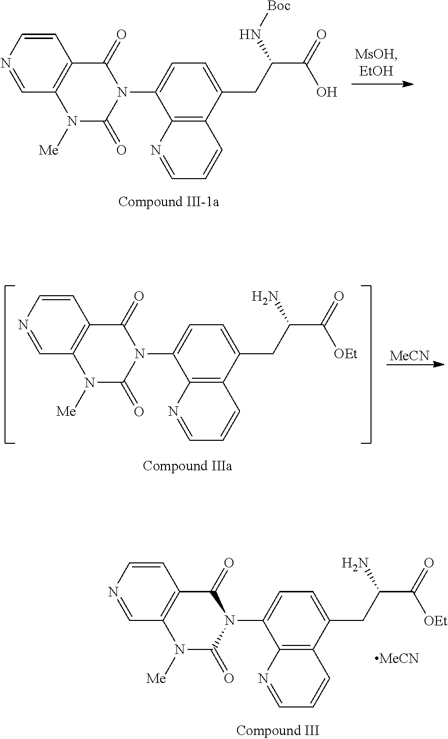

A reactor was charged with Compound III-1a (1.00 equiv, scaling factor) and ethanol (13.0 volumes) and the contents were agitated at about 75° C. Methanesulfonic acid (2.5 equiv) was charged over about 1 hour and the contents were aged at about 75° C. for about 16 hours. The reaction mixture was adjusted to about 40° C., charged with triethylamine (1.10 equiv), and the reaction volume was reduced under pressure to about 3.0 volumes. Reaction mixture was cooled to about 20° C. and charged with dichloromethane (8.0 volumes) and ammonium hydroxide solution (about 7.5 wt %, 4 volumes). The aqueous layer was discharged and the organics were washed with water (9.0 volumes). The organic stream was solvent swapped to acetonitrile at which point a slurry was observed. The organic stream was charged with additional acetonitrile (4.0 volumes) and the contents were agitated at about 50° C. Upon complete dissolution, the contents were adjusted to about 20° C. and then heated to about 50° C. over about 2 hours. Reactor contents were held at about 50° C. for about 12 hours and then cooled to about 15° C. over about 2 hours. The reactor contents were adjusted to about 20° C. and agitated for about 4 hours. Reactor contents were adjusted to about 50° C. over about 1 hour and aged for about 6 hours. The reactor contents were cooled to about 15° C. over about 6 hours and held at about 20° C. for about 4 hours. The slurry was filtered, and the cake was slurried in acetonitrile (2.5 volumes) at about 20° C. for about 12 hours. The slurry was deliquored and dried under reduced pressure at about 50° C. for about 20 hours to afford Compound III as an acetonitrile partial solvate. ¹H NMR (400 MHz, DMSO-d₆) δ 9.04 (s, 1H), 8.81 (dd, J=4.2, 1.5 Hz, 1H), 8.63 (dd, J=8.7, 1.6 Hz, 1H), 8.59 (d, J=5.0 Hz, 1H), 7.91 (dd, J=4.9, 0.7 Hz, 1H), 7.74 (d, J=7.5 Hz, 1H), 7.62 (dd, J=8.6, 4.1 Hz, 1H), 7.58 (d, J=7.6 Hz, 1H), 4.04-3.92 (m, 2H), 3.70 (t, J=7.0 Hz, 1H), 3.65 (s, 3H), 3.42 (dd, J=13.8, 6.7 Hz, 1H), 3.38-3.29 (m, 1H), 2.07 (s, 1.2H, CH₃CN), 1.99 (s, 2H), 1.05 (t, J=7.1 Hz, 3H).

### Acidic Equilibration of Compound IIIa and Crystallization of Compound III

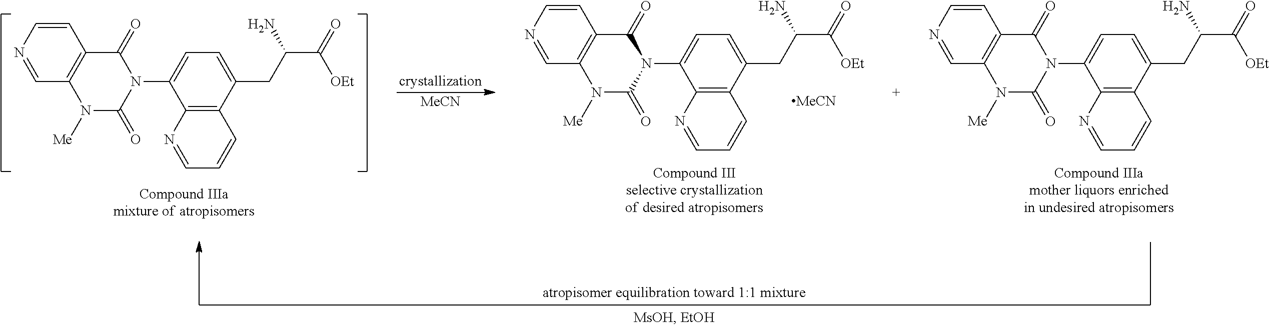

A solution of Compound IIIa in acetonitrile (1.00 equiv, scaling factor, mother liquors from Compound III crystallization) was charged to a reactor and concentrated under reduced pressure to about 3.5 volumes. The reactor contents were co-distilled with ethanol (8.2 volumes) to a reactor volume of about 3.5 volumes. Additional ethanol was charged (8.2 volumes), and the contents were adjusted to about 75° C. The reactor was charged with methanesulfonic acid (4.0 equiv) over about 30 min, and the contents were agitated at about 75° C. for about 12 hours. The reactor contents were adjusted to about 40° C., and triethylamine (4.0 equiv) was charged. The reactor volume was reduced under vacuum to about 3.5 volumes. Dichloromethane (12.0 volumes) and ammonium hydroxide solution (about 7 wt %, 4.8 volumes) were charged. The reactor contents were adjusted to about 20° C., and the aqueous layer was discharged. The organic stream was washed with water (3.5 volumes) and concentrated under reduced pressure to about 4.7 volumes. The reactor contents were co-distilled twice with acetonitrile (9.7 volumes) to about 4.7 volumes, at which point a slurry was observed. The reactor was charged with warm acetonitrile (4.6 volumes), and the contents were adjusted to about 50° C. over about 2 hours. The reaction mixture was agitated at about 50° C. for about 12 hours and then cooled to about 20° C. over about 2 hours, and then agitated for about 4 additional hours. The slurry was filtered, and the cake was re-slurried with acetonitrile (3.4 volumes) at about 20° C. for about 12 hours. The slurry was deliquored and cake dried under reduced pressure to afford Compound III as an acetonitrile partial solvate. ¹H NMR (400 MHz, DMSO-d₆) δ 9.04 (s, 1H), 8.81 (dd, J=4.2, 1.5 Hz, 1H), 8.63 (dd, J=8.7, 1.6 Hz, 1H), 8.59 (d, J=5.0 Hz, 1H), 7.91 (dd, J=4.9, 0.7 Hz, 1H), 7.74 (d, J=7.5 Hz, 1H), 7.62 (dd, J=8.6, 4.1 Hz, 1H), 7.58 (d, J=7.6 Hz, 1H), 4.04-3.92 (m, 2H), 3.70 (t, J=7.0 Hz, 1H), 3.65 (s, 3H), 3.42 (dd, J=13.8, 6.7 Hz, 1H), 3.38-3.29 (m, 1H), 2.07 (s, 1.2H, CH₃CN), 1.99 (s, 2H), 1.05 (t, J=7.1 Hz, 3H).

### Thermal Equilibration of Compound IIIa and Crystallization of Compound III

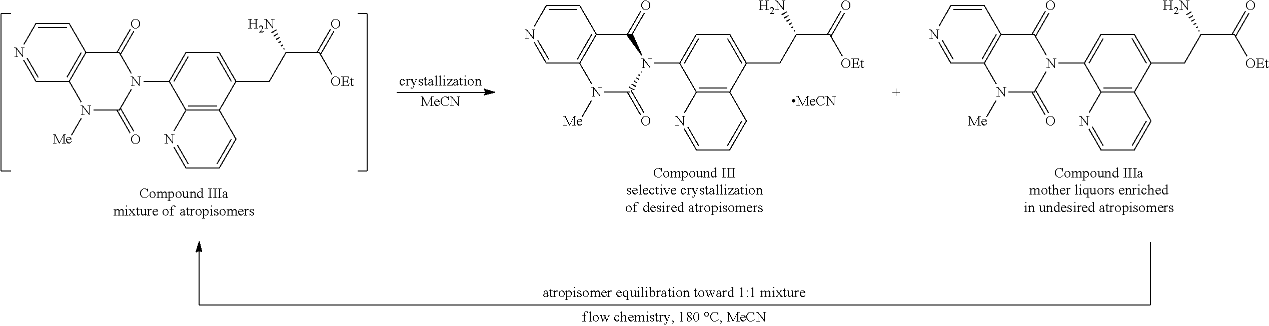

A solution of Compound IIIa (1.00 equiv, scaling factor) in dichloromethane (10 volumes) was concentrated to near dryness (about 2 volumes) under reduced pressure. The resulting mixture was co-distilled two times with acetonitrile (10 volumes), at which point a slurry is observed. The slurry was transferred to a reactor and diluted with acetonitrile to a mixture of about 8 volumes total. The mixture was heated from about 20° C. to about 55° C. over about 1 hour. The reactor contents were held at about 55° C. for about 12 hours, and then were cooled to about 20° C. over about 8 hours. The contents were then aged at about 20° C. for about 8 hours. A stainless steel HPLC filter was connected to a Vapourtec R-series flow reactor with PFA tubing, and was immersed into the crystallization reactor containing the slurry. The outlet of the flow reactor was connected to a 24-bar back pressure regulator. Liquors were circulated through the heated flow reactor set to about 180° C. with a residence time of about 2 minutes. The liquors were then circulated back into the crystallization vessel. The liquors were subjected to these continuous equilibration conditions for about 4 hours. The slurry from the crystallization reactor was then filtered and the solids dried under reduced pressure at about 40° C. to afford Compound III as an acetonitrile partial solvate. ¹H NMR (400 MHz, DMSO-d₆) δ 9.04 (s, 1H), 8.81 (dd, J=4.2, 1.5 Hz, 1H), 8.63 (dd, J=8.7, 1.6 Hz, 1H), 8.59 (d, J=5.0 Hz, 1H), 7.91 (dd, J=4.9, 0.7 Hz, 1H), 7.74 (d, J=7.5 Hz, 1H), 7.62 (dd, J=8.6, 4.1 Hz, 1H), 7.58 (d, J=7.6 Hz, 1H), 4.04-3.92 (m, 2H), 3.70 (t, J=7.0 Hz, 1H), 3.65 (s, 3H), 3.42 (dd, J=13.8, 6.7 Hz, 1H), 3.38-3.29 (m, 1H), 2.07 (s, 1.2H, CH₃CN), 1.99 (s, 2H), 1.05 (t, J=7.1 Hz, 3H).

Amide Coupling and Crystallization of Compound I (preparation of Compound I Gentisate

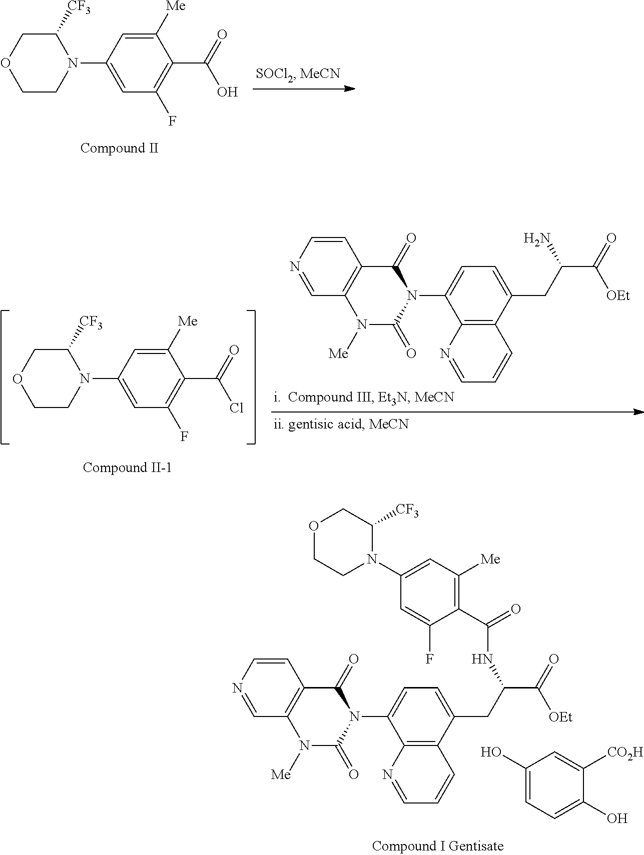

Preparation of Compound II-1. Compound II (0.98 equiv) and acetonitrile (10.0 volumes) were charged to a reactor A and the contents were agitated at about 20° C. Reactor A was charged with thionyl chloride (1.30 equiv) and the contents were agitated at about 20° C. for about 45 minutes. Reactor A contents were reduced under pressure to about 5.0 volumes and held at about 20° C. until further use.

Amide coupling. Compound III (1.00 equiv, scaling factor) and acetonitrile (10.0 volumes) were charged to a reactor B and contents were agitated at about 20° C. Reactor B was charged with triethylamine (2.00 equiv) and the reactor B contents were adjusted to about 15° C. A compound of Formula II-1 in acetonitrile in reactor A was charged to the reactor B in about 45 minutes. The resulting reaction mixture was agitated at about 20° C. for about 14 hours. Reactor B was charged with water (10.0 volumes) and isopropyl acetate (10.0 volumes). The aqueous layer was discharged, and the reactor B contents were adjusted to about 35° C. The organic stream was washed with 5% citric acid (10.0 volumes), 5% potassium bicarbonate (10.0 volumes), and 2% brine (10.0 volumes). Reactor B contents were concentrated under reduced pressure at about 40° C. to about 5.0 volumes and charged with isopropyl acetate (15.0 volumes). Reactor B contents were concentrated under reduced pressure at about 40° C. to about 5.0 volumes and charged with isopropyl acetate (5.0 volumes). Reactor B contents were agitated at about 20° C. and passed through a sock filter. Filtrate was transferred to a clean reactor and concentrated under reduced pressure at about 40° C. to about 3.0 volumes and charged with acetonitrile (10.0 volumes). Reactor B contents were concentrated under reduced pressure at about 40° C. to about 3.0 volumes and charged with acetonitrile (10.0 volumes). Reactor B contents were concentrated under reduced pressure at about 40° C. to about 3.0 volumes agitated at about 20° C.

Crystallization of Compound I Gentisate. A solution of gentisic acid (1.50 equiv) in acetonitrile (11.3 volumes) was prepared, agitated at about 40° C. for about 30 minutes and then adjusted to about 20° C. Reactor B contents were adjusted to about 40° C. About two-thirds of the gentisic acid solution was transferred to reactor B containing Compound I at about 40° C., seeded with Compound I Gentisate (0.001 equiv), and the resulting solution was agitated for about 1 hour. The reactor contents were charged with the remaining solution of gentisic acid solution, and the contents were agitated at about 40° C. for about 1 hour. The resulting reaction mixture was cooled to about 10° C. over about 9 hours and agitated at about 10° C. for about 7 hours. The slurry was filtered, washed with acetonitrile (5.0 volumes), and dried under reduced pressure at about 45° C. to afford Compound I Gentisate. ¹H NMR (400 MHz, DMSO-d₆) δ 9.16 (s, 1H), 9.04 (s, 1H), 9.01 (d, J=7.7 Hz, 1H), 8.85 (dd, J=4.2, 1.5 Hz, 1H), 8.66 (dd, J=8.7, 1.6 Hz, 1H), 8.61 (d, J=4.9 Hz, 1H), 7.92 (d, J=5.1 Hz, 1H), 7.77 (d, J=7.6 Hz, 1H), 7.72-7.63 (m, 2H), 7.17 (d, J=3.1 Hz, 1H), 6.97 (dd, J=8.9, 3.1 Hz, 1H), 6.80 (d, J=8.9 Hz, 1H), 6.76-6.64 (m, 2H), 4.98-4.69 (m, 2H), 4.20-4.07 (m, 3H), 3.96 (dd, J=11.4, 3.6 Hz, 1H), 3.76 (dd, J=7.5, 4.9 Hz, 2H), 3.66 (s, 3H), 3.61-3.49 (m, 3H), 3.44-3.16 (m, 3H), 2.10 (s, 3H), 1.17 (t, J=7.1 Hz, 3H).

### Amide Coupling and Crystallization of Compound I (Preparation of Compound I Mono-Malonate)

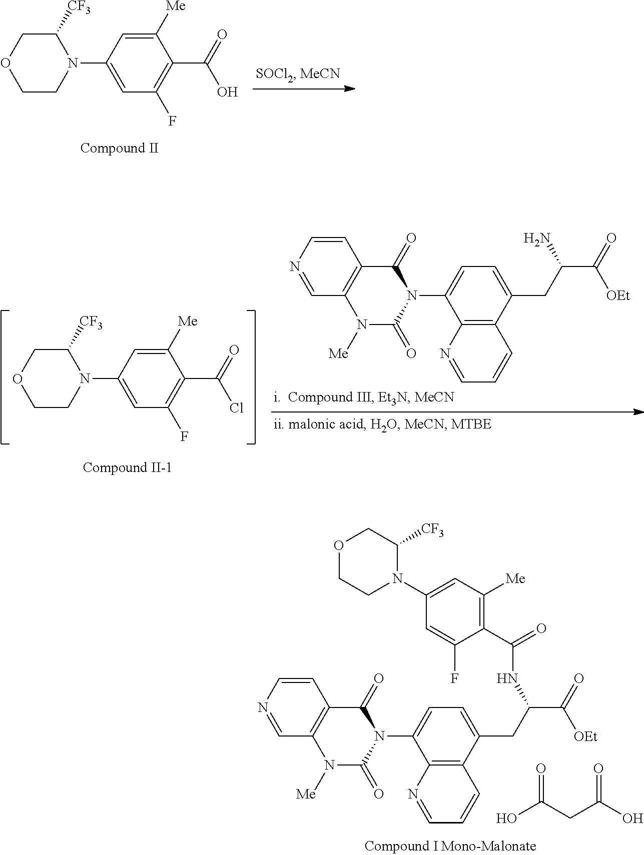

Preparation of Compound II-1. Compound II (0.95 equiv) and acetonitrile (10.0 volumes) were charged to a reactor and the contents were agitated at about 20° C. Reactor was charged with thionyl chloride (1.30 equiv) and the contents were agitated at about 20° C. for about 3 hours. Reactor contents were reduced under pressure to about 5.0 volumes and held at about 15° C. until further use.

Amide coupling. Compound III (1.00 equiv, scaling factor) and acetonitrile (10.0 volumes) were charged to a reactor and contents were agitated at about 20° C. Reactor was charged with triethylamine (2.00 equiv) and the reactor contents were adjusted to about 15° C. Compound II-1 (0.95 equiv) in acetonitrile (5.0 volumes) was charged to the reactor in about 1 hour. The resulting reaction mixture was agitated at about 20° C. for about 2 hours. Reactor was charged with water (10.0 volumes) and isopropyl acetate (10.0 volumes). The aqueous layer was discharged and the reactor contents were adjusted to about 35° C. The organic stream was washed sequentially with 5% citric acid (10.0 volumes), 5% potassium bicarbonate (10.0 volumes), and 2% brine (10.0 volumes). Reactor contents were concentrated under reduced pressure at about 40° C. to about 3.0 volumes and charged with acetonitrile (10.0 volumes). Reactor contents were concentrated under reduced pressure at about 40° C. to about 3.0 volumes and charged with acetonitrile (7.0 volumes). Reactor contents were agitated at about 20° C. and passed through a polish filtration. Filtrate was transferred to a clean reactor and concentrated under reduced pressure at about 40° C. to about 3.0 volumes. Reactor contents were charged with acetonitrile (4.5 volumes) and agitated at about 20° C.

Crystallization of Compound I Mono-Malonate. Reactor contents were adjusted to about 40° C. A solution of malonic acid (1.3 equiv) in tert-butyl methyl ether (5 volumes) was prepared. About half of the malonic acid solution was transferred to reactor containing Compound I at about 40° C. and the resulting solution was agitated for about 2 hours. Reactor was charged with water (0.3 equiv), seeded with Compound I Mono-Malonate (0.0005 equiv), and agitated at about 40° C. for about 1 hour. The reactor contents were charged with the remaining solution of malonic acid in tert-butyl methyl ether over about 2 hours and the contents were agitated at about 40° C. for about 1 hour. The resulting reaction mixture was cooled to about 20° C. over about 4 hours and agitated for about 12 hours. A solution of acetonitrile/tert-butyl methyl ether/water (2.5 volumes/2.5 volumes/1.5 equiv) was prepared. The slurry was filtered, washed with acetonitrile/tert-butyl methyl ether/water mixture (2.5 volumes/2.5 volumes/1.5 equiv), and dried under reduced pressure at about 70° C. to afford Compound I Mono-Malonate. ¹H NMR (400 MHz, DMSO-d₆) δ 12.64 (s, 2H), 9.02 (d, J=13.0 Hz, 2H), 8.85 (dd, J=4.2, 1.5 Hz, 1H), 8.65 (dd, J=8.6, 1.6 Hz, 1H), 8.60 (d, J=5.0 Hz, 1H), 7.92 (d, J=4.9 Hz, 1H), 7.77 (d, J=7.6 Hz, 1H), 7.72-7.59 (m, 2H), 6.78-6.62 (m, 2H), 4.84 (ddd, J=9.4, 7.2, 4.6 Hz, 2H), 4.23-4.05 (m, 3H), 3.95 (dd, J=11.3, 3.6 Hz, 1H), 3.79-3.73 (m, 2H), 3.65 (s, 3H), 3.60-3.50 (m, 2H), 3.36 (d, J=13.3 Hz, 1H), 3.26 (s, 3H), 2.09 (s, 3H), 1.17 (t, J=7.1 Hz, 3H).

### Amide Coupling and Crystallization of Compound I Form I

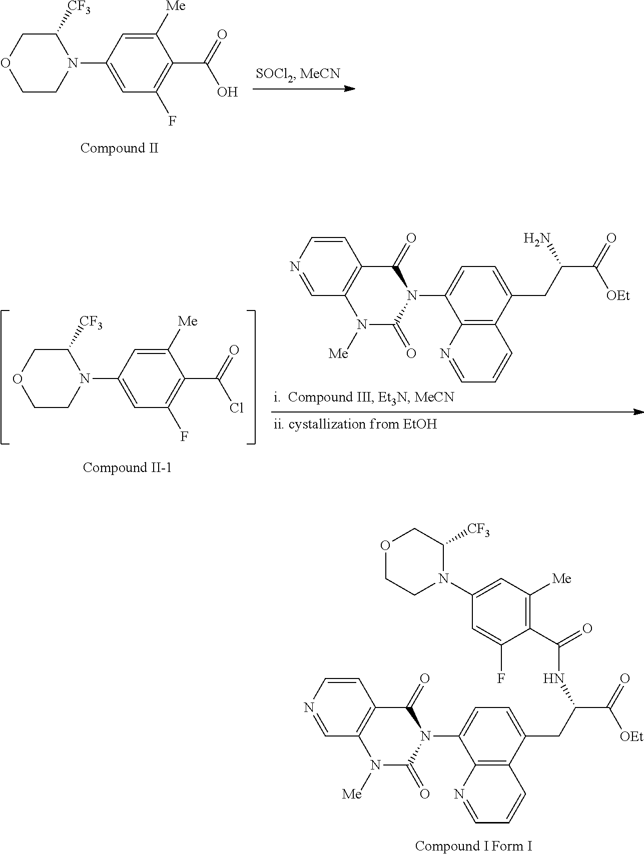

Preparation of Compound II-1. Compound II (0.95 equiv) and acetonitrile (15.3 volumes) were charged to a reactor and the contents were agitated at about 20° C. Reactor was charged with thionyl chloride (1.30 equiv) and the contents were agitated at about 20° C. for about 3 hours. Reactor contents were reduced under pressure to about 5.0 volumes and held at about 15° C. until further use.

Amide coupling. Compound III (1.00 equiv, scaling factor) and acetonitrile (7.33 volumes) were charged to a reactor and contents were agitated at about 20° C. The reactor was charged with triethylamine (2.00 equiv) and the reactor contents were adjusted to about 15° C. Compound II-1 (0.95 equiv) in acetonitrile (5.0 volumes) was charged to the reactor in about 1 hour. The resulting reaction mixture was agitated at about 20° C. for about 10 hours. The reactor was charged with water (10.0 volumes) and isopropyl acetate (10.0 volumes). The aqueous layer was discharged, and the reactor contents were adjusted to about 35° C. The organic stream was washed sequentially with 5% citric acid (10.0 volumes), 5% potassium bicarbonate (10.0 volumes), and 2% brine (10.0 volumes). Reactor contents were concentrated under reduced pressure at about 40° C. to about 3.0 volumes. Reactor contents were charged with absolute ethanol (7.9 volumes) and concentrated under reduced pressure at about 40° C. to about 4 volumes. The reactor contents were repeatedly co-distilled with absolute ethanol (8.0 volumes) to about 3 volumes until the KF was not more than 0.5 wt % and residual isopropyl acetate was not more than 3 wt %. Charged absolute ethanol (7.5 volumes).

Crystallization of Compound I Form I. Reactor contents were adjusted to about 50° C. and agitated for about 30 min. The reaction mixture was seeded with Compound I (Form I) and agitated for about 1 hour. The resulting reaction mixture was cooled to about 17° C. over about 24 hours and agitated overnight. The slurry was filtered, washed with cold ethanol (3.0 volumes) and dried under reduced pressure at about 50° C. to afford Compound I Form I. ¹H NMR (400 MHz, CDCl₃) δ 8.85 (s, 1H), 8.82 (dd, J=4.2, 1.5 Hz, 1H), 8.68 (dd, J=8.7, 1.6 Hz, 1H), 8.60 (d, J=5.0 Hz, 1H), 8.04 (dd, J=4.9, 0.7 Hz, 1H), 7.61 (d, J=7.5 Hz, 1H), 7.52 (d, J=7.6 Hz, 1H), 7.50 (dd, J=8.6, 4.2 Hz, 1H), 6.60 (dd, J=7.6, 2.7 Hz, 1H), 6.48 (d, J=2.4 Hz, 1H), 6.39 (dd, J=13.3, 2.4 Hz, 1H), 5.17 (td, J=7.7, 5.7 Hz, 1H), 4.30 (d, J=12.4 Hz, 1H), 4.18-4.00 (m, 4H), 3.86 (dd, J=14.1, 5.8 Hz, 1H), 3.81 (ddt, J=10.2, 4.2, 2.0 Hz, 1H), 3.74 (s, 3H), 3.67 (dd, J=11.7, 3.2 Hz, 1H), 3.60 (dd, J=14.1, 7.8 Hz, 2H), 3.56-3.46 (m, 1H), 3.27 (d, J=12.1 Hz, 1H), 2.40 (s, 3H), 1.11 (t, J=7.1 Hz, 3H).

### Conversion of Compound I Mono-Malonate to Compound I Amorphous

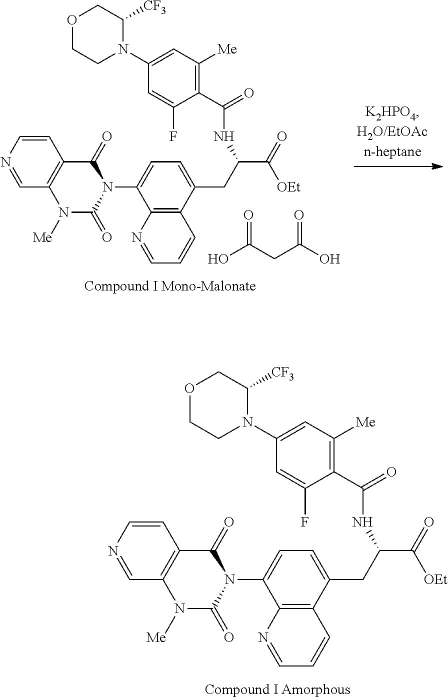

Compound I Mono-Malonate (1.00 equiv, scaling factor) and ethyl acetate (10.0 volumes) were charged to a reactor and the contents were agitated at about 20° C. Reactor was charged with a solution of 10% potassium phosphate dibasic (10.0 volumes) and agitated at about 20° C. for about 1 hour. The aqueous layer was discharged, the reactor was charged with a solution of 2% brine (10.0 volumes), and the contents were agitated at about 20° C. for about 1 hour. The aqueous layer was discharged and the reactor contents were concentrated under reduced pressure to about 3.0 volumes with a jacket of about 40° C. Reactor was charged with ethyl acetate (15.0 volumes) and the contents were concentrated under reduced pressure to about 5.0 volumes with a jacket of about 40° C. Reactor was charged with ethyl acetate (15.0 volumes) and the contents were concentrated under reduced pressure to about 5.0 volumes with a jacket of about 40° C. The contents of the reactor were adjusted to about 20° C. and charged over about 1 h via polish filter to a separate clean reactor containing n-heptane (20.0 volumes). The resulting reaction mixture was agitated at about 20° C. for about 2 hours. The slurry was then filtered, rinsed with n-heptane (5.0 volumes), and dried under reduced pressure at about 40° C. to afford amorphous Compound I (Compound I Amorphous). ¹H NMR (400 MHz, CDCl₃) δ 8.89-8.77 (m, 2H), 8.68 (dd, J=8.7, 1.6 Hz, 1H), 8.58 (d, J=4.9 Hz, 1H), 8.03 (dd, J=5.0, 0.8 Hz, 1H), 7.61 (d, J=7.5 Hz, 1H), 7.56-7.45 (m, 2H), 6.67 (dd, J=7.6, 2.6 Hz, 1H), 6.47 (d, J=2.4 Hz, 1H), 6.39 (dd, J=13.2, 2.4 Hz, 1H), 5.16 (td, J=7.6, 5.8 Hz, 1H), 4.28 (d, J=12.4 Hz, 1H), 4.14-4.03 (m, 3H), 4.01 (d, J=3.5 Hz, 1H), 3.87-3.80 (m, 2H), 3.72 (s, 3H), 3.65-3.56 (m, 2H), 3.50 (tdd, J=12.1, 3.9, 2.1 Hz, 1H), 3.29-3.21 (m, 1H), 2.39 (s, 3H), 1.11 (t, J=7.1 Hz, 3H).

### Conversion of Compound I Amorphous to Compound I Form I

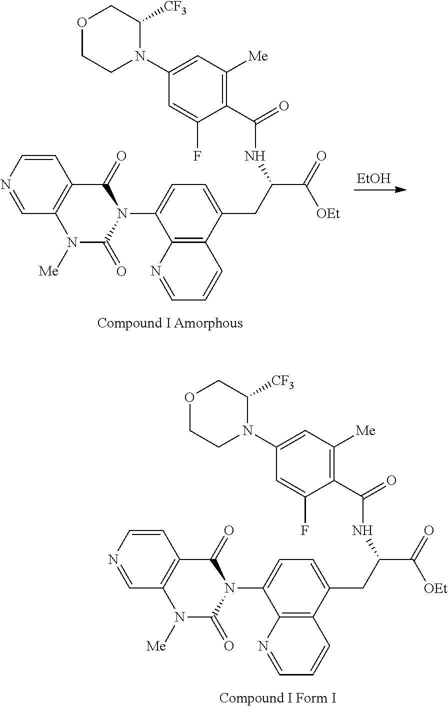

Amorphous Compound I and anhydrous ethanol (9 volumes) were charged to a reactor. The reactor contents were warmed to 50° C., and the solids dissolved with agitation. The solution was seeded with Compound I Form I, aged for 3 h, and then cooled to not more than 20° C. over 8 h. The resulting white slurry was filtered, the cake was rinsed with absolute ethanol (3 volumes), and then dried under vacuum to give Compound I Form I. ¹H NMR (400 MHz, CDCl₃) δ 8.85 (s, 1H), 8.82 (dd, J=4.2, 1.5 Hz, 1H), 8.68 (dd, J=8.6, 1.6 Hz, 1H), 8.60 (d, J=5.0 Hz, 1H), 8.04 (dd, J=4.9, 0.7 Hz, 1H), 7.61 (d, J=7.5 Hz, 1H), 7.52 (d, J=7.5 Hz, 1H), 7.50 (dd, J=8.6, 4.2 Hz, 1H), 6.60 (dd, J=7.6, 2.7 Hz, 1H), 6.48 (d, J=2.4 Hz, 1H), 6.39 (dd, J=13.2, 2.4 Hz, 1H), 5.17 (td, J=7.7, 5.8 Hz, 1H), 4.31 (d, J=12.4 Hz, 1H), 4.16-4.01 (m, 4H), 3.86 (dd, J=14.2, 5.8 Hz, 1H), 3.81 (ddt, J=10.1, 4.1, 2.0 Hz, 1H), 3.74 (s, 3H), 3.67 (td, J=11.8, 3.3 Hz, 1H), 3.60 (dd, J=14.1, 7.9 Hz, 1H), 3.52 (tdd, J=11.9, 3.2, 1.7 Hz, 1H), 3.27 (d, J=11.9 Hz, 1H), 2.40 (s, 3H), 1.11 (t, J=7.2 Hz, 3H).

### Conversion of Compound I Mono-Malonate to Compound I Form I

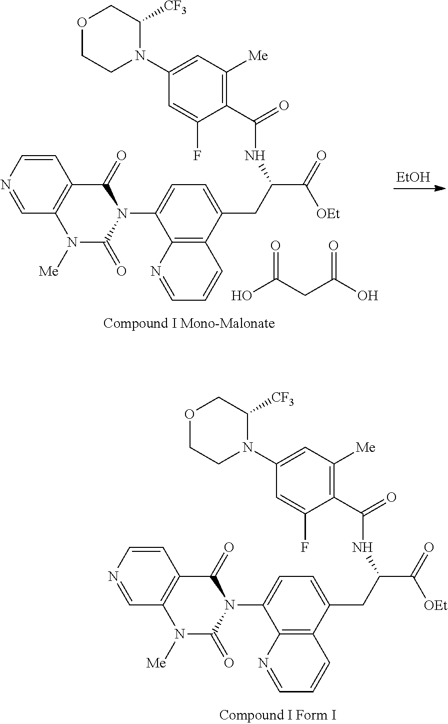

Charge Compound I Mono-Malonate and ethanol (17 volumes) to a scintillation vial and dissolved the solids with sonication. The solution was seeded with Compound I Form I and agitated at about 20° C. for about 22 h. Compound I Form I was isolated from the resulting slurry by filtration. ¹H NMR (400 MHz, CDCl₃) δ 8.84 (br s, 1H), 8.81 (dd, J=4.2, 1.6 Hz, 1H), 8.67 (dd, J=8.6, 1.6 Hz, 1H), 8.59 (d, J=5.0 Hz, 1H), 8.03 (dd, J=5.0, 0.8 Hz, 1H), 7.60 (d, J=7.5 Hz, 1H), 7.52 (d, J=7.6 Hz, 1H), 7.50 (dd, J=8.6, 4.2 Hz, 1H), 6.66 (dd, J=7.6, 2.6 Hz, 1H), 6.47 (d, J=2.4 Hz, 1H), 6.39 (dd, J=13.2, 2.4 Hz, 1H), 5.16 (td, J=7.6, 5.8 Hz, 1H), 4.29 (dd, J=12.4, 1.0 Hz, 1H), 4.15-4.01 (m, 4H), 3.85 (dd, J=14.3, 5.9 Hz, 1H), 3.79 (ddt, J=8.2, 4.1, 2.0 Hz, 1H), 3.73 (s, 3H), 3.64 (td, J=11.7, 3.2 Hz, 1H), 3.60 (dd, J=14.20, 7.8 Hz, 1H), 3.51 (m, 1H), 3.26 (d, J=12.2 Hz, 1H), 2.39 (s, 3H), 1.11 (t, J=7.1 Hz, 3H).

### Example 2: Synthesis of Compound III-1a (Synthesis of Compound III—Method A)

Compound III-1a can be prepared according to the following schemes.

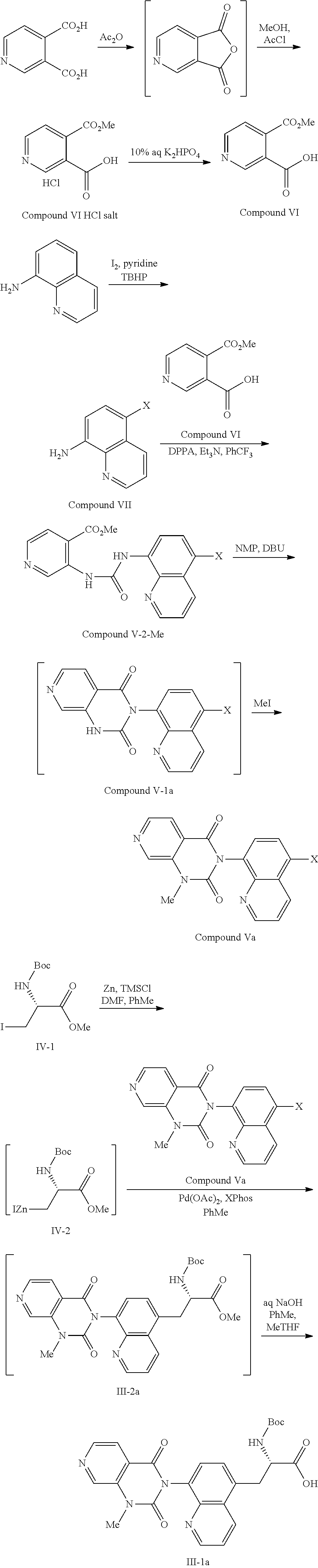

Esterification of Pyridine-3,4-dicarboxylic Acid to Compound VI HCl Salt

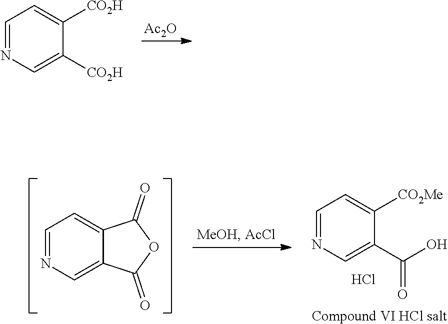

Pyridine-3,4-dicarboxylic acid (1.00 equiv, scaling factor) and acetic anhydride (2.2 equiv) were charged to a reactor. The resulting reaction mixture was agitated at about 105° C. for about 4 hours and then cooled to about 10° C. The reaction mixture was transferred to a reactor containing premixed acetyl chloride (2.3 equiv) and methanol (4.7 volumes) and the contents were agitated at about 10° C. After complete addition, the reactor contents were agitated at about 20° C. for about 4 hours. The slurry was centrifuged, washed with isopropanol (1.5 volumes), and dried under reduced pressure at about 45° C. for about 20 hours to afford Compound VI HCl salt. ¹H NMR (400 MHz, DMSO-d₆) δ 9.03 (d, J=0.8 Hz, 1H), 8.89 (d, J=4.8 Hz, 1H), 7.66 (d, J=0.8 Hz, 1H), 4.98 (br s, 2H), 3.84 (s, 311).

### Free Basing Compound VI HCl Salt to Compound VI

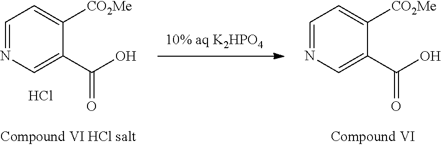 [SMILES: COC(=O)c1ccncc1C(=O)O ; Cl ; COC(=O)c1ccncc1C(=O)O]

Compound VI HCl salt (1.00 equiv, scaling factor) was charged to a reactor and the contents were adjusted to about 20° C. The reactor was charged with 10% aqueous potassium phosphate dibasic solution (about 5 volumes) to adjust the pH to about 2.0. The slurry was agitated for about 16 hours, filtered, washed with water (0.5 volumes) and dried under reduced pressure at about 45° C. for about 20 hours to afford Compound IV (free base). ¹H NMR (400 MHz, DMSO-d₆) δ 13.51 (br s, 1H), 9.02 (d, J=0.8 Hz, 1H), 8.87 (d, J=4.8 Hz, 1H), 7.67 (d, J=0.8 Hz, 1H), 3.84 (s, 311).

Iodination of quinolin-8-amine to Compound VII-I

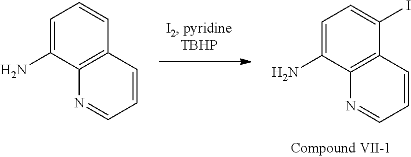 [SMILES: Nc1cccc2cccnc12 ; Nc1ccc(I)c2cccnc12]

Quinolin-8-amine (1.00 equiv, scaling factor), iodine (0.6 equiv), pyridine (3.0 equiv), and isopropyl acetate (1.5 volume) were charged to a reactor. Reactor contents were agitated at about 25° C. for about 2 hours. Reaction contents were adjusted to about 50° C. and charged with tert-butanol peroxide in water (0.55 equiv, 70% aqueous solution). The resulting reaction mixture was agitated at about 50° C. for about 2 hours. Reaction mixture was quenched with sodium thiosulfate (0.50 equiv) in water (1.0 volumes) while maintaining the internal temperature at about 50° C. After agitating for about 1 hour, reaction mixture was charged with activated carbon (1.80 equiv) in water (0.5 volumes). The temperature was adjusted to about 25° C. and the stream was filtered through celite. The aqueous layer was extracted with isopropyl acetate (0.5 volumes) and the combined organics were washed with water (1.0 volumes), concentrated under reduced pressure, and charged with N-methyl-2-pyrrolidone (0.5 volumes). The reactor contents were concentrated under reduced pressure to about 5.5 volumes and agitated at about 20° C. Reactor was charged with water (4.0 volumes) and the contents were agitated for about 2 hours. The slurry was filtered, washed with methanol (1.5 volumes) and water (3.5 volumes), and dried under reduced pressure at about 45° C. for about 20 hours to afford Compound VII-I. ¹H NMR (400 MHz, DMSO-d₆) δ 8.72 (dd, J=4.1, 1.5 Hz, 1H), 8.17 (dd, J=8.5, 1.5 Hz, 1H), 7.80 (d, J=8.1 Hz, 1H), 7.59 (dd, J=8.5, 4.1 Hz, 1H), 6.72 (d, J=8.1 Hz, 1H), 6.19 (s, 2H).

### Curtius Rearrangement of Compound VI to Compound V-3-I

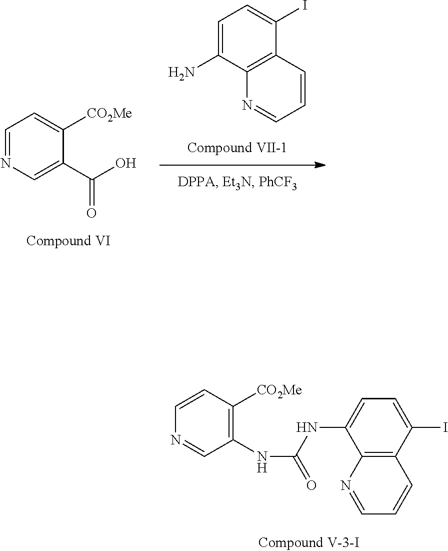 [SMILES: COC(=O)c1ccncc1C(=O)O ; Nc1ccc(I)c2cccnc12 ; COC(=O)c1ccncc1NC(=O)Nc1ccc(I)c2cccnc12]

A mixture of Compound VII-I (1.00 equiv, scaling factor), Compound VI (1.15 equiv), trifluorotoluene (6.0 volumes) and triethylamine (2.5 equiv) was warmed to about 65° C. A solution of diphenylphosphoryl azide (1.15 equiv) in trifluorotoluene (1.0 volume) was added dropwise via an addition funnel over about 2 h while maintaining a temperature of about 70° C. The addition funnel was rinsed forward with trifluorotoluene (0.5 volumes) and the reaction was stirred at about 70° C. for about 1 h and then cooled to about 20° C. over about 2 h. The mixture was filtered and the solids were rinsed with trifluorotoluene (1.0 volume), rinsed twice with tert-butyl methyl ether (1.0 volumes per rinse) and dried at 50° C. to afford Compound V-3-I. ¹H NMR (400 MHz, DMSO-d₆) δ 10.25 (s, 1H), 10.09 (s, 1H), 9.00 (s, 1H), 8.97 (dd, J=4.2, 1.6 Hz, 1H), 8.41 (d, J=5.0 Hz, 1H), 8.38 (dd, J=8.6, 1.5 Hz, 1H), 8.33 (d, J=8.4 Hz, 1H), 8.15 (d, J=8.3 Hz, 1H), 7.78 (dd, J=8.5, 4.2 Hz, 1H), 7.66 (d, J=5.0 Hz, 1H), 3.85 (s, 3H).

### Curtius Rearrangement of Compound VI to Compound V-3-Br

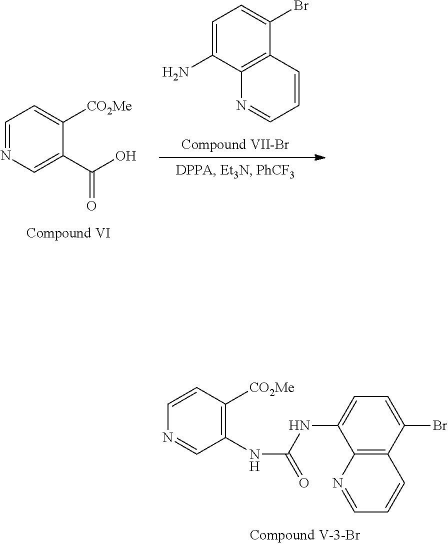 [SMILES: COC(=O)c1ccncc1C(=O)O ; Nc1ccc(Br)c2cccnc12 ; COC(=O)c1ccncc1NC(=O)Nc1ccc(Br)c2cccnc12]

Compound VII-Br (1.00 equiv, scaling factor), Compound VI (1.15 equiv), trifluorotoluene (6.0 volumes), and triethylamine (2.50 equiv) were charged to a reactor and warmed to about 65° C. The reactor contents were charged with a solution of diphenylphosphoryl azide (1.15 equiv) in trifluorotoluene (1.0 volumes) over 2 h while heating at about 65° C. The resulting mixture was cooled to about 15° C. over about 8 h and filtered. The filter cake was rinsed with trifluorotoluene (1.0 volume), then twice with MTBE (1.0 volume per rinse), and dried under reduced pressure at about 40° C. for about 20 hours to afford Compound V-3-Br. ¹H NMR (400 MHz, DMSO-d₆) δ 10.25 (s, 1H), 10.09 (s, 1H), 9.02 (s, 2H), 8.51 (dd, J=8.5, 1.4 Hz, 1H), 8.45 (d, J=8.5 Hz, 1H), 8.40 (d, J=5.0 Hz, 1H), 7.91 (d, J=8.5 Hz, 1H), 7.80 (dd, J=8.5, 4.2 Hz, 1H), 7.65 (d, J=5.0 Hz, 1H), 3.86 (s, 3H).

### Cyclization and Methylation of Compound V-3-I to Compound Va-I

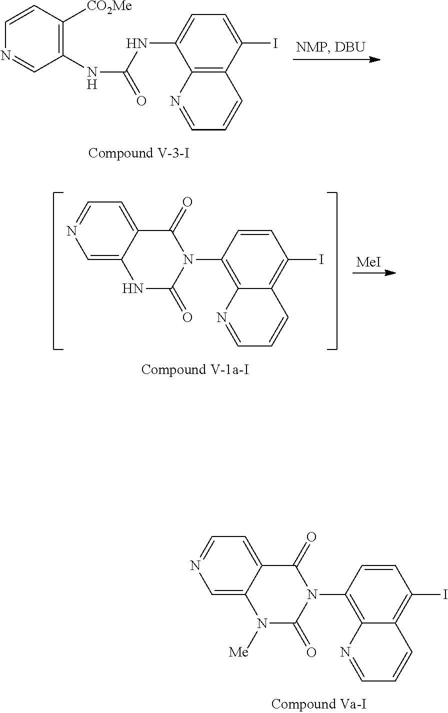

Compound V-3-I (1.00 equiv, scaling factor), 1,8-diazabicyclo[5.4.0]undec-7-ene (1.00 equiv), and N-methyl-2-pyrrolidone (6.0 volumes) were charged to a reactor. The reactor contents were agitated at about 20° C. for about 3 hours. The reactor contents were adjusted to about 0° C. and charged with iodomethane (1.10 equiv) and N-methyl-2-pyrrolidone (1.0 volumes) while maintaining the internal temperature at about 5° C. After complete addition, the resulting reaction mixture was agitated at about 20° C. for about 16 hours. Reactor was charged with water (3.5 volumes) while maintaining the internal temperature at about 20° C. and agitated for about 16 hours. The slurry was centrifuged and the cake was washed with N,N-dimethylformamide (1.0 volumes), water (0.5 volumes), isopropyl alcohol (1.5 volumes), and tert-butyl methyl ether (1.5 volumes). The cake was dried under pressure at about 50° C. for about 30 hours to afford Compound Va-I. ¹H NMR (400 MHz, DMSO-d₆) δ 9.05 (d, J=0.7 Hz, 1H), 8.84 (dd, J=4.2, 1.6 Hz, 1H), 8.60 (d, J=5.0 Hz, 1H), 8.46 (dd, J=8.6, 1.5 Hz, 1H), 8.40 (d, J=7.8 Hz, 1H), 7.92 (dd, J=5.0, 0.8 Hz, 1H), 7.72 (dd, J=8.6, 4.2 Hz, 1H), 7.66 (d, J=7.8 Hz, 1H), 3.65 (s, 3H).

### Cyclization and Methylation of Compound V-3-Br to Compound Va-Br

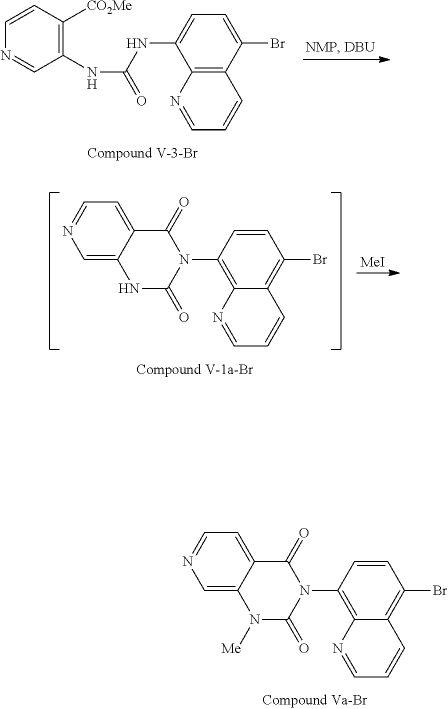

Compound V-3-Br (1.00 equiv, scaling factor), 1,8-diazabicyclo[5.4.0]undec-7-ene (2.50 equiv), and N-methyl-2-pyrrolidone (6.0 volumes) were charged to a reactor. The reactor contents were agitated at about 20° C. for about 3 hours. The reactor contents were charged with a solution of iodomethane (2.50 equiv) and N-methyl-2-pyrrolidone (0.7 volumes) over about 20 minutes. After complete addition, the resulting reaction mixture was agitated at about 20° C. for about 20 hours. The reactor was charged with water (2.3 volumes) over about 2 hours, cooled to about 10° C., charged with water (2.0 volumes) over about 2 h, cooled to about 0° C., charged with water (1.3 volumes) over about 1 h, and stirred at about 0° C. for about 18 hours. The mixture was filtered and the solids were rinsed twice with water (2.0 volumes per rinse). The solids were dried under pressure at about 50° C. to obtain Compound Va-Br. ¹H NMR (400 MHz, DMSO-d₆) δ 9.06 (s, 1H), 8.91 (dd, J=4.2, 1.6 Hz, 1H), 8.64-8.58 (m, 2H), 8.17 (d, J=8.0 Hz, 1H), 7.92 (dd, J=4.9, 0.8 Hz, 1H), 7.83 (d, J=8.0 Hz, 1H), 7.77 (dd, J=8.6, 4.2 Hz, 1H), 3.66 (s, 3H).

### Negishi Coupling and Hydrolysis to Compound III-1a

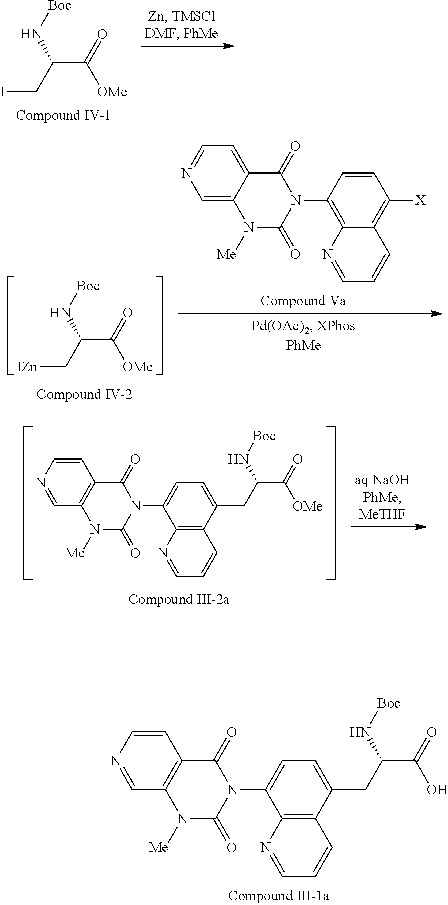

Conversion of Compound IV-1 to Organozinc Compound IV-2. This procedure uses Compound IV-1 (1.0 equiv, scaling factor) charged in two portions. A reactor with Compound IV-1 (0.10 equiv), N,N-dimethylformamide (1.0 volumes), toluene (3.0 volumes), and zinc (1.4 equiv) was charged with trimethylsilyl chloride (0.03 equiv) and aged at about 20° C. for about 1 hour. A solution of Compound IV-1 (0.90 equiv) in toluene (3.0 volumes) was added over about 7 hours. After complete addition, the resulting mixture of organozinc Compound IV-2 was aged for about 3 hours and then cooled to about −15° C. until further use.

Negishi Coupling of Compound Va-I (Compound Va, X=I) and Compound IV-2 to Compound III-2a. Compound Va-I (1.00 equiv, scaling factor), palladium(II) acetate (0.025 equiv), 2-dicyclohexylphosphino-2′,4′,6′-diisopropylbiphenyl (0.044 equiv), and toluene (7.0 volumes) were charged to a glass-lined reactor and the contents were agitated at about 20° C. Reactor was charged with organozinc Compound IV-2 solution (0.13 equiv) and the reaction mixture was agitated at about 60° C. for about 2 hours and then charged with additional organozinc Compound IV-2 solution (1.17 equiv) over about 3 hours. After complete addition, the resulting reaction mixture was agitated at about 60° C. for about 3 hours. Reactor contents were cooled to about 35° C., charged with Celite (0.35 weight ratio) and aqueous 17.5% ammonium chloride solution (7.0 volumes), and aged for about 1 hour. The resulting slurry was cooled to about 20° C., filtered, and the cake was washed with toluene (1.0 volumes). The combined filtrate was transferred to a clean glass-lined reactor and the aqueous layer was discharged. The organics were washed with aqueous 17.5% ammonium chloride solution (7.0 volumes), the phases were separated, and the organic layer was washed with an aqueous solution of N-acetyl-L-cysteine (0.50 equiv) and potassium carbonate (0.86 equiv) in water (10 volumes) at about 40° C. for about 7 hours. The reactor contents were cooled to about 20° C., the aqueous layer was discharged, and the organics were washed with water (5.0 volumes) to provide a solution of Compound III-2a in toluene.

Negishi Coupling of Compound Va-Br (Compound Va, X=Br) and Compound IV-2 to Compound III-2a. Compound Va-Br (1.00 equiv, scaling factor), palladium(II) acetate (0.025 equiv), 2-dicyclohexylphosphino-2′,4′,6′-diisopropylbiphenyl (0.044 equiv), and toluene (10.0 volumes) were charged to a glass-lined reactor and the contents were agitated at about 20° C. Reactor was charged with organozinc Compound IV-2 solution (0.3 equiv) and the reaction mixture was heated to about 65° C. Additional organozinc Compound IV-2 solution (0.9 equiv) was added over about 30 min and the mixture was stirred at about 65° C. for about 3 hours. Additional organozinc Compound IV-2 solution (0.5 equiv) was added and the mixture was heated to about 75° C. for about 4 hours and then cooled to about 15° C. over about 3 hours and aged at about 15° C. for about 16 hours. The resulting mixture was diluted with toluene (5.0 volumes), heated to about 50° C., and washed twice with aqueous 4.0 M ammonium chloride solution (6.0 volumes) at about 50° C. The organic layer was filtered through Celite (0.3 weight ratio), washed with an aqueous solution of N-acetyl-L-cysteine (0.45 equiv) and potassium carbonate (0.76 equiv) in water (10.0 volumes) at about 50° C. for about 19 hours, washed with water (6.0 volumes), and then cooled to about 20° C. to get a solution of Compound III-2a in toluene.

Hydrolysis of Compound III-2a to Compound III-1a. The solution Compound III-2a stream in toluene was charged to a reactor. The contents were adjusted to about 55° C. and reduced under pressure to about 6.0 volumes (scaling factor remains Compound Va-I from the Negishi coupling). Reactor was charged with 2-methyltetrahydrofuran (3.9 volumes) and the resulting slurry was filtered, collecting the filtrate in a reactor. Reactor contents were charged with 2-methyltetrahydrofuran (1.8 volumes) and the contents were adjusted to about 5° C. Reactor was charged with sodium hydroxide (2.0 equiv) in water (7.4 volumes) and the contents were agitated at about 5° C. for about 6 hours. The aqueous layer was discharged and the reactor contents were charged with 2-methyltetrahydrofuran (3.2 volumes) and aqueous 7% formic acid solution (1.6 volumes). The reactor contents were adjusted to about 45° C. and charged with additional aqueous 7% formic acid solution (1.6 volumes) over 4 hours. The resulting reaction mixture was cooled to about 5° C. and agitated for about 12 hours. The slurry was filtered and the cake was washed with water (8.0 volumes). The cake was then slurried in toluene (27.0 volumes) and ethanol (3.0 volumes), filtered, and dried at about 70° C. under reduced pressure for about 30 hours to afford Compound III-1a. 1H NMR (400 MHz, DMSO-d₆) δ 12.85 (s, 1H), 9.04 (s, 1H), 8.82 (d, J=4.1 Hz, 1H), 8.60 (t, J=7.3 Hz, 2H), 7.91 (dd, J=5.0, 2.1 Hz, 1H), 7.75 (d, J=7.5 Hz, 1H), 7.65 (dd, J=8.7, 3.7 Hz, 2H), 7.30 (d, J=8.3 Hz, 1H), 4.33-4.15 (m, 1H), 3.65 (d, J=1.7 Hz, 4H), 3.42-3.35 (m, 1H), 1.32 (s, 9H).

### Example 3: Synthesis of Compound III—Method B

Compound III can be prepared according to the following schemes.

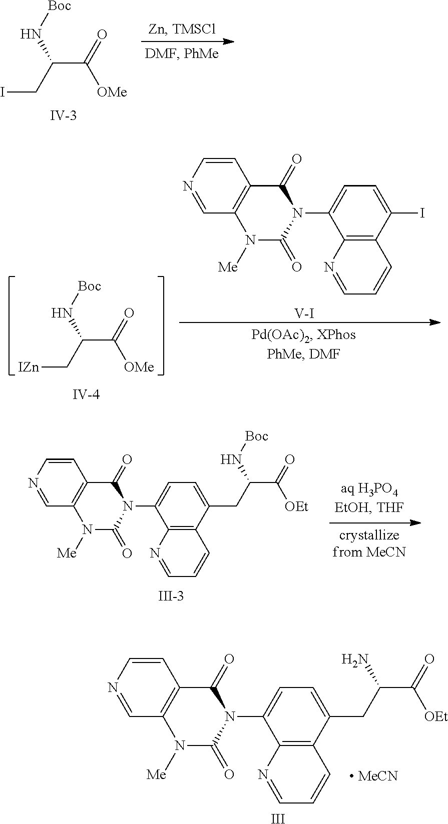

### Negishi Coupling of Compound IV-3 and Compound V-I to Compound III-3

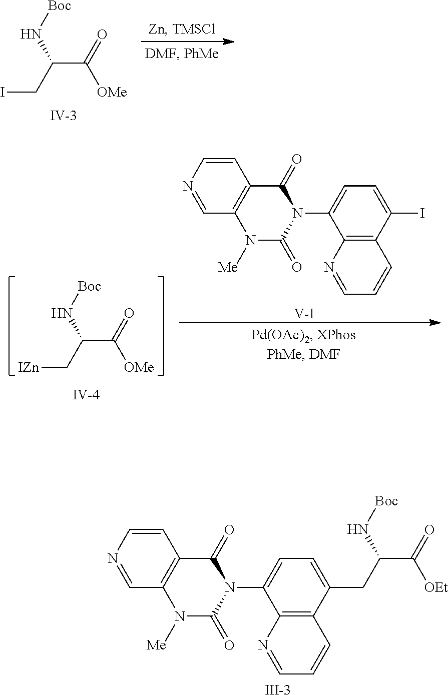

Formation of Compound IV-4. This procedure uses Compound IV-3 (1.0 equiv, scaling factor) charged in two portions. A reactor with Compound IV-3 (0.10 equiv), N,N-dimethylformamide (0.7 volumes), toluene (1.0 volumes), and zinc (3.1 equiv) was heated to about 30° C., charged with trimethylsilyl chloride (0.05 equiv), and aged for about 1 hour. A solution of Compound IV-3 (0.90 equiv) in toluene (0.7 volumes) was added over about 45 minutes, and the resulting mixture of organozinc Compound IV-4 was cooled to about 5° C. until further use.

Negishi Coupling to Compound III-3. Compound V-I (1.00 equiv, scaling factor), palladium(II) acetate (0.027 equiv), 2-dicyclohexylphosphino-2′,4′,6′-diisopropylbiphenyl (0.043 equiv), and toluene (10.0 volumes) were charged to a glass-lined reactor and the contents were agitated at about 20° C. Reactor was charged with Compound IV-4 solution (0.20 equiv) and the reaction mixture was heated to about 65° C. Additional Compound IV-4 solution (1.1 equiv) was added over about 1 hour and the mixture was stirred at about 65° C. for about 3 hours. The mixture was cooled to about 20° C., diluted with toluene (5.0 volumes), heated to about 50° C., and washed twice with aqueous 4.0 M ammonium chloride solution (6.0 volumes) at about 50° C. The organic layer was washed with an aqueous solution of N-acetyl-L-cysteine (0.47 equiv) and potassium carbonate (0.85 equiv) in water (10.0 volumes) at about 50° C. for about 20 hours, then washed with water (5.0 volumes) at about 50° C. and cooled to about 20° C., filtered, and concentrated to an oil. The concentrate was diluted with tert-butyl methyl ether (7.5 volumes) to form a slurry that was filtered to afford Compound III-3. ¹H NMR (400 MHz, CDCl₃) δ 8.85 (s, 1H), 8.83 (dd, J=4.2, 1.5 Hz, 1H), 8.61 (d, J=5.0 Hz, 1H), 8.54 (d, J=8.6 Hz, 1H), 8.05 (dd, J=4.9, 0.8 Hz, 1H), 7.62 (d, J=7.5 Hz, 1H), 7.52-7.44 (m, 2H), 5.17 (d, J=7.8 Hz, 1H), 4.70 (q, J=7.0 Hz, 1H), 4.14-4.01 (m, 2H), 3.74 (s, 3H), 3.69-3.52 (m, 2H), 1.46 (s, 9H), 1.11 (t, J=7.1 Hz, 3H).

### Deprotection of Compound III-3 to Compound III

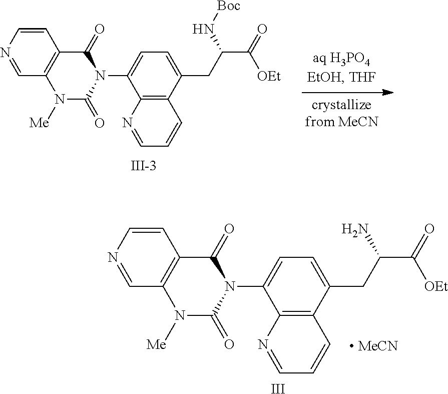

Compound III-3 (1.00 equiv, scaling factor), tetrahydrofuran (1.0 volumes), and ethanol (2.0 volumes) was charged to a reactor to yield a slurry. The mixture was warmed to 35° C. until the solids were fully dissolved, after which the mixture was cooled to about 20° C. Phosphoric acid (3.0 volumes) was charged over approximately 20 minutes. The solution was left to stir at about 20° C. for approximately 18 hours. Water (15.0 volumes) and DCM (10.0 volumes) were charged to the reactor. Ammonium hydroxide (56.0 equivalents) was charged over approximately one hour. The biphasic layer was separated and the aqueous bottom layer was discharged. To the reactor was charged water (4.0 volumes). After mixing to form a biphasic mixture, the layers were separated, and the bottom layer discharged. The remaining organic phase was distilled to approximately 4.0 volumes, after which acetonitrile (8.0 volumes) was charged. The mixture was distilled to approximately 4.0 volumes, after which acetonitrile (8.0 volumes) was charged. To the reactor was charged approximately 4.0 volumes of acetonitrile, after which the reactor jacket was set to 70° C. and hold this temperature for approximately 12 hours. The jacket temperature was reduced to 20° C. over approximately 5 hours and held at that temperature for approximately 8 hours. The slurry was then filtered and dried under reduced pressure at about 50° C. to afford Compound III as an acetonitrile partial solvate. ¹H NMR (400 MHz, DMSO-d₆) δ 9.04 (s, 1H), 8.81 (dd, J=4.2, 1.5 Hz, 1H), 8.63 (dd, J=8.6, 1.6 Hz, 1H), 8.59 (d, J=4.9 Hz, 1H), 7.91 (dd, J=5.0, 0.7 Hz, 1H), 7.74 (d, J=7.5 Hz, 1H), 7.62 (dd, J=8.6, 4.2 Hz, 1H), 7.58 (d, J=7.6 Hz, 1H), 3.98 (qd, J=7.2, 1.0 Hz, 2H), 3.69 (t, J=7.1 Hz, 1H), 3.65 (s, 3H), 3.42 (dd, J=13.8, 6.7 Hz, 1H), 3.36-3.30 (m, 3H), 2.07 (s, 1.6H, CH₃CN), 1.05 (t, J=7.1 Hz, 3H).

### Example 4: Synthesis of Compound III-3 (Synthesis of Compound III—Method C)

Compound III-3 can be prepared according to the following schemes.

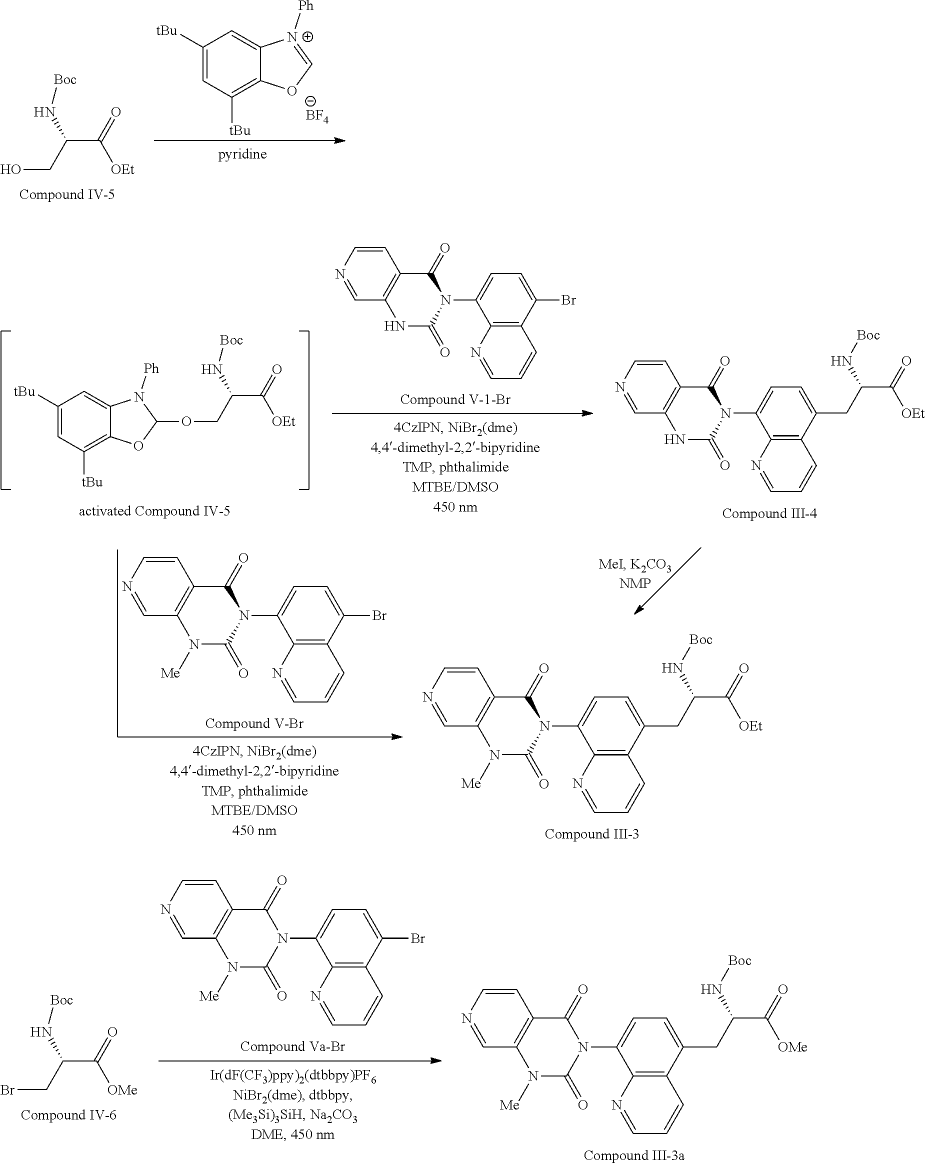

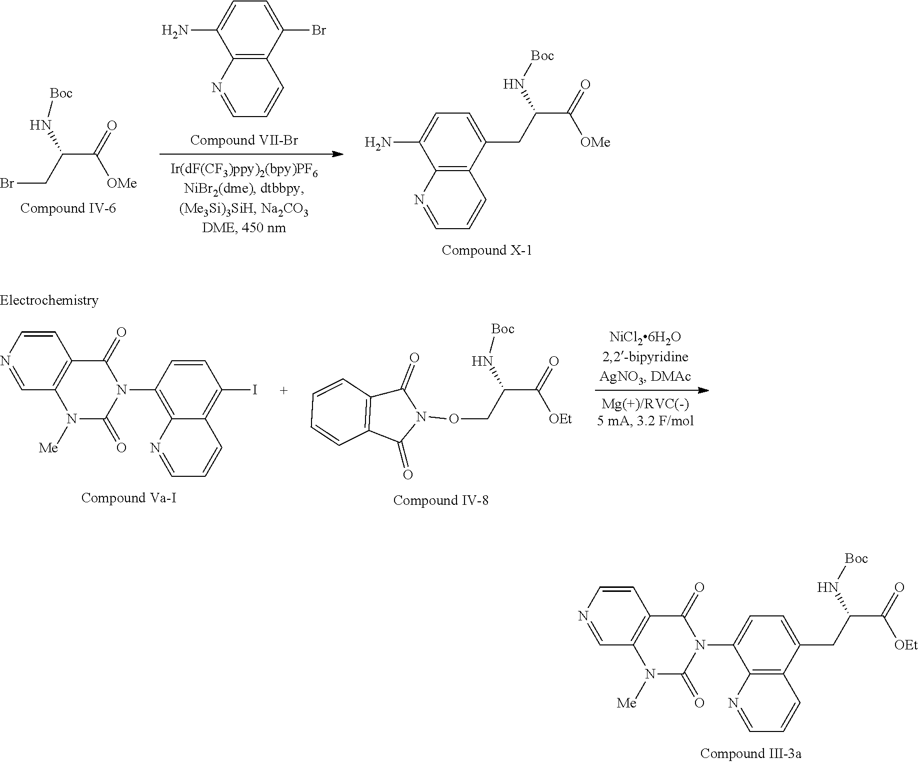 [SMILES: COC(=O)[C@H](CBr)NC(=O)OC(C)(C)C ; Nc1ccc(Br)c2cccnc12 ; COC(=O)[C@H](Cc1ccc(N)c2ncccc12)NC(=O)OC(C)(C)C ; Cn1c(=O)n(-c2ccc(I)c3cccnc23)c(=O)c2ccncc21 ; CCOC(=O)[C@H](CON1C(=O)c2ccccc2C1=O)NC(=O)OC(C)(C)C ; CCOC(=O)[C@H](Cc1ccc(-n2c(=O)c3ccncc3n(C)c2=O)c2ncccc12)NC(=O)OC(C)(C)C]

### Photochemical Coupling of Compound IV-5 and Compound V-1-Br to Compound III-4

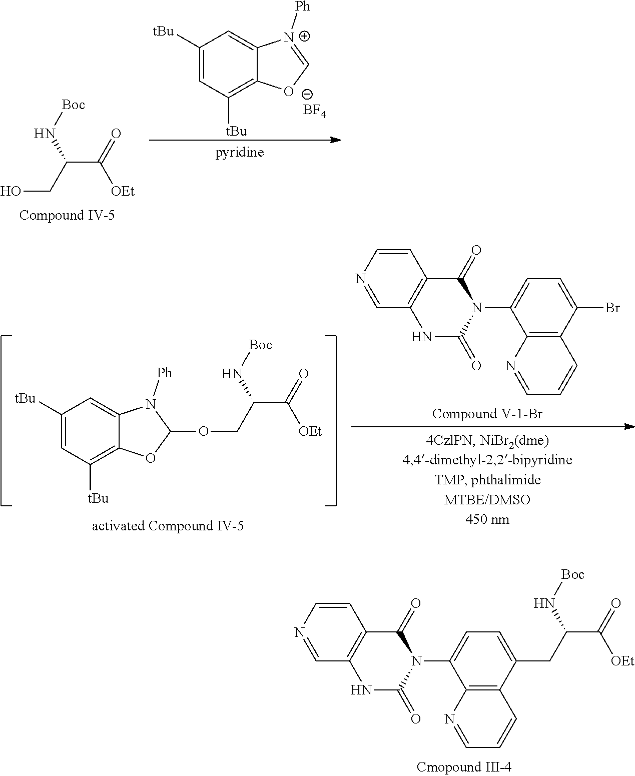

Activation of Compound IV-5. Compound IV-5 (1.85 equiv, scaling factor based on 1.00 equiv), 5,7-di-tert-butyl-3-phenylbenzo[d]oxazol-3-ium tetrafluoroborate (1.75 equiv) and magnetic stir bar were charged to vial A. In the glovebox, MTBE (15.0 volumes) was added, and the reaction stirred at about 20° C. for about 15 min. Then, a pyridine solution (1.75 equiv in MTBE (3.0 volumes)) was added dropwise at about 20° C. over about 15 min. The resulting solution stirred at about 20° C. for about 15 min. The reaction mixture was filtered via syringe filter and rinsed with MTBE (3 volumes). The filtrate and DMSO (8.0 volumes) were charged to vial B. The reaction mixture in vial B was concentrated under reduced pressure at about 25° C. to about 15 volumes.

Deoxygenative Coupling to Compound III-4. Compound V-1-Br (1.00 equiv, scaling factor), phthalimide (0.90 equiv), NiBr₂(dme) (0.090 equiv), 4,4′-dimethyl-2,2′-bipyridine (0.090 equiv), 4CzIPN (0.030 equiv) and magnetic stir bar were charged to vial C. In the glovebox, DMSO (3.0 volumes) was added, and the reaction stirred at about 20° C. for about 15 min. The DMSO solution from vial B was charged to vial C and the reaction mixture was stirred at about 20° C. for about 15 min. The reaction mixture was irradiated under 450 nm LED modules at around 25° C. for about 3 h. The reaction mixture was diluted with ethyl acetate (100.0 volumes) and washed with 3.3 wt % LiCl solution (100.0 volumes). The aqueous layer was extracted with ethyl acetate (50.0 volumes). The organic stream was combined and concentrated under reduced pressure and purified by chromatography to afford Compound III-4. ¹H NMR (400 MHz, CDCl₃) δ 10.73 (br s, 1H), 8.81 (dd, J=1.2, 4.1 Hz, 1H), 8.47 (d, J=9.2 Hz, 1H), 8.43 (d, J=5.0 Hz, 1H), 7.97 (s, 1H), 7.86 (d, J=5.2 Hz, 1H), 7.65 (d, J=7.5 Hz, 1H), 7.44 (dd, J=4.1, 8.8 Hz, 1H), 6.01 (d, J=7.6 Hz, 1H), 4.74 (dd, J=6.6, 13.4 Hz, 1H), 4.18-4.01 (m, 2H), 3.70-3.53 (m, 2H), 1.44 (s, 9H), 1.14 (t, J=7.1 Hz, 3H).

### Photochemical Coupling of Compound IV-5 and Compound V-Br to Compound III-3

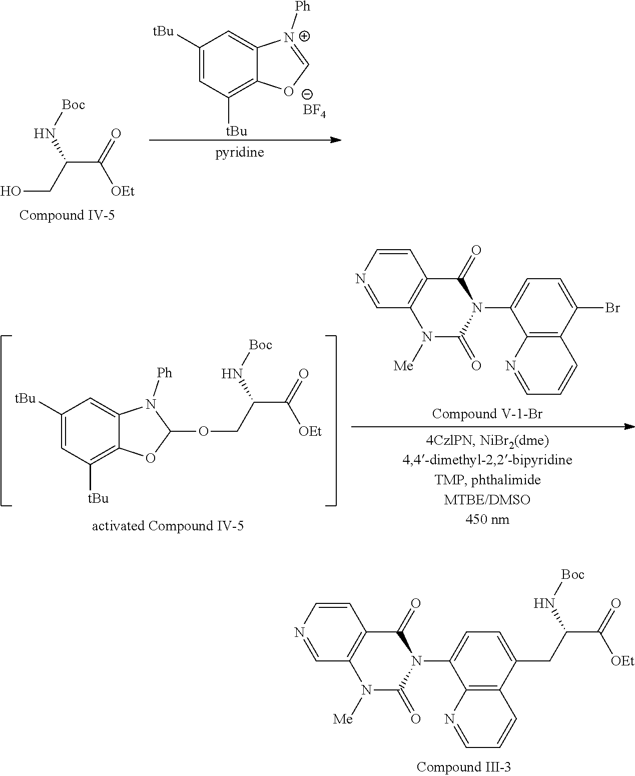

Activation of Compound IV-5. Compound IV-5 (1.85 equiv, scaling factor based on 1.00 equiv), 5,7-di-tert-butyl-3-phenylbenzo[d]oxazol-3-ium tetrafluoroborate (1.75 equiv) and magnetic stir bar were charged to vial A. In the glovebox, MTBE (15.0 volumes) was added, and the reaction stirred at about 20° C. for about 15 min. Then, a pyridine solution (1.75 equiv in MTBE (3.0 volumes)) was added dropwise at about 20° C. over about 15 mm. The resulting solution stirred at about 20° C. for about 15 min. The reaction mixture was filtered via syringe filter and rinsed with MTBE (3 volumes). The filtrate and DMSO (15.0 volumes) were charged to vial B. The reaction mixture in vial B was concentrated under reduced pressure at about 25° C. to about 15 volumes.

Deoxygenative Coupling to Compound III-3. Compound V-Br (1.00 equiv, scaling factor), phthalimide (0.90 equiv), NiBr₂(dme) (0.090 equiv), 4,4′-dimethyl-2,2′-bipyridine (0.090 equiv), 4CzIPN (0.030 equiv) and magnetic stir bar were charged to vial C. In the glovebox, DMSO (10.0 volumes) was added, and the reaction stirred at about 20° C. for about 15 min. The DMSO solution from vial B was charged to vial C and the reaction mixture was reaction stirred at about 20° C. for about 15 min. The reaction mixture was irradiated under 450 nm LED modules at around 25° C. for about 3 h. The organic stream was washed with heptane (30.0 volumes), 1:1 heptane/MTBE mixture (30.0 volumes) twice, 1:1 water/ethyl acetate mixture (60.0 volumes) and toluene (20.0 volumes). Reactor contents were concentrated under reduced pressure at about 40° C. to about 1.0 volumes and charged with isopropyl acetate (8.0 volumes). The contents of the reactor were adjusted to about 50° C. and heptane (3.0 volumes) was slowly charged to the reactor. The resulting reaction mixture was cooled to about 20° C. and agitated for about 20 hours. The slurry was filtered and the cake was dried under reduced pressure to afford Compound III-3. ¹H NMR (400 MHz, DMSO-d₆) δ 9.04 (s, 1H), 8.81 (dd, J=1.5, 4.1 Hz, 1H), 8.60-8.57 (m, 2H), 7.91 (d, J=5.0 Hz, 1H), 7.76 (d, J=7.6 Hz, 1H), 7.66-7.62 (m, 2H), 7.51 (d, J=8.0 Hz, 1H), 4.31 (td, J=9.9, 5.4 Hz, 1H), 4.11-4.01 (m, 2H), 3.65 (s, 3H), 3.58 (dd, J=14.4, 6.6 Hz, 1H), 3.45 (dd, J=14.4, 9.4 Hz, 1H), 1.35 (s, 9H), 1.04 (t, J=7.2 Hz, 3H).

### Methylation of Compound III-4 to Compound III-3

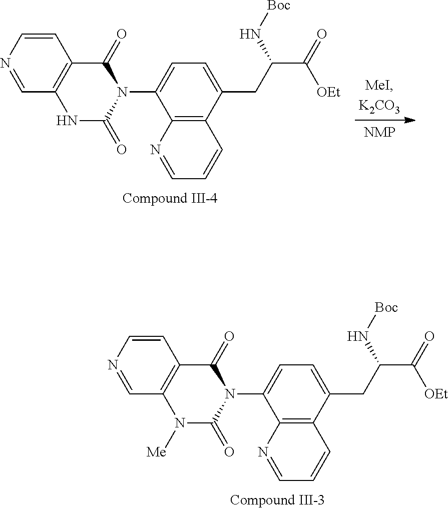

Compound III-4 (1.00 equiv, scaling factor), potassium carbonate (1.20 equiv), iodomethane (1.10 equiv) and NMP (15.0 volumes) were charged to vial A and the reaction stirred at about 20° C. for about 30 min. The reaction mixture was diluted with DCM (200.0 volumes) and washed with water (100.0 volumes). Then the organic streams were washed with 0.3 wt % LiCl solution (100.0 volumes.). The organic stream was combined and concentrated under reduced pressure to afford Compound III-3. ¹H NMR (400 MHz, DMSO-d₆) δ 9.04 (s, 1H), 8.81 (dd, J=1.5, 4.1 Hz, 1H), 8.60-8.57 (m, 2H), 7.91 (d, J=5.0 Hz, 1H), 7.76 (d, J=7.6 Hz, 1H), 7.66-7.62 (m, 2H), 7.51 (d, J=8.0 Hz, 1H), 4.31 (td, J=9.9, 5.4 Hz, 1H), 4.11-4.01 (m, 2H), 3.65 (s, 3H), 3.58 (dd, J=14.4, 6.6 Hz, 1H), 3.45 (dd, J=14.4, 9.4 Hz, 1H), 1.35 (s, 9H), 1.04 (t, J=7.2 Hz, 3H).

### Photochemical Coupling of Compound IV-6 and Compound Va-Br to Compound III-3a

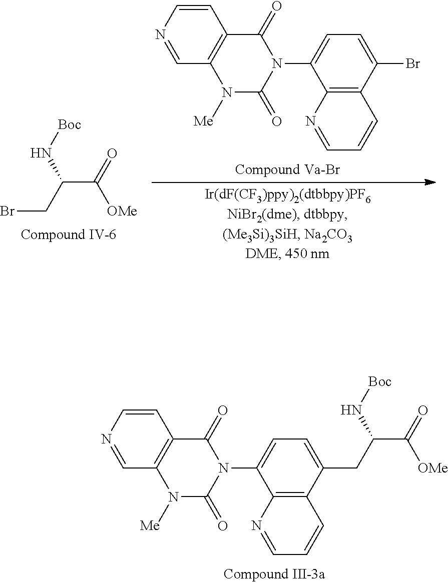 [SMILES: COC(=O)[C@H](CBr)NC(=O)OC(C)(C)C ; Cn1c(=O)n(-c2ccc(Br)c3cccnc23)c(=O)c2ccncc21 ; COC(=O)[C@H](Cc1ccc(-n2c(=O)c3ccncc3n(C)c2=O)c2ncccc12)NC(=O)OC(C)(C)C]

Compound Va-Br (1.00 equiv, scaling factor), Compound IV-6 (1.50 equiv), Ir[dF(CF₃)ppy]₂(dtbbpy)PF₆ (0.01 equiv), and anhydrous sodium carbonate (2.00 equiv) were charged to vial A. In the glovebox, DME (30.0 volumes) and tris(trimethylsilyl)silane (1.00 equiv) were charged to vial A. To a separated vial B was added NiBr₂(dme) (0.030 equiv) and 4,4′-dibutyl-2,2′-bipyridine (0.030 equiv). In the glovebox, DME (30.0 volumes) was charged to vial B and the reaction mixture was stirred at around 20° C. for about 15 minutes, after which, the reaction mixture in vial B was transferred to vial A. The reaction mixture was irradiated under 450 nm LED modules at around 25° C. for about 3 h. The reaction was quenched by exposure to air and concentrated under reduced pressure. Purification by column chromatography yielded Compound III-3a. ¹H NMR (400 MHz, CDCl₃) δ 1H NMR (400 MHz, CDCl3) δ 8.85 (d, J=1.1 Hz, 1H), 8.82 (dd, J=4.2, 1.6 Hz, 1H), 8.61 (dd, J=4.9, 0.9 Hz, 1H), 8.51 (d, J=8.6 Hz, 1H), 8.04 (ddd, J=4.9, 4.1, 0.8 Hz, 1H), 7.62 (d, J=7.5 Hz, 1H), 7.47-4.45 (m, 2H), 5.26-5.09 (m, 1H), 4.72 (q, J=7.0 Hz, 1H), 3.74 (s, 3H), 3.68-3.49 (m, 4H), 1.44 (s, 9H).

### Photochemical Coupling of Compound IV-6 and Compound VII-Br to Compound X-1

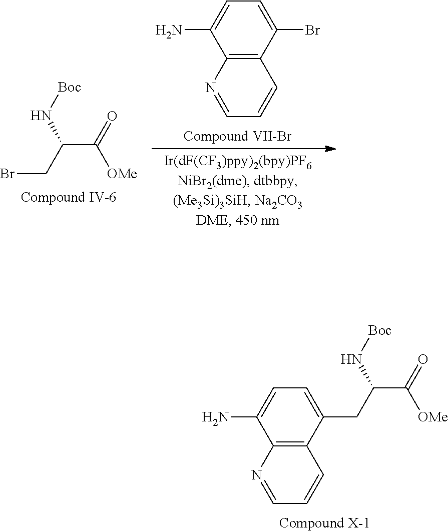 [SMILES: COC(=O)[C@H](CBr)NC(=O)OC(C)(C)C ; Nc1ccc(Br)c2cccnc12 ; COC(=O)[C@H](Cc1ccc(N)c2ncccc12)NC(=O)OC(C)(C)C]

Compound VII-Br (1.00 equiv, scaling factor), Compound IV-6 (1.50 equiv), Ir[dF(CF₃)ppy]₂(bpy)PF₆ (0.01 equiv), and anhydrous sodium carbonate (2.00 equiv) were charged to vial A. In the glovebox, tris(trimethylsilyl)silane (1.00 equiv) and 1,2-dimethoxyethane (50.0 volumes) were charged to vial A. To a separated vial B was added NiBr₂(dme) (0.030 equiv) and 4,4′-dibutyl-2,2′-bipyridine (0.030 equiv). In the glovebox, DME (5.0 volumes) was charged to vial B and the reaction mixture was stirred at around 20° C. for about 15 minutes, after which, the reaction mixture in vial B was transferred to vial A. The reaction mixture was irradiated under 450 nm LED modules at around 25° C. for about 10 h. The reaction was quenched by exposure to air and concentrated under reduced pressure. Purification by column chromatography yielded Compound X-1. ¹H NMR (400 MHz, CDCl₃) δ 8.77 (dd, J=1.7, 4.0 Hz, 1H), 8.32 (d, J=8.0 Hz, 1H), 7.42 (dd, J=4.3, 8.5 Hz, 1H), 7.09 (d, J=8.3 Hz, 1H), 6.84 (d, J=8.0 Hz, 1H), 5.04 (d, J=8.1 Hz, 1H), 4.61 (q, J=7.0 Hz, 1H), 3.61 (s, 3H), 3.50-3.28 (m, 2H), 1.40 (s, 9H).

### Electrochemical Coupling of Compound Va-I and Compound IV-8 to Compound III-3a

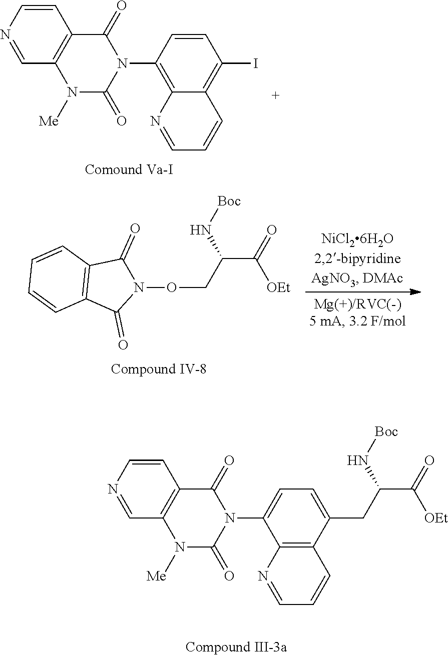 [SMILES: Cn1c(=O)n(-c2ccc(I)c3cccnc23)c(=O)c2ccncc21 ; CCOC(=O)[C@H](CON1C(=O)c2ccccc2C1=O)NC(=O)OC(C)(C)C ; CCOC(=O)[C@H](Cc1ccc(-n2c(=O)c3ccncc3n(C)c2=O)c2ncccc12)NC(=O)OC(C)(C)C]

Compound Va-I (1.00 equiv, scaling factor), Compound IV-8 (1.00 equiv), nickel(II) chloride hexahydrate (0.20 equiv), 2,2′-bipyridine (0.20 equiv) and N,N-dimethylacetamide (46.8 volumes) were charged to ElectraSyn 2.0 vial. The contents of the vial were allowed to stir for about 15 minutes. Silver nitrate (0.50 equiv) was then added to the reaction mixture. The vial was equipped with ElectraSyn 2.0 vial cap with a magnesium sacrificial anode and RVC cathode. The vial was then placed on an IKA ElectraSyn 2.0 stir plate and electrolysis was set to about 5 mA and about 3.2 F/mol at about 20° C. After completion of the reaction, the reaction mixture was purified by column chromatography to yield Compound III-3a. ¹H NMR (400 MHz, DMSO-d₆) δ 9.04 (s, 1H), 8.81 (dd, J=1.5, 4.1 Hz, 1H), 8.60-8.57 (m, 2H), 7.91 (d, J=5.0 Hz, 1H), 7.76 (d, J=7.6 Hz, 1H), 7.66-7.62 (m, 2H), 7.51 (d, J=8.0 Hz, 1H), 4.31 (td, J=9.9, 5.4 Hz, 1H), 4.11-4.01 (m, 2H), 3.65 (s, 3H), 3.58 (dd, J=14.4, 6.6 Hz, 1H), 3.45 (dd, J=14.4, 9.4 Hz, 1H), 1.35 (s, 9H), 1.04 (t, J=7.2 Hz, 3H).

### Example 5: Synthesis of Compound IIIa (Synthesis of Compound III Via Erlenmeyer Reaction—Method D)

### Reductive Carbonylation of Compound Va-I to Compound V-4a

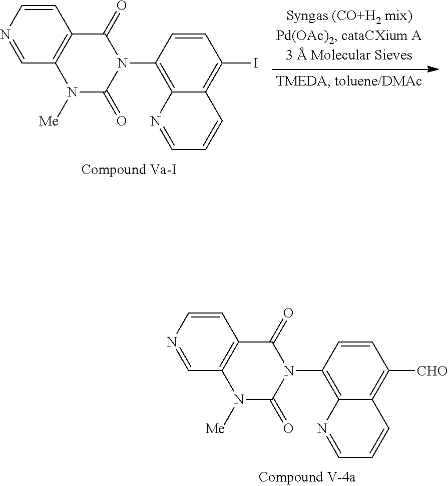 [SMILES: Cn1c(=O)n(-c2ccc(I)c3cccnc23)c(=O)c2ccncc21 ; Cn1c(=O)n(-c2ccc(C=O)c3cccnc23)c(=O)c2ccncc21]

Compound Va-I (1.00 equiv, scaling factor), CataCXium A (0.075 equiv), palladium(II) acetate (0.025 equiv), powdered molecular sieves 3 Å (20 wt %), and toluene (7.8 volumes) were charged to a pressure reactor vessel. The reactor vessel was sealed and N,N-dimethylacetamide (2.6 volumes) was charged via syringe. The reactor vessel was purged with nitrogen, charged with N,N,N′,N′-tetramethylethylenediamine (1.00 equiv), purged with nitrogen, pressurized to about 150 psig with Syngas (mixture of carbon monoxide and hydrogen gas), and agitated at about 85° C. for about 20 hours. The resulting reaction mixture was cooled to about 20° C., vented, filtered, and the solids were rinsed with toluene (6.0 volumes). The filtrate was washed with water (4×4.0 volumes) and the combined aqueous layers were back extracted with toluene (6.0 volumes). The combined organic layers were dried over sodium sulfate, filtered, and concentrated under reduced pressure to afford crude material. Crude material was dissolved in THF (10.0 volumes) and agitated at about 45° C. Reaction mixture was charged with sodium bisulfite (1.00 equiv) in water (1.0 volume) at a rate of about 1.5 mL/min and agitated for about 3 hours. The slurry was filtered, the cake was washed with THF (6.0 volumes) and dried under reduced pressure to afford the bisulfite adduct. The bisulfite adduct, 1M aqueous sodium carbonate (3.00 equiv), and dichloromethane (10.0 volumes) were charged to a reactor and the mixture was agitated at about 20° C. for about 3 hours. The aqueous layer was back extracted with dichloromethane (3×6.0 volumes) and the combined organic layers were dried over sodium sulfate, filtered, and concentrated under reduced pressure to afford Compound V-4a. ¹H NMR (400 MHz, DMSO-d₆) δ 10.48 (s, 1H), 9.54 (dd, J=8.7, 1.4 Hz, 1H), 9.07 (s, 1H), 8.94 (dd, J=4.1, 1.4 Hz, 1H), 8.62 (d, J=5.0 Hz, 1H), 8.45 (d, J=7.6 Hz, 1H), 8.13 (d, J=7.5 Hz, 1H), 7.93 (d, J=5.0 Hz, 1H), 7.79 (dd, J=8.7, 4.2 Hz, 1H), 3.67 (s, 3H).

### Conversion of Compound V-4a to Compound III-8a-Et

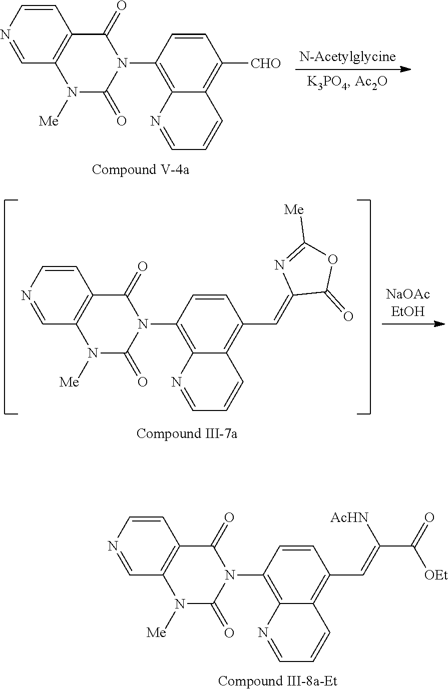

Compound V-4a (1.00 equiv, scaling factor), N-Acetylglycine (2.0 equiv), potassium phosphate tribasic (1.0 equiv), and acetic anhydride (5.0 volumes) were charged to a reaction vessel and the resulting mixture was agitated at about 85° C. for about 4 hours. The reaction mixture was cooled to about −5° C., slowly charged with ethanol (10.0 volumes), sodium acetate (3.0 equiv), and agitated for about 15 minutes. The resulting reaction mixture was then agitated at about 60° C. for about 3 hours. The mixture was cooled to about 20° C. and concentrated under reduced pressure at about 50° C. The crude residue was dissolved with dichloromethane (33 volumes) and washed with water (4×17 volumes). The organic stream was then dried over anhydrous sodium sulfate, filtered, and concentrated under reduced pressure. The residue was dissolved with ethanol (5.0 volumes), diluted with water (10.0 volumes), charged with sodium bisulfite (2.0 equiv) and agitated at about 20° C. for about 1 hour. The aqueous mixture was further diluted with water (10.0 volumes) and extracted with dichloromethane (3×17 volumes). The organic stream was washed with sat. aqueous sodium chloride (17 volumes), dried over sodium sulfate, filtered, and concentrated under reduced pressure. The crude solids were purified by chromatography to provide Compound III-8a-Et. ¹H NMR (400 MHz, DMSO-d₆) δ 9.78 (s, 1H), 9.05 (s, 1H), 8.86 (dd, J=4.1, 1.3 Hz, 1H), 8.61 (d, J=4.9 Hz, 1H), 8.54-8.44 (m, 1H), 7.92 (d, J=4.9 Hz, 1H), 7.88 (t, J=5.7 Hz, 2H), 7.62 (q, J=4.1 Hz, 2H), 4.25 (q, J=7.1 Hz, 2H), 3.66 (s, 3H), 1.91 (s, 3H), 1.29 (t, J=7.1 Hz, 3H).

### Asymmetric Hydrogenation of Compound III-8a-Et to Compound III-9a-Et

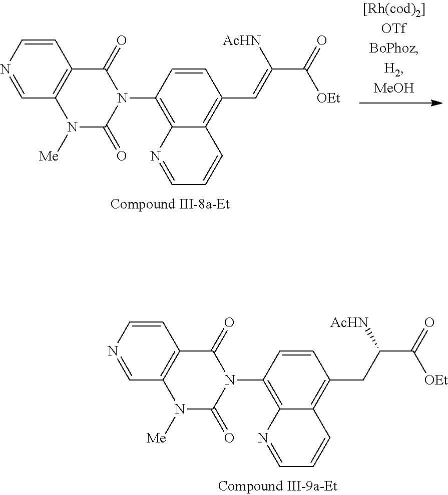 [SMILES: CCOC(=O)/C(=C/c1ccc(-n2c(=O)c3ccncc3n(C)c2=O)c2ncccc12)NC(C)=O ; CCOC(=O)[C@H](Cc1ccc(-n2c(=O)c3ccncc3n(C)c2=O)c2ncccc12)NC(C)=O]

Compound III-8a-Et (1.00 equiv, scaling factor), bis(1,5-cyclooctadiene)rhodium(I) trifluoromethanesulfonate (0.015 equiv), (1R)-1-(diphenylphosphino)2-[(1R)-1-[(diphenylphosphino)methylamino]ethyl]ferrocene (0.015 equiv), and methanol (12.0 volumes) were charged to a pressure reactor vessel. The reactor vessel was purged with nitrogen, pressurized to about 40 psig of hydrogen gas, and agitated at about 50° C. for about 20 hours. The resulting reaction mixture was cooled to about 20° C., vented, and concentrated under reduced pressure to afford crude product. The crude product was dissolved with acetone (10.0 volumes), charged with N-acetylcysteine (1.00 equiv) and N,N-diisopropylethylamine (1.20 equiv), and agitated at about 50° C. for about 16 hours. The reaction mixture was cooled to about 20° C. and filtered over a pad of celite. The filtrate was diluted with water (38 volumes) and extracted with dichloromethane (3×38 volume). The organic stream was washed with sat. aqueous sodium chloride and the organics were dried sodium sulfate, filtered, and concentrated under reduced pressure to afford Compound III-9a-Et. ¹H NMR (400 MHz, DMSO-d₆) δ 9.04 (s, 1H), 8.87-8.79 (m, 1H), 8.68-8.50 (m, 3H), 7.91 (d, J=4.9 Hz, 1H), 7.76 (d, J=7.5 Hz, 1H), 7.65 (dd, J=8.6, 4.1 Hz, 1H), 7.59 (d, J=7.6 Hz, 1H), 4.60 (q, J=7.5 Hz, 1H), 4.01 (q, J=7.0 Hz, 2H), 3.65 (s, 3H), 3.52 (ddt, J=22.7, 14.3, 7.6 Hz, 2H), 1.84 (s, 3H), 1.06 (td, J=7.1, 1.7 Hz, 3H).

### Deacetylation of Compound III-9a-Et to Compound IIIa

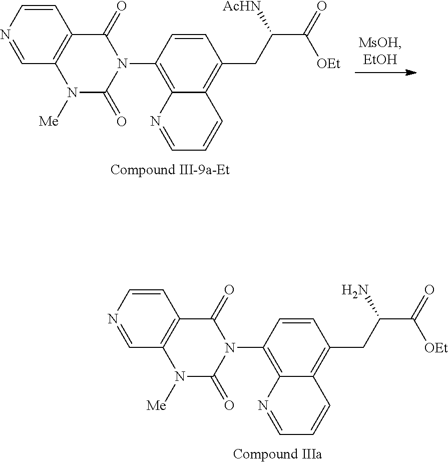 [SMILES: CCOC(=O)[C@H](Cc1ccc(-n2c(=O)c3ccncc3n(C)c2=O)c2ncccc12)NC(C)=O ; CCOC(=O)[C@@H](N)Cc1ccc(-n2c(=O)c3ccncc3n(C)c2=O)c2ncccc12]

Compound III-9a-Et (1.00 equiv, scaling factor), ethanol (13 volumes), and methanesulfonic acid (6.0 equiv) were charged to a reaction vial and the contents were agitated at about 75° C. for about 65 hours. The resulting reaction mixture was cooled to about 20° C. and was washed with aqueous ammonium hydroxide (8.0 equiv) and water (10.0 volumes). The mixture was extracted with dichloromethane (26.0 volumes), and the organics were concentrated under reduced pressure to afford Compound IIIa. ¹H NMR (400 MHz, DMSO) δ 9.05 (s, 1H), 8.82 (dd, J=4.2, 1.6 Hz, 1H), 8.67-8.57 (m, 2H), 7.92 (dd, J=4.9, 0.7 Hz, 1H), 7.74 (d, J=7.5 Hz, 1H), 7.66-7.55 (m, 2H), 3.99 (qt, J=7.1, 1.0 Hz, 2H), 3.71 (t, J=7.1 Hz, 1H), 3.66 (s, 3H), 3.43 (dd, J=13.8, 6.7 Hz, 1H), 3.39-3.32 (m, 1H), 2.24 (s, 2H), 1.06 (td, J=7.1, 1.9 Hz, 3H).

### Conversion of Compound V-4a to Compound III-8a-Me

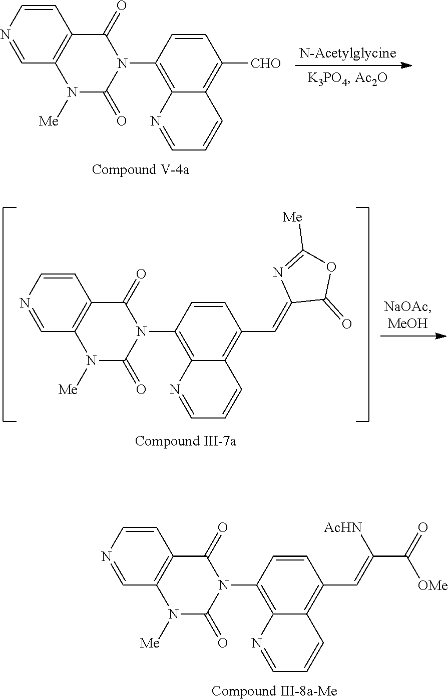

Compound V-4a (1.00 equiv, scaling factor), N-Acetylglycine (2.00 equiv), potassium phosphate tribasic (1.00 equiv), and acetic anhydride (5.0 volumes) were charged to a reaction vessel and the resulting mixture was agitated at about 85° C. for about 12 hours. The reaction mixture was cooled to about −5° C., charged with methanol (10.0 volumes), sodium acetate (3.00 equiv), and agitated for about 15 minutes. The resulting reaction mixture was then agitated at about 60° C. for about 3 hours. The mixture was cooled to about 20° C. and concentrated under reduced pressure at about 50° C. The crude residue was dissolved into dichloromethane (23 volumes) and washed with water (4×14 volumes). The organic stream was then washed with sat. aqueous sodium chloride (10.0 volumes) and concentrated under reduced pressure. The residue was dissolved in methanol (5.0 volumes), diluted with water (10.0 volumes), charged with sodium bisulfite (1.00 equiv) and agitated at about 20° C. for about 2 hours. The aqueous mixture was further diluted with water (9.1 volumes) and extracted with dichloromethane (4×9.1 volumes). The organic stream was washed with sat. aqueous sodium chloride (9.1 volumes), dried over sodium sulfate, filtered, and concentrated under reduced pressure. The crude solids were triturated with ethyl acetate (9.1 volumes) at about 20° C. for about 12 hours. The slurry was filtered and the cake was dried under reduced pressure at about 60° C. to afford Compound III-8a-Me. ¹H NMR (400 MHz, DMSO-d₆) δ 9.81 (s, 1H), 9.05 (s, 1H), 8.86 (dd, J=4.1, 1.3 Hz, 1H), 8.61 (d, J=4.9 Hz, 1H), 8.53-8.45 (m, 1H), 7.92 (d, J=4.9 Hz, 1H), 7.88 (t, J=6.0 Hz, 2H), 7.68-7.59 (m, 2H), 3.79 (s, 3H), 3.66 (s, 3H), 1.91 (s, 3H).

### Asymmetric Hydrogenation of Compound III-8a-Me to Compound III-9a-Me

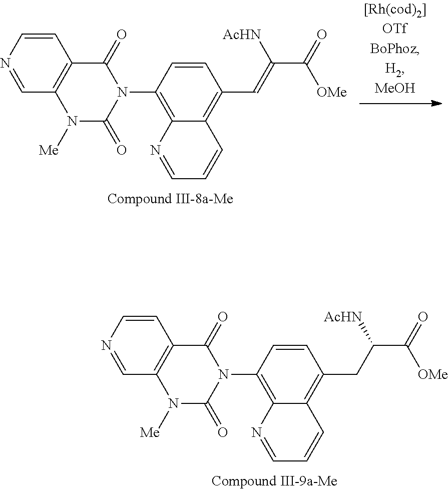 [SMILES: COC(=O)/C(=C/c1ccc(-n2c(=O)c3ccncc3n(C)c2=O)c2ncccc12)NC(C)=O ; COC(=O)[C@H](Cc1ccc(-n2c(=O)c3ccncc3n(C)c2=O)c2ncccc12)NC(C)=O]

Compound III-8a-Me (1.00 equiv, scaling factor), bis(1,5-cyclooctadiene)rhodium(I) trifluoromethanesulfonate (0.015 equiv), (1R)-1-(diphenylphosphino)2-[(1R)-1-[(diphenylphosphino)methylamino]ethyl]ferrocene (0.015 equiv), and methanol (12.0 volumes) were charged to a pressure reactor vessel. The reactor vessel was purged with nitrogen, pressurized to about 40 psig of hydrogen gas, and agitated at about 50° C. for about 20 hours. The resulting reaction mixture was cooled to about 20° C., vented, and concentrated under reduced pressure to afford crude product. The crude product was dissolved in acetone (10.0 volumes), charged with N-acetylcysteine (1.00 equiv) and N,N-diisopropylethylamine (1.20 equiv), and agitated at about 50° C. for about 12 hours. The reaction mixture was cooled to about 20° C. and filtered over a pad of celite. The filtrate was diluted with water (20.0 volumes) and extracted with dichloromethane (4×15.0 volume). The organic stream was washed with sat. aqueous sodium chloride and the organics were dried sodium sulfate, filtered, and concentrated under reduced pressure to afford crude product that was triturated overnight in a 1:1 v/v mixture of isopropanol and tert-butyl methyl ether (10.0 volumes). The slurry was filtered and dried to afford Compound III-9a-Me. ¹H NMR (400 MHz, DMSO-d₆) δ 9.04 (s, 1H), 8.88-8.77 (m, 1H), 8.65-8.58 (m, 2H), 8.57 (d, J=7.6 Hz, 1H), 7.91 (d, J=4.9 Hz, 1H), 7.76 (d, J=7.5 Hz, 1H), 7.65 (dd, J=8.6, 4.1 Hz, 1H), 7.59 (d, J=7.6 Hz, 1H), 4.63 (q, J=8.3 Hz, 1H), 3.65 (d, J=1.1 Hz, 3H), 3.64-3.53 (m, 4H), 3.47 (dd, J=14.3, 9.1 Hz, 1H), 1.82 (s, 3H).

### Deacetylation and Transesterification of Compound III-9a-Me to Compound IIIa

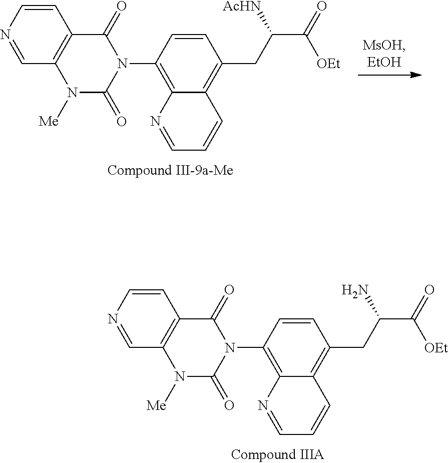 [SMILES: CCOC(=O)[C@H](Cc1ccc(-n2c(=O)c3ccncc3n(C)c2=O)c2ncccc12)NC(C)=O ; CCOC(=O)[C@@H](N)Cc1ccc(-n2c(=O)c3ccncc3n(C)c2=O)c2ncccc12]

Compound III-9a-Me (1.00 equiv, scaling factor), ethanol (13 volumes), and methanesulfonic acid (6.0 equiv) were charged to a reaction vial and the contents were agitated at about 75° C. for about 65 hours. The resulting reaction mixture was cooled to about 20° C. and was washed with aqueous ammonium hydroxide (8.0 equiv) and water (10.0 volumes). The mixture was extracted with dichloromethane (26.0 volumes), and the organics were concentrated under reduced pressure to afford Compound IIIa. ¹H NMR (400 MHz, DMSO) δ 9.05 (s, 1H), 8.82 (dd, J=4.2, 1.6 Hz, 1H), 8.67-8.57 (m, 2H), 7.92 (dd, J=4.9, 0.7 Hz, 1H), 7.74 (d, J=7.5 Hz, 1H), 7.66-7.55 (m, 2H), 3.99 (qt, J=7.1, 1.0 Hz, 2H), 3.71 (t, J=7.1 Hz, 1H), 3.66 (s, 3H), 3.43 (dd, J=13.8, 6.7 Hz, 1H), 3.39-3.32 (m, 1H), 2.24 (s, 2H), 1.06 (td, J=7.1, 1.9 Hz, 3H).

### Example 6: Synthesis of Compound III-8a-Me (Synthesis of Compound III Via Heck Reaction—Method E)

### Acylation of DL-Serine-OMe HCl to Compound IV-9-Me

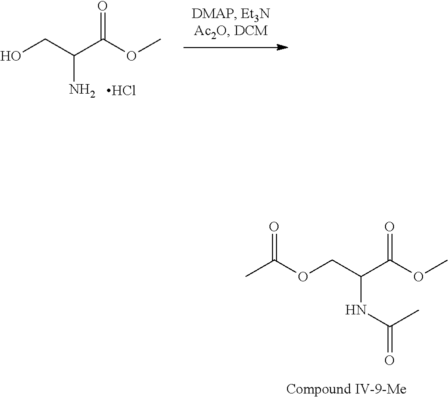

DL-Serine-OMe HCl (1.00 equiv, scaling factor), 4-(dimethylamino)pyridine (0.05 equiv), and dichloromethane (12.0 volumes) were charged to a reactor and the slurry was agitated at about 10° C. The slurry was charged with triethylamine (3.00 equiv) and cooled to about 5° C. The reactor contents were charged with acetic anhydride (5.00 equiv) over about 30 minutes. After complete addition, the slurry was agitated overnight at about 15° C. Reaction mixture was quenched with aqueous 5% NaHCO₃ (6.0 volumes) and the organic layer was washed with water (6.0 volumes). Organics were dried over Mg₂SO₄, filtered, and concentrated under reduced pressure to afford Compound IV-9-Me. ¹H NMR (400 MHz, DMSO-d₆) δ 8.43 (d, J=7.8 Hz, 1H), 4.59 (ddd, J=7.8, 6.0, 4.5 Hz, 1H), 4.26 (dd, J=11.3, 4.6 Hz, 1H), 4.19 (dd, J=11.3, 6.0 Hz, 1H), 3.65 (s, 3H), 2.01 (s, 3H), 1.87 (s, 3H).

### Heck Coupling to Prepare Compound III-8a-Me

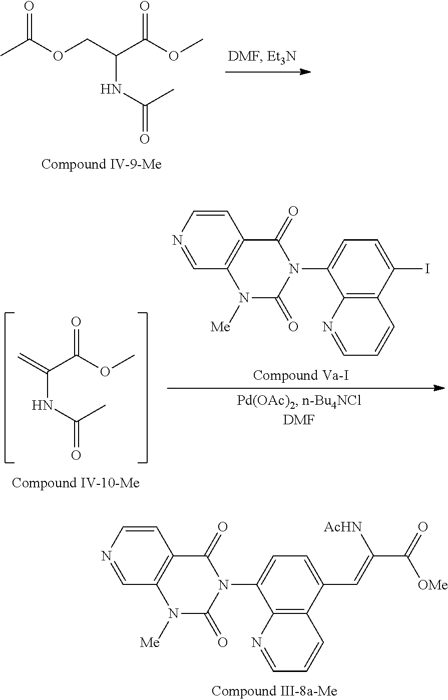

A reaction vial was charged with Compound Va-I (1.00 equiv, scaling factor), palladium(II) acetate (0.05 equiv), tetrabutylammonium chloride (1.00 equiv), and N,N-dimethylformamide (10.0 volumes). Vial contents were agitated at about 20° C. and charged with Compound IV-9-Me (1.10 equiv) and triethylamine (3.50 equiv). Resulting reaction mixture was agitated overnight at about 90° C. Reaction mixture was cooled to about 20° C., concentrated under reduced pressure, and purified by column chromatography to afford Compound III-8a-Me. 11H NMR (400 MHz, DMSO-d₆) δ 9.81 (s, 1H), 9.05 (s, 1H), 8.86 (dd, J=4.1, 1.3 Hz, 1H), 8.61 (d, J=4.9 Hz, 1H), 8.53-8.45 (m, 1H), 7.92 (d, J=4.9 Hz, 1H), 7.88 (t, J=6.0 Hz, 2H), 7.68-7.59 (m, 2H), 3.79 (s, 3H), 3.66 (s, 3H), 1.91 (s, 3H).

### Example 7: Synthesis of Compound III-3 (Synthesis of Compound III Via Asymmetric Cyclization—Method F)

### Synthesis of Compound III-10

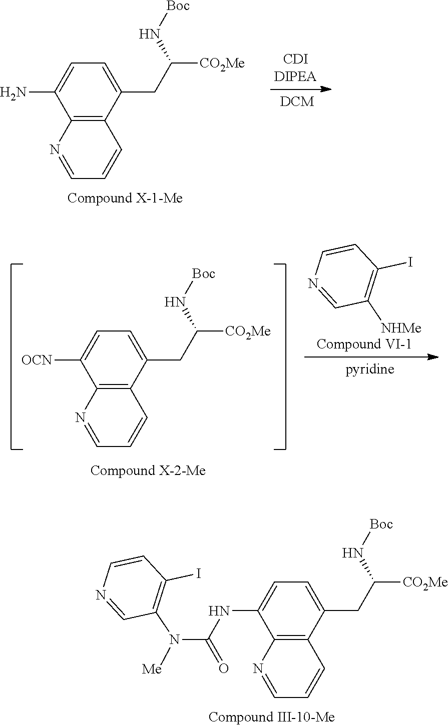

A mixture of Compound X-1-Me (1.00 equiv, scaling factor) and 1,1′-carbonyldiimidazole (1.2 equiv) in dichloromethane (8 volumes) was charged with N,N-diisopropylethylamine and stirred at about 20° C. for about 3 hours. Compound VI-1 (1.0 equiv) was added, and the mixture was concentrated under reduced pressure to dryness. The mixture was redissolved in pyridine (16 volumes) and heated at about 100° C. for about 22 hours. The mixture was cooled to about 20° C., concentrated under reduced pressure, and purified by chromatography to afford Compound III-10-Me. ¹H NMR (300 MHz, CDCl₃) δ 8.98 (br s, 1H), 8.64 (s, 1H), 8.48 (dd, J=4.4, 1.8 Hz, 1H), 8.46 (d, J=7.7 Hz, 1H), 8.35 (d, J=8.6 Hz, 1H), 8.29 (d, J=5.2 Hz, 1H), 7.99 (d, J=5.1 Hz, 1H), 7.37 (dd, J=8.6, 4.2 Hz, 1H), 7.30-7.24 (m, 1H), 5.03 (d, J=7.9 Hz, 1H), 4.60 (q, J=6.8 Hz, 1H), 3.62 (s, 3H), 3.47 (dd, J=14.3, 6.5 Hz, 1H), 3.42-3.35 (m, 1H), 3.40 (s, 3H), 1.40 (br s, 9H).

### Carbonylative Cyclization of Compound III-10-Me to Compound III-2

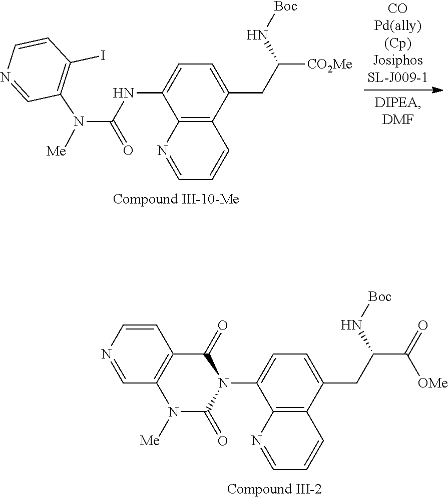

A mixture of Compound III-10-Me (1.00 equiv, scaling factor), allyl(cyclopentadienyl)palladium(II) (0.04 equiv), Josiphos ligand SL-J013-1 (CAS 187733-50-2; 0.04 equiv), N,N-diisopropylethylamine (4 equiv), and N,N-dimethylformamide (15 volumes) was pressurized to about 450 psi carbon monoxide and heated at about 70° C. for about 68 hours. The mixture was cooled to about 20° C. and purified via chromatography to afford Compound III-2. ¹H NMR (400 MHz, CDCl3) δ 8.86 (s, 1H), 8.83 (dd, J=4.2, 1.5 Hz, 1H), 8.61 (d, J=5.0 Hz, 1H), 8.51 (br d, J=8.6 Hz, 1H), 8.05 (dd, J=5.0, 0.8 Hz, 1H), 7.62 (d, J=7.5 Hz, 1H), 7.48 (dd, J=8.6, 4.2 Hz, 1H), 7.46 (d, J=7.5 Hz, 1H), 5.23 (d, J=7.8 Hz, 1H), 4.72 (q, J=7.0 Hz, 1H), 3.74 (s, 3H), 3.63 (br s, 3H), 3.61-3.58 (m, 2H), 1.45 (br s, 9H).

### Example 8: Synthesis of Compound III-4 (Synthesis of Compound III Via Asymmetric Cyclization—Method G)

### Negishi Coupling of Compound IV-3 and Compound VII-1 to Compound X-2

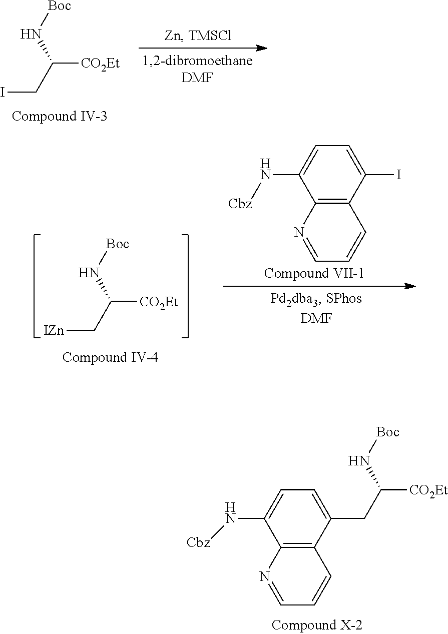

Formation of Compound IV-4. A reactor containing zinc powder (6.0 equiv), 1,2-dibromoethane (0.2 equiv), and N,N-dimethylformamide (3.6 volumes) was charged with trimethylsilyl chloride (0.04 equiv) followed by a solution of Compound IV-3 (2.00 equiv) in N,N-dimethylformamide (3.5 volumes). The resulting mixture of Compound IV-4 was stirred overnight at about 20° C.

Negishi coupling. A separate reactor containing Compound VII-1 (1.00 equiv, scaling factor), N,N-dimethylformamide (5.0 volumes), tris(dibenzylideneacetone)dipalladium(0) (0.04 equiv), and 2-dicyclohexylphosphino-2′,6′-dimethoxybiphenyl (0.08 equiv) was heated to about 55° C. and the organozinc solution prepared above was added dropwise. The reaction mixture was stirred at about 55° C. for about 1 hour and then cooled to about 20° C. and charged with water (10 volumes) and ethyl acetate (75 volumes) and stirred for about 20 minutes. The mixture was filtered through a pad of celite and the filtrate was washed three times with water (50 volumes per wash), then washed two times with 10% aqueous lithium chloride solution (50 volumes per wash), then washed one time with saturated sodium chloride solution (50 volumes). The resulting organics were concentrated under reduced pressure. The crude was purified by column chromatography to afford Compound X-2. ¹H NMR (300 MHz, CDCl₃) δ 9.31 (s, 1H), 8.78 (dd, J=4.2, 1.6 Hz, 1H), 8.44 (d, J=7.5 Hz, 1H), 8.34 (d, J=7.9 Hz, 1H), 7.55-7.33 (m, 5H), 7.30 (d, J=8.3 Hz, 1H), 5.28 (s, 2H), 5.10 (d, J=8.0 Hz, 1H), 4.61 (dt, J=6.9, 6.9 Hz, 1H), 4.07 (m, 2H), 3.48 (dd, J=14.1, 6.6 Hz, 1H), 3.43 (dd, J=13.9, 6.6 Hz, 1H) 1.41 (s, 9H), 1.12 (t, J=7.1 Hz, 3H).

### Deprotection of Compound X-2 to Compound X-3

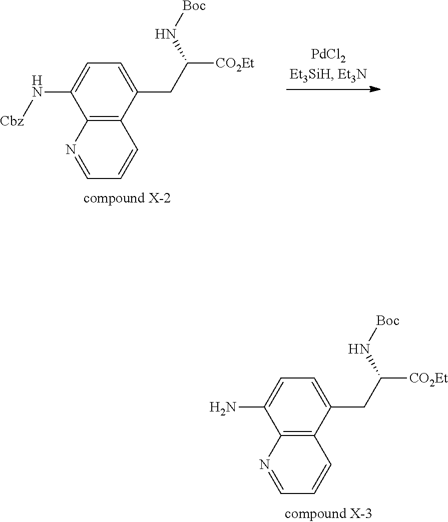 [SMILES: CCOC(=O)[C@H](Cc1ccc(NC(=O)OCc2ccccc2)c2ncccc12)NC(=O)OC(C)(C)C ; CCOC(=O)[C@H](Cc1ccc(N)c2ncccc12)NC(=O)OC(C)(C)C]

A mixture of compound X-2 (1.00 equiv, scaling factor), triethylsilane (10 equiv), and triethylamine (12 equiv) in dichloromethane (5 volumes) and methanol (5 volumes) was sparged with nitrogen for about 10 minutes. Palladium(II) chloride (0.50 equiv) was added and the resulting mixture was stirred at about 20° C. for about 2 hours. The mixture was concentrated under reduced pressure and purified by chromatography to afford compound X-3. ¹H NMR (400 MHz, CDCl₃) δ 8.76 (dd, J=4.1, 1.6 Hz, 1H), 8.33 (d, J=8.6 Hz, 1H), 7.41 (dd, J=8.6, 4.1 Hz, 1H), 7.11 (d, J=7.7 Hz, 1H), 6.83 (d, J=7.7 Hz, 1H), 5.04 (d, J=8.1 Hz, 1H), 4.97 (s, 2H), 4.58 (dt, J=7.0, 6.9 Hz, 1H), 4.10-4.01 (m, 2H), 3.41 (dd, J=14.0, 6.8 Hz, 1H), 3.35 (dd, J=14.2, 6.6 Hz, 1H), 1.40 (s, 9H), 1.12 (t, J=7.2 Hz, 3H).

### Curtius Rearrangement of Compound X-3 to Compound III-5

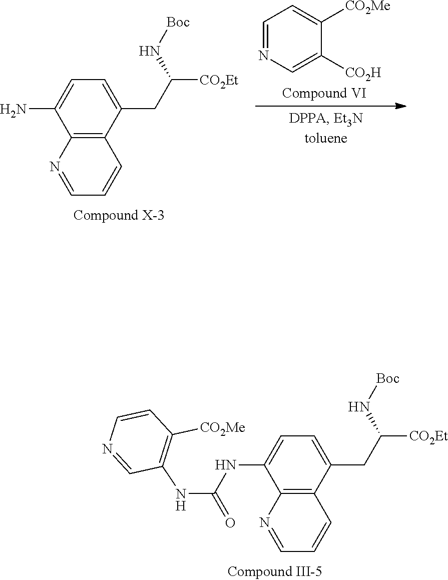 [SMILES: CCOC(=O)[C@H](Cc1ccc(N)c2ncccc12)NC(=O)OC(C)(C)C ; COC(=O)c1ccncc1C(=O)O ; CCOC(=O)[C@H](Cc1ccc(NC(=O)Nc2cnccc2C(=O)OC)c2ncccc12)NC(=O)OC(C)(C)C]

Compound X-3 (1.00 equiv, scaling factor), Compound VI (1.5 equiv), and toluene (8 volumes) were charged to a reactor and warmed to about 80° C. Triethylamine (3 equiv) was added, followed by a solution of diphenylphosphoryl azide (1.2 equiv) in toluene (4 volumes), and the resulting mixture was heated at about 80° C. for about 2 hours. The mixture was cooled to about 20° C., concentrated under reduced pressure and purified by chromatography to afford Compound III-5. ¹H NMR (400 MHz, CDCl₃) δ 10.34 (s, 1H), 9.96 (d, J=0.7 Hz, 1H), 9.44 (s, 1H), 8.84 (dd, J=4.3, 1.5 Hz, 1H), 8.51 (d, J=8.0 Hz, 1H), 8.48 (d, J=8.8 Hz, 1H), 8.37 (d, J=5.1 Hz, 1H), 7.79 (dd, J=5.1, 0.7 Hz, 1H), 7.52 (dd, J=8.6, 4.2 Hz, 1H), 7.32 (d, J=7.9 Hz, 1H), 5.11 (d, J=7.9 Hz, 1H), 4.62 (q, J=6.9 Hz, 1H), 4.09 (m, 2H), 4.02 (s, 3H), 3.50 (dd, J=14.1, 6.6 Hz, 1H), 3.45 (dd, J=14.3, 6.0 Hz, 1H), 1.42 (s, 9H), 1.13 (t, J=7.1 Hz, 3H).

### Asymmetric Cyclization of Compound III-5 to Compound III-4

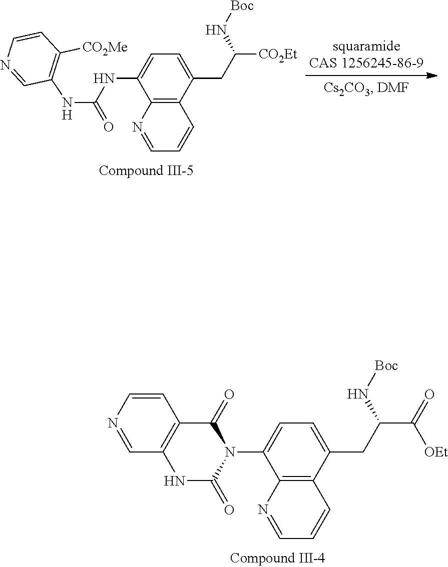

A round bottom flask was charged with Compound III-5 (1.00 equiv, scaling factor), Cs₂CO₃ (0.04 equiv), and DMF (10.0 volumes). The squaramide organocatalyst (3-[[3,5-bis(trifluoromethyl)phenyl]amino]-4-[(8a,9S)-cinchonan-9-ylamino]-3-Cyclobutene-1,2-dione, CAS 1256245-86-9) (0.06 equiv) was charged to the flask. The reaction mixture was stirred for about four days at about 20° C. under a nitrogen atmosphere. The reaction mixture was dissolved in distilled water (100.0 volumes) and transferred to a separatory funnel along with DCM (175.0 volumes). The combined organic phase was washed with distilled water (150.0 volumes) and 10 wt % aqueous LiCl solution (150.0 volumes). The organic phase was dried with anhydrous Na₂SO₄ for about 1 hour before the solids were removed by vacuum filtration. The filtrate was deposited onto silica (20 parts) under vacuum and purified by column chromatography. Selected fractions were concentrated to give Compound III-4. ¹H NMR (300 MHz, d6-DMSO) δ 11.93 (s, 1H), 8.84 (dd, J=4.1, 1.5 Hz, 1H), 8.71 (d, J=0.8 Hz, 1H), 8.58 (dd, J=8.7, 1.6 Hz, 1H), 8.47 (d, J=5.1 Hz, 1H), 7.85-7.73 (m, 2H), 7.70-7.58 (m, 2H), 7.51 (d, J=7.9 Hz, 1H), 4.39-4.19 (m, 1H), 4.11-4.03 (m, 2H), 3.59 (dd, J=14.3, 5.4 Hz, 1H), 3.50-3.39 (m, 1H), 1.34 (s, 9H), 1.12 (t, J=7.1 Hz, 3H).

### Example 9: Synthesis of Compound V—Method A

### Activation of Compound VI-2 to Compound VI-3

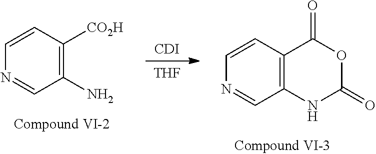 [SMILES: Nc1cnccc1C(=O)O ; "CDI" ; O=c1[nH]c2cnccc2c(=O)o1 ; "THF"]

A reactor was charged with Compound VI-2 (1.00 equiv, scaling factor), 1,1′-carbonyldiimidazole (1.5 equiv) and tetrahydrofuran (10.0 volumes). The reactor contents were agitated at about 20° C. for about 18 hours, the resulting slurry was filtered. The cake was washed with about 10.0 volumes of tetrahydrofuran followed by a re-slurry in tetrahydrofuran (10 volumes) at about 20° C. for about 24 hours. The slurry was filtered, rinsed with about 10 volumes of tetrahydrofuran, deliquored and dried under reduced pressure at about 40° C. for about 72 hours to afford Compound VI-3. ¹H NMR (400 MHz, DMSO-d₆) δ 8.70 (s, 1H), 8.48 (d, J=5.2 Hz, 1H), 7.87 (d, J=5.2 Hz, 1H).

### Esterification of Compound VI-3

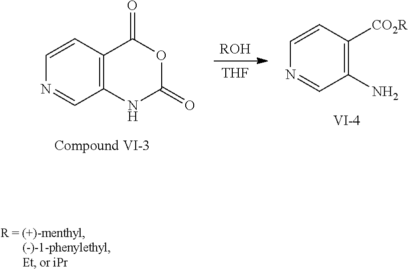

A reactor was charged with Compound VI-3 (1 equiv, scaling factor), respective alcohol ((+)-menthol, (−)-phenylethanol, ethanol or isopropanol) (1.5 equiv) and tetrahydrofuran (10.0 volumes) and contents were agitated at about 50° C. for about 24 hours. The reactor contents were concentrated to dryness under reduced pressure and purified by column chromatography to afford the corresponding esterification products (a compound of Formula VI-4, wherein R is (+)-menthyl, (−)-1-phenylethyl, Et, or iPr).

A compound of Formula VI-4, wherein R is (+)-menthyl: ¹H NMR (400 MHz, CDCl₃) δ 8.19 (s, 1H), 7.93 (d, J=5.2 Hz, 1H), 7.59 (d, J=5.2 Hz, 1H), 5.66 (s, 2H), 4.92 (td, J=10.9, 4.4 Hz, 1H), 2.21-2.05 (m, 1H), 2.02-1.87 (m, 1H), 1.80-1.48 (m, 5H), 1.28-1.04 (m, 3H), 1.02-0.83 (m, 10H), 0.85-0.76 (m, 5H).

A compound of Formula VI-4, wherein R is (−)-1-phenylethyl: ¹H NMR (400 MHz, CDCl₃) δ 8.17 (s, 1H), 7.93 (d, J=5.2 Hz, 1H), 7.67 (d, J=5.2 Hz, 1H), 7.46-7.23 (m, 7H), 6.09 (q, J=6.6 Hz, 1H), 5.64 (s, 1H), 1.67 (d, J=6.6 Hz, 4H).

A compound of Formula VI-4, wherein R is Et: ¹H NMR (400 MHz, CDCl3) δ 8.19 (s, 1H), 7.93 (d, J=5.2 Hz, 1H), 7.63-7.57 (m, 1H), 5.64 (s, 3H), 4.37 (q, J=7.1 Hz, 2H), 1.40 (t, J=7.1 Hz, 3H).

A compound of Formula VI-4, wherein R is iPr: ¹H NMR (400 MHz, CDCl₃) δ 8.18 (s, 1H), 7.92 (d, J=5.2 Hz, 1H), 7.59 (d, J=5.2 Hz, 1H), 5.66 (s, 2H), 5.24 (hept, J=6.3 Hz, 1H), 1.37 (d, J=6.3 Hz, 6H).

### Activation of the Amine to Phenyl Carbamates

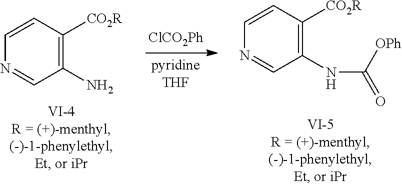

A reactor was charged with a compound of Formula VI-4 (wherein R is (+)-menthyl, (−)-1-phenylethyl, Et, or iPr) (1.0 equiv, scaling factor), pyridine (1.1 equiv) and tetrahydrofuran (10 volumes), the contents were agitated at about 20° C. Phenyl chloroformate (1.1 equiv) was charged dropwise over about 15 minutes then the temperature was adjusted to about 40° C. for about 18 hours. About 10 volumes of dichloromethane and about 10 volumes of water were charged to the reactor. The aqueous layer was discharged then the organics were washed again with about 10 volumes of water. The organic stream was then concentrated to dryness under reduced pressure and purified via column chromatography to afford the corresponding phenyl carbamates (a compound of Formula VI-5, wherein R is (+)-menthyl, (−)-1-phenylethyl, Et, or iPr).

A compound of Formula VI-5, wherein R is (+)-menthyl: ¹H NMR (400 MHz, CDCl3) δ 10.58 (s, 1H), 9.81 (s, 1H), 8.43 (d, J=5.1 Hz, 1H), 7.84-7.78 (m, 1H), 7.41 (dd, J=8.4, 7.4 Hz, 2H), 7.30-7.18 (m, 4H), 5.00 (td, J=10.9, 4.4 Hz, 1H), 2.20-2.10 (m, 1H), 1.94 (pd, J=7.0, 2.7 Hz, 1H), 1.83-1.71 (m, 2H), 1.68-1.50 (m, 3H), 1.28-1.08 (m, 3H), 0.96 (dd, J=6.8, 4.6 Hz, 7H), 0.82 (d, J=7.0 Hz, 3H).

A compound of Formula VI-5, wherein R is (−)-1-phenylethyl: ¹H NMR (400 MHz, CDCl₃) δ 10.45 (s, 1H), 9.80 (s, 1H), 8.44 (d, J=5.1 Hz, 1H), 7.90 (d, J=5.1 Hz, 1H), 7.49-7.31 (m, 7H), 7.30-7.16 (m, 3H), 6.16 (q, J=6.6 Hz, 1H), 1.74 (d, J=6.6 Hz, 3H).

A compound of Formula VI-5, wherein R is Et: ¹H NMR (400 MHz, CDCl₃) δ 10.50 (s, 1H), 9.81 (s, 1H), 8.43 (d, J=5.1 Hz, 1H), 7.83 (d, J=5.2 Hz, 1H), 7.46-7.36 (m, 2H), 7.30-7.18 (m, 4H), 4.47 (q, J=7.1 Hz, 2H), 1.46 (t, J=7.1 Hz, 3H).

A compound of Formula VI-5, wherein R is iPr: ¹H NMR (400 MHz, CDCl₃) δ 10.55 (s, 1H), 9.80 (s, 1H), 8.43 (d, J=5.1 Hz, 1H), 7.81 (d, J=5.0 Hz, 1H), 7.46-7.37 (m, 4H), 7.24-7.18 (m, 5H), 5.32 (hept, J=6.3 Hz, 1H), 1.47-1.40 (m, 11H).

### Conversion of Phenyl Carbamates to Ureas

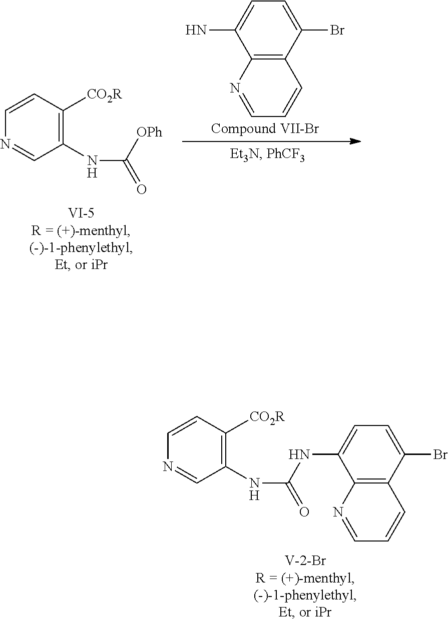

General procedure: A reactor was charged with respective phenyl carbamate (a compound of Formula VI-5, wherein R is (+)-menthyl, (−)-1-phenylethyl, Et, or iPr) (1.0 equiv, scaling factor), Compound VII-Br (1.2 equiv), triethylamine (2 equiv) and trifluorotoluene (10 volumes). The reactor contents were agitated at about 70° C. for about 24 hours. The resulting slurry was filtered then the wet cake was rinsed with about 10 volumes of trifluorotoluene followed by about 10 volumes of tert-butyl methyl ether. The solids were dried under reduced pressure at about 40° C. for about 4 hours to afford the corresponding ureas (e.g., a compound of Formula V-2-Br, wherein R is (+)-menthyl, (−)-1-phenylethyl, Et, or iPr).

A compound of Formula V-2-Br, wherein R is (+)-menthyl: ¹H NMR (400 MHz, DMSO) δ 10.15 (s, 1H), 10.10 (s, 1H), 9.03 (dd, J=4.2, 1.6 Hz, 1H), 8.84 (s, 1H), 8.53 (dd, J=8.6, 1.6 Hz, 1H), 8.44 (dd, J=6.8, 1.8 Hz, 2H), 7.91 (d, J=8.5 Hz, 1H), 7.82 (dd, J=8.6, 4.2 Hz, 1H), 7.63 (d, J=5.0 Hz, 1H), 4.81 (td, J=10.9, 4.4 Hz, 1H), 2.02 (d, J=12.0 Hz, 1H), 1.87 (qt, J=6.8, 3.7 Hz, 1H), 1.64-1.55 (m, 2H), 1.48-1.37 (m, 2H), 1.11-0.92 (m, 2H), 0.80 (d, J=7.0 Hz, 3H), 0.71 (d, J=6.9 Hz, 3H), 0.64 (d, J=6.5 Hz, 3H).

A compound of Formula V-2-Br, wherein R is (−)-1-phenylethyl: ¹H NMR (400 MHz, DMSO) δ 10.18 (s, 1H), 10.15 (s, 1H), 9.02 (dd, J=4.2, 1.6 Hz, 1H), 8.92 (s, 1H), 8.53 (dd, J=8.6, 1.6 Hz, 1H), 8.47-8.38 (m, 2H), 7.91 (d, J=8.5 Hz, 1H), 7.82 (dd, J=8.6, 4.2 Hz, 1H), 7.73 (d, J=5.0 Hz, 1H), 7.46-7.39 (m, 2H), 7.34-7.14 (m, 3H), 6.02 (q, J=6.5 Hz, 1H), 1.55 (d, J=6.6 Hz, 3H).

A compound of Formula V-2-Br, wherein R is Et: ¹H NMR (400 MHz, DMSO) δ 10.23 (s, 1H), 10.12 (s, 1H), 9.03 (dd, J=4.2, 1.6 Hz, 1H), 8.96 (s, 1H), 8.54 (dd, J=8.6, 1.6 Hz, 1H), 8.49-8.39 (m, 2H), 7.93 (d, J=8.5 Hz, 1H), 7.82 (dd, J=8.5, 4.2 Hz, 1H), 7.67 (d, J=5.0 Hz, 1H), 4.31 (q, J=7.1 Hz, 2H), 1.26 (t, J=7.1 Hz, 3H).

A compound of Formula V-2-Br, wherein R is iPr: ¹H NMR (400 MHz, DMSO) δ 10.20 (s, 1H), 10.13 (s, 1H), 9.04 (dd, J=4.2, 1.6 Hz, 1H), 8.91 (s, 1H), 8.53 (dd, J=8.5, 1.6 Hz, 1H), 8.50-8.38 (m, 2H), 7.93 (d, J=8.5 Hz, 1H), 7.82 (dd, J=8.6, 4.2 Hz, 1H), 7.65 (d, J=5.0 Hz, 1H), 5.13 (p, J=6.3 Hz, 1H), 1.27 (d, J=6.2 Hz, 6H).

### Cyclization of a Compound of Formula V-2-Br to Compound V-1-Br

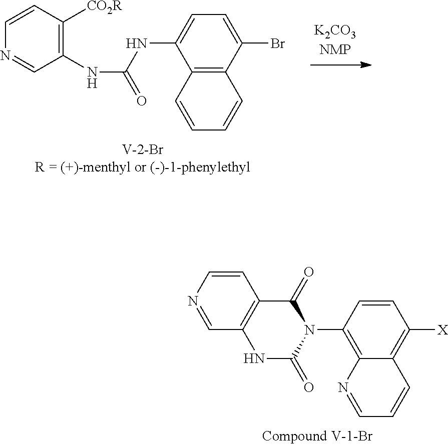

A reactor was charged with a compound of Formula V-2-Br (wherein R is (+)-menthyl or (−)-1-phenylethyl) (1.0 equiv, scaling factor), potassium carbonate (2 equiv) and 1-methyl-2-pyrrolidinone (5 volumes). The reactor contents were agitated at about 20° C. for about 46 hours. The potassium carbonate was then filtered off and about 10 volumes of about 5 wt % acetic acid in water was charged to the reactor. The reactor contents were agitated at about 20° C. for about 2 hours then the resulting slurry was filtered. The filter cake was then washed with about 10 volumes of water, about 10 volumes of isopropanol and about 10 volumes of tert-butyl methyl ether to afford Compound V-1-Br.

Compound V-1-Br prepared from a compound of Formula V-2-Br wherein R is (+)-menthyl: ¹H NMR (400 MHz, DMSO) δ 11.99 (s, 1H), 8.92 (dd, 1H), 8.73 (s, 1H), 8.61 (dd, J=8.5, 1.4 Hz, 1H), 8.49 (d, J=5.1 Hz, 1H), 8.16 (d, 1H), 7.85 (d, J=7.9 Hz, 2H), 7.77 (dd, J=8.6, 4.2 Hz, 1H).

Compound V-1-Br prepared from a compound of Formula V-2-Br wherein R is (−)-1-phenylethyl: ¹H NMR (400 MHz, DMSO) δ 11.99 (s, 1H), 8.92 (dd, J=4.2, 1.6 Hz, 1H), 8.72 (s, 1H), 8.61 (dd, J=8.6, 1.6 Hz, 1H), 8.49 (d, J=5.0 Hz, 1H), 8.17 (d, J=7.9 Hz, 1H), 7.89-7.81 (m, 2H), 7.77 (dd, J=8.6, 4.2 Hz, 1H).

### Cyclization of a Compound of Formula V-2-Br to Compound V-1-Br

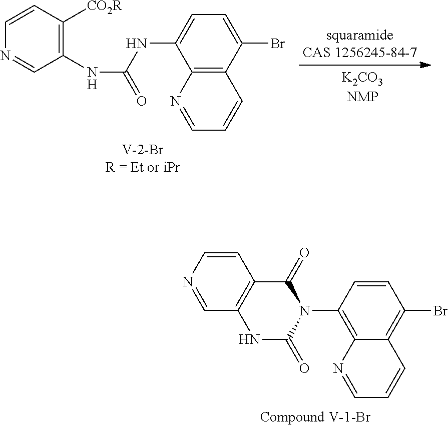

A reactor was charged with potassium carbonate (0.04 equiv), organocatalyst CAS 1256245-84-7 (0.06 equiv) and 1-methyl-2-pyrrolidinone (5 volumes), the reactor contents were agitated at about 20° C. for about 30 minutes. A compound of Formula V-2-Br (wherein R is Et or iPr) (1.0 equiv, scaling factor) was then charged to the reactor and the contents were agitated at about 20° C. for about 48 hours. The potassium carbonate was then filtered off and about 10 volumes of about 5 wt % acetic acid in water was charged to the reactor. The reactor contents were agitated at about 20° C. for about 2 hours then the resulting slurry was filtered. The filter cake was then washed with about 10 volumes of water, about 10 volumes of isopropanol and about 10 volumes of tert-butyl methyl ether to afford Compound V-1-Br.

Compound V-1-Br prepared from a compound of Formula V-2-Br wherein R is Et: ¹H NMR (400 MHz, DMSO) δ 11.99 (s, 1H), 8.92 (dd, J=4.2, 1.6 Hz, 1H), 8.73 (s, 1H), 8.61 (dd, J=8.6, 1.6 Hz, 1H), 8.49 (d, J=5.1 Hz, 1H), 8.17 (d, J=8.0 Hz, 1H), 7.89-7.79 (m, 2H), 7.77 (dd, J=8.6, 4.2 Hz, 1H).

Compound V-1-Br prepared from a compound of Formula V-2-Br wherein R is iPr: ¹H NMR (400 MHz, DMSO) δ 11.99 (s, 1H), 8.92 (dd, J=4.3, 1.6 Hz, 1H), 8.72 (s, 1H), 8.61 (dd, J=8.7, 1.6 Hz, 1H), 8.49 (d, J=5.1 Hz, 1H), 8.17 (d, J=7.9 Hz, 1H), 7.87-7.82 (m, 2H), 7.77 (dd, J=8.6, 4.2 Hz, 1H).

### Cyclization of a Compound of Formula V-3 to a Compound of Formula V-1

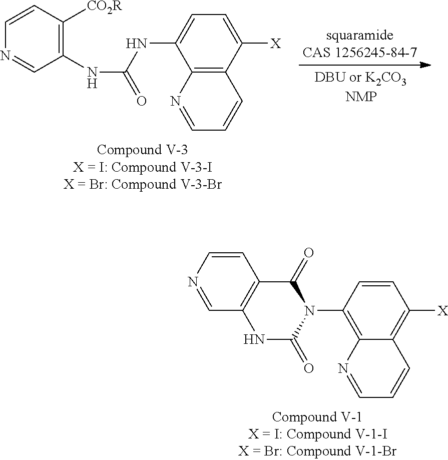

Compound V-3-I to Compound V-1-I. A reactor was charged with the squaramide organocatalyst (3-[[3,5-Bis(trifluoromethyl)phenyl]amino]-4-[[(8a,9S)-6′-Methoxycinchonan-9-yl]amino]-3-Cyclobutene-1,2-dione, CAS 1256245-84-7) (0.06 equiv), 1,8-diazabicyclo[5.4.0]undec-7-ene (0.04 equiv) and 1-methyl-2-pyrrolidinone (5 volumes). The reactor contents were agitated at about 20° C. for about 30 minutes. Compound V-3-I (1.0 equiv, scaling factor) was charged to the reactor and the contents were agitated at about 20° C. for about 25 hours. About 3 volumes of water were charged and then the reactor contents were agitated at about 20° C. for about 2 hours. The resulting slurry was filtered then the wet cake was rinsed with about 2 volumes of isopropanol followed by about 5 volumes of tert-butyl methyl ether to afford Compound V-1-I. ¹H NMR (400 MHz, DMSO) δ 11.98 (s, 1H), 8.72 (s, 1H), 8.48 (d, J=5.1 Hz, 1H), 8.46 (dd, J=8.5, 1.6 Hz, 1H), 8.40 (d, J=7.8 Hz, 1H), 7.83 (dd, J=5.1, 0.8 Hz, 1H), 7.72 (dd, J=8.6, 4.2 Hz, 1H), 7.67 (d, J=7.7 Hz, 1H).

Compound V-3-Br to Compound V-1-Br. A reactor was charged with a squaramide organocatalyst (3-[[3,5-bis(trifluoromethyl)phenyl]amino]-4-[[(8a,9S)-6′-Methoxycinchonan-9-yl]amino]-3-Cyclobutene-1,2-dione, CAS 1256245-84-7) (0.06 equiv), potassium carbonate (0.04 equiv) and 1-methyl-2-pyrrolidinone (5 volumes). The reactor contents were agitated at about 20° C. for about 30 minutes. Compound V-3-Br (1 equiv, scaling factor) was charged to the reactor and the contents were agitated at about 20° C. for about 25 hours. About 3 volumes of water were charged and then the reactor contents were agitated at about 20° C. for about 2 hours. The resulting slurry was filtered then the wet cake was rinsed with about 2 volumes of isopropanol followed by about 5 volumes of tert-butyl methyl ether to afford Compound V-1-Br. ¹H NMR (400 MHz, DMSO) δ 12.00 (s, 1H), 8.92 (dd, J=4.2, 1.6 Hz, 1H), 8.73 (s, 1H), 8.62 (dd, J=8.6, 1.6 Hz, 1H), 8.49 (d, J=5.1 Hz, 1H), 8.17 (d, J=8.0 Hz, 1H), 7.89-7.80 (m, 2H), 7.77 (dd, J=8.6, 4.2 Hz, 1H).

### Methylation of a Compound of Formula V-1 to a Compound of Formula V

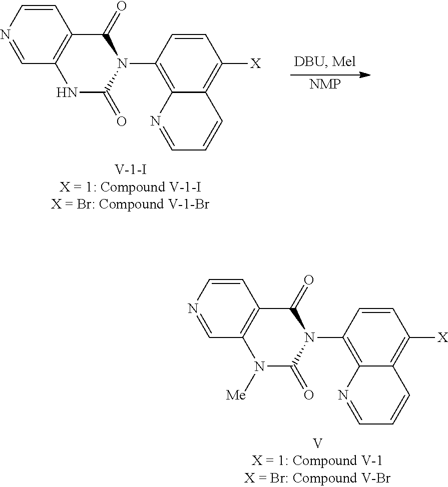

A reactor was charged with a compound of Formula V-1 (1.0 equiv, scaling factor), 1,8-diazabicyclo[5.4.0]undec-7-ene (1.6 equiv), iodomethane (1.6 equiv) and 1-methyl-2-pyrrolidinone (10 volumes). The reactor contents were agitated at about 20° C. for about 22 hours. About 2 volumes of water was charged to the reactor and the contents were agitated at about 20° C. for about 2 hours. The resulting slurry was filtered then the wet cake was rinsed with about 2 volumes of isopropanol followed by about 2 volumes of tert-butyl methyl ether to afford a compound of Formula V.

Compound V-I: ¹H NMR (400 MHz, DMSO) δ 9.05 (s, 1H), 8.84 (dd, J=4.3, 1.5 Hz, 1H), 8.61 (d, J=4.9 Hz, 1H), 8.46 (dd, J=8.6, 1.6 Hz, 1H), 8.41 (d, J=7.8 Hz, 1H), 7.92 (d, J=5.0 Hz, 1H), 7.72 (dd, J=8.6, 4.2 Hz, 1H), 7.66 (d, J=7.8 Hz, 1H), 3.66 (s, 3H).

Compound V-Br: ¹H NMR (400 MHz, DMSO) δ 9.05 (s, 1H), 8.91 (dd, J=4.3, 1.6 Hz, 1H), 8.65-8.58 (m, 2H), 8.17 (d, J=8.0 Hz, 1H), 7.93 (dd, J=4.9, 0.7 Hz, 1H), 7.83 (d, J=7.9 Hz, 1H), 7.77 (dd, J=8.6, 4.2 Hz, 1H), 3.66 (s, 3H).

### Example 10: Synthesis of Compound V—Method B

Conversion of Pyridine-3,4-dicarboxylic Acid to Threonine Esters

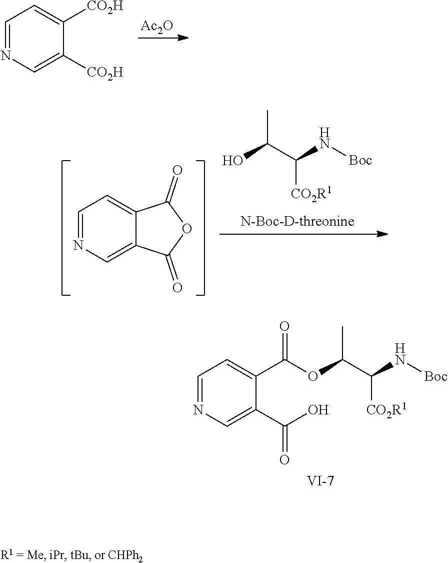

Procedure for a compound of Formula VI-7 wherein R¹ is Me and a compound of Formula VI-7 wherein R¹ is iPr: A mixture of pyridine-3,4-dicarboxylic acid (1.00 equiv, scaling factor) and acetic anhydride (1.8-1.9 equiv) was heated at about 100° C. for about 2 hours. The mixture was cooled to about 20° C. and transferred to a separate mixture of N-Boc-D-threonine (2.50 equiv) in dichloromethane (3.8 volumes) and rinsed forward with dichloromethane (2.3 volumes). The reaction contents were aged overnight at about 40° C. Reaction contents were cooled to about 20° C., absorbed onto silica, concentrate to dryness in vacuo, and purified by chromatography to afford a compound of Formula VI-7 (wherein R¹ is Me or iPr).

Procedure for a compound of Formula VI-7 wherein R¹ is tBu: A mixture of pyridine-3,4-dicarboxylic acid (1.00 equiv, scaling factor) and acetic anhydride (1.9 equiv) was heated at about 100° C. for about 2 hours. The mixture was cooled to about 20° C. and transferred to a separate mixture of N-Boc-D-threonine (2.0 equiv) in toluene (6.7 volumes). The reaction contents were aged overnight at about 20° C. Reaction contents were concentrated to dryness in vacuo, and purified by chromatography to afford a compound of Formula VI-7 (wherein R¹ is tBu). ¹H NMR (300 MHz, CDCl₃) δ 9.18 (s, 1H), 8.86 (d, J=5.1 Hz, 1H), 7.44 (d, J=5.1 Hz, 1H), 5.83-5.53 (m, 2H), 5.39 (d, J=9.5 Hz, 1H), 4.43 (dd, J=9.9, 2.5 Hz, 1H), 1.49-1.45 (m, 21H).

Procedure for a compound of Formula VI-7 wherein R¹ is CHPh₂: A mixture of pyridine-3,4-dicarboxylic acid (1.2 equiv) and acetic anhydride (2.4 equiv) was heated at about 110° C. for about 3 hours. The mixture was cooled to about 20° C. and the solvent removed under vacuum. A separate mixture of N-Boc-D-threonine (1.0 equiv, scaling factor), 4-(dimethylamino)pyridine (0.05 equiv), and triethylamine (1.5 equiv) in tetrahydrofuran (5.4 volumes) was precooled to about 0° C. and charged with a solution of the anhydride (furo[3,4-c]pyridine-1,3-dione) in tetrahydrofuran (1.1 volumes). The mixture was stirred at 50° C. for about 12 hours, then cooled to about 20° C., diluted with ethyl acetate (13.5 volumes), washed three times with 10% aqueous citric acid solution (5.4 volumes per wash), washed two times with water (8.1 volumes per wash), and washed with saturated aqueous sodium chloride solution (5.4 volumes). The organic layer was dried over sodium sulfate, filtered, concentrated, and purified by chromatography to afford a compound of Formula VI-7 wherein R¹ is CHPh₂. ¹H NMR (300 MHz, CDCl₃) δ 9.10 (s, 1H), 8.62 (d, J=5.0 Hz, 1H), 7.36-7.28 (m, 10H), 6.91 (s, 1H), 6.61 (d, J=5.0 Hz, 1H), 5.56-5.45 (m, 2H), 4.70 (dd, J=9.7, 2.5 Hz, 1H), 2.11 (s, 1H), 1.46 (d, J=6.4 Hz, 3H), 1.43 (s, 9H).

### Curtius Rearrangement of Threonine Esters

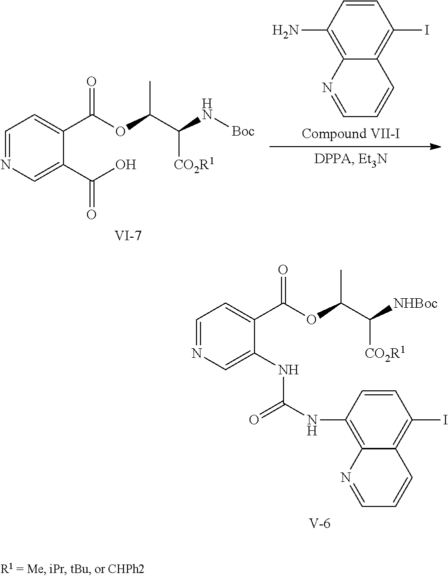

General procedure: A mixture of Compound VII-I (1.00 equiv, scaling factor) and threonine ester (a compound of Formula VI-7, wherein R¹ is Me, iPr, tBu, or CHPh₂) (1.1-1.2 equiv), trifluorotoluene (10-20 volumes), and triethylamine (1.5-2.5 equiv) was heated to about 65° C. Diphenylphophoryl azide (1.05-1.30 equiv) in trifluorotoluene (0.7-1.2 volumes) was added and the contents were aged for at least 2 hours at about 60° C. The resulting mixture was cooled to about 20° C., absorbed onto silica, concentrated to dryness in vacuo, and purified by chromatography to afford a compound of Formula V-6 (wherein R¹ is Me, iPr, tBu, or CHPh₂).

A compound of Formula V-6, wherein R is Me: ¹H NMR (400 MHz, DMSO-d6) δ 10.32 (s, 1H), 10.12 (s, 1H), 9.05 (s, 1H), 8.96 (dd, J=4.2, 1.6 Hz, 1H), 8.41 (d, J=5.1 Hz, 1H), 8.37 (dd, J=8.5, 1.6 Hz, 1H), 8.33 (d, J=8.4 Hz, 1H), 8.14 (d, J=8.4 Hz, 1H), 7.89 (d, J=5.1 Hz, 1H), 7.77 (dd, J=8.6, 4.2 Hz, 1H), 7.64 (d, J=9.0 Hz, 1H), 5.51-5.36 (m, 1H), 4.49 (dd, J=9.1, 3.7 Hz, 1H), 3.61 (s, 3H), 1.39 (s, 9H), 1.31 (d, J=6.5 Hz, 3H).

A compound of Formula V-6, wherein R is iPr: ¹H NMR (400 MHz, DMSO-d6) δ 10.36 (s, 1H), 10.12 (s, 1H), 9.08 (s, 1H), 8.97 (dd, J=4.3, 1.6 Hz, 1H), 8.41 (d, J=5.1 Hz, 1H), 8.37 (dd, J=8.6, 1.6 Hz, 1H), 8.33 (d, J=8.4 Hz, 1H), 8.14 (d, J=8.4 Hz, 1H), 7.92 (d, J=5.1 Hz, 1H), 7.77 (dd, J=8.5, 4.2 Hz, 1H), 7.57 (d, J=9.1 Hz, 1H), 5.50 (dt, J=10.1, 5.0 Hz, 1H), 4.86 (hept, J=6.3 Hz, 1H), 4.42 (dd, J=9.0, 3.7 Hz, 1H), 1.38 (s, 9H), 1.31 (d, J=6.4 Hz, 3H), 1.11 (d, J=6.2 Hz, 3H), 1.02 (d, J=6.2 Hz, 3H).

A compound of Formula V-6, wherein R is tBu: ¹H NMR (400 MHz, CDCl₃) δ 10.11 (s, 1H), 9.51 (s, 1H), 8.79 (dd, J=4.3, 1.6 Hz, 1H), 8.39 (dtd, J=6.8, 3.3, 1.8 Hz, 4H), 8.09 (d, J=8.3 Hz, 1H), 7.73 (dd, J=5.1, 0.7 Hz, 1H), 7.55 (dd, J=8.5, 4.2 Hz, 1H), 5.61 (qd, J=6.4, 3.4 Hz, 1H), 5.41-5.32 (m, 1H), 4.54 (d, J=9.1 Hz, 1H), 1.46 (d, J=1.1 Hz, 3H), 1.43-1.42 (m, 18H).

A compound of Formula V-6 wherein R is CHPh₂: ¹H NMR (400 MHz, CDCl₃) δ 9.93 (s, 1H), 9.46 (d, J=7.1 Hz, 1H), 8.80 (dd, J=4.3, 1.6 Hz, 1H), 8.44-8.38 (m, 2H), 8.22 (d, J=5.1 Hz, 1H), 8.09 (d, J=8.3 Hz, 1H), 7.55 (dd, J=8.5, 4.2 Hz, 1H), 7.33 (dd, J=5.2, 0.7 Hz, 1H), 7.30-7.26 (m, 5H), 7.21-7.16 (m, 2H), 7.07 (d, J=7.8 Hz, 2H), 6.92 (s, 2H), 5.64 (qd, J=6.4, 3.0 Hz, 1H), 5.46 (d, J=9.1 Hz, 1H), 4.81 (dd, J=9.2, 3.0 Hz, 1H), 1.71 (s, 1H), 1.47 (s, 3H), 1.46 (s, 9H).

### Cyclization of Threonine Esters

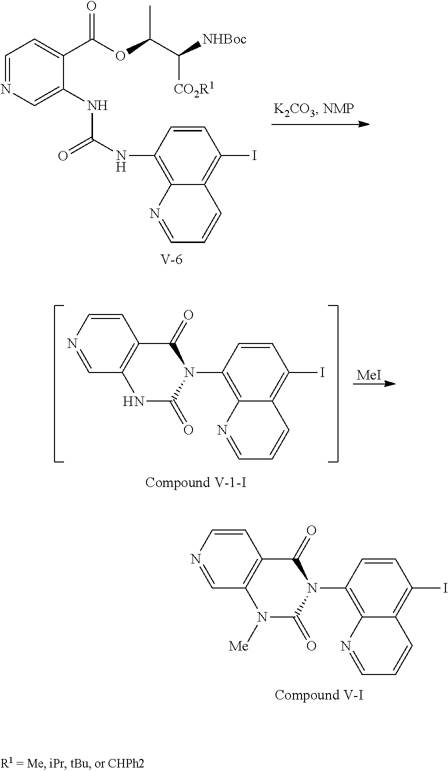

General procedure: A suitable reaction flask was charged with threonine ester (a compound of Formula V-6, wherein R¹ is Me, iPr, tBu, or CHPh₂) (1.00 equiv, scaling factor) and potassium carbonate (2.00 equiv). The reaction flask was degassed with N₂ 3 times, charged with N-methyl-2-pyrrolidone (6 volumes), and aged at about 20° C. for about 1 h. Reaction mixture was charge iodomethane (1.20 equiv) in NMP (0.5 volumes) and age overnight at about 20° C. Charge reaction contents with water (4.0 volumes) and filter slurry to afford Compound of V-I. ¹H NMR (400 MHz, DMSO-d₆) δ 9.05 (s, 1H), 8.84 (dd, J=4.2, 1.5 Hz, 1H), 8.60 (d, J=5.0 Hz, 1H), 8.46 (dd, J=8.6, 1.6 Hz, 1H), 8.40 (d, J=7.8 Hz, 1H), 7.92 (d, J=4.9 Hz, 1H), 7.72 (dd, J=8.6, 4.2 Hz, 1H), 7.66 (d, J=7.8 Hz, 1H), 3.65 (s, 3H).

### Example 11: Synthesis of Compound Va (Synthesis of Compound V—Method C)

### Amide Coupling of Isonicotinic Acid and Compound VII-Br to Compound XI HCl Salt

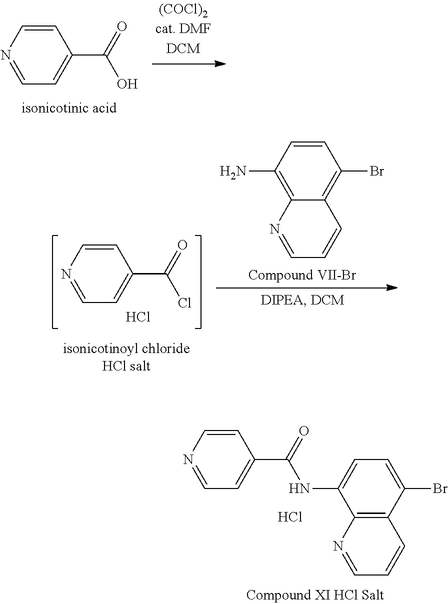

A reaction flask was charged with isonicotinic acid (1.00 equiv, scaling factor), dichloromethane (15.0 volumes), and N,N-dimethylformamide (0.10 equiv). The reaction mixture was agitated at about 0° C. and charged with oxalyl chloride (1.20 equiv) over several minutes. After complete addition, the reaction mixture was allowed to warm up overnight to about 20° C. Reaction mixture was concentrated under reduced pressure to about 13.0 volumes, charged with dichloromethane (10.0 volumes), and cooled to about 0° C. Reaction mixture was charged with N,N-diisopropylethylamine (2.00 equiv) and Compound VII-Br (1.10 equiv). Resulting slurry was allowed to warm up overnight to about 20° C. The slurry was filtered, washed twice with dichloromethane (3.0 volumes), and dried under reduced pressure at about 60° C. to afford Compound XI HCl salt. 11H NMR (400 MHz, DMSO-d₆) δ 10.88 (s, 1H), 9.06 (dd, J=4.2, 1.3 Hz, 1H), 8.95 (d, J=6.2 Hz, 2H), 8.65-8.54 (m, 2H), 8.12 (d, J=6.2 Hz, 2H), 8.05 (d, J=8.4 Hz, 1H), 7.86 (dd, J=8.5, 4.2 Hz, 1H), 5.52 (s, 1H).

### Free Basing of Compound XI HCl Salt to Compound XI

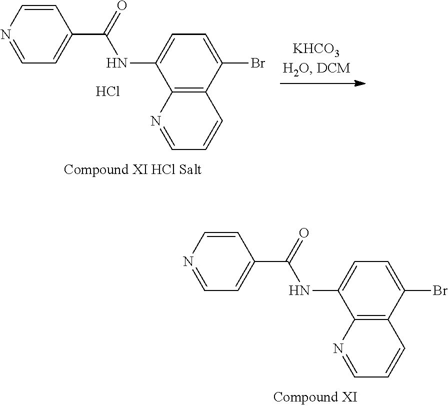 [SMILES: O=C(Nc1ccc(Br)c2cccnc12)c1ccncc1 ; Cl ; O=C(Nc1ccc(Br)c2cccnc12)c1ccncc1]

A reaction vial was charged with Compound XI HCl salt (1.00 equiv, scaling factor) and dichloromethane (40.0 volumes). The vial was charged with potassium bicarbonate (1.20 equiv) in water (25.0 volumes) and the resulting reaction mixture was agitated at about 20° C. The organics were washed with 5% brine (25.0 volumes), dried over MgSO₄, and concentrated under reduced pressure to afford Compound XI. ¹H NMR (400 MHz, DMSO-d₆) δ 10.78 (s, 1H), 9.06 (dd, J=4.2, 1.3 Hz, 1H), 8.91-8.82 (m, 2H), 8.62 (d, J=8.4 Hz, 1H), 8.58 (dd, J=8.5, 1.3 Hz, 1H), 8.04 (d, J=8.4 Hz, 1H), 7.98-7.91 (m, 2H), 7.85 (dd, J=8.5, 4.2 Hz, 1H).

### C—H Activation of Compound XI to Compound V-1a-Br

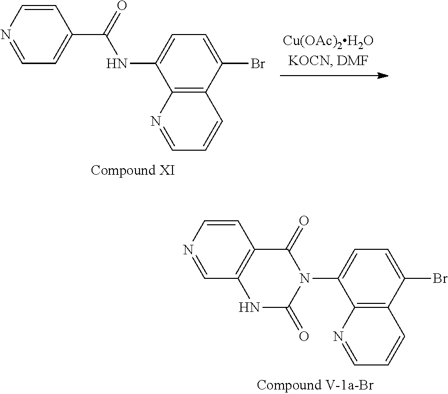 [SMILES: O=C(Nc1ccc(Br)c2cccnc12)c1ccncc1 ; O=c1[nH]c2cnccc2c(=O)n1-c1ccc(Br)c2cccnc12]

A reaction vial was charged with Compound XI (1.00 equiv, scaling factor), copper(II) acetate monohydrate (1.50 equiv), potassium cyanate (3.50 equiv), and N,N-dimethylformamide (20.0 volumes). The contents were agitated overnight at about 80° C. The reaction mixture was cooled to about 20° C., filtered through celite, and concentrated under reduced pressure. The crude was purified by column chromatography to afford Compound V-1a-Br. ¹H NMR (400 MHz, DMSO-d₆) δ 11.99 (s, 1H), 8.92 (dd, J=4.1, 1.3 Hz, 1H), 8.72 (s, 1H), 8.61 (dd, J=8.6, 1.4 Hz, 1H), 8.49 (d, J=5.1 Hz, 1H), 8.16 (d, J=8.0 Hz, 1H), 7.87-7.81 (m, 2H), 7.77 (dd, J=8.6, 4.2 Hz, 1H).

### Methylation of Compound V-1a-Br to Compound Va-Br

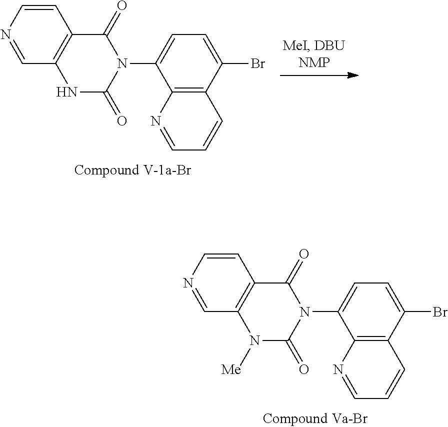 [SMILES: O=c1[nH]c2cnccc2c(=O)n1-c1ccc(Br)c2cccnc12 ; Cn1c(=O)n(-c2ccc(Br)c3cccnc23)c(=O)c2ccncc21]

A mixture of Compound V-1a-Br (1.00 equiv, scaling factor), 1,8-diazabicyclo[5.4.0]undec-7-ene (2.5 equiv) and N-methyl-2-pyrrolidone (6.0 volumes) at about 25° C. was dosed with a solution of iodomethane (2.5 equiv) in N-methyl-2-pyrrolidone (1.0 volume) over about 0.5 hours and the resulting reaction mixture was agitated overnight at about 20° C. Reactor contents were charged with water (2.5 volumes) over about 2 hours. Reactor contents were adjusted to about 10° C. and charged with additional water (2.0 volumes) over about 2 hours. Reactor contents were adjusted to about 0° C., charged with additional water (1.3 volumes) over about 1 hour, and the contents were aged overnight. The slurry was filtered, washed with water (2×2.0 volumes), and the solids were dried under reduced pressure at about 50° C. for about 72 hours to afford Compound Va-Br. ¹H NMR (400 MHz, DMSO-d₆) δ 9.06 (s, 1H), 8.91 (dd, J=4.2, 1.6 Hz, 1H), 8.64-8.58 (m, 2H), 8.17 (d, J=8.0 Hz, 1H), 7.92 (dd, J=4.9, 0.8 Hz, 1H), 7.83 (d, J=8.0 Hz, 1H), 7.77 (dd, J=8.6, 4.2 Hz, 1H), 3.66 (s, 3H).

### Example 12: Synthesis of Compound XX-1 (Model Studies)

### Curtius Rearrangement of Compound XX-2 to Compound XX-4

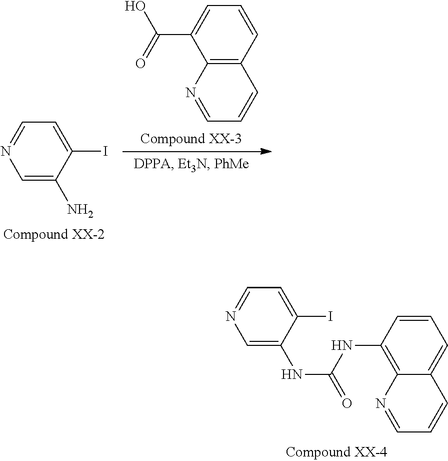 [SMILES: O=C(O)c1cccc2cccnc12 ; Nc1cnccc1I ; O=C(Nc1cnccc1I)Nc1cccc2cccnc12]

Compound XX-2 (1.00 equiv, scaling factor), Compound XX-3 (1.15 equiv), and toluene (16.0 volumes) were charged to a reactor and the contents were agitated at about 30° C. Reactor contents were heated to about 60° C. and a solution of diphenylphosphoryl azide (1.14 equiv) in toluene (2.0 volumes) was charged over about 2 hours. Reaction mixture was aged overnight at about 80° C. Reaction mixture was cooled to about 20° C. and charged with water (33.0 volumes). Resulting slurry was diluted with toluene (27.0 volumes) and heated to about 70° C. Slurry was filtered and dried under reduced pressure at about 40° C. The solids were purified by column chromatography to afford Compound XX-4. ¹H NMR (400 MHz, DMSO-d₆) δ 10.24 (s, 1H), 9.36 (s, 1H), 8.95 (dd, J=4.2, 1.6 Hz, 1H), 8.74 (s, 1H), 8.54 (dd, J=6.7, 2.3 Hz, 1H), 8.41 (dd, J=8.3, 1.5 Hz, 1H), 7.94 (q, J=5.1 Hz, 2H), 7.65 (dd, J=8.3, 4.2 Hz, 1H), 7.57 (q, J=8.2, 7.7 Hz, 2H).

### Carbonylative Cyclization of Compound XX-4 to Compound XX-1

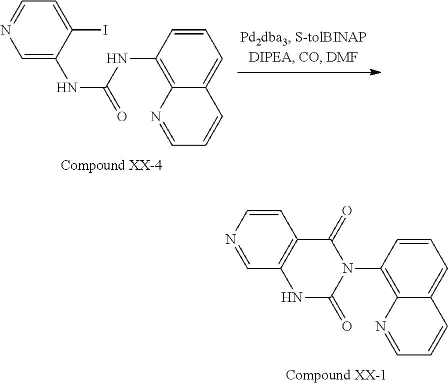 [SMILES: O=C(Nc1cnccc1I)Nc1cccc2cccnc12 ; O=c1[nH]c2cnccc2c(=O)n1-c1cccc2cccnc12]

A reaction vial was charged with Compound XX-4 (1.00 equiv, scaling factor), tris(dibenzylideneacetone)dipalladium(0) (0.075 equiv), (S)-(−)-2,2′-p-tolyl-phosphino)-1,1′-binaphthyl (0.10 equiv), and N,N-dimethylformamide (15.0 volumes). Reaction vial was purged three times with N₂, charged with N,N-diisopropylethylamine (1.50 equiv) and placed in an autoclave. The vial was purged three times with N₂ and then pressurized to about 150 psi with CO gas. The sealed reaction vessel was agitated overnight at about 65° C. The reaction vessel was cooled to about 20° C., vented, and the organics were concentrated under reduced pressure. The crude was purified by column chromatography to afford Compound XX-1. ¹H NMR (400 MHz, DMSO-d₆) δ 11.94 (s, 1H), 8.84 (dd, J=4.1, 1.6 Hz, 1H), 8.72 (s, 1H), 8.54-8.45 (m, 2H), 8.18-8.09 (m, 1H), 7.89-7.80 (m, 2H), 7.80-7.72 (m, 1H), 7.60 (dd, J=8.3, 4.2 Hz, 1H).

### Example 13: Synthesis of Compound XX-5 (Model Studies)

### Conversion of Compound XX-6 to Compound XX-6 Tetrabutylammonium Salt

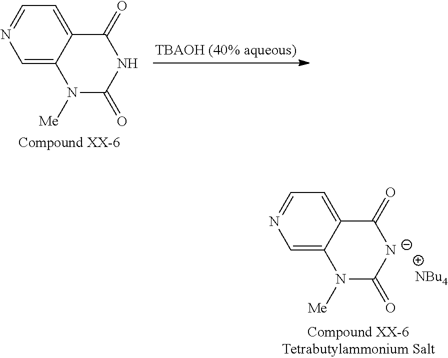 [SMILES: Cn1c(=O)[nH]c(=O)c2ccncc21 ; Cn1c(=O)[n-]c(=O)c2ccncc21]

A vial was charged with 40% aqueous TBAOH solution (1.0 equiv) and Compound XX-6 (1.00 equiv, scaling factor). The reaction mixture was stirred at about 20° C. for about 22 h. The reaction mixture was concentrated to dryness under vacuum, and the resulting solids were dried in vacuum oven at about 50° C. to give Compound XX-6 Tetrabutylammonium Salt. ¹H NMR (300 MHz, DMSO-d₆) δ 8.49 (d, J=0.7 Hz, 1H), 8.17 (d, J=4.8 Hz, 1H), 7.58 (dd, J=4.8, 0.8 Hz, 1H), 3.36 (s, 3H), 3.25-3.03 (m, 8H), 1.56 (p, J=7.5 Hz, 8H), 1.30 (h, J=7.3 Hz, 8H), 0.92 (t, J=7.3 Hz, 12H).

### Atroposelective Chan-Evans-Lam Coupling of Compound XX-6 Tetrabutylammonium Salt and Compound XX-7

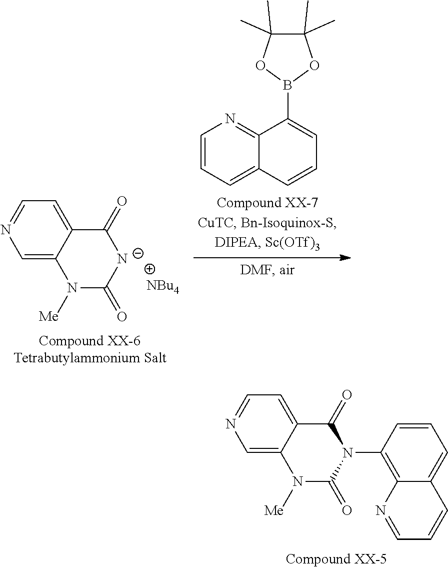

Under and atmosphere of dry nitrogen, CuTC (0.50 equiv), Bn-Isoquinox-S (0.50 equiv) and THF (5.0 volumes) were weighed into a vial. The solution was agitated for about 15 minutes. The solvent was removed under vacuum at a temperature not exceeding 30° C. Sc(OTf)₃ (3.0 equiv) was charged as a THF slurry. The solvent was removed under vacuum at a temperature not exceeding 30° C. Next, Compound XX-6 Tetrabutylammonium Salt (1.00 equiv, scaling factor) and Compound XX-7 (1.0 equiv) were charged to the vial as THF solutions. The solvent was removed under vacuum at a temperature not exceeding 30° C. DIPEA (3 equiv) was added as a solution in DMF (14.1 volumes). The vial was removed from the inert atmosphere and pressurized to 60 psi with air, then agitated at about 70° C. for about 19 h. The reaction mixture was cooled to about 20° C. and concentrated to dryness under vacuum to give Compound XX-5. 1H NMR (400 MHz, DMSO-d₆) δ 9.05 (s, 1H), 8.83 (dd, J=4.1, 1.7 Hz, 1H), 8.61 (d, J=4.9 Hz, 1H), 8.51 (dd, J=8.2, 1.7 Hz, 1H), 8.15 (dd, J=8.1, 1.5 Hz, 1H), 7.93 (dd, J=4.9, 0.8 Hz, 1H), 7.85 (dd, J=7.3, 1.5 Hz, 1H), 7.77 (dd, J=8.2, 7.3 Hz, 1H), 7.62 (dd, J=8.2, 4.1 Hz, 1H), 3.66 (s, 3H).

### Example 13: Synthesis of Compound IX—Method A

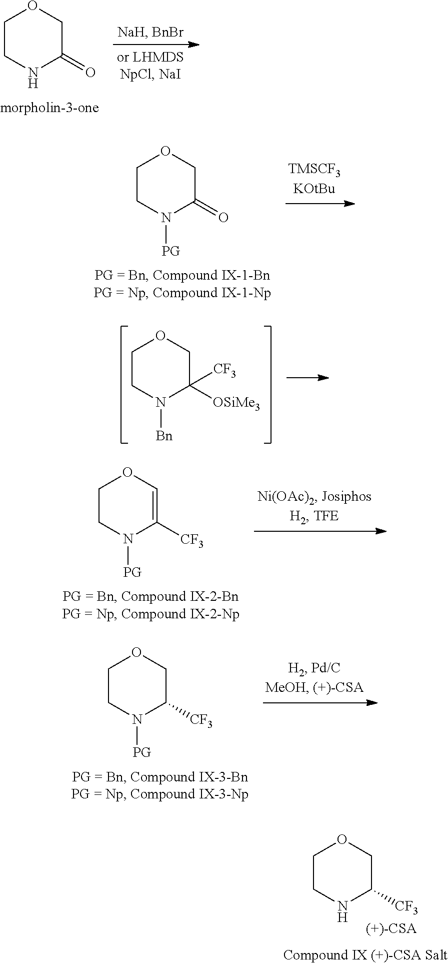

### Benzylation of Morpholin-3-one to Compound IX-1-Bn

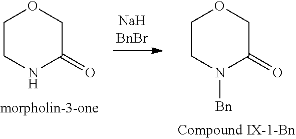 [SMILES: O=C1COCCN1 ; O=C1COCCN1Cc1ccccc1]

Morpholin-3-one (1.00 equiv, scaling factor) and tetrahydrofuran (20.0 volumes) were charged to a reactor and the temperature was adjusted to about 20° C. Sodium hydride (1.13 equiv) was charged in about 4 portions over about 1.5 hours. The mixture was cooled to about 10° C. and bromobenzene (1.07 equiv) was charged over about 45 min. The resulting reaction mixture was then warmed to about 20° C. and agitated for about 20 h. The reaction mixture was quenched with water (20.0 volumes) over about 1 hour and then charged with ethyl acetate (20.0 volumes). The organics were washed with 2% brine (20.0 volumes), dried, filtered, and concentrated to dryness under reduced pressure. The recovered residue was purified by chromatography to afford Compound IX-1-Bn. ¹H NMR (400 MHz, CDCl₃) δ 7.66-6.94 (m, 5H), 4.60 (s, 2H), 4.22 (s, 2H), 3.88-3.70 (m, 2H), 3.32-3.14 (m, 2H).

### (1-Naphthyl)methylation of Morpholin-3-one to Compound IX-1-Np

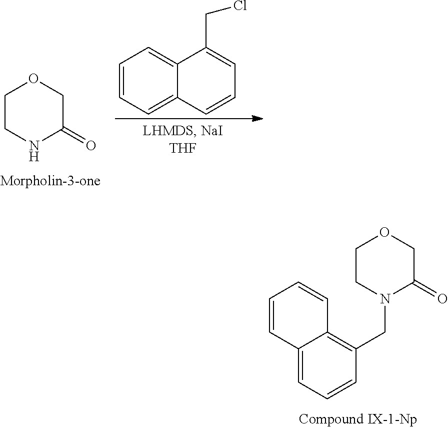 [SMILES: O=C1COCCN1 ; ClCc1cccc2ccccc12 ; O=C1COCCN1Cc1cccc2ccccc12]

Morpholin-3-one (1.00 equiv, scaling factor 1) and sodium iodide (0.20 equiv) were charged to a reactor. The reactor was inerted. Tetrahydrofuran (20.0 volumes) was charged and the temperature was adjusted to about 5° C. Lithium bis(trimethylsilyl)amide (1 M in tetrahydrofuran, 1.13 equiv) was charged over about 5 minutes. The resulting reaction mixture was agitated for about 20 minutes. 1-(Chloromethyl)naphthalene (1.07 equiv) was charged to the reactor. The resulting reaction was agitated for about 1 hour then warmed to about 40° C. and agitated for about 18 hours. The reaction mixture was cooled to about 20° C. and quenched with 10% v/v aqueous hydrochloric acid (12 volumes). Ethyl acetate (30 volumes) was charged to the reactor. The layers were allowed to settle and the layers were separated. The aqueous layer was extracted twice with ethyl acetate (30 volumes). The organic layers were combined and washed with 2% brine (20 volumes). The organic layer was concentrated to dryness under reduced pressure to provide Compound IX-1-Np. ¹H NMR (400 MHz, CDCl₃) δ 8.13-8.11 (m, 1H), 7.89-7.83 (m, 2H), 7.58-7.50 (m, 2H), 7.45-7.37 (m, 2H), 5.12 (s, 2H), 4.28 (s, 2H), 3.78-3.75 (m, 2H), 3.17-3.14 (m, 2H).

### Trifluoromethylation of Compound IX-1-Bn to Compound IX-2-Bn

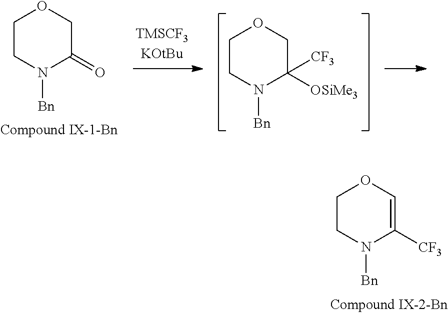

Compound IX-1-Bn (1.00 equiv, scaling factor) in toluene (4.0 volumes) was charged to a reactor and the temperature was adjusted to about 0° C. Reaction mixture was then charged with potassium tert-butoxide (0.20 equiv). (Trifluoromethyl)trimethylsilane (5.00 equiv) was charged in about 30 min and the reaction mixture was agitated at about 0° C. for about 4 hours. The resulting reaction mixture was quenched with water (5.0 volumes) in about 15 min and then the reaction temperature was adjusted to about 20° C. The organics were washed with 2% brine (5.0 volumes) and solvent swapped to tetrahydrofuran (6.0 volumes). The organic stream was cooled to about 0° C. and charged with tetrabutylammonium fluoride (1.00 equiv, 1.0 M solution in tetrahydrofuran). The resulting reaction mixture was agitated at about 20° C. for about 1.5 hours. The reaction mixture was quenched with saturated ammonium chloride (10.0 volumes) and washed with water (10.0 volumes). The organic stream was dried, filtered, and concentrated to dryness under reduced pressure. The recovered residue was purified by column chromatography to afford Compound IX-2-Bn. ¹H NMR (400 MHz, CDCl₃) δ 7.40-7.27 (m, 5H), 6.86 (d, J=1.3 Hz, 1H), 3.92 (s, 2H), 3.91-3.83 (m, 2H), 2.94-2.72 (m, 2H).

### Trifluoromethylation of Compound IX-1-Np to Compound IX-2-Np

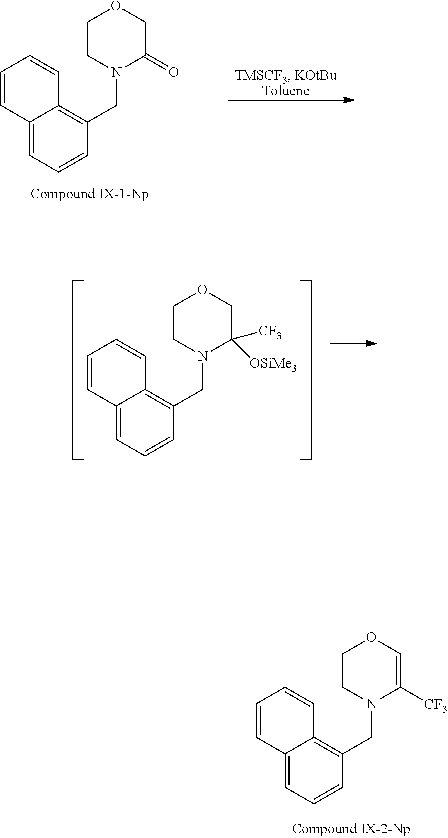

Compound IX-1-Np (1.00 equiv, scaling factor) and potassium tert-butoxide (0.20 equiv) were charged to a reactor. The reactor was inerted. Toluene (7.50 volumes) was charged to the reactor and the temperature was adjusted to about 0° C. (Trifluoromethyl)trimethylsilane (5 equiv) was charged to the reactor over about 30 minutes via syringe. The resulting reaction was held at 0° C. for about 2 hours then warmed to 20° C. and held for about 16 hours. The reaction was quenched with water (5 volumes) over about 15 minutes. The aqueous layer is discharged and the organic layer was washed with 2% brine (5 volumes). The reaction was solvent swapped into toluene and concentrated to dryness. The residue was taken up in toluene (10 volumes) and concentrated to dryness 3 times. The residue was purified by chromatography on silica gel to provide Compound IX-2-Np. ¹H NMR (400 MHz, CD₃CN) δ 8.26-8.24 (m, 1H), 7.92 (dd, J=7.90, 1.71 Hz, 1H), 7.86 (d, J=8.20 Hz, 1H), 7.60-7.47 (m, 4H), 7.06 (d, J=1.31 Hz, 1H), 4.39 (s, 2H), 4.06-4.04 (m, 2H), 2.76 (m, 2H). ¹⁹F NMR (176 MHz, CD₃CN) δ −63.97

### Asymmetric Hydrogenation of a Compound of Formula IX-2 to a Compound of Formula IX-3

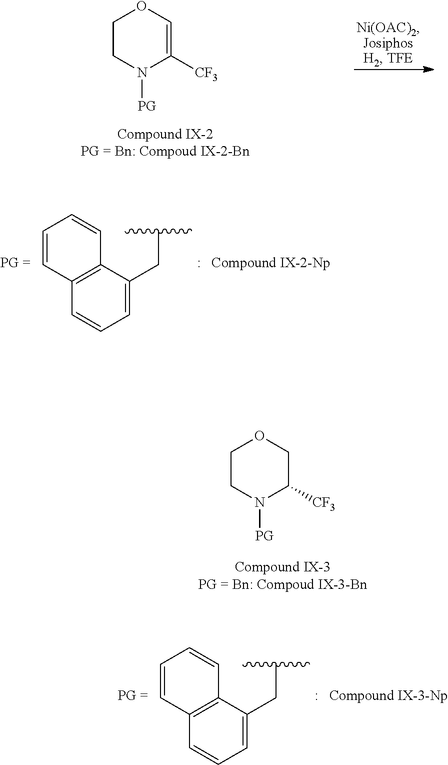

a compound of Formula IX-2 (1.00 equiv, scaling factor), Ni(OAc)₂ (0.10 equiv), and Josiphos Ligand (CAS 277306-29-3, 0.10 equiv) were charged to a reactor. The reactor was inerted and 2,2,2-trifluoroethanol (20.0 volumes) was charged to the reactor. The reactor was closed and purged with H₂ three times. The reactor was pressurized to about 30 bar H₂ and agitated at about 70° C. for about 48 hours. The resulting reaction mixture was vented, filtered, and concentrated to dryness under reduced pressure, then diluted with ethyl acetate (20.0 volumes). The resulting ethyl acetate solution was washed with 4% aq. NH₄Cl (20.0 volumes), 4% aq. NaHCO₃ (20.0 volumes) and 4% aq. NaCl (20.0 volumes). The organic stream was concentrated under reduced pressure to dryness and then purified by chromatography on silica gel to obtain a compound of Formula IX-3.

Compound IX-3-Bn (PG=Bn): ¹H NMR (400 MHz, CDCl₃) δ 7.35-7.25 (m, 5H), 4.03 (dd, J=12.0, 3.3 Hz, 1H), 3.94 (dd, J=13.7, 1.7 Hz, 1H), 3.88-3.74 (m, 3H), 3.64 (td, J=10.0, 3.4 Hz, 1H), 3.16-3.06 (m, 1H), 3.05-2.95 (m, 1H), 2.44-2.37 (m, 1H).

Compound IX-3-Np (PG=Np): ¹H NMR (400 MHz, CDCl₃) δ 8.20 (d, J=5.4 Hz, 1H), 7.87 (d, J=4.8 Hz, 1H), 7.80 (d, J=5.4 Hz, 1H), 7.53-7.49 (m, 2H), 7.46-7.39 (m, 2H), 4.41 (d, J=8.9 Hz, 1H), 4.23 (d, J=8.9 Hz, 1H), 4.01 (dd, J=7.9, 2.2 Hz, 1H), 3.80 (d, J=1.1 Hz, 1H), 3.78-3.72 (m, 1H), 3.60-3.55 (m, 1H), 3.17-3.10 (m, 1H), 3.05-2.95 (m, 1H), 2.45 (d, J=8.0 Hz, 1H).

### Deprotection of Compound IX-3-Bn to Compound IX (+)-CSA Salt

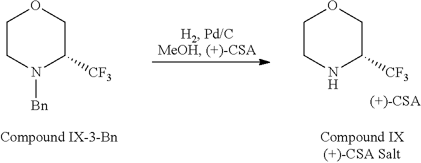 [SMILES: FC(F)(F)[C@H]1COCCN1Cc1ccccc1 ; "H2, Pd/CMeOH, (+)-CSA" ; FC(F)(F)[C@H]1COCCN1]

Compound IX-3-Bn (1.00 equiv, scaling factor) and Palladium on Carbon (0.10 equiv) were charged to the reactor. The reactor was sealed and charged with methanol (5.0 volumes). The reactor was purged with H₂ three times and pressurized to about 5 bar H₂ and agitated at about 40° C. for about 48 hours. The resulting reaction mixture was cooling to about 20° C., vented, filtered, and rinsed with acetonitrile (4.0 volumes). The organics were concentrated under reduced pressure and charged with acetonitrile (5.0 volumes) and (+)-CSA ((+)-Camphorsulfonic acid, 1.00 equiv) were charged. The contents were agitated at about 20° C. for about 30 minutes and then cooling to about 0° C. The resulting slurry was agitated at about 0° C. for about 30 minutes and filtered then dried to afford Compound IX (+)-CSA Salt. ¹H NMR (400 MHz, Methanol-d₄) δ 4.50-4.40 (m, 1H), 4.27 (dd, J=12.7, 3.7 Hz, 1H), 4.08 (dt, J=12.9, 2.9 Hz, 1H), 3.89-3.76 (m, 2H), 3.48 (dt, J=13.1, 2.7 Hz, 1H), 3.43-3.34 (m, 1H), 3.28 (d, J=9.2 Hz, 1H), 2.77 (d, J=14.8 Hz, 1H), 2.69-2.59 (m, 1H), 2.34 (dt, J=18.4, 3.6 Hz, 1H), 2.10-1.97 (m, 2H), 1.89 (d, J=18.4 Hz, 1H), 1.67-1.57 (m, 1H), 1.47-1.37 (m, 1H), 1.12 (s, 3H), 0.86 (s, 3H). ¹⁹F NMR (376 MHz, Methanol-d₄) δ −73.68 (d, J=6.9 Hz).

### Deprotection of Compound IX-3-Np to Compound IX (+)-CSA Salt

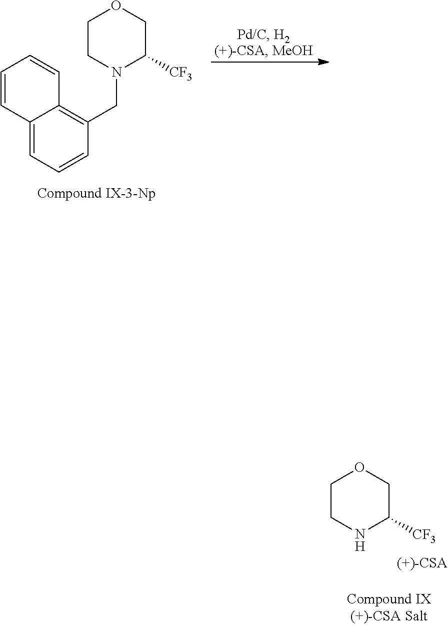 [SMILES: FC(F)(F)[C@H]1COCCN1Cc1cccc2ccccc12 ; "Pd/C, H2(+)-CSA, MeOH" ; FC(F)(F)[C@H]1COCCN1]

Compound IX-3-Np (1.00 equiv, scaling factor) and Palladium on Carbon (0.080 equiv), (+)-CSA ((+)-Camphorsulfonic acid, 1.50 equiv), and methanol (4.0 volumes) were charged to a pressure reactor, flushed with nitrogen for about 5 times, then flushed with hydrogen for about 5 times. The reactor was pressurized to about 8 bar H₂ and agitated at about 40° C. for about 3 days. The resulting reaction mixture was vented, filtered, and the organics were concentrated under reduced pressure. The resulting reaction mixture was charged with tetrahydrofuran (10.0 volumes). The contents were agitated at about 20° C. for about 30 minutes. The resulting slurry was filtered and rinsed with tetrahydrofuran (3.0 volumes) then dried to afford Compound IX (+)-CSA Salt. ¹H NMR (600 MHz, CDCl₃) δ 4.19 (d, J=5.6 Hz, 2H), 4.18-4.16 (m, 1H), 4.11-3.96 (m, 2H), 3.63 (d, J=12.5 Hz, 1H), 3.45-3.35 (m, 1H), 3.26 (d, J=14.6 Hz, 1H), 2.82 (d, J=14.6 Hz, 1H), 2.50-2.41 (m, 1H), 2.32 (dd, J=14.6, 3.5 Hz, 1H), 2.08 (t, J=4.2 Hz, 1H), 2.02-1.91 (m, 1H), 1.89 (d, J=18.3 Hz, 1H), 1.81-1.77 (m, 1H), 1.43-1.40 (m, 1H), 1.04 (s, 3H), 0.84 (s, 3H).

### Example 14: Synthesis of Compound IX—Method B

### Hydrogenation and Debenzylation of Compound IX-2-Bn to Compound IXa HCl Salt

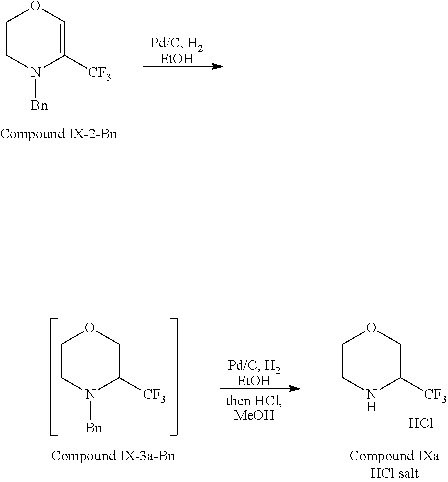

Compound IX-2-Bn (1.00 equiv, scaling factor) and Palladium on Carbon (0.10 equiv) were charged to a glass scintillation vial. The vial was placed in an autoclave and charged with ethanol (100.0 volumes). The autoclave was closed and purged with H₂ three times. The autoclave was pressurized to about 1 bar H₂ and agitated at about 20° C. for about 20 hours. The resulting reaction mixture was vented, filtered, and charged with hydrochloric acid (5.0 equiv) in methanol (20.0 volumes). The organics were concentrated under reduced pressure to afford Compound IXa HCl salt. ¹H NMR (400 MHz, DMSO-d₆) δ 10.74 (s, 2H), 4.61-4.45 (m, 1H), 4.13 (dd, J=12.4, 3.6 Hz, 1H), 3.95 (dt, J=12.5, 3.4 Hz, 1H), 3.88-3.71 (m, 2H), 3.29-3.20 (m, 1H), 3.14 (ddd, J=13.2, 9.9, 3.7 Hz, 1H). ¹⁹F NMR (376 MHz, DMSO-d₆) δ −69.80 (d, J=7.7 Hz).

### Freebasing of Compound IXa HCl Salt and Classical Resolution to Compound IX (+)-CSA Salt

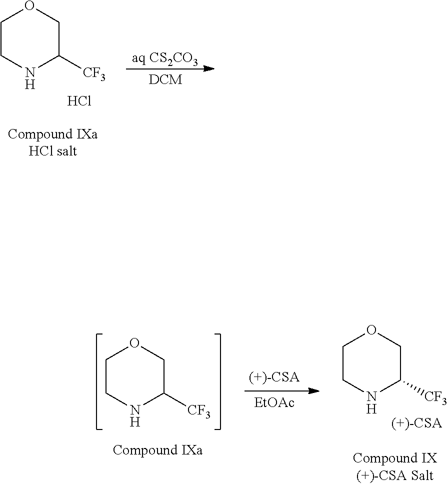

Compound IXa HCl Salt (1.00 equiv, scaling factor) and dichloromethane (15.0 volumes) were charged to the reactor A. Cesium carbonate (2.00 equiv) and water (5.0 volumes) were charged to the reactor B. The solution from reactor B was slowly charged to reactor A and the resulting reaction mixture was agitated at about 20° C. for about 15 minutes. Then the aqueous layer was washed with dichloromethane (15.0 volumes). The two dichloromethane streams were combined and (+)-CSA (1.00 equiv) was charged to the solution. The resulting reaction mixture was agitated at about 20° C. for about 15 minutes and concentrated down under reduced pressure. Ethyl acetate (15.0 volumes) was charged to the reactor and concentrated down under reduced pressure. Then Ethyl acetate (10.0 volumes) was charged to the reactor. The resulting reaction mixture was heated up to about 40° C., agitated at about 40° C. for about 30 minutes and cooling down to about 20° C. over about 1 hour. The resulting slurry was agitated at about 20° C. for about 30 minutes and filtered. The filtered cake and methyl ethyl ketone (13.0 volumes) were charged to reactor. The resulting reaction mixture was heated up to about 60° C., agitated at about 60° C. for about 30 minutes and cooling down to about 20° C. over about 4 hour. The resulting slurry was agitated at about 20° C. for about 10 hours and filtered to afford Compound IX (+)-CSA Salt. ¹H NMR (400 MHz, Methanol-d₄) δ 4.50-4.40 (m, 1H), 4.27 (dd, J=12.7, 3.7 Hz, 1H), 4.08 (dt, J=12.9, 2.9 Hz, 1H), 3.89-3.76 (m, 2H), 3.48 (dt, J=13.1, 2.7 Hz, 1H), 3.43-3.34 (m, 1H), 3.28 (d, J=9.2 Hz, 1H), 2.77 (d, J=14.8 Hz, 1H), 2.69-2.59 (m, 1H), 2.34 (dt, J=18.4, 3.6 Hz, 1H), 2.10-1.97 (m, 2H), 1.89 (d, J=18.4 Hz, 1H), 1.67-1.57 (m, 1H), 1.47-1.37 (m, 1H), 1.12 (s, 3H), 0.86 (s, 3H). ¹⁹F NMR (376 MHz, Methanol-d₄) δ −73.68 (d, J=6.9 Hz).

### Reduction of Compound IX-2-Bn to Compound IX-3a-Bn via Hydrogen Atom Transfer

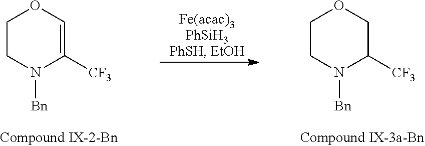 [SMILES: FC(F)(F)C1=COCCN1Cc1ccccc1 ; "Fe(acac)3PhSiH3PhSH, EtOH" ; FC(F)(F)C1COCCN1Cc1ccccc1]

A mixture of Compound IX-2-Bn (1.00 equiv, scaling factor), phenylsilane (2 equiv), and iron(III) acetylacetonate (0.10 equiv) was inerted with nitrogen and charged with ethanol (40 volumes) and 0.50 M thiophenol in ethanol (0.10 equiv). The resulting mixture was stirred at about 20° C. for about 24 hours. The reaction was diluted with ethyl acetate (80 volumes) and the combined organic phase was washed with 10% brine (40 volumes). The organic layer was filtered through silica gel eluting with ethyl acetate (160 volumes) and concentrated under reduced pressure to afford Compound IX-3a-Bn. ¹H NMR (400 MHz, CDCl₃) 7.35-7.25 (m, 5H), 4.03, (dd, J=11.9, 3.1 Hz, 1H), 3.94 (dq, J=13.7, 1.7 Hz, 1H), 3.87-3.75 (m, 3H), 3.65 (td, J=10.4, 3.3 Hz, 1H), 3.15-3.07 (m, 1H), 3.05-2.97 (m, 1H), 2.44-2.38 (m, 1H).

### Reduction of Aminal Compound IX-4a to Compound IX-3a-Bn

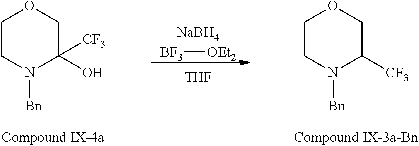

A mixture of Compound IX-4a (1.00 equiv, scaling factor) and tetrahydrofuran (10 volumes) was cooled to about 0° C. Sodium borohydride (1.2 equiv) was charged to the mixture followed by boron trifluoride diethyl etherate (1.0 equiv). The mixture was stirred for about 18 hours during while warming to about 20° C. The mixture was diluted with ethyl acetate (30 volumes) and washed with saturated aqueous sodium bicarbonate (5 volumes) and then 2% brine (5 volumes). The organics were concentrated and the residue was purified by chromatography to afford Compound IX-3a-Bn. ¹H NMR (400 MHz, CDCl₃) 7.35-7.25 (m, 5H), 4.03, (dd, J=11.9, 3.1 Hz, 1H), 3.94 (dq, J=13.7, 1.7 Hz, 1H), 3.87-3.75 (m, 3H), 3.65 (td, J=10.4, 3.3 Hz, 1H), 3.15-3.07 (m, 1H), 3.05-2.97 (m, 1H), 2.44-2.38 (m, 1H).

### Example 15: Synthesis of Compound IX—Method C (Synthesis of a Compound of Formula IX-5a)

A compound of Formula IX-5a can be prepared according to the following schemes

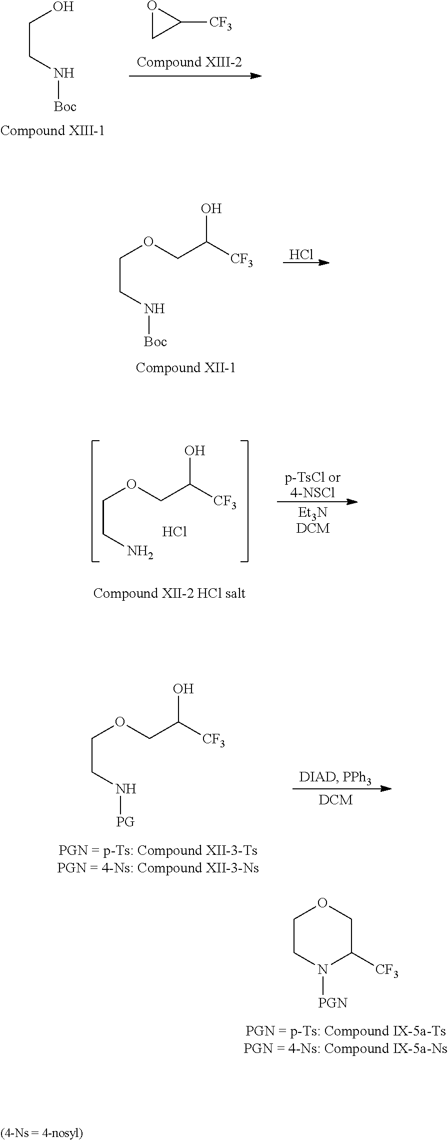

Epoxide opening to Compound XII-1

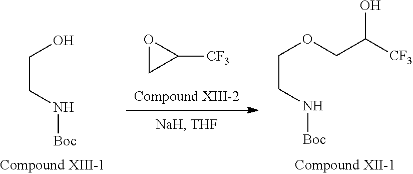 [SMILES: CC(C)(C)OC(=O)NCCO ; FC(F)(F)C1CO1 ; CC(C)(C)OC(=O)NCCOCC(O)C(F)(F)F]

Compound XIII-1 (1.00 equiv, scaling factor) and DMF (20 volumes) were charged to a reactor and the temperature was adjusted to about 0° C. Sodium hydride (60% dispersion in mineral oil; 1.2 equiv) was added and the mixture was warmed to about 20° C. and stirred for about 30 minutes. The temperature was adjusted to about 0° C., 2-(trifluoromethyl)oxirane (Compound XIII-2, 1.0 equiv) was added, and the mixture was warmed to about 20° C. and stirred for about 15 hours. The reaction mixture was quenched with saturated aqueous NH₄Cl solution (14 volumes) and diluted with EtOAc (14 volumes). The organic layer was separated, and the aqueous layer was back extracted with EtOAc (14 volumes). The combined organic layers were washed three times with water (14 volumes each), concentrated to dryness, and the residue was purified by chromatography to afford Compound XII-1. ¹H NMR (400 MHz, DMSO) δ 6.77 (br s, 1H), 6.31 (br s, 1H), 4.10 (br s, 1H), 3.59 (dd, J=10.7, 4.2 Hz, 1H), 3.48 (m, 1H), 3.43 (t, J=6.0 Hz, 2H), 3.07 (q, J=5.9 Hz, 2H) 1.38 (s, 9H).

### Deprotection of Compound XII-1 and Conversion to Compound XII-3-Ts/Compound XII-3-Ns

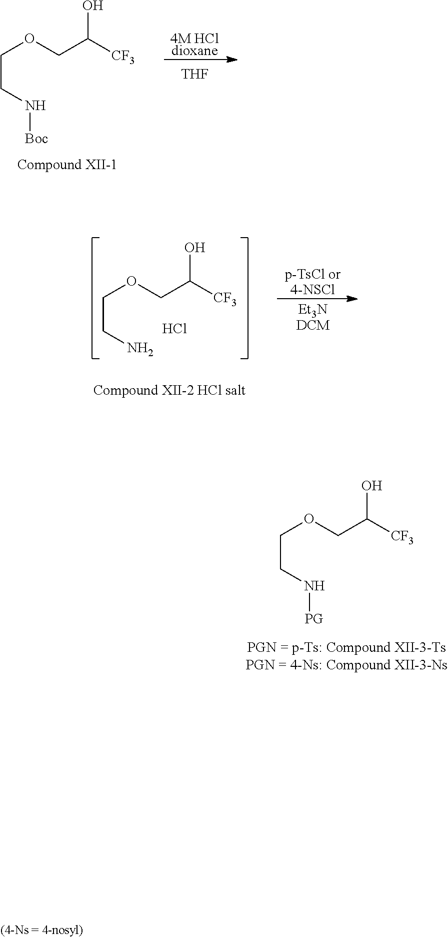

Compound XII-1 (1.00 equiv, scaling factor) and THF (7 volumes) were charged to a reactor and the temperature was adjusted to about 0° C. 4M HCl in dioxane (7.0 equiv) was added and the mixture was warmed to about 20° C. and stirred for about 6 hours. The reaction mixture was concentrated to dryness and the residue was charged with DCM (8 volumes) and concentrated again to dryness. The residue was again charged with DCM (8 volumes) and concentrated to dryness to afford Compound XII-2 HCl salt.

Compound XII-2 HCl salt (1.00 equiv, scaling factor), triethylamine (3 equiv), and DCM (16 volumes) were charged to a reactor and the temperature was adjusted to about 0° C. The corresponding sulfonyl chloride (p-TsCl or 4-NsCl; 1.05 equiv) was added and the mixture was warmed to about 20° C. and stirred for about 6 hours. The reaction mixture was quenched with 10% aqueous NH₄Cl solution (8 volumes) and diluted with water (16 volumes). The organic layer was separated and washed twice with 10% aqueous NaCl solution (8 volumes each), dried over Na₂SO₄, and concentrated to dryness. The residue was purified by chromatography to afford Compound XII-3-Ts or Compound XII-3-Ns. Compound XII-3-Ts (PGN=p-Ts): ¹H NMR (400 MHz, DMSO) δ 7.67 (d, J=8.4 Hz, 2H), 7.59 (br s, 1H), 7.39 (d, J=8.1 Hz, 2H), 6.30 (br s, 1H), 4.13-3.97 (m, 1H), 3.52 (dd, J=10.7, 4.0 Hz, 1H), 3.46-3.38 (m, 3H), 2.88 (t, J=5.8 Hz, 2H), 2.38 (s, 3H). Compound XII-3-Ns (PGN=4-Ns): ¹H NMR (400 MHz, DMSO) δ 8.40 (d, J=8.8 Hz, 2H), 8.12 (br s, 1H), 8.05 (d, J=9.2 Hz, 2H), 6.30 (br s, 1H), 4.08-3.97 (m, 1H), 3.53-3.36 (m, 4H), 3.00 (q, J=5.7 Hz, 2H).

### Cyclization of Compound XII-3-Ts or Compound XII-3-Ns to Compound IX-5a-Ts or Compound IX-5a-Ns

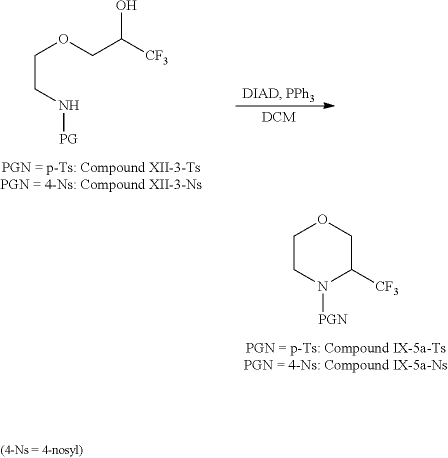

Compound XII-3-Ts (PGN=p-Ts) or Compound XII-3-Ns (PGN=4-Ns) (1.00 equiv, scaling factor), triphenylphosphine (1.5 equiv), and dichloromethane (30 volumes) were charged to a reactor and the temperature was adjusted to about 0° C. Diisopropyl azodicarboxylate (DIAD; 1.5 equiv) was added and the mixture was warmed to about 20° C. and stirred for about 18 hours. The reaction mixture was concentrated to dryness and the residue was purified by chromatography to afford Compound IX-5a-Ts or Compound IX-5a-Ns. Compound IX-5a-Ts (PGN=p-Ts): ¹H NMR (400 MHz, DMSO) δ 7.82 (d, J=8.2 Hz, 2H), 7.45 (d, J=7.7 Hz, 2H), 4.71 (qd, J=8.9, 4.2 Hz, 1H), 3.99 (d, J=13.2 Hz, 1H), 3.67 (dd, J=11.8, 3.5 Hz, 1H), 3.60-3.49 (m, 1H), 3.37-3.23 (m, 2H), 2.85 (td, J=12.0, 3.2 Hz, 1H), 2.42 (s, 3H). Compound IX-5a-Ns (PGN=4-Ns): ¹H NMR (400 MHz, DMSO) δ 8.43 (d, J=5.6 Hz, 2H), 8.21 (d, J=6.0 Hz, 2H), 4.89-4.76 (m, 1H), 4.02 (d, J=13.3 Hz, 1H), 3.72 (dd, J=11.9, 3.5 Hz, 1H), 3.66 (d, J=14.2 Hz, 1H), 3.44-3.34 (m, 1H), 3.02-2.91 (m, 2H).

### Example 16: Synthesis of Compound II—Method A

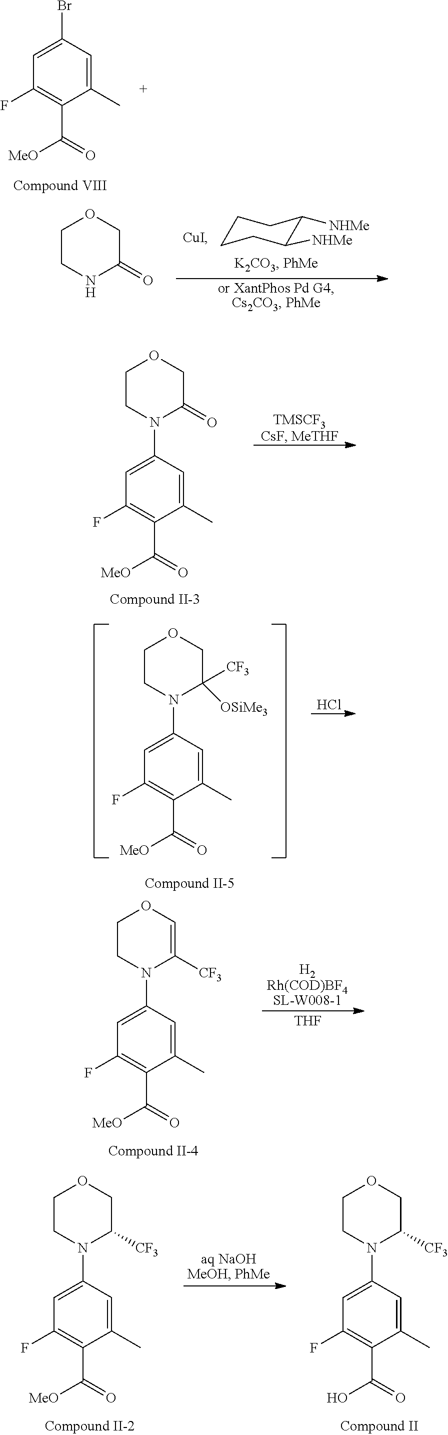

### Coupling of Compound VIII and Morpholin-3-one

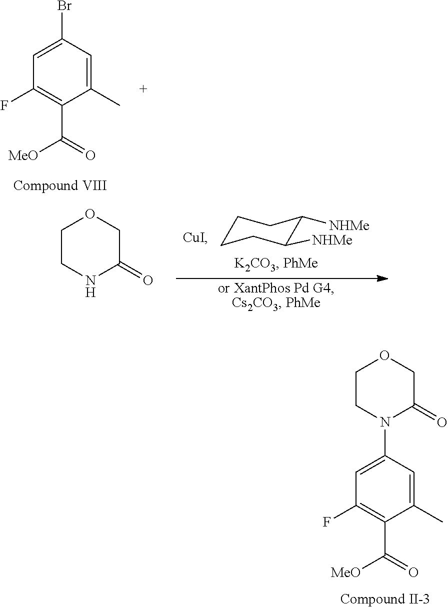

Palladium-catalyzed coupling. Compound VIII (1.00 equiv, scaling factor), XantPhos Pd G4 (0.01 equiv), morpholin-3-one (1.10 equiv), cesium carbonate (1.05 equiv) and toluene (11.0 volumes) was charged to a reactor and the contents were agitated at about 85° C. for about 19 hours. The reactor temperature was set to about 40° C. A solution of 8% sodium phosphate monobasic in water was added in portions over about 1 hour until pH about 7-8. The mixture was stirred for about 30 minutes. The aqueous layer was discharged. A solution of 5% sodium sulfate was charged. After about 1 hour the bottom layer was discharged. The remaining organic phase was polish filtered. The filtrate was transferred to a reactor. The mixture was distilled to between four and five volumes. The mixture was warmed to approximately 65° C. Heptane (6.0 volumes) was charged to the reactor over about 10 minutes to yield a thin slurry. The mixture was set to cool to approximately 10° C. over about 7 hours then held for about 10 hours. The slurry was filtered and dried under reduced pressure at about 50° C. to afford Compound II-3. ¹H NMR (400 MHz, DMSO-d6) δ 7.36 (dd, J=11.7, 2.0 Hz, 1H), 7.28 (br s, 1H), 4.23 (s, 2H), 3.97 (dd, J=5.9, 4.1 Hz, 2H), 3.87 (s, 3H), 3.78 (dd, J=6.1, 4.1 Hz, 2H), 2.35 (s, 3H).

Copper-catalyzed coupling. Compound VIII (1.0 equiv, scaling factor), morpholin-3-one (1.3 equiv), cuprous iodide (0.3 equiv), potassium carbonate (1.1 equiv), and toluene (7.0 volumes) were charged to a reactor. The reactor contents were purged of oxygen by evacuating and then backfilling with nitrogen (repeat 3×). Trans-N,N′-bismethyl-1,2-cyclohexanediamine (0.6 equiv) was charged via syringe, and the reactor contents were adjusted to about 100° C. and agitated for about 15 h before adjusting the temperature to about 20° C. Toluene (7.5 volumes) and water (20.3 volumes) were charged to the reactor then the mixture was acidified, first with acetic acid (4.3 volumes) and then hydrochloric acid (2.1 volumes). The resulting thick slurry was filtered, and the cake was rinsed with toluene (10.5 volumes). The combined filtrate a rinse was a biphasic mixture which was charged to the reactor, and the lower aqueous phase was discharged. Water (10.0 volumes) was charged to the reactor, and the mixture was agitated for about 5 minutes before being discharged to the aqueous phase. Two additional water washes were performed using 10.0 volumes of water. A 5 wt % aqueous solution of sodium chloride (10.0 volumes) was charged to the reactor and the mixture was agitated for about 5 min before discharging to the aqueous phase. Silica gel (2 parts) was charged to the organic phase remaining in the reactor and the suspension was agitated for about 1 h. The suspension was filtered, and the cake was rinsed with ethyl acetate (17.2 volumes). The combined filtrate and rinse were concentrated rotary evaporation, then taken up in ethyl acetate (5.8 volumes) by warming to dissolution at about 50° C. and then the solution was charged to a reactor. Heptane (7.3 volumes) was charged to the reactor contents at about 50° C., and the solution was seeded with Compound II-3. The temperature of the reactor contents was adjusted from about 50° C. to about 0° C. over not less than 12 h. The resulting slurry was filtered, the cake was rinsed with heptane (4.4 volumes) and dried under vacuum at 50° C. for not less than 24 h, providing Compound II-3. ¹H NMR (400 MHz, CDCl₃) δ 7.07-7.02 (m, 2H), 4.32 (s, 1H), 4.02 (dd, J=5.8, 4.3 Hz, 2H), 3.92 (s, 3H), 3.74 (dd, J=5.8, 4.3 Hz, 2H), 2.41 (s, 3H).

### Trifluoromethylation and Elimination of Compound II-3 to Compound II-4

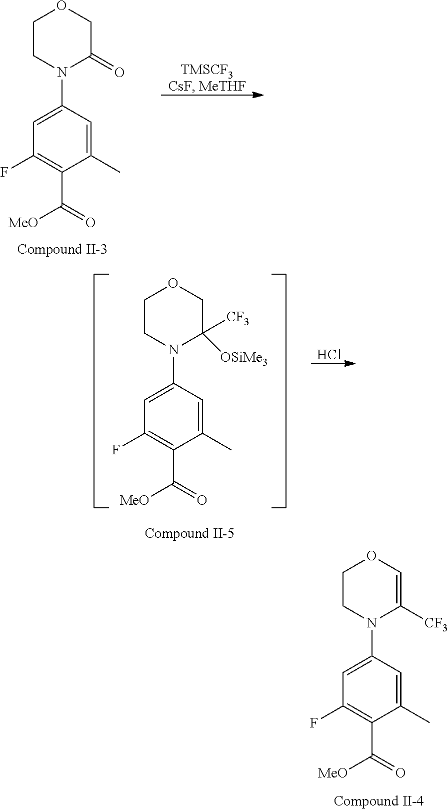

Compound II-3 (1.0 equiv, scaling factor), cesium fluoride (0.20 equiv) and 2-methyltetrahydrofuran (30.0 volumes) were charged to a reactor. The reactor contents were cooled to about −15° C., and (trifluoromethyl)trimethylsilane was charged to the agitated slurry dropwise. The temperature of the reactor contents was adjusted from about −15° C. to about 15° C. over about 3 h. A solution of hydrochloric acid in dioxane (4 M, 2.0 equiv) was charged to the reactor, and the reactor contents were agitated at about 15° C. for about 20 h. The reactor contents were warmed to about 20° C. and a solution of 10 wt % aqueous potassium bicarbonate (14.0 volumes) was added and agitated for not less than 15 min before discharging the lower aqueous phase. A solution of 10 wt % aqueous sodium chloride (14.0 volumes) was added and agitated for about 15 min before discharging the lower aqueous phase. Azeotropic distillations to a minimum of 7.0 volumes were performed with 2-methyltetrahydrofuran (11.0 volumes) until the water content was reduced to not more than 0.3 wt %. The reactor contents were diluted with additional 2-methyltetrahydrofuran (11.0 volumes), and then the solution was polish-filtered. The filtrate was concentrated by rotary evaporation, and the residue was taken up in a mixture of dichloromethane/heptane (1:1 v/v, 12.8 volumes). The solution was charged to a reactor along with silica gel (1.3 parts) and the suspension was adjusted to about 25° C. and agitated for about 30 min. The suspension was filtered, and the cake was rinsed sequentially with a mixture of dichloromethane/heptane (1:1 v/v, 12.8 volumes), followed by dichloromethane (12.8 volumes). The combined filtrate and rinse were concentrated rotary evaporation, then taken up in a solution of tert-butyl methyl ether/heptane (5:95 v/v, 10.2 volumes), warming to dissolution at about 60° C. and then charging the solution to a reactor. The temperature of the reactor contents was adjusted from about 60° C. to about 0° C. over not less than 12 h. The resulting slurry was filtered, the cake was rinsed with heptane (4.4 volumes) and dried under vacuum at about 70° C. for not less than 20 h, providing Compound II-4. ¹H NMR (400 MHz, CDCl₃) δ 7.11 (q, J=1.2 Hz, 1H), 6.72 (d, J=2.2 Hz, 1H), 6.66 (dd, J=11.4, 2.2 Hz, 1H), 4.03-3.98 (m, 2H), 3.91 (s, 3H), 3.53-3.46 (m, 2H), 2.40 (s, 3H).

### Asymmetric Hydrogenation of Compound II-4 to Compound II-2

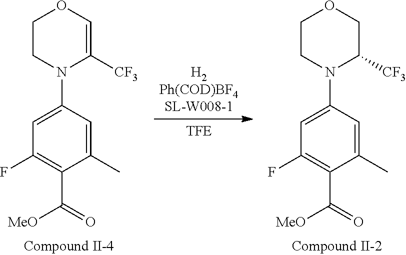 [SMILES: COC(=O)c1c(C)cc(N2CCOC=C2C(F)(F)F)cc1F ; COC(=O)c1c(C)cc(N2CCOC[C@@H]2C(F)(F)F)cc1F]

Solutions of Rh(COD)₂BF₄ (0.10 equiv) and (R)-(+)-1-[(R)-2-(2′-Dicyclohexylphosphinophenyl)ferrocenyl]ethylbis(3,5-trifluoromethylphenyl) phosphine (SL-W008-1) (0.11 equiv) in tetrahydrofuran (33.0 volumes) were combined in a vial and concentrated to dryness under an atmosphere of nitrogen. A solution of Compound II-4 (1.00 equiv, scaling factor) in 2,2,2-trifluoroethanol (31.0 volumes) was charged to the premixed catalyst. The reaction mixture was sealed in a high-pressure reactor under an atmosphere of nitrogen. The reactor was purged by pressurizing to about 120 psi with H₂ and releasing the pressure (repeated 3×). The reactor was pressurized to about 120 psi H₂ and warmed to about 60° C. for about 3 days. The reactor was depressurized, and the contents cooled to about 20° C. After evaluating conversion and purity, the reaction mixture was concentrated by rotary evaporation. The residue was purified by chromatography to give Compound II-2. ¹H NMR (400 MHz, CDCl₃) δ 6.46 (d, J=2.5 Hz, 1H), 6.40 (dd, J=13.2, 2.5 Hz, 1H), 4.31 (dt, J=12.5, 1.1 Hz, 1H), 4.14 (qd, J=8.1, 3.8 Hz, 1H), 4.05 (ddt, J=11.2, 3.8, 1.2 Hz, 1H), 3.88 (s, 3H), 3.81 (ddq, J=12.5, 4.2, 2.1 Hz, 1H), 3.65 (dddd, J=12.2, 11.2, 3.3, 0.9 Hz, 1H), 3.52 (tdq, J=12.2, 3.4, 1.6 Hz, 1H), 3.30 (br d, J=12.4 Hz, 1H), 2.42 (s, 3H).

### Example 17: Synthesis of Compound II—Method B

Compound II can be prepared according to the following schemes:

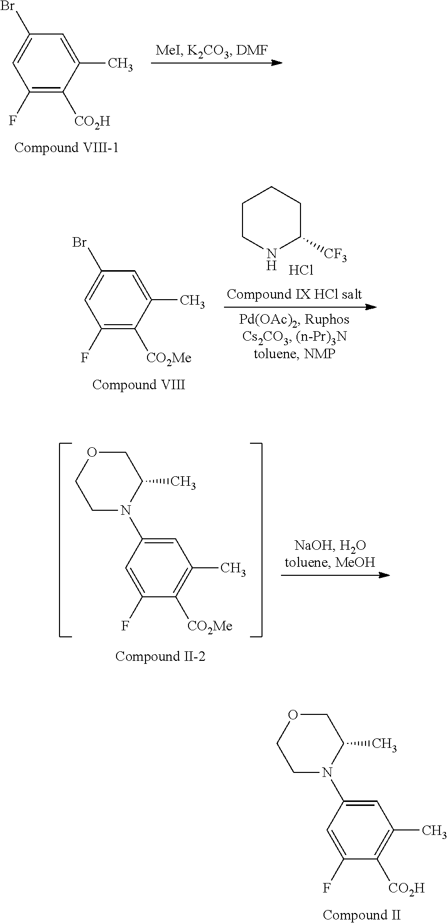

### Repurification of Compound IX HCl Salt

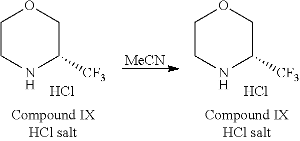 [SMILES: "MeCN" ; FC(F)(F)[C@H]1COCCN1 ; FC(F)(F)[C@H]1COCCN1 ; Cl ; Cl]

Compound IX HCl Salt can be prepared according to the procedure disclosed in Tetrahedron, 2013, 69, 3796-3804, which is incorporated herein by reference in its entirety.

Compound IX HCl Salt (1.00 equiv, scaling factor) and acetonitrile (5.0 volumes) were charged to a reactor and the contents were agitated at about 45° C. for about 12 hours. The slurry was filtered, and the cake was washed with acetonitrile (2×5.0 volumes). The solids were dried under reduced pressure at about 50° C. to afford repurified Compound IX HCl Salt. ¹H NMR (500 MHz; DMSO-d₆; Me₄Si), δ: 12.0-10.0 (2H, broad s); 4.60-4.46 (1H, m); 4.12 (1H, dd, J=16.0, 4.0 Hz); 3.94 (1H, d, J=16.0 Hz); 3.86 (1H, d, J=12.5 Hz); 3.82-3.75 (1H, m); 3.23 (1H, d, J=16.0 Hz); 3.61 (1H, dd, J=12.5, 4.0 Hz). ¹⁹F NMR (375 MHz; DMSO-d₆; CFCl₃), δ: −76.35 (d, ³J(F,H)=7.5 Hz).

### Esterification of Compound VIII-1 to Compound VIII

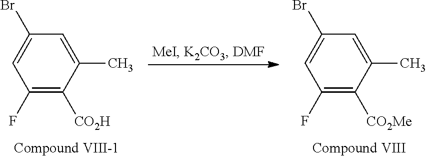

Compound VIII-1 (1.00 equiv, scaling factor), potassium carbonate (2.00 equiv), and N,N-dimethylformamide (5.0 volumes) were charged to a reactor. The reactor contents were adjusted to about 15° C. and then slowly charged with methyl iodide (1.20 equiv). The resulting mixture was agitated for about 12 hours. The reaction mixture was slowly charged with water (5.0 volumes) and agitated at about 15° C. for about 2 hours. The slurry was then filtered, and the cake was dried under reduced pressure at about 20° C. to afford Compound VIII. ¹H NMR (400 MHz, CDCl₃) δ 7.14-7.19 (m, 2H), 3.93 (s, 3H), 2.39 (s, 3H).

### Amide Coupling and Transesterification to Compound II

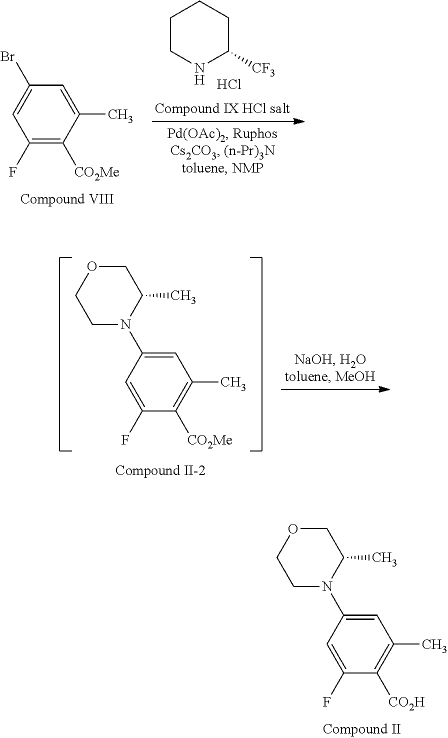

Compound VIII (1.00 equiv, scaling factor), Compound IX HCl Salt (1.10 equiv), cesium carbonate (3.00 equiv), toluene (5.0 volumes), N-methyl-2-pyrrolidone (2.0 volumes), and N,N-dipropylpropan-1-amine (1.50 equiv) were charged to a reactor and the contents were adjusted to about 20° C. Palladium acetate (0.03 equiv) and RuPhos (0.06 equiv) were charged to the reactor and the reaction mixture was agitated at about 95° C. for about 12 hours. The resulting reaction mixture was adjusted to about 60° C., charged with L-cysteine (0.50 equiv) and water (5.0 volumes), and agitated for about 12 hours. Reactor contents were cooled to about 40° C. and the organic layer was washed with water (5.0 volumes) and 2% brine (5.0 volumes). The organic layer was concentrated under reduced pressure to afford crude Compound II-2. Crude Compound II-2 and methanol (3.0 volumes) were charged to a reactor and the contents were adjusted to about 20° C. Sodium hydroxide (3.00 equiv) and water (3.0 volumes) were charged and the reactor contents were agitated at about 65° C. for about 12 hours. The resulting reaction mixture was cooled to about 20° C., extracted with tert-butyl methyl ether (2×5.0 volumes), and the organic layer was discharged. The aqueous containing layer was acidified with 2N hydrochloric acid solution (5.0 volume) and the resulting reaction mixture pH was adjusted to about 2. The mixture was extracted with tert-butyl methyl ether (10.0 volumes) and the organics were concentrated under reduced pressure. Acetonitrile (3.0 volumes) was charged and the reaction mixture was agitated at about 35° C. for about 1 hour. The slurry was filtered, and the filtrate was concentrated to dryness under reduced pressure. Isopropanol (2.5 volumes) was charged and the reaction mixture was agitated at about 45° C. Water (4.5 volumes) was slowly charged and the resulting reaction mixture was agitated for about 1 hour. The slurry was cooled to about 35° C., filtered, and the cake was dried under reduced pressure at about 45° C. to afford Compound II. ¹H NMR (400 MHz, DMSO-d₆) δ 12.87 (s, 1H), 6.73-6.77 (m, 2H), 4.89 (dd, J=8.8, 3.7 Hz, 1H), 4.16 (d, J=12.7 Hz, 1H), 3.96 (dd, J=11.6, 3.8 Hz, 1H), 3.55 (td, J=11.6, 3.3 Hz, 1H), 3.43 (dd, J=13.3, 2.9 Hz, 1H), 3.39-3.19 (m, 2H), 2.34 (s, 3H).

### Example 18: Synthesis of Compound X and Derivatives

### Enzyme-Catalyzed Coupling to Compound X

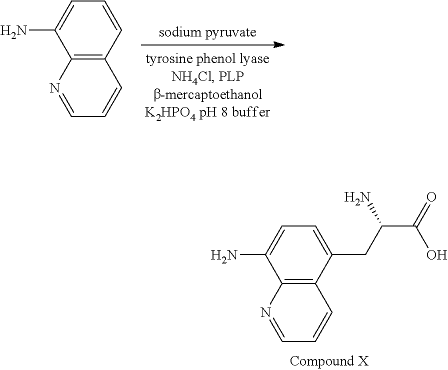 [SMILES: Nc1cccc2cccnc12 ; Nc1ccc(C[C@H](N)C(=O)O)c2cccnc12]

A mixture of 8-aminoquinoline (1.00 equiv, scaling factor), potassium phosphate buffer (pH 8.0, 100 mM, 690 volumes), ammonium chloride (3.0 equiv), sodium pyruvate (6.0 equiv), pyridoxal phosphate (0.10 equiv), beta-mercaptoethanol (0.50 equiv) and tyrosine phenol lyase (0.10 equiv) was incubated at about 25° C. for about 24 hours. A sample of the mixture was quenched with acetonitrile (50 vol %) and mixed, then diluted with water (100 vol %) and mixed, and then centrifuged to provide a solution of Compound X. The presence of Compound X was confirmed by UPLC retention time and HRMS relative to a reference sample of Compound X prepared from different chemical synthetic methods. HRMS m/z: [M+H]⁺ calcd for C₁₂H₁₃N₃O₂, 232.1081, found 232.1079. A fragment at 215.0816 m/z [C₁₂H₁₁N₂O₂⁺] (corresponding neutral mass loss of 17.0266) and a fragment at 158.0839 m/z [C₁₀H₁₀N₂⁺] (corresponding neutral mass loss of 74.0242) were observed to be consistent with fragmentation pattern of the Compound X reference sample.

### Synthesis of Compound X-3

### Negishi Coupling of Compound IV-4 and Compound VII-2 to Compound X-4

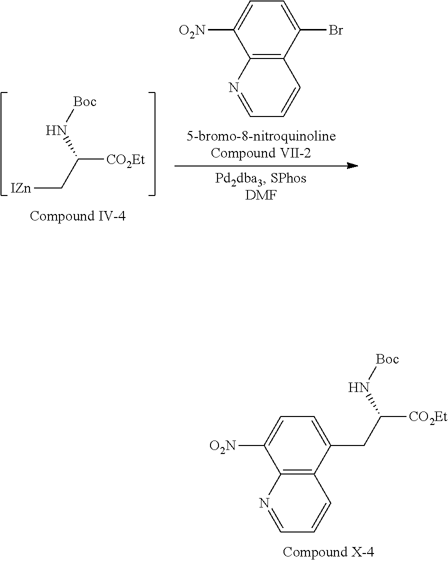

A reactor containing 5-bromo-8-nitroquinoline (1.0 equiv, scaling factor), N,N-dimethylformamide (5.0 volumes), tris(dibenzylideneacetone)dipalladium(0) (0.04 equiv), and 2-dicyclohexylphosphino-2′,6′-dimethoxybiphenyl (0.08 equiv) was heated to about 55° C. A solution of Compound IV-4 (2.0 equiv) in DMF (3.6 ml) was added dropwise and the mixture was heated at about 55° C. for about 1 hour. The resulting mixture was cooled to about 20° C. and filtered, and the filtered solids were rinsed three times with ethyl acetate (10 volumes per rinse). Water (50 volumes) was added to the filtrate, and the aqueous layer was extracted twice with ethyl acetate (50 volumes per extraction). The combined organic layers were washed three times with saturated sodium chloride solution (50 volumes per wash), dried over magnesium sulfate, filtered, and concentrated. The crude product was purified by column chromatography to give Compound X-4. ¹H NMR (300 MHz, CDCl₃) δ 9.09 (dd, J=4.2, 1.6 Hz, 1H), 8.60 (d, J=8.5, 1H), 7.94 (d, J=7.8 Hz, 1H), 7.61 (dd, J=8.7, 4.1 Hz, 1H), 7.41 (d, J=7.7 Hz, 1H), 5.15 (d, J=7.6 Hz, 1H), 4.66 (m, 1H), 4.11 (m, 2H), 3.60 (m, 2H), 1.40 (s, 9H), 1.15 (t, J=7.1 Hz, 3H).

### Nitro Reduction of Compound X-4 to Compound X-3

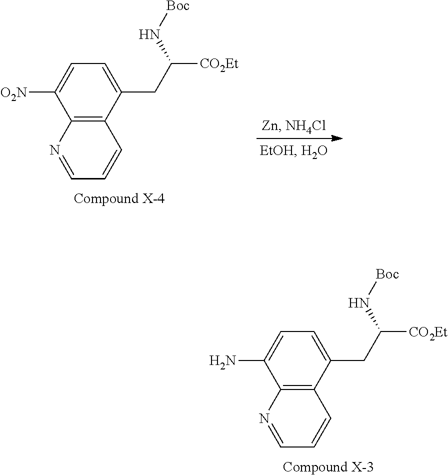 [SMILES: CCOC(=O)[C@H](Cc1ccc([N+](=O)[O-])c2ncccc12)NC(=O)OC(C)(C)C ; CCOC(=O)[C@H](Cc1ccc(N)c2ncccc12)NC(=O)OC(C)(C)C]

A mixture of Compound X-4 (1.0 equiv, scaling factor), zinc powder (10 equiv), ammonium chloride (6.0 equiv), and 70:30 ethanol/water (25 volumes) were stirred at about 20° C. for about 16 hours. The mixture was filtered, rinsed three times with methanol (50 volumes per rinse), and concentrated to dryness. The concentrate was dissolved in ethyl acetate (100 volumes), washed with water (100 volumes), and washed with saturated sodium chloride solution (100 volumes). The organic layer was dried with magnesium sulfate and concentrated to afford Compound X-3. ¹H NMR (300 MHz, CDCl₃) δ 8.78 (dd, J=4.1, 1.7 Hz, 1H), 8.36 (d, J=8.4 Hz, 1H), 7.43 (dd, J=8.7, 4.2 Hz, 1H), 7.12 (d, J=7.7 Hz, 1H), 6.87 (d, J=7.6 Hz, 1H), 5.00 (m, 3H), 4.58 (m, 1H), 4.07 (m, 2H), 3.41 (m, 2H), 1.41 (s, 9H), 1.13 (t, J=7.1 Hz, 3H).

Unless otherwise defined, all technical and scientific terms used herein have the same meaning as commonly understood by one of ordinary skill in the art to which this disclosure belongs.

The embodiments illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the embodiments claimed.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A process for preparing Compound I 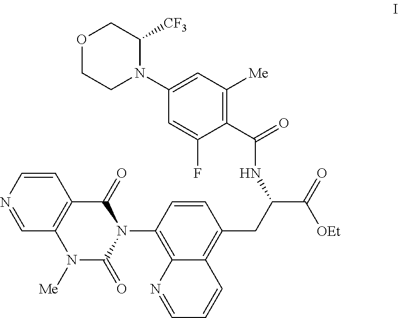
or a salt thereof, comprising:
coupling Compound II 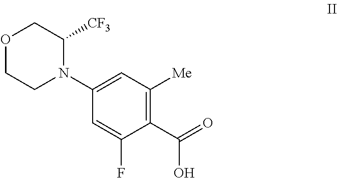 [SMILES: Cc1cc(N2CCOC[C@@H]2C(F)(F)F)cc(F)c1C(=O)O]
or a salt thereof with Compound III 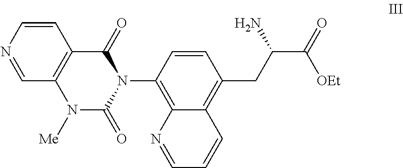
or a salt thereof in the presence of a coupling agent to form Compound I.

2. The process of claim 1, wherein the coupling agent is thionyl chloride.

3. (canceled)

4. The process of claim 1, which occurs in the presence of a base and a solvent.

5. The process of claim 4, wherein the base is triethylamine and/or the solvent is acetonitrile.

6.-8. (canceled)

9. A process for preparing Compound III 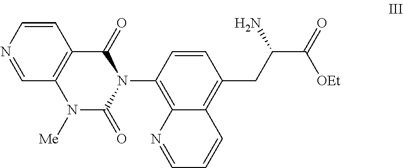
or a salt thereof, comprising:
(a) contacting Compound III-1a 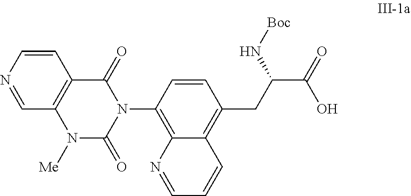 [SMILES: Cn1c(=O)n(-c2ccc(C[C@H](NC(=O)OC(C)(C)C)C(=O)O)c3cccnc23)c(=O)c2ccncc21]
or a salt thereof with ethanol in the presence of an acid to form Compound IIIa 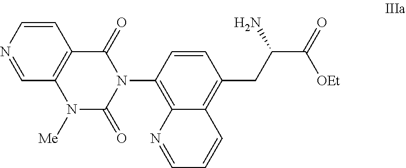 [SMILES: CCOC(=O)[C@@H](N)Cc1ccc(-n2c(=O)c3ccncc3n(C)c2=O)c2ncccc12]
or a salt thereof; and
(b) crystallizing Compound IIIa from a solvent to provide Compound III as a single atropisomer.

10.-17. (canceled)

18. The process of claim 9, wherein step (b) further comprises equilibrating Compound IIIa prior to crystallization, wherein Compound IIIa represents a mixture of atropisomers including Compound III, wherein the equilibration step is performed under acidic or thermal condition.

19. (canceled)

20. The process of claim 1, further comprising crystallizing Compound I, optionally wherein the crystallization is performed in the presence of malonic acid and the crystallization step provides Compound I Mono-Malonate.

21.-23. (canceled)

24. The process of claim 9, wherein Compound III-1a 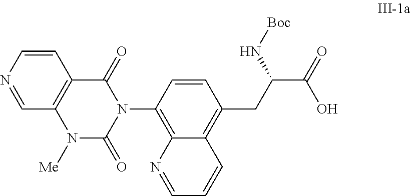 [SMILES: Cn1c(=O)n(-c2ccc(C[C@H](NC(=O)OC(C)(C)C)C(=O)O)c3cccnc23)c(=O)c2ccncc21]
or a salt thereof is prepared by a process comprising:
(a) contacting Compound IV-1 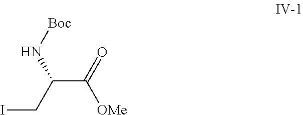 [SMILES: COC(=O)[C@H](CI)NC(=O)OC(C)(C)C]
or a salt thereof with zinc; and
(b) contacting a product from step (a) with a compound of Formula Va 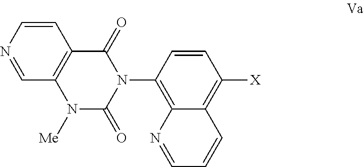
wherein X is Br or I,
or a salt thereof in the presence of a catalytic system to form Compound III-2a 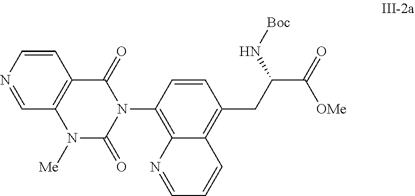 [SMILES: COC(=O)[C@H](Cc1ccc(-n2c(=O)c3ccncc3n(C)c2=O)c2ncccc12)NC(=O)OC(C)(C)C]
or a salt thereof; and
(c) subjecting Compound III-2a 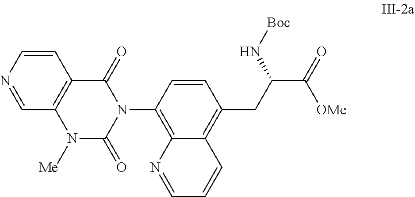 [SMILES: COC(=O)[C@H](Cc1ccc(-n2c(=O)c3ccncc3n(C)c2=O)c2ncccc12)NC(=O)OC(C)(C)C]
or a salt thereof to a hydrolysis condition to form Compound III-1a or a salt thereof.

25. The process of claim 24, wherein:
step (a) occurs in the presence of a solvent and an activator selected from methanesulfonic acid, iodine, 1,2-dibromoethane, and silyl chlorides,
the catalytic system in step (b) comprises a palladium or nickel catalyst, a ligand, and a solvent; and/or
the hydrolysis condition in step (c) comprises a base and a solvent.

26.-42. (canceled)

43. The process of claim 24, wherein a compound of Formula Va 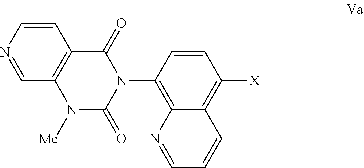
wherein X is Br or I,
or a salt thereof is prepared by a process comprising:
(a) contacting Compound VI 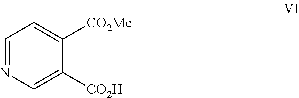 [SMILES: COC(=O)c1ccncc1C(=O)O]
or a salt thereof with a compound of Formula VII 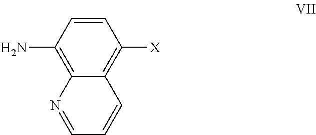
wherein X is Br or I,
or a salt thereof in the presence of an azide source or a deoxo-fluor to form a compound of Formula V-3 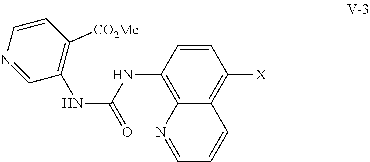
wherein X is Br or I,
or a salt thereof;
(b) subjecting the compound of Formula V-3 or a salt thereof into a cyclization condition to provide a compound of Formula V-1a 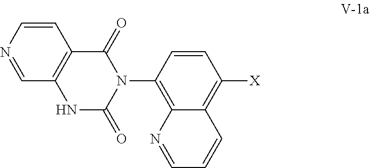
wherein X is Br or I,
or a salt thereof; and
(c) contacting the compound of Formula V-1a or a salt thereof with a methylation agent to form the compound of Formula Va.

44. The process of claim 43, wherein:
step (a) occurs in the presence of an azide source;
step (a) occurs in the presence of a solvent and a base;
the cyclization condition in step (b) comprises a solvent and a base; and/or
step (c) occurs in the presence of a base and a solvent.

45.-64. (canceled)

65. The process of claim 1, wherein Compound III 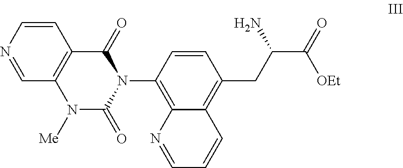
or a salt thereof is prepared by a process comprising:
(a) contacting Compound IV-3 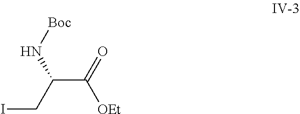 [SMILES: CCOC(=O)[C@H](CI)NC(=O)OC(C)(C)C]
or a salt thereof with zinc;
(b) contacting a product from step (a) with Compound V-I 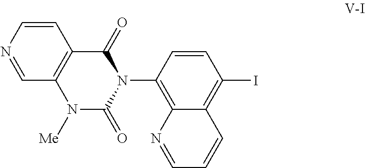
or a salt thereof in the presence of a catalytic system to form Compound III-3 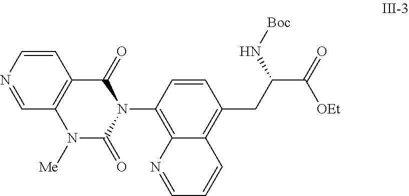
or a salt thereof; and
(c) subjecting Compound III-3 or a salt thereof to a deprotection condition to form Compound III or a salt thereof.

66. The process of claim 65, wherein:
step (a) occurs in the presence of a solvent and an activator selected from methanesulfonic acid, iodine, 1,2-dibromoethane, and silyl chlorides,
the catalytic system in step (b) comprises a palladium or nickel catalyst, a ligand, and a solvent; and/or
the deprotection condition in step (c) comprises an acid and a solvent.

67.-82. (canceled)

83. The process of claim 1, wherein Compound III 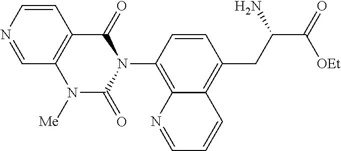
or a salt thereof is prepared by a process comprising:
(a) contacting Compound IV-5 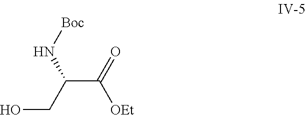 [SMILES: CCOC(=O)[C@H](CO)NC(=O)OC(C)(C)C]
or a salt thereof with a benzoxazolium salt activator;
(b1) contacting a product from step (a) with Compound V-1-Br 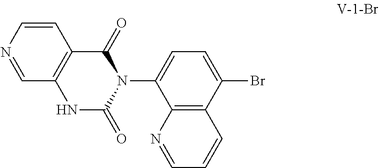
or a salt thereof in the presence of a photosensitizer to provide Compound III-4 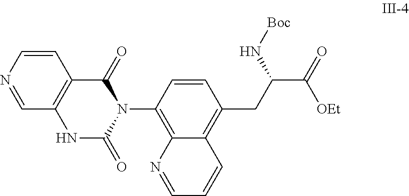
or a salt thereof; or
(b2) contacting the product from step (a) with Compound V-Br 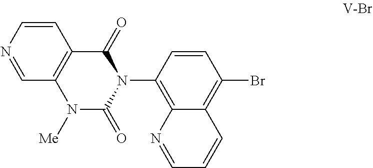
or a salt thereof in the presence of a photosensitizer to provide Compound III-3 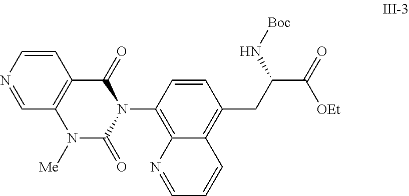
or a salt thereof; and
(c) when step (b1) occurs, contacting Compound III-4 or a salt thereof with a methylation agent to form Compound III-3 or a salt thereof; and
(d) converting Compound III-3 or a salt thereof to Compound III or a salt thereof.

84. The process of claim 83, wherein:
step (a) occurs in the presence of a solvent and a base;
the photosensitizer in step (b1) or (b2) is 4CzIPN;
step (b1) or (b2) occurs under a light having a wavelength of about 450 nm;
step (b1) or (b2) occurs in the presence of a nickel co-catalyst, a ligand, a base, a solvent, and an optional additive that is phthalimide or succinimide or a derivative thereof; and/or
step (c) occurs in the presence of a base and a solvent.

85.-109. (canceled)

110. A process for preparing a compound of Formula V-1 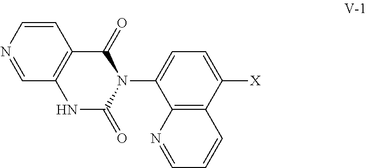
wherein R is a C₁₋₃ alkyl and X is Br or I,
or a salt thereof, comprising:
subjecting a compound of Formula V-2 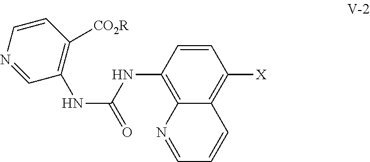
wherein R is a C₁₋₃ alkyl and X is Br or I,
or a salt thereof into a cyclization condition to form a compound of Formula V-1 or a salt thereof.

111. The process of claim 110, wherein the cyclization condition comprises an organocatalyst selected from squaramides, chiral guanidines, and chiral phase transfer catalysts; a base; and a solvent.

112.-116. (canceled)

117. The process of claim 1, wherein Compound III 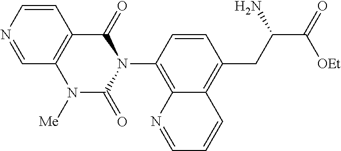
or a salt thereof is prepared by a process comprising:
(a) subjecting Compound III-5 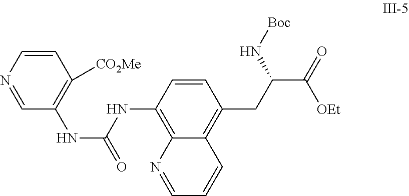 [SMILES: CCOC(=O)[C@H](Cc1ccc(NC(=O)Nc2cnccc2C(=O)OC)c2ncccc12)NC(=O)OC(C)(C)C]
or a salt thereof into a cyclization condition to form Compound III-4 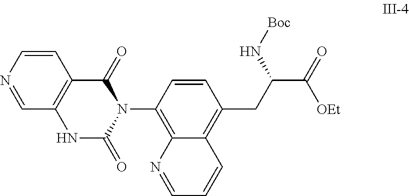
or a salt thereof; and
(b) converting Compound III-4 or a salt thereof to Compound III or a salt thereof.

118. The process of claim 117, wherein the cyclization condition comprises an organocatalyst selected from squaramides, chiral guanidines, and chiral phase transfer catalysts; a base; and a solvent.

119.-124. (canceled)

125. The process of claim 1, wherein Compound II 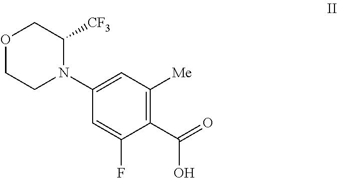 [SMILES: Cc1cc(N2CCOC[C@@H]2C(F)(F)F)cc(F)c1C(=O)O]
or a salt thereof is prepared by a process comprising:
(a) contacting Compound VIII 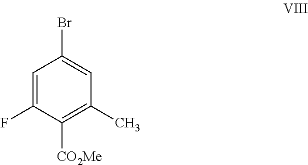
or a salt thereof with Compound IX 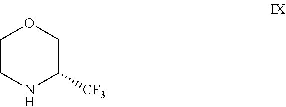
or a salt thereof in the presence of a catalytic system to provide Compound II-2 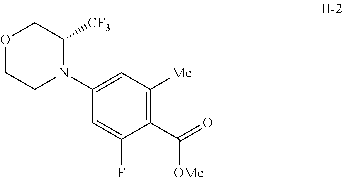 [SMILES: COC(=O)c1c(C)cc(N2CCOC[C@@H]2C(F)(F)F)cc1F]
or a salt thereof; and
(b) subjecting Compound II-2 or a salt thereof into a hydrolysis condition to form Compound II or a salt thereof.

126. The process of claim 125, wherein the catalytic system comprises a palladium catalyst, a ligand, a base, a solvent, and an optional phase transfer catalyst.

127.-140. (canceled)

141. The process of claim 125, wherein Compound IX is prepared by a process comprising contacting Compound IXa free base 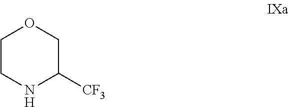
with a chiral acid that is optically pure to form Compound IX chiral acid salt, optionally wherein the chiral acid is (+)-Camphorsulfonic acid and the Compound IX chiral acid salt is Compound IX (+)-Camphorsulfonic acid salt.

142. (canceled)

143. The process of claim 125, wherein Compound IX 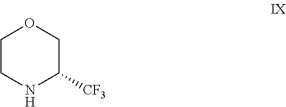
or a salt thereof is prepared by a process comprising:
(a) contacting compound of Formula IX-1 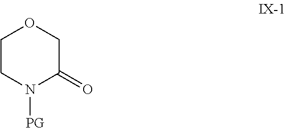
wherein PG is benzyl or 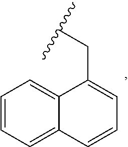 [SMILES: *Cc1cccc2ccccc12]
or a salt thereof with a trifluoromethylation agent to form a compound of Formula IX-2 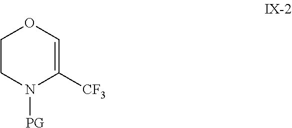
wherein PG is benzyl or 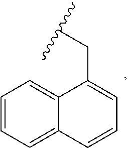 [SMILES: *Cc1cccc2ccccc12]
or a salt thereof;
(b) subjecting the compound of Formula IX-2 or a salt thereof to an asymmetric hydrogenation condition to form a compound of Formula IX-3 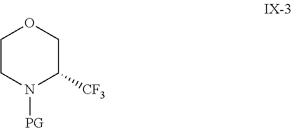
wherein PG is benzyl or 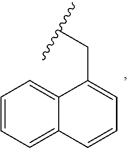 [SMILES: *Cc1cccc2ccccc12]
or a salt thereof; and
(c) subjecting the compound of formula IX-3 or a salt thereof to a deprotection condition to form Compound IX or a salt thereof, wherein the deprotection condition in step (c) comprises a chiral acid.

144. The process of claim 143, wherein:
the asymmetric hydrogenation condition in step (b) comprises a nickel catalyst, a ligand, a hydrogen source, a solvent, and an optional additive that is an acid;
the deprotection condition in step (c) comprises a reductant, a catalyst, and a solvent: wherein the reductant is a hydrogen source; and the catalyst is a solid-supported transition metal.

145.-165. (canceled)

166. A process for preparing Compound II, 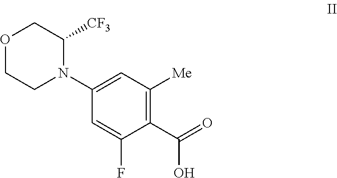 [SMILES: Cc1cc(N2CCOC[C@@H]2C(F)(F)F)cc(F)c1C(=O)O]
or a salt thereof, comprising:
(a) contacting Compound VIII 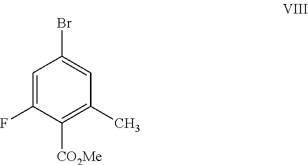
or a salt thereof with morpholin-3-one in the presence of a catalytic system to form Compound II-3 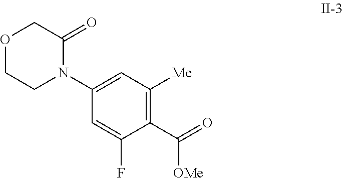 [SMILES: COC(=O)c1c(C)cc(N2CCOCC2=O)cc1F]
or a salt thereof;
(b) contacting Compound II-3 with a trifluoromethylation agent;
(c) subjecting a product from step (b) into an elimination condition to form Compound II-4 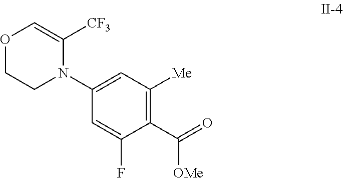 [SMILES: COC(=O)c1c(C)cc(N2CCOC=C2C(F)(F)F)cc1F]
or a salt thereof;
(d) subjecting Compound II-4 into an asymmetric hydrogenation condition to form Compound II-2 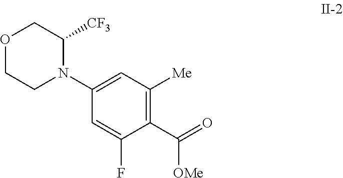 [SMILES: COC(=O)c1c(C)cc(N2CCOC[C@@H]2C(F)(F)F)cc1F]
or a salt thereof.

167. The process of claim 166, wherein:
the catalytic system in step (a) comprises a catalyst selected from a copper catalyst and a palladium catalyst, a ligand, a base, and a solvent;
step (b) occurs in the presence of a solvent and an initiator that is a fluoride source, a carboxylate, or an alkoxide,
the elimination condition in step (c) comprises an acid and a solvent; and/or
the asymmetric hydrogenation condition in step (d) comprises a rhodium catalyst, a ligand, a hydrogen source, a solvent, and an optional additive.

168.-189. (canceled)

190. The process of claim 166, further comprising subjecting Compound II-2 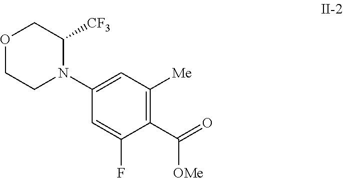 [SMILES: COC(=O)c1c(C)cc(N2CCOC[C@@H]2C(F)(F)F)cc1F]
or a salt thereof into a hydrolysis condition to form Compound II 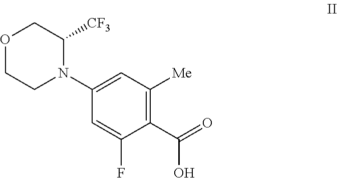 [SMILES: Cc1cc(N2CCOC[C@@H]2C(F)(F)F)cc(F)c1C(=O)O]
or a salt thereof.

191. A process for preparing Compound X 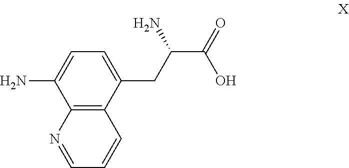 [SMILES: Nc1ccc(C[C@H](N)C(=O)O)c2cccnc12]
or a salt thereof, comprising:
contacting quinolin-8-amine with an ammonia source and a pyruvate source to form Compound X or a salt thereof.

192.-193. (canceled)

194. The process of claim 191, which occurs in the presence of an enzyme catalyst, a cofactor, an additive, and a buffer; optionally wherein the enzyme catalyst is Tyrosine phenol lyase M24, the cofactor is pyridoxal phosphate, and the additive is beta-mercaptoethanol.

195.-199. (canceled)

200. A process for preparing Compound X-3 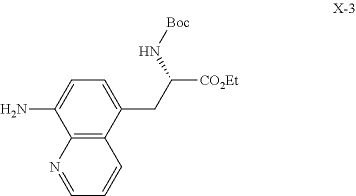 [SMILES: CCOC(=O)[C@H](Cc1ccc(N)c2ncccc12)NC(=O)OC(C)(C)C]
or a salt thereof, comprising:
(a) contacting Compound IV-4 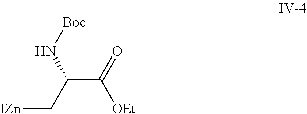
or a salt thereof with Compound VII-2 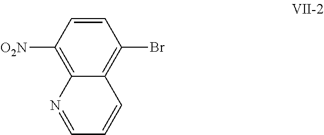 [SMILES: O=[N+]([O-])c1ccc(Br)c2cccnc12]
or a salt thereof in the presence of a catalytic system to provide Compound X-4 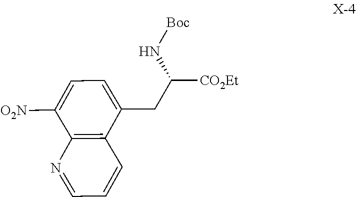 [SMILES: CCOC(=O)[C@H](Cc1ccc([N+](=O)[O-])c2ncccc12)NC(=O)OC(C)(C)C]
or a salt thereof; and
(b) subjecting Compound X-4 or a salt thereof to a reductive condition to provide Compound X-3 or a salt thereof.

201. The process of claim 200, wherein:
the catalytic system in step (a) comprises a palladium or nickel catalyst, a ligand, and a solvent, and/or
the reductive condition in step (b) comprises a reducing agent and a solvent.

202.-211. (canceled)

212. A process for preparing Compound III 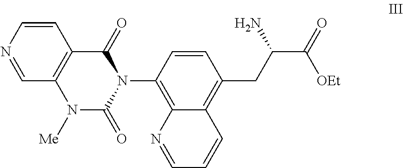
or a salt thereof, comprising:
(a) contacting Compound III-1a 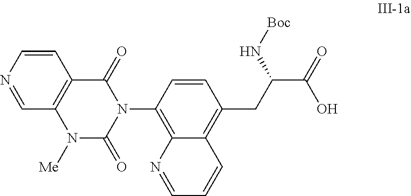 [SMILES: Cn1c(=O)n(-c2ccc(C[C@H](NC(=O)OC(C)(C)C)C(=O)O)c3cccnc23)c(=O)c2ccncc21]
or a salt thereof with ethanol in the presence of an acid to form Compound IIIa 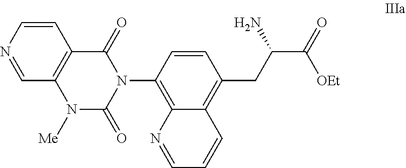 [SMILES: CCOC(=O)[C@@H](N)Cc1ccc(-n2c(=O)c3ccncc3n(C)c2=O)c2ncccc12]
or a salt thereof, wherein Compound IIIa represents a mixture of atropisomers including Compound III; and
(b) equilibrating Compound IIIa under acidic or thermal condition followed by crystallizing Compound IIIa from a solvent to provide Compound III as a single atropisomer.

213. A process for preparing Compound II 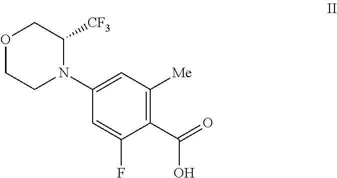 [SMILES: Cc1cc(N2CCOC[C@@H]2C(F)(F)F)cc(F)c1C(=O)O]
or a salt thereof, comprising:
(a) contacting Compound VIII 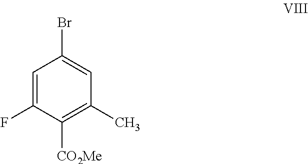
or a salt thereof with morpholin-3-one in the presence of a catalytic system to form Compound II-3 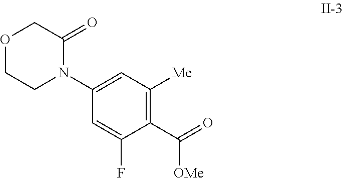 [SMILES: COC(=O)c1c(C)cc(N2CCOCC2=O)cc1F]
or a salt thereof, wherein the catalytic system in step (a) comprises a catalyst selected from a copper catalyst and a palladium catalyst, a ligand, a base, and a solvent;
(b) contacting Compound II-3 with a trifluoromethylation agent, wherein step (b) occurs in the presence of a solvent and an initiator that is a fluoride source, a carboxylate, or an alkoxide;
(c) subjecting a product from step (b) into an elimination condition to form Compound II-4 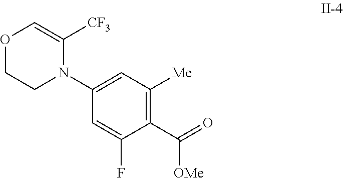 [SMILES: COC(=O)c1c(C)cc(N2CCOC=C2C(F)(F)F)cc1F]
or a salt thereof, wherein the elimination condition in step (c) comprises an acid and a solvent; and
(d) subjecting Compound II-4 into an asymmetric hydrogenation condition to form Compound II-2 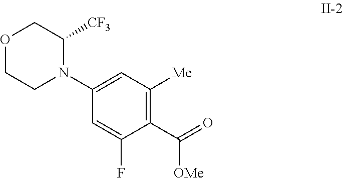 [SMILES: COC(=O)c1c(C)cc(N2CCOC[C@@H]2C(F)(F)F)cc1F]
or a salt thereof, wherein the asymmetric hydrogenation condition in step (d) comprises a rhodium catalyst, a ligand, a hydrogen source, a solvent, and an optional additive.

214. A process for preparing Compound II 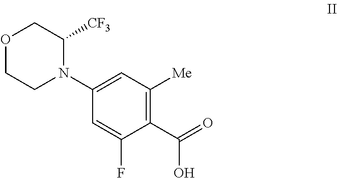 [SMILES: Cc1cc(N2CCOC[C@@H]2C(F)(F)F)cc(F)c1C(=O)O]
or a salt thereof, comprising:
(a) contacting Compound VIII 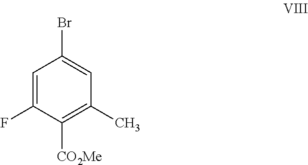
or a salt thereof with morpholin-3-one in the presence of a catalytic system to form Compound II-3 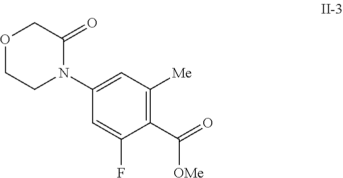 [SMILES: COC(=O)c1c(C)cc(N2CCOCC2=O)cc1F]
or a salt thereof;
(b) contacting Compound II-3 with a trifluoromethylation agent;
(c) subjecting a product from step (b) into an elimination condition to form Compound II-4 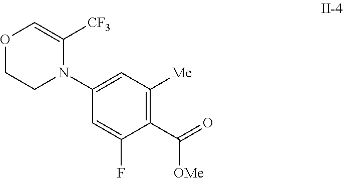 [SMILES: COC(=O)c1c(C)cc(N2CCOC=C2C(F)(F)F)cc1F]
or a salt thereof;
(d) subjecting Compound II-4 into an asymmetric hydrogenation condition to form Compound II-2 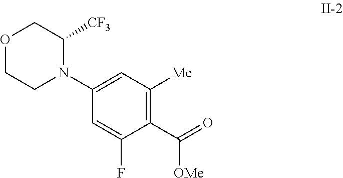 [SMILES: COC(=O)c1c(C)cc(N2CCOC[C@@H]2C(F)(F)F)cc1F]
or a salt thereof; and
(e) subjecting Compound II-2 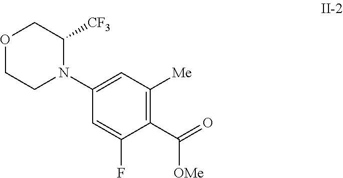 [SMILES: COC(=O)c1c(C)cc(N2CCOC[C@@H]2C(F)(F)F)cc1F]
or a salt thereof into a hydrolysis condition to form Compound II 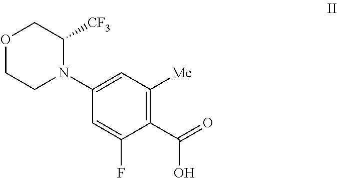 [SMILES: Cc1cc(N2CCOC[C@@H]2C(F)(F)F)cc(F)c1C(=O)O]
or a salt thereof.
